(19) United States
(12) Patent Application Publication
(10) Pub. No.: US 2026/0306629 A1
(43) Pub. Date: Oct. 8, 2026

(54) ANTI-CD19 CAR T CELLS FOR TREATING B-CELL MALIGNANCIES AND AUTOIMMUNE DISEASES

(71) Applicant: Sana Biotechnology, Inc., Seattle, WA (US)

(72) Inventors: Hosein KOUROS-MEHR, Seattle, WA (US); Paul BRUNETTA, Seattle, WA (US); Diane Marie DAHILL, Seattle, WA (US); Alexandra BOWDEN, Seattle, WA (US)

(21) Appl. No.: 19/479,064

(22) PCT Filed: Apr. 26, 2024
(86) PCT No.: PCT/US2024/026671

§ 371 Date: Oct. 27, 2025

Related U.S. Application Data
(60) Provisional application No. 63/462,957, filed on Apr. 28, 2023.
(60) Provisional application No. 63/507,064, filed on Jun. 8, 2023.
(60) Provisional application No. 63/589,286, filed on Oct. 10, 2023.
(60) Provisional application No. 63/595,230, filed on Nov. 1, 2023.
(60) Provisional application No. 63/618,817, filed on Jan. 8, 2024.

(51) Int. Cl. A61K 40/11; A61K 40/31; A61K 40/41; A61K 40/42; A61P 35/00; A61P 37/00

(52) U.S. Cl. CPC A61K 40/11; A61K 40/31; A61K 40/416; A61K 40/4211; A61P 35/00; A61P 37/00

(57) ABSTRACT

The present disclosure provides methods of treating a subject having or suspected of having a B cell malignancy or a B cell mediated disorder by administering hypoimmune allogenic CD19-directed CAR T cells. Also disclosed are pharmaceutical compositions comprising hypoimmune allogenic CD19-directed CAR T cells, for use in treating a subject having or suspected of having a B cell malignancy or a B cell mediated disorder.

### CROSS REFERENCE TO RELATED APPLICATIONS

This application claims priority to and the benefit of U.S. Provisional Application No. 63/462,957, filed Apr. 28, 2023; U.S. Provisional Application No. 63/507,064, filed Jun. 8, 2023; U.S. Provisional Application No. 63/589,286, filed Oct. 10, 2023; U.S. Provisional Application No. 63/595,230, filed Nov. 1, 2023; and U.S. Provisional Application No. 63/618,817, filed Jan. 8, 2024; the contents of which are incorporated herein by reference in their entirety.

### BACKGROUND

B cell lymphomas encompass a group of lymphoma subtypes categorized within 2 broad categories: non-Hodgkin lymphoma (NHL) and Hodgkin lymphoma (HL), with NHLs being the majority of diagnosed cases. The therapeutic landscape varies for each distinct tumor subtype, but generally there are limited effective therapies in relapsed and/or refractory (r/r) settings across B cell lymphomas.

Despite recent advancement, NHL remains an unmet medical need. In 2020, there were 123,000 new cases of NHL reported with 50,000 attributed deaths in Europe. NHL is a heterogeneous group of cancers originating in B lymphocytes, T lymphocytes, or natural killer (NK) cells. In the US, B cell lymphomas represent approximately 85% of all NHL cases.

There is an urgent need to develop therapies for such patients.

### SUMMARY

Off-the-shelf CAR-T cells and other therapeutic cells can offer advantages over autologous cell-based strategies, including ease of manufacturing, quality control, and avoidance of malignant contamination and T cell dysfunction. However, the vigorous host-versus-graft immune response against histoincompatible T cells prevents expansion and persistence of allogenic CAR-T cells and mitigates the efficacy of this approach.

There is substantial evidence in both animal models and human patients that hypoimmunogenic cell transplantation is a scientifically feasible and clinically promising approach to the treatment of numerous disorders, conditions, and diseases.

There remains a need for novel approaches, compositions and methods for producing cell-based therapies that avoid detection by the recipient's immune system.

In one aspect, the present disclosure provides methods of treating a subject having or suspected of having a B cell malignancy or a B cell mediated disorder, the method comprising: administering to the subject a composition comprising a population of engineered hypoimmunogenic T cells, wherein the hypoimmunogenic T cells comprise one or more modifications that: (a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules, (b) increase expression of CD47 encoded by a first exogenous polynucleotide, wherein the increased expression of (b) is relative to a control or wild-type T cell that does not comprise the modification, and (c) express a CD19-specific chimeric antigen receptor (CAR) encoded by a second exogenous polynucleotide.

In some embodiments, one or more modifications reduce expression of one or more MHC class I molecules, one or more MHC class II molecules, and/or one or more TCR molecules in the hypoimmunogenic T cells, relative to control or wild-type T cells. In some embodiments, expression of one or more MHC class I molecules, one or more MHC class II molecules, and one or more TCR molecules is reduced in the hypoimmunogenic T cells relative to a control or wild-type T cell. In some embodiments, a control or wild-type T cells do not comprise the modifications. In some embodiments, one or more modifications reduce cell surface expression of the one or more MHC class I molecules relative to a control or wild-type T cell. In some embodiments, one or more modifications reduce cell surface trafficking of one or more MHC class I molecules relative to a control or wild-type T cell. In some embodiments, one or more modifications reduce a function of one or more MHC class I molecules. In some embodiments, one or more MHC class I molecules is one or more human leukocyte antigen (HLA) class I molecules. In some embodiments, one or more HLA class I molecules is HLA-A, HLA-B, HLA-C, or a combination thereof.

In some embodiments, one or more molecules that regulate cell surface expression of one or more MHC class I molecules, regulate cell surface trafficking of the one or more MHC class I molecules, and/or reduce a function of one or more MHC class I molecules are beta-2 microglobulin (B2M). In some embodiments, one or more modifications comprise a modification that inactivates or disrupts one or more alleles of B2M. In some embodiments, a modification that inactivates or disrupts one or more alleles of B2M reduces mRNA expression of the B2M gene. In some embodiments, a modification that inactivates or disrupts one or more alleles of B2M reduces protein expression of B2M. In some embodiments, a modification that inactivates or disrupts one or more alleles of B2M comprises: inactivation or disruption of one allele of the B2M gene; inactivation or disruption of both alleles of the B2M gene; or inactivation or disruption of all B2M coding alleles in a hypoimmunogenic T cell. In some embodiments, a modification that inactivates or disrupts one or more alleles of B2M comprises an indel in the B2M gene. In some embodiments, a modification that inactivates or disrupts one or more alleles of B2M comprises a frameshift mutation or a deletion of a contiguous stretch of genomic DNA of the B2M gene.

In some embodiments, one or more modifications reduce cell surface expression of the one or more MHC class II molecules relative to a control or wild-type cell. In some embodiments, one or more modifications reduce cell surface trafficking of the one or more MHC class II molecules relative to a control or wild-type cell. In some embodiments, one or more modifications reduce a function of one or more MHC class II molecules. In some embodiments, function of the one more MHC class II molecules is antigen presentation. In some embodiments, one or more MHC class II molecules is one or more human leukocyte antigen (HLA) class II molecules. In some embodiments, one or more HLA class II molecules is HLA-DP, HLA-DQ, HLA-DR, or a combination thereof.

In some embodiments, one or more molecules that regulate expression of the one or more MHC class II molecules are Class II Major Histocompatibility Complex Transactivator (CIITA). In some embodiments, one or more modifications comprise a modification that inactivates or disrupts one or more alleles of CIITA. In some embodiments, a modification that inactivates or disrupts one or more alleles of CIITA reduces mRNA expression of the CIITA gene. In some embodiments, a modification that inactivates or disrupts one or more alleles of CIITA reduces protein expression of CIITA. In some embodiments, a modification that inactivates or disrupts one or more alleles of CIITA comprises: inactivation or disruption of one allele of the CIITA gene; inactivation or disruption of both alleles of the CIITA gene; or inactivation or disruption of all CIITA coding alleles in the hypoimmunogenic T cell. In some embodiments, a modification that inactivates or disrupts one or more alleles of CIITA comprises an indel in a CIITA gene. In some embodiments, a modification that inactivates or disrupts one or more alleles of CIITA comprises a frameshift mutation or a deletion of a contiguous stretch of genomic DNA of a CIITA gene.

In some embodiments, expression of HLA-A, HLA-B, HLA-C, HLA-DP, HLA-DQ, and HLA-DR are reduced in a hypoimmunogenic T cell. In some embodiments, one or more modifications reduce cell surface protein expression of one or more TCR molecules relative to the control or wild-type cell. In some embodiments, one or more modifications reduce cell surface trafficking of the one or more TCR molecules relative to a control or wild-type cell. In some embodiments, one or more modifications reduce a function of one or more TCR molecules. In some embodiments, a function of one or more TCR molecules is antigen recognition. In some embodiments, one or more TCR molecules is T cell receptor alpha constant (TRAC), T cell receptor beta constant (TRBC), or a combination thereof. In some embodiments, one or more TCR molecules comprise TRAC. In some embodiments, one or more modifications comprise a modification that inactivates or disrupts one or more alleles of TRAC. In some embodiments, a modification that inactivates or disrupts one or more alleles of TRAC reduces mRNA expression of a TRAC gene. In some embodiments, a modification that inactivates or disrupts one or more alleles of TRAC reduces protein expression of TRAC. In some embodiments, a modification that inactivates or disrupts one or more alleles of TRAC comprises: inactivation or disruption of one allele of the TRAC gene; inactivation or disruption of both alleles of the TRAC gene; or inactivation or disruption of all TRAC coding alleles in a hypoimmunogenic T cell. In some embodiments, a modification inactivation or disruption that inactivates or disrupts one or more alleles of TRAC comprises an indel in the TRAC gene. In some embodiments, inactivation or disruption comprises a frameshift mutation or a deletion of a contiguous stretch of genomic DNA of the TRAC gene.

In some embodiments, one or more modifications increase expression of one or more tolerogenic factors encoded by a third exogenous polynucleotide, wherein the increased expression of the one or more tolerogenic factors is relative to a control or wild-type T cell, and wherein the one or more tolerogenic factors comprise CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or a combination thereof.

In another aspect, the present disclosure provides methods of treating a subject having or suspected of having a B cell malignancy or a B cell mediated disorder, the method comprising: administering to the subject a composition comprising a population of engineered hypoimmunogenic T cells comprising: (a) reduced expression of beta-2 microglobulin (B2M), Class II Major Histocompatibility Complex Transactivator (CIITA), and T cell receptor alpha constant (TRAC) relative to a control or wild-type T cells, (b) increased expression of CD47 encoded by a first exogenous polynucleotide, wherein the increased expression of (b) is relative to the control or wild-type T cells, and (c) expression of a CD19-specific chimeric antigen receptor (CAR) encoded by a second exogenous polynucleotide.

In some embodiments, a composition is administered to the subject at a dosage of about 5×10⁶ cells to about 400×10⁶ cells. In some embodiments, a composition is administered to the subject at a dosage of about 20×10⁶ cells to about 400×10⁶ cells. In some embodiments, a composition is administered to the subject at a dosage of at least about 30×10⁶ cells, at least about 60×10⁶ cells, at least about 90×10⁶ cells, at least about 120×10⁶ cells, or at least about 200×10⁶ cells. In some embodiments, a composition is administered to the subject at a dosage of no more than about 200×10⁶ cells. In some embodiments, a composition is administered to the subject at a dosage of no more than about 400×10⁶ cells. In some embodiments, a composition is administered to the subject at a dosage of about 5×10⁶ CAR+ cells to about 400×10⁶ CAR+ cells. In some embodiments, a composition is administered to the subject at a dosage of about 20×10⁶ CAR+ cells to about 400×10⁶ CAR+ cells. In some embodiments, a composition is administered to the subject at a dosage of at least about 30×10⁶ CAR+ cells, at least about 60×10⁶ CAR+ cells, at least about 90×10⁶ CAR+ cells, at least about 120×10⁶ CAR+ cells, or at least about 200×10⁶ CAR+ cells. In some embodiments, a composition is administered to the subject at a dosage of no more than about 200×10⁶ CAR+ cells. In some embodiments, a composition is administered to the subject at a dosage of no more than about 400×10⁶ CAR+ cells. In some embodiments, a total volume of about 1.25 mL to about 100 mL of the composition is administered. In some embodiments, a total volume of about 5 mL to about 100 mL of the composition is administered. In some embodiments, a total volume of at least about 7.5 mL, at least about 15 mL, at least about 30 mL, or at least about 50 mL of the composition is administered. In some embodiments, a composition comprises a concentration of engineered hypoimmunogenic T cells of at least 1.25×10⁶ cells/mL. In some embodiments, a composition comprises a concentration of engineered hypoimmunogenic T cells of at least 4.0×10⁶ cells/mL. In some embodiments, a composition comprises a concentration of engineered hypoimmunogenic T cells of at least 1.2×10⁶ CAR+ cells/mL. In some embodiments, a composition comprises a concentration of engineered hypoimmunogenic T cells of at least 4.0×10⁶ CAR+ cells/mL.

In some embodiments, a composition of the present disclosure comprises fully edited hypoimmunogenic CAR+ cells as described herein. In some embodiments, fully edited hypoimmunogenic CAR+ cells comprise one or more modifications that: (a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules, (b) increase expression of CD47 encoded by a first exogenous polynucleotide, wherein the increased expression of (b) is relative to a control or wild-type T cell that does not comprise the modification, and (c) express a CD19-specific chimeric antigen receptor (CAR) encoded by a second exogenous polynucleotide. In some embodiments, a composition of the present disclosure comprises partially edited CAR+ cells as described herein. In some embodiments, partially edited CAR+ cells are missing one or more modifications that: (a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules, (b) increase expression of CD47 encoded by a first exogenous polynucleotide, wherein the increased expression of (b) is relative to a control or wild-type T cell that does not comprise the modification, and (c) express a CD19-specific chimeric antigen receptor (CAR) encoded by a second exogenous polynucleotide, relative to fully edited hypoimmunogenic CAR+ cells as described herein. In some embodiments, a composition of the present disclosure comprises fully edited hypoimmunogenic CAR+ cells and partially edited CAR+ cells. In some embodiments, a composition of the present disclosure comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% fully edited hypoimmunogenic CAR+ cells. In some embodiments, a composition of the present disclosure comprises 100% fully edited hypoimmunogenic CAR+ cells. In some embodiments, a composition of the present disclosure comprises about 20% to about 40% fully edited hypoimmunogenic CAR+ cells. In some embodiments, a composition of the present disclosure comprises about 30% to about 50% fully edited hypoimmunogenic CAR+ cells. In some embodiments, a composition of the present disclosure comprises about 40% to about 60% fully edited hypoimmunogenic CAR+ cells. In some embodiments, a composition of the present disclosure comprises about 50% to about 70% fully edited hypoimmunogenic CAR+ cells. In some embodiments, a composition of the present disclosure comprises about 60% to about 80% fully edited hypoimmunogenic CAR+ cells. In some embodiments, a composition of the present disclosure comprises about 70% to about 90% fully edited hypoimmunogenic CAR+ cells. In some embodiments, a composition of the present disclosure comprises about 80% to about 99% fully edited hypoimmunogenic CAR+ cells.

In some embodiments, methods of the present disclosure provide administering a composition in at least one dose, at least two doses, at least three doses, at least four doses, at least five doses, at least six doses, at least seven doses, at least eight doses, at least ten doses, at least eleven doses, or at least twelve doses. In some embodiments, methods of the present disclosure provide administering the composition in a single dose. In some embodiments, methods of the present disclosure provide administering the composition in two doses.

In some embodiments, engineered hypoimmunogenic T cells comprise CD8+ and/or CD4+ T cells. In some embodiments, engineered hypoimmunogenic T cells comprise allogeneic T cells. In some embodiments, engineered hypoimmunogenic T cells comprise primary T cells. In some embodiments, engineered hypoimmunogenic T cells comprise stem cell-derived T cells.

In some embodiments, a CAR comprises an anti-CD19 antigen binding domain, a CD8α hinge domain, a CD8α transmembrane domain, a 4-1BB costimulatory domain, and a CD3ζ intracellular signaling domain. In some embodiments, an antigen binding domain comprises: (i) a light chain amino acid sequence comprising a light chain CDR1, a light chain CDR2, and a light chain CDR3, wherein the light chain CDR1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and (ii) a heavy chain amino acid sequence comprising a heavy chain CDR1, a heavy chain CDR2, and a heavy chain CDR3 amino acid, wherein the heavy chain CDR1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28. In some embodiments, a light chain amino acid sequence comprises a light chain variable region and the heavy chain amino acid sequence comprises a heavy chain variable region, wherein the light chain variable region comprises an amino acid sequence at least 90% identical to SEQ ID NO: 20, and wherein the heavy chain variable region comprises an amino acid sequence comprises an amino acid sequence at least 90% identical to SEQ ID NO: 25. In some embodiments, a CD8α hinge domain comprises an amino acid sequence at least 90% identical to SEQ ID NO: 9, the CD8α transmembrane domain comprises an amino acid sequence at least 90% identical to SEQ ID NO: 14, the 4-1BB costimulatory domain comprises an amino acid sequence at least 90% identical to SEQ ID NO: 16, and the CD3ζ intracellular signaling domain comprises an amino acid sequence at least 90% identical to SEQ ID NO: 18. In some embodiments, an antigen binding domain comprises a linker comprising the amino acid sequence at least 90% identical to SEQ ID NO: 24. In some embodiments, an antigen binding domain comprises a linker comprising the amino acid sequence of SEQ ID NO: 24. In some embodiments, an antigen binding domain comprises an amino acid sequence at least 90% identical to SEQ ID NO: 19. In some embodiments, an antigen binding domain comprises the amino acid sequence of SEQ ID NO: 19.

In some embodiments, engineered hypoimmunogenic T cells are B2M^{indel/indel}, CIITA^{indel/indel}, and/or TRAC^{indel/indel} cells. In some embodiments, a population of engineered hypoimmunogenic T cells is a heterogeneous population comprising B2M^{indel/indel} cells, CIITA^{indel/indel} cells, TRAC^{indel/indel} cells, B2M^{indel/indel}/CIITA^{indel/indel} cells, B2M^{indel/indel}/TRAC^{indel/indel} cells, CIITA^{indel/indel}/TRAC^{indel/indel} cells, and/or B2M^{indel/indel}/CIITA^{indel/indel}/TRAC^{indel/indel} cells.

In some embodiments, a composition is administered intravenously.

In some embodiments, a B cell malignancy is a lymphoma or a leukemia. In some embodiments, a B cell malignancy is selected from the group consisting of: Non-Hodgkin's Lymphoma (NHL), Chronic Lymphocytic Leukemia (CLL), large B cell lymphoma (LBCL), transplant ineligible large B cell lymphoma (LBCL), diffuse LBCL (DLBCL), high-grade B cell lymphoma (HGBCL), primary mediastinal B cell lymphoma (PMBCL), mantle cell lymphoma (MCL), follicular lymphoma (FL), marginal zone lymphoma (MZL), or small lymphocytic lymphoma (SLL).

In some embodiments, a subject is diagnosed to have NHL or CLL. In some embodiments, a subject is diagnosed to have NHL based on the World Health Organization 2016 criteria. In some embodiments, a subject is confirmed to have NHL based on histological analysis of a biopsy sample collected from the subject. In some embodiments, a subject has: a single lesion measuring greater than 7.5 cm in diameter; or a sum of the product of lymph node diameter is greater than 5000 mm³.

In some embodiments, a subject is diagnosed to have CLL based on the International Workshop on CLL (iwCLL) criteria. In some embodiments, a subject has: a lymph node greater than or equal to 10 cm in diameter; or a lymph node greater than or equal to 5 cm in diameter and an absolute lymphocyte count greater than or equal to 25×10⁹ lymphocytes/L.

In some embodiments, a subject is diagnosed as being a high risk CCL patient. In some embodiments, a method of the present disclosure further comprises: detecting one or more biomarkers, and identifying a subject as being a high risk CCL patient based on the one or more biomarkers. In some embodiments, one or more biomarkers are selected from the group consisting of: del(17p), mutated TP53, unmutated immunoglobulin heavy-chain variable (IGHV) gene, and complex karyotype.

In some embodiments, a subject previously received an alkylating agent, a purine nucleoside, chemoimmunotherapy, a Bruton's tyrosine kinase (BTK) inhibitor, a PI3K inhibitor, a B cell lymphoma 2 (BCL2) inhibitor, a CD20 monoclonal antibody, a bispecific monoclonal antibody, or a combination thereof. In some embodiments, an alkylating agent comprises chlorambucil or cyclophosphamide. In some embodiments, a purine nucleoside is selected from the group consisting of: fludarabine, pentostatin, and cladribine. In some embodiments, a chemoimmunotherapy is selected from the group consisting of: FCR (fludarabine, cyclophosphamide, and rituximab), BR (bendamustine and rituximab), alemtuzumab, and lenalidomide. In some embodiments, a BTK inhibitor is selected from the group consisting of: ibrutinib, acalabrutinib, zanubrutinib, and pirtobrutinib. In some embodiments, a PI3K inhibitor comprises idelalisib or duvelisi. In some embodiments, a BCL2 inhibitor comprises venetoclax. In some embodiments, a CD20 monoclonal antibody comprises obinutuzumab. In some embodiments, a bispecific monoclonal antibody is selected from the group consisting of Mosunetuzumab (CD20×CD3 mAb), Glofitamab (CD20⁽²⁾×CD3), Odronextamab (CD20×CD3 mAb), and Epcoritamab (CD20×CD3 mAb).

In some embodiments, a subject is diagnosed to have CLL and is further administered a BTK inhibitor. In some embodiments, a BTK inhibitor is selected from the group consisting of: ibrutinib, acalabrutinib, zanubrutinib, and pirtobrutinib.

In some embodiments, a subject is diagnosed to have CLL and is administered the composition comprising the population of engineered hypoimmunogenic T cells as a first-line therapy.

In some embodiments, a B cell malignancy or the B cell mediated disorder is relapsed or refractory following at least one therapeutic standard of care regimen. In some embodiments, a B cell malignancy or the B cell mediated disorder is relapsed or refractory following at least two therapeutic standard of care regimens. In some embodiments, a B cell malignancy or the B cell mediated disorder is relapsed or refractory following at least three therapeutic standard of care regimens. In some embodiments, a B cell malignancy or the B cell mediated disorder is relapsed or refractory following autologous stem cell transplant (ASCT). In some embodiments, a subject: did not respond to a first line therapy; had progressive B cell malignancy or B cell mediated disorder as a best response to a last administered therapeutic regimen; or had stable disease as a best response to the last administered therapeutic regimen. In some embodiments, a subject: had disease progression or relapse within twelve months of ASCT; or the subject had no response to or relapsed following administration of a last line of therapy.

In some embodiments, a subject has adequate hepatic function, renal function, bone marrow function, cardiac function, and/or pulmonary function.

In some embodiments, a B cell malignancy is relapsed and/or refractory. In some embodiments, a B cell malignancy is relapsed and/or refractory NHL. In some embodiments, a B cell malignancy is relapsed and/or refractory CLL. In some embodiments, a B cell malignancy is relapsed and/or refractory after at least 2 regimens of therapy. In some embodiments, a B cell malignancy is relapsed and/or refractory after at least 3 regimens of therapy. In some embodiments, a B cell malignancy is relapsed and/or refractory after autologous stem cell transplant (ASCT). In some embodiments, a B cell malignancy is relapsed and/or refractory, wherein relapsed and/or refractory comprises one or more of: no response to first line therapy, progressive disease as best response to most recent therapy regimen, stable disease as best response to most recent therapy with duration no longer than 6 months from last dose of therapy, disease progression or relapse within 12 months of ASCT, no response to or relapse after last line of therapy post-ASCT.

In some embodiments, a B cell mediated disorder is an autoimmune condition. In some embodiments, an autoimmune condition is selected from the group consisting of: systemic lupus erythematosus (SLE), lupus nephritis, CNS lupus, anti-neutrophilic cytoplasmic autoantibody (ANCA) associated vasculitis, granulomatous polyangiitis, microscopic polyangiitis, multiple sclerosis (relapsing and/or progressive), pemphigus vulgaris, autoimmune blistering skin diseases, membranous nephropathy, anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, autoimmune hemolytic anemia, amyloidosis, scleroderma, idiopathic inflammatory myositis, and immune-mediated necrotizing myopathy. In some embodiments, a subject has: a moderate to severe autoimmune condition; a relapsed or refractory autoimmune condition; and/or the presence of autoantibodies. In some embodiments, a subject is diagnosed to have systemic lupus erythematosus (SLE), extrarenal systemic lupus erythematosus (ERL), lupus nephritis (LN), ANCA-associated vasculitis (AAV), or membranous nephropathy (MN).

In some embodiments, a subject has not received a prior CD19-directed therapy. In some embodiments, a subject has received a prior CD19-directed therapy. In some embodiments, a prior CD19-directed therapy is or comprises a CD19-directed CAR T cell treatment. In some embodiments, a prior CD19-directed therapy is or comprises a CD19-directed antibody treatment. In some embodiments, a prior CD19-directed therapy is or comprises a CD19-directed CAR natural killer (NK) cell treatment.

In some embodiments, a subject has received a prior bispecific antibody therapy. In some embodiments, a bispecific antibody therapy is selected from the group consisting of: Mosunetuzumab (CD20×CD3 mAb), Glofitamab (CD20⁽²⁾×CD3), Odronextamab (CD20×CD3 mAb), and Epcoritamab (CD20×CD3 mAb).

In some embodiments, a subject does not have a history of primary central nervous system (CNS) lymphoma or CNS metastases. In some embodiments, absence of primary CNS lymphoma or CNS metastases is based on a brain magnetic resonance imaging (MRI) performed within 3 months prior to administering the composition to the subject. In some embodiments, absence of CNS lymphoma or CNS metastases is based on a lumbar puncture performed within 1 month prior to administering the composition to the subject.

In some embodiments, a subject does not have a history of Richter's transformation of chronic leukemic lymphoma, small lymphocytic lymphoma, or lymphoplasmacytic lymphoma. In some embodiments, a subject does not have a fungal, bacterial, viral, or other infection that is uncontrolled or requiring intravenous (IV) antimicrobials for management. In some embodiments, a subject does not have acute symptoms of COVID-19 infection. In some embodiments, a subject does not have a known history of infection with HIV, hepatitis B virus, and/or hepatitis C virus. In some embodiments, a subject does not have an active autoimmune disease and/or any other disease requiring immunosuppressive therapy or corticosteroid therapy. In some embodiments, a subject has not received greater than 10 mg/day of prednisone or equivalent dose of another corticosteroid. In some embodiments, a subject does not have a primary immunodeficiency or a history of a concomitant genetic syndrome associated with bone marrow failure. In some embodiments, a primary immunodeficiency or the concomitant genetic syndrome associated with bone marrow failure comprises Fanconi anemia, Kostmann syndrome, or Shwachman-Diamond syndrome.

In some embodiments, a subject has not been treated with a systemic cancer therapy within 14 days or five half-lives of administration of the composition. In some embodiments, a subject has not been treated with a systemic cancer therapy comprising a biologic therapy within 28 days of administration of the composition. In some embodiments, a subject has not been treated with any investigational product within 28 days of administration of the composition. In some embodiments, a subject has not been treated with radiotherapy within 14 days of administration of the composition. In some embodiments, a subject has not been treated with major surgery within 28 days of administration of the composition. In some embodiments, a subject has not been treated with an autologous hematopoietic stem cell transplant (HSCT) within six weeks of administration of the composition. In some embodiments, a subject has not been treated with an allogeneic HSCT at any point prior to administration of the composition.

In some embodiments, a subject has not had a cardiac condition within one year of administration of the composition. In some embodiments, a cardiac condition comprises myocardial infarction, cardiac angioplasty or stenting, cardiac arrhythmia requiring medication, unstable angina, New York Heart Association Class II or greater congestive heart failure, cardiac atrial or ventricular lymphoma involvement, or a clinically significant cardiac disease. In some embodiments, a subject does not have a history of thromboembolic disease. In some embodiments, a subject does not have a history of non-line associated, clinically significant deep-vein thrombosis, proximal deep vein thrombosis, or pulmonary embolism requiring therapeutic anticoagulation within 6 months of administration of the composition.

In some embodiments, a subject does not have a history or presence of a CNS disorder within 12 months of administration of the composition. In some embodiments, a CNS disorder comprises a seizure disorder, a cerebrovascular ischemia/hemorrhage, a dementia, a cerebellar disease, or any autoimmune disease with CNS involvement.

In some embodiments, a subject does not have clinically significant ascites. In some embodiments, clinically significant ascites comprises physically positive ascites or ascites requiring puncture intervention or medication intervention for control.

In some embodiments, a subject does not have a history of organ transplantation and/or is not awaiting organ transplantation.

In some embodiments, a subject does not have an indwelling line or drain. In some embodiments, an indwelling line or drain comprises a percutaneous nephrostomy tube, an indwelling Foley catheter, a biliary drain, a G/J-tube, a pleural catheter, a peritoneal catheter, or a pericardial catheter.

In some embodiments, a subject has not received a live vaccine within 6 weeks of administration of the composition.

In some embodiments, a subject does not have an unresolved toxicity greater than Grade 1 from a previous anticancer therapy.

In some embodiments, a subject does not have a history of additional malignancy other than nonmelanoma skin cancer or carcinoma in situ. In some embodiments, a subject does not have a history of additional malignancy, or has been disease-free from the additional malignancy for at least two years prior to administration of the composition.

In some embodiments, a subject does not have a severe immediate hypersensitivity to any of the components of the composition.

In some embodiments, a subject does not have an admission or evidence of illicit drug use, drug abuse, or alcohol abuse.

In some embodiments, a subject is 18-85 years of age. In some embodiments, a subject is 18-80 years of age. In some embodiments, a subject is 18-75 years of age.

In some embodiments, methods of the present disclosure further comprise: administering to a subject a lymphodepleting chemotherapy regimen, wherein the step of administering the lymphodepleting chemotherapy regimen occurs prior to the step of administering the composition. In some embodiments, a composition is administered 2-7 days after completion of the lymphodepleting chemotherapy regimen. In some embodiments, a composition is administered 3 days after completion of the lymphodepleting chemotherapy regimen. In some embodiments, a lymphodepleting chemotherapy is or comprises cyclophosphamide and fludarabine. In some embodiments, cyclophosphamide is administered at a dose of about 500 mg/m². In some embodiments, cyclophosphamide is administered over approximately 60 minutes. In some embodiments, cyclophosphamide is administered daily for 3 days. In some embodiments, cyclophosphamide is administered to the subject 5 days, 4 days, and 3 days prior to the administration of the composition. In some embodiments, one liter of 0.9% sodium chloride is administered within 24 hours prior to administration of cyclophosphamide. In some embodiments, fludarabine is administered at a dose of about 30 mg/m². In some embodiments, a subject has moderate impairment of renal function, and wherein fludarabine is administered at a dose of about 24 mg/m² with monitoring. In some embodiments, a subject has a creatinine clearance of about 50-70 mL/min/1.73 m². In some embodiments, fludarabine is administered over approximately 30 minutes. In some embodiments, fludarabine is administered daily for 3 days. In some embodiments, fludarabine is administered to the subject 5 days, 4 days, and 3 days prior to the administration of the composition. In some embodiments, one liter of 0.9% sodium chloride is administered at completion of administration of fludarabine. In some embodiments, cyclophosphamide and fludarabine are administered concurrently. In some embodiments, a lymphodepleting chemotherapy regimen is administered intravenously. In some embodiments, a lymphodepleting chemotherapy regimen further comprises administration of sodium 2-mercaptoethanesulfonate.

In some embodiments, methods of the present disclosure do not comprise administering to a subject one or more corticosteroids for at least 5 days prior to administration of the composition. In some embodiments, one or more corticosteroids comprise greater than or equal to 5 mg/day of prednisone or an equivalent dose of another corticosteroid. In some embodiments, a subject has computed tomography (CT) scans with contrast that are contraindicating for administration of systemic corticosteroids. In some embodiments, a subject undergoes MRI with contrast and noncontrast CT scans during the five days prior to administration of the composition. In some embodiments, methods of the present disclosure do not comprise administering to a subject one or more corticosteroids or other immunosuppressive drugs for at least three months after administration of the composition. In some embodiments, methods of the present disclosure comprise administering to a subject one or more corticosteroids or other immunosuppressive drugs after administration of the composition for management of composition-related toxicities. In some embodiments, methods of the present disclosure do not comprise administering to the subject one or more corticosteroids, other immunosuppressive drugs, or other immunomodulatory medication at least one month prior to administration of the composition and at least two years after administration of the composition unless medically necessary. In some embodiments, an immunomodulatory medication comprises non-steroidal anti-inflammatory agents. In some embodiments, methods of the present disclosure do not comprise administering to a subject a lymphodepleting chemotherapy regimen prior to administration of the composition.

In some embodiments, methods of the present disclosure further comprise: administering to a subject a premedication therapy regimen, wherein a step of administering the premedication therapy regimen occurs prior to the step of administering a composition. In some embodiments, a premedication therapy is or comprises acetaminophen (paracetamol) and antihistamine. In some embodiments, a premedication therapy does not comprise corticosteroids or tocilizumab.

In some embodiments, methods of the present disclosure further comprise: administering to the subject a post-treatment therapy regimen, wherein a step of administering a post-treatment therapy regimen occurs after a step of administering a composition. In some embodiments, a post-treatment therapy is or comprises tocilizumab.

In some embodiments, methods of the present disclosure further comprise: collecting a sample from the subject after administration of the composition and determining: CAR T enumeration, CAR T cellular kinetics, CD47 expression, presence of replication competent lentivirus, T cell clonality, immunophenotype, and/or a cytokine profile.

In some embodiments, methods of the present disclosure further comprise modulating a subsequent administration of the composition, a post-treatment therapy, and/or one or more additional anti-cancer therapies based on the CAR T enumeration, CAR T cellular kinetics, CD47 expression, presence of replication competent lentivirus, T cell clonality, immunophenotype, and/or cytokine profile.

In some embodiments, methods of the present disclosure further comprise collecting a sample from a subject after administration of a composition and determining the presence of a: hypoimmunogenic T cell-mediated NK response, hypoimmunogenic T cell-mediated T response, hypoimmunogenic T cell-mediated humoral response, and/or humoral immunogenic response.

In some embodiments, methods of the present disclosure further comprise modulating a subsequent administration of the composition, a post-treatment therapy, and/or one or more additional anti-cancer therapies based on the presence of the hypoimmunogenic T cell-mediated NK response, hypoimmunogenic T cell-mediated T response, hypoimmunogenic T cell-mediated humoral response, and/or humoral immunogenic response.

In some embodiments, methods of the present disclosure further comprise: collecting a sample from the subject after administration of the composition and performing: single cell RNA sequencing, a minimal residual disease (MRD) assay a circulating tumor DNA (ctDNA) assay, and/or bulk RNA sequencing. In some embodiments, methods of the present disclosure further comprise modulating a subsequent administration of a composition, a post-treatment therapy, and/or one or more additional anti-cancer therapies based on the single cell RNA sequencing, an MRD assay, a ctDNA assay, or bulk RNA sequencing.

In some embodiments, methods of the present disclosure further comprise collecting a sample from a subject after administration of a composition and determining the presence of a cellular and/or humoral immune response directed towards one or more CD19-specific CAR peptides. In some embodiments, a cellular and/or humoral immune response is determined using ELISPOT or immunohistochemistry analysis. In some embodiments, methods of the present disclosure further comprise modulating a subsequent administration of a composition, a post-treatment therapy, and/or one or more additional anti-cancer therapies based on the presence of a cellular and/or humoral immune response.

In some embodiments, methods of the present disclosure further comprise: (i) obtaining first sequencing data for a first plurality of DNA molecules, (ii) determining, based on the first sequencing data, a first cancer profile, (iii) obtaining second sequencing data for a second plurality of DNA molecules, (iv) determining, based on the second sequencing data, a second cancer profile, and (v) detecting, based on a comparison of the first cancer profile and the second cancer profile a cancer status of the subject. In some embodiments, a cancer status is a minimal residual disease (MRD) status. In some embodiments, MRD status is based on identification of cancer specific markers in a first cancer profile and identification of the same cancer specific markers in a second cancer profile. In some embodiments, identification of cancer specific markers in a first cancer profile, a second cancer profile, or both, comprises comparing markers in the first cancer profile, the second cancer profile, or both to a reference cancer profile.

In some embodiments, a cancer status is a circulating tumor DNA (ctDNA) status. In some embodiments, ctDNA status is based on identification of cancer specific markers in a first cancer profile and identification of the same cancer specific markers in a second cancer profile. In some embodiments, identification of cancer specific markers in a first cancer profile, a second cancer profile, or both, comprises comparing the markers in the first cancer profile, the second cancer profile, or both to a reference cancer profile.

In some embodiments, methods of the present disclosure further comprise modulating a subsequent administration of a composition, a post-treatment therapy, and/or one or more additional anti-cancer therapies based in part on a change in the cancer status of the subject.

In some embodiments, methods of the present disclosure further comprise (i) obtaining first sequencing data for a first plurality of DNA molecules, (ii) determining, based on the first sequencing data, a first B-cell/T-cell profile for the patient, (iii) obtaining second sequencing data for a second plurality of DNA molecules, (iv) determining, based on the second sequencing data, a second B-cell/T-cell profile for the patient, (v) detecting, based on a comparison of the first B-cell/T-cell profile and the second B-cell/T-cell profile a change in the B-cell/T-cell status for a patient. In some embodiments, B-cell/T-cell status is the identity of B-cells and T-cells in the subject. In some embodiments, detection of the identity of the B-cells and T-cells in the subject is based on detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile and detection of the same rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the second B-cell/T-cell profile. In some embodiments, detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both, comprises comparing the detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both to a reference. In some embodiments, B-cell/T-cell status is the quantity of B-cells and T-cells in the subject. In some embodiments, detection of the quantity of the B-cells and T-cells in the subject is based on detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile and detection of the same rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the second B-cell/T-cell profile. In some embodiments, detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both, comprises comparing the detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both to a reference.

In some embodiments, methods of the present disclosure further comprise modulating a subsequent administration of the composition, a post-treatment therapy, and/or one or more additional anti-cancer therapies based in part on a change in the subject's status, the identity of B-cells and T-cells in the subject, the quantity of B-cells and T-cells in the subject, or a combination thereof.

In some embodiments, methods of the present disclosure further comprise (i) obtaining a first sample from the subject before administration of the composition; (ii) obtaining a first set of sequencing data for a first plurality of DNA molecules obtained from the first sample; (iii) determining a first cancer profile based on the first set of sequencing data; (iv) obtaining a second sample from the subject after administration of the composition; (v) obtaining a second set of sequencing data for a second plurality of DNA molecules obtained from the second sample; (vi) determining a second cancer profile based on the second set of sequencing data; and (vii) comparing the first cancer profile and the second cancer profile to determine a positive minimal residual disease (MRD) status or a negative MRD status in the subject.

In some embodiments, methods of the present disclosure further comprise (i) obtaining a first sample from the subject before administration of the composition; (ii) obtaining a first set of sequencing data for a first plurality of DNA molecules obtained from the first sample; (iii) determining a first B cell receptor (BCR)/TCR profile based on the first set of sequencing data; (iv) obtaining a second sample from the subject after administration of the composition; (v) obtaining a second set of sequencing data for a second plurality of DNA molecules obtained from the second sample; (vi) determining a second BCR/TCR profile based on the second set of sequencing data; and (vii) comparing the first BCR/TCR profile and the second BCR/TCR profile to determine a positive MRD status or a negative MRD status in the subject.

In some embodiments, methods of the present disclosure further comprise (i) obtaining a first sample from the subject before administration of the composition; (ii) obtaining a first set of sequencing data for a first plurality of DNA molecules obtained from the first sample; (iii) determining a first B cell/T cell profile based on the first set of sequencing data; (iv) obtaining a second sample from the subject after administration of the composition; (v) obtaining a second set of sequencing data for a second plurality of DNA molecules obtained from the second sample; (vi) determining a second B cell/T cell profile based on the second set of sequencing data; and (vii) comparing the first B cell/T cell profile and the second B Cell/T cell profile to determine a positive MRD status or negative MRD status in the subject.

In some embodiments, methods of the present disclosure further comprise (i) obtaining a first sample from the subject before administration of the composition; (ii) obtaining a first set of sequencing data for a first plurality of DNA molecules obtained from the first sample; (iii) determining a first B cell/T cell quantity based on the first set of sequencing data; (iv) obtaining a second sample from the subject after administration of the composition; (v) obtaining a second set of sequencing data for a second plurality of DNA molecules obtained from the second sample; (vi) determining a second B cell/T cell quantity based on the second set of sequencing data; and (vii) comparing the first B cell/T cell quantity and the second B Cell/T cell quantity to determine a positive MRD status or negative MRD status in the subject.

In some embodiments, if the subject is determined to have a positive MRD status, the subject is subsequently administered one or more additional anti-cancer therapies. In some embodiments, one or more additional anti-cancer therapies comprise a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments, methods of the present disclosure further comprise collecting a sample from a subject after administration of the composition and determining disease progression. In some embodiments, disease progression is determined by an increased diameter measurement of one or more malignant lesions as visualized via radiography. In some embodiments, a subject is administered one or more additional anti-cancer therapies in the presence of disease progression. In some embodiments, one or more additional anti-cancer therapies comprise a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments, engineered hypoimmunogenic T cells elicit reduced immune recognition when administered to the subject relative to the control or wild-type cells. In some embodiments, engineered hypoimmunogenic T cells elicit a reduced adaptive immune response when administered to the subject relative to the control or wild-type cells. In some embodiments, engineered hypoimmunogenic T cells elicit a reduced T cell response when administered to the subject relative to the control or wild-type cells. In some embodiments, engineered hypoimmunogenic T cells elicit a reduced innate immune response when administered to the subject relative to the control or wild-type cells. In some embodiments, engineered hypoimmunogenic T cells elicit a reduced natural killer (NK) cell response when administered to the subject relative to the control or wild-type cells. In some embodiments, engineered hypoimmunogenic T cells elicit a reduced macrophage response when administered to the subject relative to the control or wild-type cells. In some embodiments, engineered hypoimmunogenic T cells elicit a reduced graft-versus-host reaction (GvHR) when administered to the subject relative to the control or wild-type cells. In some embodiments, engineered hypoimmunogenic T cells persist in the subject for a longer duration relative to the control or wild-type cells. In some embodiments, engineered hypoimmunogenic T cells have greater expansion in the subject relative to the control or wild-type cells.

In some embodiments, engineered hypoimmunogenic T cells have an improved: (i) objective response; (ii) duration of response; (iii) time to next treatment; (iv) progression-free survival; (v) event-free survival; (vi) overall survival; (vii) MRD negativity, or combination thereof, relative to an untreated subject and/or a subject treated with a standard of care therapy. In some embodiments, an event-free survival duration is at least about 6, 7, 8, 9, 10, 11, 12, 13, 14, 15, 16, 17, 18, 19, 20, 21, 22, 23, or 24 months. In some embodiments, an event-free survival duration is about 10 months. In some embodiments, a progression-free survival duration is at least about 6, 7, 8, 9, 10, 11, 12, 13, 14, 15, 16, 17, 18, 19, 20, 21, 22, 23, or 24 months. In some embodiments, a progression-free survival duration is about 15 months. In some embodiments, a complete response rate (CRR) is about 30%, 35%, 40%, 45%, 50%, 55%, 60%, 65%, 70%, 75%, 80%, 85%, 90%, 95%, 97%, 98%, 99%, or 100%. In some embodiments, a CRR is about 65%. In some embodiments, a CRR is about 50%. In some embodiments, a CRR is about 30%. In some embodiments, a durable CRR is at least about 20%, 25%, 30%, 35%, 40%, 45%, 50%, 55%, 60%, 65%, 70%, 75%, 80%, 85%, 90%, 95%, 97%, 98%, 99%, or 100%. In some embodiments, a durable CRR is about 30%. In some embodiments, a durable CRR is about 40%. In some embodiments, a durable CRR is about 50%. In some embodiments, a durable CRR is about 60%. In some embodiments, an overall response rate (ORR) is about 30%, 35%, 40%, 45%, 50%, 55%, 60%, 65%, 70%, 75%, 80%, 85%, 90%, 95%, 97%, 98%, 99%, or 100%. In some embodiments, an ORR is about 70%. In some embodiments, an ORR is about 80%. In some embodiments, a median duration of response (mDOR) is at least about 6, 7, 8, 9, 10, 11, 12, 13, 14, 15, 16, 17, 18, 19, 20, 21, 22, 23, or 24 months. In some embodiments, an mDOR is at least about 12 months. In some embodiments, an MRD negativity is about 30%, 35%, 40%, 45%, 50%, 55%, 60%, 65%, 70%, 75%, 80%, 85%, 90%, 95%, 97%, 98%, 99%, or 100%. In some embodiments, an MRD negativity is about 70%. In some embodiments, a B cell malignancy is LBCL and the composition comprising a population of engineered hypoimmunogenic T cells is a second-line therapy. In some embodiments, a B cell malignancy is transplant-ineligible LBCL and the composition comprising a population of engineered hypoimmunogenic T cells is a second-line therapy. In some embodiments, a B cell malignancy is LBCL and the composition comprising a population of engineered hypoimmunogenic T cells is a third-line therapy. In some embodiments, a B cell malignancy is CLL and the composition comprising a population of engineered hypoimmunogenic T cells is a third-line therapy.

In some embodiments, methods of the present disclosure further comprise (i) obtaining a biological sample from a subject; and (ii) performing an analysis of a biological sample to determine, a CD19 expression status, a CD20 expression status, a CD22 expression status, or a combination thereof. In some embodiments, methods of the present disclosure further comprise: (i) obtaining a biological sample from the subject; and (ii) performing an analysis of the biological sample to determine: a donor-specific antibody evaluation status, a soluble marker of inflammation status, a soluble marker of B cell activation status, a serum immunoglobulin status, a proteinuria level status, a serum creatinine status, an estimated glomerular filtration status, an autoantibody status, or combination thereof. In some embodiments, a biological sample was collected prior to the step of administering a composition. In some embodiments, a biological sample was collected after the step of administering the composition.

In some embodiments, a positive CD19 expression status, CD20 expression status, CD22 expression status, or combination thereof, is prognostic of an improved, (i) objective response; (ii) duration of response; (iii) time to next treatment; (iv) progression-free survival; (v) event-free survival; (vi) overall survival; or combination thereof, relative to the subject prior to administration of the composition.

In some embodiments, a subject is a human.

In another aspect, the present disclosure provides articles of manufacture comprising a composition of a cell therapy, or one of a plurality of compositions of a cell therapy, comprising T cells expressing an anti-CD 19 chimeric antigen receptor (CAR), and instructions for administering the cell therapy, wherein the instructions specify administering the T cell composition according to the methods of the present disclosure.

In another aspect, the present disclosure provides pharmaceutical compositions comprising a population of engineered hypoimmunogenic T cells comprising: (a) reduced expression of beta-2-microglobulin (B2M), Class II Major Histocompatibility Complex Transactivator (CIITA), and T cell receptor alpha constant (TRAC) relative to a control or wild-type T cells, (b) increased expression of CD47 encoded by a first exogenous polynucleotide relative to the control or wild-type T cells, and (c) expression of a CD19-specific chimeric antigen receptor (CAR) encoded by a second exogenous polynucleotide. In some embodiments, pharmaceutical compositions of the present disclosure further comprise one or more excipients comprising: a cryopreservation medium; an isotonic electrolyte solution; or a combination thereof. In some embodiments, pharmaceutical compositions of the present disclosure further comprise dimethylsulfoxide (DMSO). In some embodiments, pharmaceutical compositions of the present disclosure further comprise human serum albumin (HSA). In some embodiments, an isotonic electrolyte solution comprises: sodium chloride; sodium gluconate; sodium acetate trihydrate; potassium chloride; magnesium chloride; or a combination thereof. In some embodiments, a pharmaceutical composition comprises a concentration of 7.5% DMSO (v/v) and 1.2% HSA (w/v). In some embodiments, pharmaceutical composition comprises a concentration of 7.5% DMSO (v/v) and 0.3% HSA (w/v). In some embodiments, pharmaceutical compositions of the present disclosure comprise partially edited CAR+ cells, wherein the partially edited CAR+ cells comprise one or more modifications that: (a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules, or (b) increase expression of CD47 encoded by a first exogenous polynucleotide, wherein the increased expression of (b) is relative to a control or wild-type T cell that does not comprise the modification, and express a CD19-specific chimeric antigen receptor (CAR) encoded by a second exogenous polynucleotide.

### BRIEF DESCRIPTION OF THE DRAWINGS

The drawings are for illustration purposes only, not for limitation.

FIG. 1 shows a chart illustrating a study and subject treatment schema.

FIG. 2 shows a chart illustrating exemplary grading and management of cytokine release syndrome.

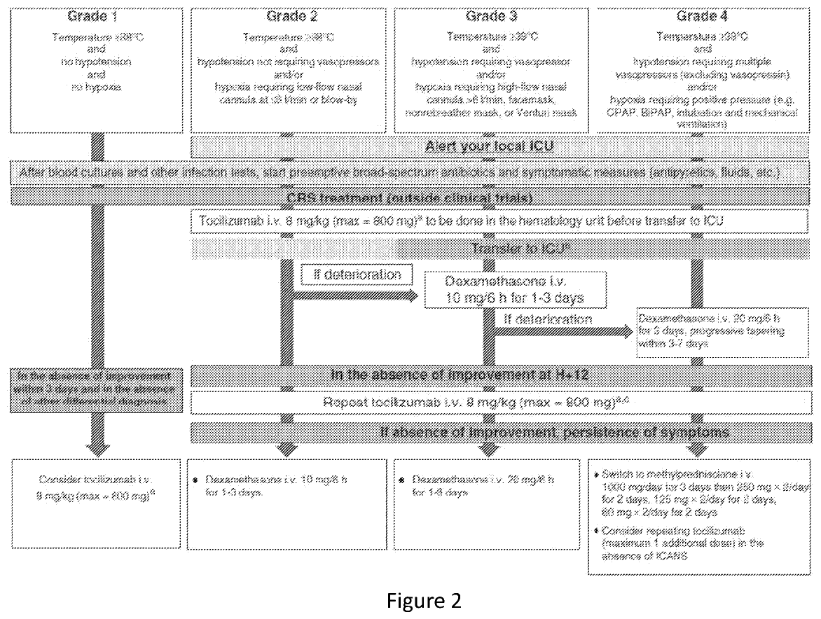

FIG. 3 shows a chart illustrating exemplary grading and management of immune effector cell-associated neurotoxicity syndrome.

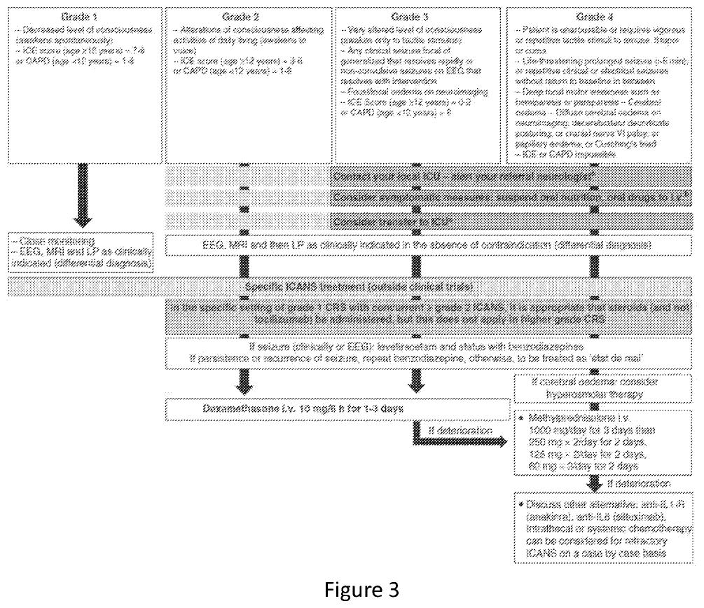

FIG. 4 shows a flow chart illustrating a treatment algorithm for second-line treatment of large B cell lymphomas (LBCLs).

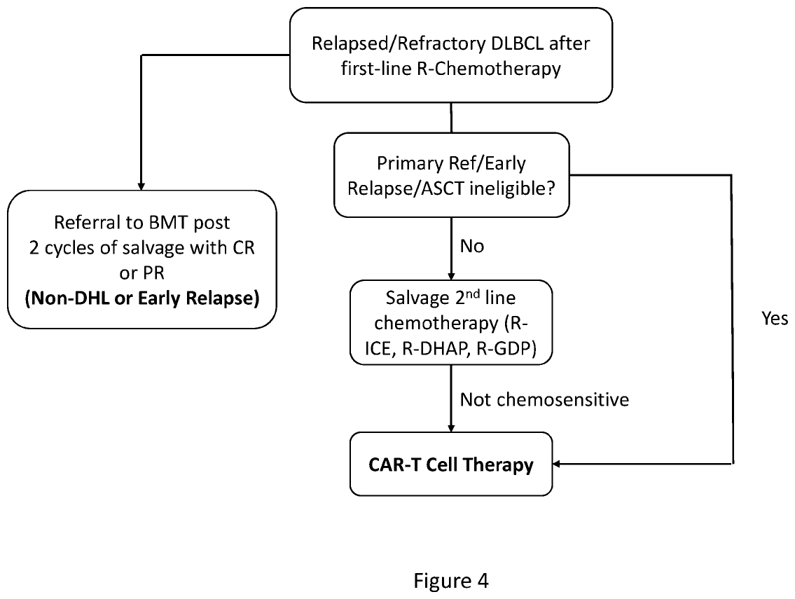

FIG. 5 shows a graph illustrating exemplary percentages of CD19+ cells before and after treatment with a T cell drug product of the present disclosure.

FIG. 6 shows graphs illustrating exemplary T cell activation against subpopulations of cells present in a T cell drug product of the present disclosure.

FIG. 7 shows graphs illustrating exemplary donor-specific antibody levels against subpopulations of cells present in a T cell drug product of the present disclosure.

FIG. 8 shows graphs illustrating exemplary killing of subpopulations of cells present in a T cell drug product of the present disclosure by a subject's NK cells.

FIG. 9 shows exemplary images of ISH for WPRE (left) and immunohistochemistry for B2M (right), identifying CAR+ cells from a T cell drug product of the present disclosure in bone marrow taken from a subject.

FIG. 10 shows graphs illustrating exemplary killing of subpopulations of cells present in a T cell drug product of the present disclosure by a subject's cells.

FIG. 11 shows graphs illustrating killing of subpopulations of cells present in a T cell drug product of the present disclosure by complement-dependent cytotoxicity (CDC).

FIG. 12 shows graphs illustrating killing of subpopulations of cells present in a T cell drug product of the present disclosure by antibody-dependent cellular cytotoxicity (ADCC).

FIG. 13 shows a graph illustrating exemplary percentages of CD19+ cells before and after treatment with a T cell drug product of the present disclosure.

FIG. 14 shows graphs illustrating exemplary T cell activation against subpopulations of cells present in a T cell drug product of the present disclosure.

FIG. 15 shows graphs illustrating exemplary donor-specific antibody levels against subpopulations of cells present in a T cell drug product of the present disclosure.

FIG. 16 shows graphs illustrating exemplary killing of subpopulations of cells present in a T cell drug product of the present disclosure by a subject's NK cells.

FIG. 17 shows graphs illustrating exemplary killing of subpopulations of cells present in a T cell drug product of the present disclosure by a subject's cells.

FIG. 18 shows graphs illustrating killing of subpopulations of cells present in a T cell drug product of the present disclosure by complement-dependent cytotoxicity (CDC).

FIG. 19 shows graphs illustrating killing of subpopulations of cells present in a T cell drug product of the present disclosure by antibody-dependent cellular cytotoxicity (ADCC).

FIG. 20 shows exemplary expression data of PD-L1 in an ALL cell line and a CLL cell line and expression of PD-1 in a T cell drug product of the present disclosure.

FIG. 21 shows graphs illustrating exemplary killing in an ALL cell line and a CLL cell line at several effector to target (E/T) ratios, with the effector cells being a T cell drug product of the present disclosure.

FIG. 22 shows graphs illustrating exemplary killing in an ALL cell line and a CLL cell line at several effector to target (E/T) ratios, with the effector cells being PD-1 positive or PD-1 negative cells from a T cell drug product of the present disclosure.

FIG. 23 shows graphs illustrating exemplary killing in an ALL cell line and a MCL cell line at several effector to target (E/T) ratios, with the effector cells being a T cell drug product of the present disclosure.

FIG. 24 shows exemplary expression data of PD-L1 in an ALL cell line and an MCL cell line.

FIG. 25 shows a graph illustrating exemplary percentages of CD19+ cells before and after treatment with a T cell drug product of the present disclosure.

FIG. 26 shows graphs illustrating exemplary T cell activation against subpopulations of cells present in a T cell drug product of the present disclosure.

FIG. 27 shows graphs illustrating exemplary donor-specific antibody levels against subpopulations of cells present in a T cell drug product of the present disclosure.

FIG. 28 shows graphs illustrating exemplary killing of subpopulations of cells present in a T cell drug product of the present disclosure by a subject's NK cells.

FIG. 29 shows graphs illustrating exemplary killing of subpopulations of cells present in a T cell drug product of the present disclosure by a subject's cells.

FIG. 30 shows graphs illustrating killing of subpopulations of cells present in a T cell drug product of the present disclosure by complement-dependent cytotoxicity (CDC).

FIG. 31 shows graphs illustrating killing of subpopulations of cells present in a T cell drug product of the present disclosure by antibody-dependent cellular cytotoxicity (ADCC).

FIG. 32 shows a graph illustrating exemplary percentages of CD19+ cells before and after treatment with a T cell drug product of the present disclosure.

FIG. 33 shows graphs illustrating exemplary T cell activation against subpopulations of cells present in a T cell drug product of the present disclosure.

FIG. 34 shows graphs illustrating exemplary donor-specific antibody levels against subpopulations of cells present in a T cell drug product of the present disclosure.

FIG. 35 shows graphs illustrating exemplary killing of subpopulations of cells present in a T cell drug product of the present disclosure by a subject's NK cells.

FIG. 36 shows graphs illustrating exemplary killing of subpopulations of cells present in a T cell drug product of the present disclosure by a subject's cells.

FIG. 37 shows graphs illustrating killing of subpopulations of cells present in a T cell drug product of the present disclosure by complement-dependent cytotoxicity (CDC).

FIG. 38 shows graphs illustrating killing of subpopulations of cells present in a T cell drug product of the present disclosure by antibody-dependent cellular cytotoxicity (ADCC).

FIG. 39 shows a graph illustrating a summary of disease progression for Subject 1, Subject 2, Subject 3, and Subject 4, including the statuses of complete response (CR), partial response (PR), stable disease (SD), and progressive disease (PD).

### DETAILED DESCRIPTION

### I. Introduction

Described herein are engineered or modified immune evasive cells, including but not limited to human immune evasive cells. To overcome the problem of a subject's immune rejection of these primary and/or stem cell-derived transplants, the inventors have developed and describe herein hypoimmunogenic cells (e.g., hypoimmunogenic pluripotent cells, differentiated cells derived from such, and primary cells) that represent a viable source for any transplantable cell type. Such cells are protected from adaptive and/or innate immune rejection upon administration to a recipient subject. Advantageously, the cells disclosed herein are not rejected by the recipient subject's immune system, regardless of the subject's genetic make-up, as they are protected from adaptive and innate immune rejection upon administration to a recipient subject. In some embodiments, the engineered and/or hypoimmunogenic cells do not express major histocompatibility complex (MHC) class I and class II antigens and/or T-cell receptors. In certain embodiments, the engineered and/or hypoimmunogenic cells do not express MHC I and II antigens and/or T-cell receptors and overexpress CD47 proteins. In certain embodiments, the engineered and/or hypoimmunogenic cells such as engineered and/or hypoimmunogenic T cells do not express MHC I and II antigens and/or T-cell receptors, overexpress CD47 proteins and express exogenous CARs.

In some embodiments, hypoimmunogenic cells outlined herein are not subject to an innate immune cell rejection. In some instances, hypoimmunogenic cells are not susceptible to NK cell-mediated lysis. In some instances, hypoimmunogenic cells are not susceptible to macrophage engulfment. In some embodiments, hypoimmunogenic cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation, including, e.g., pluripotency, as well as being capable of engraftment and functioning similarly to a corresponding native cell.

The technology disclosed herein utilizes expression of tolerogenic factors and modulation (e.g., reduction or elimination) of MHC I, MHC II, and/or TCR expression in human cells. In some embodiments, genome editing technologies utilizing rare-cutting endonucleases (e.g., CRISPR/Cas) are also used to reduce or eliminate expression of genes involved in an immune response (e.g., by deleting genomic DNA of genes involved in an immune response or by insertions of genomic DNA into such genes, such that gene expression is impacted) in the cells. In some embodiments, genome editing technologies or other gene modulation technologies are used to insert tolerance-inducing (tolerogenic) factors in human cells, rendering the cells and their progeny (include any differentiated cells prepared therefrom) able to evade immune recognition upon engrafting into a recipient subject. As such, the cells described herein exhibit modulated expression of one or more genes and factors that affect MHC I, MHC II, and/or TCR expression and evade the recipient subject's immune system.

The genome editing techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The practice of the numerous embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### II. Definitions

As described in the present disclosure, the following terms will be employed, and are defined as indicated below.

The term “autoimmune disease” refers to any disease or disorder in which the subject mounts an immune response against its own tissues and/or cells. Autoimmune disorders can affect almost every organ system in the subject (e.g., human), including, but not limited to, diseases of the nervous, gastrointestinal, and endocrine systems, as well as skin and other connective tissues, cycs, blood and blood vessels. Examples of autoimmune diseases include, but are not limited to Hashimoto's thyroiditis, Systemic lupus erythematosus, Sjogren's syndrome, Graves' disease, Scleroderma, Rheumatoid arthritis, Multiple sclerosis, Myasthenia gravis and Diabetes.

The term “best response rate” or “BOR” is defined as an incidence of subjects with a complete response, partial response, stable disease, and/or progressive disease.

The term “cancer” as used herein is defined as a hyperproliferation of cells whose unique trait (e.g., loss of normal controls) results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues of the malignant type, unless otherwise specifically indicated and does not include a benign type tissue.

The term “chronic infectious disease” refers to a disease caused by an infectious agent wherein the infection has persisted. Such a disease may include hepatitis (A, B, or C), herpes virus (e.g., VZV, HSV-1, HSV-6, HSV-II, CMV, and EBV), and HIV/AIDS. Non-viral examples may include chronic fungal diseases such Aspergillosis, Candidiasis, Coccidioidomycosis, and diseases associated with Cryptococcus and Histoplasmosis. None limiting examples of chronic bacterial infectious agents may be Chlamydia pneumoniae, Listeria monocytogenes, and Mycobacterium tuberculosis. In some embodiments, the disorder is human immunodeficiency virus (HIV) infection. In some embodiments, the disorder is acquired immunodeficiency syndrome (AIDS).

As used herein, “clinically effective amount” refers to an amount sufficient to provide a clinical benefit in the treatment and/or management of a disease, disorder, or condition. In some embodiments, a clinically effective amount is an amount that has been shown to produce at least one improved clinical endpoint to the standard of care for the disease, disorder, or condition. In some embodiments, a clinically effective amount is an amount that has been demonstrated, for example in a clinical trial, to be sufficient to provide statistically significant and meaningful effectiveness for treating the disease, disorder, or condition. In some embodiments, the clinically effective amount is also a therapeutically effective amount. In other embodiments, the clinically effective amount is not a therapeutically effective amount.

In some embodiments, an alteration or modification (including, for example, genetic alterations or modifications) described herein results in reduced expression of a target or selected polynucleotide sequence. In some embodiments, an alteration or modification described herein results in reduced expression of a target or selected polypeptide sequence. In some embodiments, an alteration or modification described herein results in increased expression of a target or selected polynucleotide sequence. In some embodiments, an alteration or modification described herein results in increased expression of a target or selected polypeptide sequence.

The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. In some embodiments, the cells are engineered to have reduced expression of one or more targets relative to an unaltered or unmodified wild-type cell.

The term “donor subject” refers to an animal, for example, a human from whom cells can be obtained. The “non-human animals” and “non-human mammals” as used interchangeably herein, includes mammals such as rats, mice, rabbits, sheep, cats, dogs, cows, pigs, and non-human primates. The term “donor subject” also encompasses any vertebrate including but not limited to mammals, reptiles, amphibians and fish. However, advantageously, the donor subject is a mammal such as a human, or other mammals such as a domesticated mammal, e.g., dog, cat, horse, and the like, or production mammal, e.g., cow, sheep, pig, and the like. A “donor subject” can also refer to more than one donor, for example one or more humans or non-human animals or non-human mammals.

The term “duration of response” or “DOR” is defined for subjects who experience a best objective response (complete response or partial response) and is the time from the first objective response to disease progression or death due to any cause, whichever occurs first. In some embodiments, if the progression or death is not observed, the DOR will be censored at the last evaluable assessment date on or prior to the censoring events defined as (i) ongoing without event, (ii) lost to follow-up, (iii) withdrew consent, (iv) new anticancer therapy (including stem cell transplant), and/or (v) adequate assessments no longer available.

The term “endogenous” refers to a referenced molecule or polypeptide that is naturally present in the cell. Similarly, the term when used in reference to expression of an encoding nucleic acid refers to expression of an encoding nucleic acid naturally contained within the cell and not exogenously introduced. Similarly, the term when used in reference to a promoter sequence refers to a promoter sequence naturally contained within the cell and not exogenously introduced.

The term “engineered cell” as used herein refers to a cell that has been altered in at least some way by human intervention, including, for example, by genetic alterations or modifications such that the engineered cell differs from a wild-type cell.

The term “event-free survival” or “EFS” is the time from infusion of hypoimmune allogenic CD19-directed CAR T cells of the present disclosure to the earliest of date of death from any cause, disease progression or relapse, or new anticancer therapy (including stem cell transplant). In some embodiments, when a subject does not have any of the above events prior to data cutoff, EFS is censored at the last adequate response assessment date on or prior to the earliest censoring event (except for stem cell transplant). In some embodiments, the censoring reason could be (i) ongoing without event, (ii) lost to follow-up, (iii) withdrew consent, (iv) stem cell transplant (subjects who proceed to stem cell transplant after infusion of hypoimmune allogenic CD19-directed CAR T cells of the present disclosure will be censored at the time of stem cell transplant), and/or (v) event after at least 2 missing scheduled disease assessments.

As used herein, the term “exogenous” in the context of a polynucleotide or polypeptide being expressed is intended to mean that the referenced molecule or the referenced polypeptide is introduced into the cell of interest. The polypeptide can be introduced, for example, by introduction of an encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. An exogenous polynucleotide can be inserted into at least one allele of the cell using viral transduction, for example, with a vector. In some embodiments, the vector is a pseudotyped, self-inactivating lentiviral vector that carries exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, exogenous polynucleotide is inserted into at least one allele of the cell using a lentivirus based viral vector. In some embodiments, the exogenous polynucleotide is inserted into a safe harbor or target locus of at least one allele of the cell.

An “exogenous” molecule is a molecule, construct, factor and the like that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of neurons is an exogenous molecule with respect to an adult neuron cell. An exogenous molecule can comprise, for example, a functioning version of a malfunctioning endogenous molecule or a malfunctioning version of a normally-functioning endogenous molecule.

An exogenous molecule or factor can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule or construct can be the same type of molecule as an endogenous molecule, e.g., an exogenous protein or nucleic acid. In such instances, the exogenous molecule is introduced into the cell at greater concentrations than that of the endogenous molecule in the cell. In some instances, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and/or locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and/or glycosylation.

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids sequences Genetic modifications also include, for example, reduced or increased transcription, reduced or increased mRNA stability, reduced or increased translation, and reduced or increased protein stability.

As used herein, the terms “grafting”, “administering,” “introducing”, “implanting” and “transplanting” as well as grammatical variations thereof are used interchangeably in the context of the placement of cells (e.g., cells described herein) into a subject, by a method or route which results in localization or at least partial localization of the introduced cells at a desired site or systemic introduction (e.g., into circulation). The cells can be implanted directly to the desired site, or alternatively be administered by any appropriate route which results in delivery to a desired location in the subject where at least a portion of the implanted cells or components of the cells remain viable. The period of viability of the cells after administration to a subject can be as short as a few hours, e. g. twenty-four hours, to a few days, to as long as several years. In some embodiments, the cells can also be administered (e.g., injected) a location other than the desired site, such as in the brain or subcutaneously, for example, in a capsule to maintain the implanted cells at the implant location and avoid migration of the implanted cells.

By “HLA” or “human leukocyte antigen” complex is a gene complex encoding the MHC proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

As used herein to characterize a cell, the term “hypoimmunogenic” generally means that such cell is less prone to innate or adaptive immune rejection by a subject into which such cells are transplanted, e.g., the cell is less prone to allorejection by a subject into which such cells are transplanted. For example, relative to a cell of the same cell type that does not comprise the modifications, such a hypoimmunogenic cell may be about 2.5%, 5%, 10%, 20%, 30%, 40%, 50%, 60%, 70%, 80%, 90%, 95%, 97.5%, 99% or more less prone to innate or adaptive immune rejection by a subject into which such cells are transplanted. In some embodiments, genome editing technologies are used to modulate the expression of MHC I and MHC II genes, and thus, contribute to generation of a hypoimmunogenic cell. In some embodiments, a hypoimmunogenic cell evades immune rejection in an MHC-mismatched allogeneic recipient. In some instance, differentiated cells produced from the hypoimmunogenic stem cells outlined herein evade immune rejection when administered (e.g., transplanted or grafted) to an MHC-mismatched allogeneic recipient. In some embodiments, a hypoimmunogenic cell is protected from T cell-mediated adaptive immune rejection and/or innate immune cell rejection. Detailed descriptions of hypoimmunogenic cells, methods of producing thereof, and methods of using thereof are found in WO2016183041 filed May 9, 2015; WO2018132783 filed Jan. 14, 2018; WO2018176390 filed Mar. 20, 2018; WO2020018615 filed Jul. 17, 2019; WO2020018620 filed Jul. 17, 2019; PCT/US2020/44635 filed Jul. 31, 2020; U.S. 62/881,840 filed Aug. 1, 2019; U.S. 62/891,180 filed Aug. 23, 2019; U.S. 63/016,190, filed Apr. 27, 2020; and U.S. 63/052,360 filed Jul. 15, 2020, the disclosures including the examples, sequence listings and figures are incorporated herein by reference in their entirety.

Hypoimmunogenicity of a cell can be determined by evaluating the immunogenicity of the cell such as the cell's ability to elicit adaptive and innate immune responses or to avoid eliciting such adaptive and innate immune responses. Such immune response can be measured using assays recognized by those skilled in the art. In some embodiments, an immune response assay measures the effect of a hypoimmunogenic cell on T cell proliferation, T cell activation, T cell killing, donor specific antibody generation, NK cell proliferation, NK cell activation, and macrophage activity. In some cases, hypoimmunogenic cells and derivatives thereof undergo decreased killing by T cells and/or NK cells upon administration to a subject. In some instances, the cells and derivatives thereof show decreased macrophage engulfment compared to an unmodified or wild-type cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

“Immune signaling factor” as used herein refers to, in some cases, a molecule, protein, peptide and the like that activates immune signaling pathways.

“Immunosuppressive factor” or “immune regulatory factor” or “tolerogenic factor” as used herein include hypoimmunity factors, complement inhibitors, and other factors that modulate or affect the ability of a cell to be recognized by the immune system of a host or recipient subject upon administration, transplantation, or engraftment. These may be in combination with additional genetic modifications.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In some embodiments, the reference level, also referred to as the basal level, is 0.

In some embodiments, the alteration is an indel. As used herein, “indel” refers to a mutation resulting from an insertion, deletion, or a combination thereof. As will be appreciated by those skilled in the art, an indel in a coding region of a genomic sequence will result in a frameshift mutation, unless the length of the indel is a multiple of three. In some embodiments, the alteration is a point mutation. As used herein, “point mutation” refers to a substitution that replaces one of the nucleotides. A gene editing (e.g., CRISPR/Cas) system of the present disclosure can be used to induce an indel of any length or a point mutation in a target polynucleotide sequence.

As used herein, “knock down” refers to a reduction in expression of the target mRNA or the corresponding target protein. Knock down is commonly reported relative to levels present following administration or expression of a noncontrol molecule that does not mediate reduction in expression levels of RNA (e.g., a non-targeting control shRNA, siRNA, or miRNA). In some embodiments, knock down of a target gene is achieved by genetic modification, including use of gene editing systems (e.g., CRISPR/Cas).

Knock down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock down include RNA solution hybridization, nuclease protection, northern hybridization, gene expression monitoring with a microarray, antibody binding, radioimmunoassay, and fluorescence activated cell analysis. Those skilled in the art will readily appreciate how to use the gene editing systems (e.g., CRISPR/Cas) of the present disclosure to knock out a target polynucleotide sequence or a portion thereof based upon the details described herein.

By “knock in” or “knock-in” herein is meant a genetic modification resulting from the insertion of a DNA sequence into a chromosomal locus in a host cell. This causes initiation of or increased levels of expression of the knocked in gene, portion of gene, or nucleic acid sequence inserted product, e.g., an increase in RNA transcript levels and/or encoded protein levels. As will be appreciated by those in the art, this can be accomplished in several ways, including inserting or adding one or more additional copies of the gene or portion thereof to the host cell or altering a regulatory component of the endogenous gene increasing expression of the protein is made or inserting a specific nucleic acid sequence whose expression is desired. This may be accomplished by modifying a promoter, adding a different promoter, adding an enhancer, adding other regulatory elements, or modifying other gene expression sequences.

As used herein, “knock out” or “knock-out” includes deleting all or a portion of a target polynucleotide sequence in a way that interferes with the translation or function of the target polynucleotide sequence. For example, a knock out can be achieved by altering a target polynucleotide sequence by inducing an insertion or a deletion (“indel”) in the target polynucleotide sequence, including in a functional domain of the target polynucleotide sequence (e.g., a DNA binding domain). Those skilled in the art will readily appreciate how to use the gene editing systems (e.g., CRISPR/Cas) of the present disclosure to knock out a target polynucleotide sequence or a portion thereof based upon the details described herein.

In some embodiments, a genetic modification or alteration results in a knock out or knock down of the target polynucleotide sequence or a portion thereof. Knocking out a target polynucleotide sequence or a portion thereof using a gene editing system (e.g., CRISPR/Cas) of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject) or for changing the genotype or phenotype of a cell.

“Modulation” of gene expression refers to a change in the expression level of a gene. Modulation of expression can include, but is not limited to, gene activation and gene repression. Modulation may also be complete, i.e., wherein gene expression is totally inactivated or is activated to wild-type levels or beyond; or it may be partial, wherein gene expression is partially reduced, or partially activated to some fraction of wild-type levels.

The methods provided herein can be used to alter a target polynucleotide sequence in a cell. The present disclosure contemplates altering target polynucleotide sequences in a cell for any purpose. In some embodiments, the target polynucleotide sequence in a cell is altered to produce a mutant cell. As used herein, a “mutant cell” refers to a cell with a resulting genotype that differs from its original genotype. In some instances, a “mutant cell” exhibits a mutant phenotype, for example when a normally functioning gene is altered using the gene editing systems (e.g., CRISPR/Cas) systems of the present disclosure. In other instances, a “mutant cell” exhibits a wild-type phenotype, for example when a gene editing system (e.g., CRISPR/Cas) system of the present disclosure is used to correct a mutant genotype. In some embodiments, the target polynucleotide sequence in a cell is altered to correct or repair a genetic mutation (e.g., to restore a normal phenotype to the cell). In some embodiments, the target polynucleotide sequence in a cell is altered to induce a genetic mutation (e.g., to disrupt the function of a gene or genomic element).

The term “native cell” as used herein refers to a cell that is not otherwise modified (e.g., engineered). In some embodiments, a native cell is a naturally occurring wild-type or a control cell.

The term “objective response rate” or “ORR” is defined as a proportion of subjects with a best overall response of complete response or partial response as determined by an investigator and/or central assessment. Subjects who do not meet the criteria for an objective response by an analysis cutoff date will be considered non-responders. In some embodiments, disease assessments obtained after infusion and up through an observation of progression or start of new anti-cancer therapy will be used. In some embodiments, response assessment in non-Hodgkin lymphoma subjects will be based on the Lugano classification criteria and in chronic lymphocytic leukemia subjects based on the International Workshop on Chronic Lymphocytic Leukemia criteria.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

The term “overall survival” or “OS” is defined as the time from infusion of hypoimmune allogenic CD19-directed CAR T cells of the present disclosure to death from any cause. In some embodiments, subjects who have not died by the analysis data cutoff date will have survival time censored at their last date known to be alive. In some embodiments, for subjects alive or dead after the data cutoff date, survival time will be censored at the data cutoff date.

The term “progression free survival” or “PFS” is defined as the time of infusion of hypoimmune allogenic CD19-directed CAR T cells of the present disclosure to disease progression or death from any cause, whichever occurs first. In some embodiments, if the progression or death is not observed, the PFS will be censored at the last evaluable assessment date on or prior to the censoring events defined as (i) ongoing without event, (ii) lost to follow up, (iii) withdrew consent, (iv) new anticancer therapy (including stem cell transplant), and/or (v) adequate assessments no longer available.

As used herein, “promoter,” “promoter sequence,” or “promoter region” refers to a DNA regulatory region/sequence capable of binding RNA polymerase and involved in initiating transcription of a downstream coding or non-coding sequence. In some examples, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes.

In some embodiments, the engineered and hypoimmunogenic cells described are propagated from a primary T cell or a progeny thereof. As used herein, the term “propagated from a primary T cell or a progeny thereof” encompasses the initial primary T cell that is isolated from the donor subject and any subsequent progeny thereof. As used herein, the term “progeny” encompasses, e.g., a first-generation progeny, i.e., the progeny is directly derived from, obtained from, obtainable from or derivable from the initial primary T cell by, e.g., traditional propagation methods. The term “progeny” also encompasses further generations such as second, third, fourth, fifth, sixth, seventh, or more generations, i.e., generations of cells which are derived from, obtained from, obtainable from or derivable from the former generation by, e.g., traditional propagation methods. The term “progeny” also encompasses modified cells that result from the modification or alteration of the initial primary T cell or a progeny thereof.

The term “recipient patient” refers to an animal, for example, a human to whom treatment, including prophylactic treatment, with the cells as described herein, is provided. For treatment of those infections, conditions or disease states, which are specific for a specific animal such as a human patient, the term patient refers to that specific animal. The term “recipient patient” also encompasses any vertebrate including but not limited to mammals, reptiles, amphibians and fish. However, advantageously, the recipient patient is a mammal such as a human, or other mammals such as a domesticated mammal, e.g., dog, cat, horse, and the like, or production mammal, e.g., cow, sheep, pig, and the like.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example but are not limited to, the timing of transcription, amount or level of transcription, RNA processing or stability, and/or translation of the related structural nucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. It is recognized that since in most cases the exact boundaries of regulatory sequences have not been completely defined, nucleotide sequences of different lengths may have identical regulatory or promoter activity.

As used herein, a “target” can refer to a gene, a portion of a gene, a portion of the genome, or a protein that is subject to regulatable reduced expression by the methods described herein.

As used herein, “therapeutically effective amount” refers to an amount sufficient to provide a therapeutic benefit in the treatment and/or management of a disease, disorder, or condition. In some embodiments, a therapeutically effective amount is an amount sufficient to ameliorate, palliate, stabilize, reverse, slow, attenuate or delay the progression of a disease, disorder, or condition, or of a symptom or side effect of the disease, disorder, or condition. In some embodiments, the therapeutically effective amount is also a clinically effective amount. In other embodiments, the therapeutically effective amount is not a clinically effective amount.

The term “time to next treatment” or “TTNT” is defined as the time from infusion of hypoimmune allogenic CD19-directed CAR T cells of the present disclosure to the next anticancer treatment (including stem cell transplant) or death from any cause, whichever occurs first. In some embodiments, when subsequent anticancer treatment is not received (e.g., continuing on current treatment) and subjects are still alive by the analysis data cutoff date, subjects will be censored at their last available follow-up date before the cutoff date.

As used herein, the term “treating” and “treatment” includes administering to a subject a therapeutically or clinically effective amount of cells described herein so that the subject has a reduction in at least one symptom of the disease or an improvement in the disease, for example, beneficial or desired therapeutic or clinical results. For purposes of this technology, beneficial or desired therapeutic or clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a condition, disease or disorder are alleviated by at least 5%, at least 10%, at least 20%, at least 30%, at least 40%, or at least 50% upon treatment of the condition, disease or disorder.

For purposes of this technology, beneficial or desired therapeutic or clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and/or viral vector-mediated transfer.

In some embodiments, the cells are engineered to have reduced or increased expression of one or more targets relative to an unaltered or unmodified wild-type cell. In some embodiments, the cells are engineered to have constitutive reduced or increased expression of one or more targets relative to an unaltered or unmodified wild-type cell. In some embodiments, the cells are engineered to have regulatable reduced or increased expression of one or more targets relative to an unaltered or unmodified wild-type cell. In some embodiments, the cells comprise increased expression of CD47 relative to a wild-type cell or a control cell of the same cell type. By “wild-type” or “wt” or “control” in the context of a cell means any cell found in nature. Examples of wild type or control cells include primary cells and T cells found in nature. However, by way of example, in the context of an engineered cell, as used herein, “wild-type” or “control” can also mean an engineered cell that may contain nucleic acid changes resulting in reduced expression of MHC I and/or II and/or T-cell receptors, but did not undergo the gene editing procedures to result in overexpression of CD47 proteins. For example, as used herein, “wild-type” or “control” means an engineered cell that comprises reduced or knocked out expression of B2M, CIITA, and/or TRAC. Also as used herein, “wild-type” or “control” means an engineered cell that comprises reduced or knocked out expression of B2M, CIITA, TRAC, and/or TRBC. As used herein, “wild-type” or “control” also means an engineered cell that may contain nucleic acid changes resulting in overexpression of CD47 proteins, but did not undergo the gene editing procedures to result in reduced expression of MHC I and/or II and/or T-cell receptors. In the context of an iPSC or a progeny thereof, “wild-type” or “control” also means an iPSC or progeny thereof that may contain nucleic acid changes resulting in pluripotency but did not undergo the gene editing procedures of the present disclosure to achieve reduced expression of MHC I and/or II and/or T-cell receptors, and/or overexpression of CD47 proteins. In the context of a primary T cell or a progeny thereof, “wild-type” or “control” also means a primary T cell or progeny thereof that may contain nucleic acid changes resulting in reduced expression of MHC I and/or II and/or T-cell receptors, but did not undergo the gene editing procedures to result in overexpression of CD47 proteins. For example, as used herein, “wild-type” or “control” means a primary T cell or progeny thereof that comprises reduced or knocked out expression of B2M, CIITA, and/or TRAC. Also as used herein, “wild-type” or “control” means a primary T cell or progeny thereof that comprises reduced or knocked out expression of B2M, CIITA, TRAC, and/or TRBC. Also in the context of a primary T cell or a progeny thereof, “wild-type” or “control” also means a primary T cell or progeny thereof that may contain nucleic acid changes resulting in overexpression of CD47 proteins, but did not undergo the gene editing procedures to result in reduced expression of MHC I and/or II and/or T-cell receptors. In some embodiments, the cells are engineered to have regulatable reduced or increased expression of one or more targets relative to a cell of the same cell type that does not comprise the modifications. In some embodiments, the wild-type cell or the control cell is a starting material. In some embodiments, the starting material is a primary cell collected from a donor. In some embodiments, the starting material is a primary blood cell collected from a donor, e.g., via a leukopak. For example, unmodified T cells obtained from a donor is a starting material that are considered wild-type or control cells as contemplated herein. In another example, an iPSC cell line starting material is a starting material that is considered a wild-type or control cell as contemplated herein. In some embodiments, the starting material is otherwise modified or engineered to have altered expression of one or more genes to generate the engineered cell.

It is noted that the claims may be drafted to exclude any optional element. As such, this statement is intended to serve as antecedent basis for use of such exclusive terminology as “solely,” “only,” and the like in connection with the recitation of claim elements, or use of a “negative” limitation. As will be apparent to those of skill in the art upon reading this disclosure, each of the individual embodiments described and illustrated herein has discrete components and features readily separated from or combined with the features of any of the other several embodiments without departing from the scope or spirit of the present disclosure. Any recited method may be carried out in the order of events recited or in any other order that is logically possible. Although any methods and materials similar or equivalent to those described herein may also be used in the practice or testing of the present disclosure, representative illustrative methods and materials are now described.

Unless defined otherwise, all technical and scientific terms used herein have the same meaning as commonly understood by one of ordinary skill in the art to which this technology belongs. Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges and are also encompassed within the present disclosure, subject to any specifically excluded limit in the stated range. Where the stated range includes one or both of the limits, ranges excluding either or both of those included limits are also included in the present disclosure. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number. The term about is used herein to mean plus or minus ten percent (10%) of a value. For example, “about 100” refers to any number between 90 and 110.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present disclosure will be limited only by the appended claims. It should also be understood that the headers used herein are not limiting and are merely intended to orient the reader, but the subject matter generally applies to the technology disclosed herein.

### III. Detailed Description of the Embodiments

A. Methods of Treatment with Hypoimmunogenic T Cells

As is described in further detail herein, provided herein are methods for treating a patient with a disease, disorder, or condition through administration of hypoimmunogenic cells, particularly hypoimmunogenic T cells. As will be appreciated, for all the multiple embodiments described herein related to the timing and/or combinations of therapies, administration of the cells is accomplished by a method or route which results in at least partial localization of the introduced cells at a desired site. Cells can be infused, implanted, or transplanted directly to the desired site, or alternatively be administered by any appropriate route which results in delivery to a desired location in the subject where at least a portion of the implanted cells or components of the cells remain viable.

Provided herein are methods for treating a patient with a disease, disorder, or condition of the present disclosure, which includes administration of a population of hypoimmunogenic cells (e.g., primary T cells) to a subject, e.g., a human patient. For instance, a population of hypoimmunogenic primary T cells such as, but limited to, CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells that express CD45RA (TEMRA cells), tissue-resident memory (Trm) cells, virtual memory T cells, innate memory T cells, memory stem cell (Tsc), γΔ T cells, and any other subtype of T cell is administered to a patient to treat a disease, disorder, or condition of the present disclosure.

In some embodiments, pharmaceutical compositions as described herein are co-administered with a therapeutic agent that that binds to and/or interacts with one or more receptors selected from the group consisting of CD94, KIR2DL4, PD-1, an inhibitory NK cell receptor, and an activating NK receptor. In some instances, a therapeutic agent binds to a receptor on the surface of an NK cell, including one or more subpopulations of NK cells. In some embodiments, a therapeutic agent is selected from the group consisting of an antibody and fragments and variants thereof, an antibody mimetic, a small molecule, a blocking peptide, and a receptor antagonist. In some embodiments, pharmaceutical compositions as described herein are administered as part of a combination therapy comprising a Bruton tyrosine kinase (BTK) inhibitor. In some embodiments, a BTK inhibitor is selected from a group consisting of: ibrutinib, acalabrutinib, zanubrutinib, and pirtobrutinib.

### 1. Dosing and Formulations

Any therapeutically effective amount of cells described herein can be included in a pharmaceutical composition of the present disclosure, depending on the indication being treated. Non-limiting examples of the cells include primary T cells. In some embodiments, a pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least 30×10⁶ cells. In some embodiments, the pharmaceutical composition includes approximately 30×10⁶ cells. In some embodiments, the pharmaceutical composition includes at least 60×10⁶ cells. In some embodiments, the pharmaceutical composition includes approximately 60×10⁶ cells. In some embodiments, the pharmaceutical composition includes at least 90×10⁶ cells. In some embodiments, the pharmaceutical composition includes approximately 90×10⁶ cells. In some embodiments, the pharmaceutical composition includes at least 120×10⁶ cells. In some embodiments, the pharmaceutical composition includes approximately 120×10⁶ cells. In some embodiments, the pharmaceutical composition includes at least 200×10⁶ cells. In some embodiments, the pharmaceutical composition includes approximately 200×10⁶ cells. In some embodiments, the pharmaceutical composition includes at least about 1×10² to 5×10², 5×10² to 1×10³, 1×10³ to 5×10³, 5×10³ to 1×10⁴, 1×10⁴ to 5×10⁴, 5×10⁴ to 1×10⁵, 1×10⁵ to 5×10⁵, 5×10⁵ to 1×10⁶, 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, or 1×10¹⁰ to 5×10¹⁰ cells. In some embodiments, the pharmaceutical composition includes from about 1.0×10⁶ to about 2.5×10⁸ cells. In some embodiments, the pharmaceutical composition includes from about 2.0×10⁶ to about 2.0×10⁸ cells. In some embodiments, a pharmaceutical composition of the present disclosure comprises from about 30×10⁶ cells to about 200×10⁶ cells. In some embodiments, a pharmaceutical composition of the present disclosure comprises from about 20×10⁶ cells to about 400×10⁶ cells.

In some embodiments, a pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ CAR+ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ CAR+ cells. In some embodiments, the pharmaceutical composition includes up to about 6.0×10⁸ CAR+ cells. In some embodiments, the pharmaceutical composition includes up to about 8.0×10⁸ CAR+ cells. In some embodiments, the pharmaceutical composition includes at least 30×10⁶ CAR+ cells. In some embodiments, the pharmaceutical composition includes approximately 30×10⁶ CAR+ cells. In some embodiments, the pharmaceutical composition includes at least 60×10⁶ CAR+ cells. In some embodiments, the pharmaceutical composition includes approximately 60×10⁶ CAR+ cells. In some embodiments, the pharmaceutical composition includes at least 120×10⁶ CAR+ cells. In some embodiments, the pharmaceutical composition includes approximately 120×10⁶ CAR+ cells. In some embodiments, the pharmaceutical composition includes at least 200×10⁶ CAR+ cells. In some embodiments, the pharmaceutical composition includes approximately 200×10⁶ CAR+ cells. In some embodiments, the pharmaceutical composition includes at least about 1×10² to 5×10², 5×10² to 1×10³, 1×10³ to 5×10³, 5×10³ to 1×10⁴, 1×10⁴ to 5×10⁴, 5×10⁴ to 1×10⁵, 1×10⁵ to 5×10⁵, 5×10⁵ to 1×10⁶, 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, or 1×10¹⁰ to 5×10¹⁰ CAR+ cells. In some embodiments, the pharmaceutical composition includes from about 1.0×10⁶ to about 2.5×10⁸ CAR+ cells. In some embodiments, the pharmaceutical composition includes from about 2.0×10⁶ to about 2.0×10⁸ CAR+ cells. In some embodiments, a pharmaceutical composition of the present disclosure comprises from about 30×10⁶ CAR+ cells to about 200×10⁶ CAR+ cells. In some embodiments, a pharmaceutical composition of the present disclosure comprises about 5×10⁶ CAR+ cells to at least about 400×10⁶ CAR+ cells. In some embodiments, a pharmaceutical composition of the present disclosure comprises about 20×10⁶ CAR+ cells to about 400×10⁶ CAR+ cells. In some embodiments, a pharmaceutical composition of the present disclosure comprises at least about 30×10⁶ CAR+ cells, at least about 60×10⁶ CAR+ cells, at least about 120×10⁶ CAR+ cells, or at least about 200×10⁶ CAR+ cells. In some embodiments, a pharmaceutical composition of the present disclosure comprises no more than about 200×10⁶ CAR+ cells. In some embodiments, a pharmaceutical composition of the present disclosure comprises about 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, 110×10⁶, 111×10⁶, 112×10⁶, 113×10⁶, 114×10⁶, 115×10⁶, 116×10⁶, 117×10⁶, 118×10⁶, 119×10⁶, 120×10⁶, 121×10⁶, 122×10⁶, 123×10⁶, 124×10⁶, 125×10⁶, 126×10⁶, 127×10⁶, 128×10⁶, 129×10⁶, 130×10⁶, 131×10⁶, 132×10⁶, 133×10⁶, 134×10⁶, 135×10⁶, 136×10⁶, 137×10⁶, 138×10⁶, 139×10⁶, 140×10⁶, 141×10⁶, 142×10⁶, 143×10⁶, 144×10⁶, 145×10⁶, 146×10⁶, 147×10⁶, 148×10⁶, 149×10⁶, 150×10⁶, 151×10⁶, 152×10⁶, 153×10⁶, 154×10⁶, 155×10⁶, 156×10⁶, 157×10⁶, 158×10⁶, 159×10⁶, 160×10⁶, 161×10⁶, 162×10⁶, 163×10⁶, 164×10⁶, 165×10⁶, 166×10⁶, 167×10⁶, 168×10⁶, 169×10⁶, 170×10⁶, 171×10⁶, 172×10⁶, 173×10⁶, 174×10⁶, 175×10⁶, 176×10⁶, 177×10⁶, 178×10⁶, 179×10⁶, 180×10⁶, 181×10⁶, 182×10⁶, 183×10⁶, 184×10⁶, 185×10⁶, 186×10⁶, 187×10⁶, 188×10⁶, 189×10⁶, 190×10⁶, 191×10⁶, 192×10⁶, 193×10⁶, 194×10⁶, 195×10⁶, 196×10⁶, 197×10⁶, 198×10⁶, 199×10⁶, or 200×10⁶ CAR+ cells.

In some embodiments, a pharmaceutical composition of the present disclosure comprises fully edited hypoimmunogenic CAR+ cells as described herein. In some embodiments, fully edited hypoimmunogenic CAR+ cells comprise one or more modifications that: (a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and (iii) one or more T-cell receptor (TCR) molecules and/or one or more molecules that regulate expression of the one or more TCR molecules, (b) increase expression of CD47 encoded by a first exogenous polynucleotide, wherein the increased expression of (b) is relative to a control or wild-type T cell that does not comprise the modification, and (c) express a CD19-specific chimeric antigen receptor (CAR) encoded by a second exogenous polynucleotide. In some embodiments, a pharmaceutical composition of the present disclosure comprises partially edited CAR+ cells as described herein. In some embodiments, partially edited CAR+ cells are missing one or more modifications that: (a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules, and/or (b) increase expression of CD47 encoded by a first exogenous polynucleotide, wherein the increased expression of (b) is relative to a control or wild-type T cell that does not comprise the modification, relative to fully edited hypoimmunogenic CAR+ cells as described herein. In some embodiments, partially edited CAR+ cells (i) are missing one or more modifications that inactivate or disrupt one or more alleles of one or more major histocompatibility complex (MHC) class I molecules and/or one or more molecules that regulate expression of the one or more MHC class I molecules, and (ii) comprise one or more modifications that inactivate one or more MHC class II molecules and/or one or more molecules that regulate expression of the one or more MHC class II molecules, relative to fully edited hypoimmunogenic CAR+ cells as described herein. In some embodiments, partially edited CAR+ cells (i) comprise one or more modifications that inactivate or disrupt one or more alleles of one or more major histocompatibility complex (MHC) class I molecules and/or one or more molecules that regulate expression of the one or more MHC class I molecules, and (ii) are missing one or more modifications that inactivate one or more MHC class II molecules and/or one or more molecules that regulate expression of the one or more MHC class II molecules, relative to fully edited hypoimmunogenic CAR+ cells as described herein. In some embodiments, partially edited CAR+ cells (i) are missing one or more modifications that inactivate or disrupt one or more alleles of one or more major histocompatibility complex (MHC) class I molecules and/or one or more molecules that regulate expression of the one or more MHC class I molecules, and (ii) are missing one or more modifications that inactivate one or more MHC class II molecules and/or one or more molecules that regulate expression of the one or more MHC class II molecules, relative to fully edited hypoimmunogenic CAR+ cells as described herein. In some embodiments, a pharmaceutical composition of the present disclosure comprises fully edited hypoimmunogenic CAR+ cells and partially edited CAR+ cells. In some embodiments, a pharmaceutical composition of the present disclosure comprises at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% fully edited hypoimmunogenic CAR+ cells. In some embodiments, a pharmaceutical composition of the present disclosure comprises 100% fully edited hypoimmunogenic CAR+ cells. In some embodiments, a pharmaceutical composition of the present disclosure comprises about 20% to about 40% fully edited hypoimmunogenic CAR+ cells. In some embodiments, a pharmaceutical composition of the present disclosure comprises about 30% to about 50% fully edited hypoimmunogenic CAR+ cells. In some embodiments, a pharmaceutical composition of the present disclosure comprises about 40% to about 60% fully edited hypoimmunogenic CAR+ cells. In some embodiments, a pharmaceutical composition of the present disclosure comprises about 50% to about 70% fully edited hypoimmunogenic CAR+ cells. In some embodiments, a pharmaceutical composition of the present disclosure comprises about 60% to about 80% fully edited hypoimmunogenic CAR+ cells. In some embodiments, a pharmaceutical composition of the present disclosure comprises about 70% to about 90% fully edited hypoimmunogenic CAR+ cells. In some embodiments, a pharmaceutical composition of the present disclosure comprises about 80% to about 99% fully edited hypoimmunogenic CAR+ cells. In some embodiments, a pharmaceutical composition of the present disclosure comprises less than 5%, less than 10%, less than 15%, less than 20%, less than 25%, less than 30%, less than 35%, less than 40% partially edited CAR+ cells. In some embodiments, a pharmaceutical composition of the present disclosure comprises about 1% to about 10% partially edited CAR+ cells. In some embodiments, a pharmaceutical composition of the present disclosure comprises about 5% to about 10% partially edited CAR+ cells. In some embodiments, a pharmaceutical composition of the present disclosure comprises about 5% to about 15% partially edited CAR+ cells. In some embodiments, a pharmaceutical composition of the present disclosure comprises about 5% to about 20% partially edited CAR+ cells. In some embodiments, a pharmaceutical composition of the present disclosure comprises about 10% to about 30% partially edited CAR+ cells. In some embodiments, a pharmaceutical composition of the present disclosure comprises about 20% to about 40% partially edited CAR+ cells.

In some embodiments, a pharmaceutical composition has a volume of at least 1.0, 1.25, 1.5, 1.75, 2.0, 2.5, 3.0, 3.5, 4.0, 4.5, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 mL. In exemplary embodiments, a pharmaceutical composition has a volume of up to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 mL. In exemplary embodiments, a pharmaceutical composition has a volume of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 mL. In some embodiments, a pharmaceutical composition has a volume of from about 1-50 mL, 50-100 mL, 100-150 mL, 150-200 mL, 200-250 mL, 250-300 mL, 300-350 mL, 350-400 mL, 400-450 mL, or 450-500 mL. In some embodiments, a pharmaceutical composition has a volume of from about 1-50 mL, 50-100 mL, 100-150 mL, 150-200 mL, 200-250 mL, 250-300 mL, 300-350 mL, 350-400 mL, 400-450 mL, or 450-500 mL. In some embodiments, a pharmaceutical composition has a volume of from about 1-10 mL, 10-20 mL, 20-30 mL, 30-40 mL, 40-50 mL, 50-60 mL, 60-70 mL, 70-80 mL, 70-80 mL, 80-90 mL, or 90-100 mL. In some embodiments, a pharmaceutical composition has a volume that ranges from about 1.25 mL to about 100 mL. In some embodiments, a pharmaceutical composition has a volume that ranges from about 5 mL to about 80 mL. In some embodiments, a pharmaceutical composition has a volume that ranges from about 10 mL to about 70 mL. In some embodiments, a pharmaceutical composition has a volume that ranges from about 10 mL to about 50 mL. In some embodiments, a pharmaceutical composition has a volume of at least about 7.5 mL, at least about 15 mL, at least about 30 mL, or at least about 50 mL.

In some embodiments, a specific amount/dosage regimen will vary depending on the weight, gender, age and health of a subject; the formulation, the biochemical nature, bioactivity, bioavailability and the side effects of the cells and the number and identity of the cells in the complete therapeutic regimen.

In some embodiments, a therapeutically effective dose or a clinically effective dose of the pharmaceutical composition includes about 1.0×10⁵ to about 2.5×10⁸ cells at a volume of about 10 mL to 50 mL and the pharmaceutical composition is administered as a single therapeutically effective dose or clinically effective dose. In some cases, the therapeutically effective dose or clinically effective dose includes about 1.0×10⁵ to about 2.5×10⁸ primary T cells described herein at a volume of about 10 mL to 50 mL. In some embodiments, a therapeutically effective dose or clinically effective dose includes about 1.0×10⁵ to about 2.5×10⁸ primary T cells as disclosed herein at a volume of about 10 mL to 50 mL.

In some embodiments, a therapeutically effective dose or a clinically effective dose of the pharmaceutical composition comprises a concentration of engineered hypoimmunogenic T cells of at least 1.25×10⁶ cells/mL. In some embodiments, a therapeutically effective dose or a clinically effective dose of the pharmaceutical composition comprises a concentration of engineered hypoimmunogenic T cells of at least 4.0×10⁶ cells/mL. In some embodiments, a therapeutically effective dose or a clinically effective dose of the pharmaceutical composition comprises a concentration of engineered hypoimmunogenic T cells of at least 1.2×10⁶ CAR+ cells/mL. In some embodiments, a therapeutically effective dose or a clinically effective dose of the pharmaceutical composition comprises a concentration of engineered hypoimmunogenic T cells of at least 4.0×10⁶ CAR+ cells/mL.

In some embodiments, the pharmaceutical composition is administered as a single therapeutically effective dose or clinically effective dose of from about 1.0×10⁵ to about 1.0×10⁷ cells (such as primary T cells) per kg body weight for subjects 50 kg or less. In some embodiments, the pharmaceutical composition is administered as a single therapeutically effective dose or clinically effective dose of from about 0.5×10⁵ to about 1.0×10⁷, about 1.0×10⁵ to about 1.0×10⁷, about 1.0×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0×10⁵ to about 5.0×10⁶, about 4.0×10⁵ to about 5.0×10⁶, about 5.0×10⁵ to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the therapeutically effective dose or clinically effective dose is 0.5×10⁵, 0.6×10⁵, 0.7×10⁵, 0.8×10⁵, 0.9×10⁵, 1.0×10⁵, 1.1×10⁵, 1.2×10⁵, 1.3×10⁵, 1.4×10⁵, 1.5×10⁵, 1.6×10⁵, 1.7×10⁵, 1.8×10⁵, 1.9×10⁵, 2.0×10⁵, 2.1×10⁵, 2.2×10⁵, 2.3×10⁵, 2.4×10⁵, 2.5×10⁵, 2.6×10⁵, 2.7×10⁵, 2.8×10⁵, 2.9×10⁵, 3.0×10⁵, 3.1×10⁵, 3.2×10⁵, 3.3×10⁵, 3.4×10⁵, 3.5×10⁵, 3.6×10⁵, 3.7×10⁵, 3.8×10⁵, 3.9×10⁵, 4.0×10⁵, 4.1×10⁵, 4.2×10⁵, 4.3×10⁵, 4.4×10⁵, 4.5×10⁵, 4.6×10⁵, 4.7×10⁵, 4.8×10⁵, 4.9×10⁵, 5.0×10⁵, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 0.5×10⁷, 0.6×10⁷, 0.7×10⁷, 0.8×10⁷, 0.9×10⁷, or 1.0×10⁷ cells per kg body weight for subjects 50 kg or less. In some embodiments, a therapeutically effective dose or clinically effective dose is from about 0.2×10⁶ to about 5.0×10⁶ cells per kg body weight for subjects 50 kg or less. In certain embodiments, a therapeutically effective dose or clinically effective dose is at a range that is lower than from about 0.2×10⁶ to about 5.0×10⁶ cells per kg body weight for subjects 50 kg or less. In some embodiments, a single therapeutically effective dose or clinically effective dose is at a volume of about 10 mL to 50 mL. In some embodiments, a therapeutically effective dose or clinically effective dose is administered intravenously. In some embodiments, a therapeutically effective dose or clinically effective dose is administered to the nervous system of a subject. In some embodiments, the nervous system is the central nervous system (CNS). In some embodiments, the nervous system is the peripheral nervous system (PNS). In some embodiments, a therapeutically effective dose or clinically effective dose is administered intrathecally.

In exemplary embodiments, cells are administered in a single therapeutically effective dose of from about 1.0×10⁶ to about 5.0×10⁸ cells (such as primary T cells) for subjects above 50 kg. In some embodiments, a pharmaceutical composition is administered as a single therapeutically effective dose or clinically effective dose of from about 0.5×10⁶ to about 1.0×10⁹, about 1.0×10⁶ to about 1.0×10⁹, about 1.0×10⁶ to about 1.0×10⁹, about 5.0×10⁶ to about 1.0×10⁹, about 1.0×10⁷ to about 1.0×10⁹, about 5.0×10⁷ to about 1.0×10⁹, about 1.0×10⁶ to about 5.0×10⁷, about 1.0×10⁶ to about 1.0×10⁷, about 1.0×10⁶ to about 5.0×10⁷, about 1.0×10⁷ to about 5.0×10⁸, about 2.0×10⁷ to about 5.0×10⁸, about 3.0×10⁷ to about 5.0×10⁸, about 4.0×10⁷ to about 5.0×10⁸, about 5.0×10⁷ to about 5.0×10⁸, about 6.0×10⁷ to about 5.0×10⁸, about 7.0×10⁷ to about 5.0×10⁸, about 8.0×10⁷ to about 5.0×10⁸, or about 9.0×10⁷ to about 5.0×10⁸ cells per kg body weight for subjects 50 kg or less. In some embodiments, a therapeutically effective dose or clinically effective dose is 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, 5.0×10⁶, 5.1×10⁶, 5.2×10⁶, 5.3×10⁶, 5.4×10⁶, 5.5×10⁶, 5.6×10⁶, 5.7×10⁶, 5.8×10⁶, 5.9×10⁶, 6.0×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7.0×10⁶, 7.1×10⁶, 7.2×10⁶, 7.3×10⁶, 7.4×10⁶, 7.5×10⁶, 7.6×10⁶, 7.7×10⁶, 7.8×10⁶, 7.9×10⁶, 8.0×10⁶, 8.1×10⁶, 8.2×10⁶, 8.3×10⁶, 8.4×10⁶, 8.5×10⁶, 8.6×10⁶, 8.7×10⁶, 8.8×10⁶, 8.9×10⁶, 9.0×10⁶, 9.1×10⁶, 9.2×10⁶, 9.3×10⁶, 9.4×10⁶, 9.5×10⁶, 9.6×10⁶, 9.7×10⁶, 9.8×10⁶, 9.9×10⁶, 1.0×10⁷, 1.1×10⁷, 1.2×10⁷, 1.3×10⁷, 1.4×10⁷, 1.5×10⁷, 1.6×10⁷, 1.7×10⁷, 1.8×10⁷, 1.9×10⁷, 2.0×10⁷, 2.1×10⁷, 2.2×10⁷, 2.3×10⁷, 2.4×10⁷, 2.5×10⁷, 2.6×10⁷, 2.7×10⁷, 2.8×10⁷, 2.9×10⁷, 3.0×10⁷, 3.1×10⁷, 3.2×10⁷, 3.3×10⁷, 3.4×10⁷, 3.5×10⁷, 3.6×10⁷, 3.7×10⁷, 3.8×10⁷, 3.9×10⁷, 4.0×10⁷, 4.1×10⁷, 4.2×10⁷, 4.3×10⁷, 4.4×10⁷, 4.5×10⁷, 4.6×10⁷, 4.7×10⁷, 4.8×10⁷, 4.9×10⁷, 5.0×10⁷, 5.1×10⁷, 5.2×10⁷, 5.3×10⁷, 5.4×10⁷, 5.5×10⁷, 5.6×10⁷, 5.7×10⁷, 5.8×10⁷, 5.9×10⁷, 6.0×10⁷, 6.1×10⁷, 6.2×10⁷, 6.3×10⁷, 6.4×10⁷, 6.5×10⁷, 6.6×10⁷, 6.7×10⁷, 6.8×10⁷, 6.9×10⁷, 7.0×10⁷, 7.1×10⁷, 7.2×10⁷, 7.3×10⁷, 7.4×10⁷, 7.5×10⁷, 7.6×10⁷, 7.7×10⁷, 7.8×10⁷, 7.9×10⁷, 8.0×10⁷, 8.1×10⁷, 8.2×10⁷, 8.3×10⁷, 8.4×10⁷, 8.5×10⁷, 8.6×10⁷, 8.7×10⁷, 8.8×10⁷, 8.9×10⁷, 9.0×10⁷, 9.1×10⁷, 9.2×10⁷, 9.3×10⁷, 9.4×10⁷, 9.5×10⁷, 9.6×10⁷, 9.7×10⁷, 9.8×10⁷, 9.9×10⁷, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, or 5.0×10⁸ cells per kg body weight for subjects 50 kg or less. In certain embodiments, cells are administered in a single therapeutically effective dose or clinically effective dose of about 1.0×10⁷ to about 2.5×10⁸ cells for subjects above 50 kg. In some embodiments, cells are administered in a single therapeutically effective dose or clinically effective dose of a range that is less than about 1.0×10⁷ to about 2.5×10⁸ cells for subjects above 50 kg. In some embodiments, cells are administered in a single therapeutically effective dose or clinically effective dose of a range that is higher than about 1.0×10⁷ to about 2.5×10⁸ cells for subjects above 50 kg. In some embodiments, a dose is administered intravenously. In some embodiments, a therapeutically effective dose or clinically effective dose is administered to the nervous system of a subject. In some embodiments, the nervous system is the central nervous system (CNS). In some embodiments, the nervous system is the peripheral nervous system (PNS). In some embodiments, a therapeutically effective dose or clinically effective dose is administered intrathecally. In some embodiments, a single therapeutically effective dose or clinically effective dose is at a volume of about 10 mL to 50 mL. In some embodiments, a therapeutically effective dose or clinically effective dose is administered intravenously. In some embodiments, a therapeutically effective dose or clinically effective dose is administered to the nervous system of a subject. In some embodiments, the nervous system is the central nervous system (CNS). In some embodiments, the nervous system is the peripheral nervous system (PNS). In some embodiments, a therapeutically effective dose or clinically effective dose is administered intrathecally.

In exemplary embodiments, the therapeutically effective dose or clinically effective dose is administered intravenously at a rate of about 1 to 50 mL per minute, 1 to 40 mL per minute, 1 to 30 mL per minute, 1 to 20 mL per minute, 10 to 20 mL per minute, 10 to 30 mL per minute, 10 to 40 mL per minute, 10 to 50 mL per minute, 20 to 50 mL per minute, 30 to 50 mL per minute, 40 to 50 mL per minute. In numerous embodiments, a pharmaceutical composition is stored in one or more infusion bags for intravenous administration. In some embodiments, a dose is administered completely in no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single therapeutically effective dose or clinically effective dose of the pharmaceutical composition is present in a single infusion bag. In other embodiments, a single therapeutically effective dose or clinically effective dose of a pharmaceutical composition is divided into 2, 3, 4 or 5 separate infusion bags.

In some embodiments, cells described herein are administered in a plurality of doses such as 2, 3, 4, 5, 6 or more doses, wherein the plurality of doses together constitute a therapeutically effective dose or clinically effective dose regimen. In some embodiments, each dose of a plurality of doses is administered to the subject ranging from 1 to 24 hours apart. In some instances, a subsequent dose is administered from about 1 hour to about 24 hours (e.g., about 1, 2, 3, 4, 5, 6, 7, 8, 9, 10, 11, 12, 13, 14, 15, 16, 17, 18, 19, 20, 21, 22, 23 or about 24 hours) after an initial or preceding dose. In some embodiments, each dose of a plurality of doses is administered to the subject ranging from about 1 day to 28 days apart. In some instances, a subsequent dose is administered from about 1 day to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or about 28 days) after an initial or preceding dose. In certain embodiments, each dose of a plurality of doses is administered to the subject ranging from 1 week to about 6 weeks apart. In certain instances, a subsequent dose is administered from about 1 week to about 6 weeks (e.g., about 1, 2, 3, 4, 5, or 6 weeks) after an initial or preceding dose. In several embodiments, each dose of a plurality of doses is administered to the subject ranging from about 1 month to about 12 months apart. In several instances, a subsequent dose is administered from about 1 month to about 12 months (e.g., about 1, 2, 3, 4, 5, 6, 7, 8, 9, 10, 11, or 12 months) after an initial or preceding dose.

In some embodiments, a subject is administered a first dosage regimen at a first time point, and then subsequently administered a second dosage regimen at a second time point. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different.

In some embodiments, CD19 specific (CD19) CAR-T cells described herein are administered to a subject at a dose of about 50×10⁶ to about 200×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, 110×10⁶, 111×10⁶, 112×10⁶, 113×10⁶, 114×10⁶, 115×10⁶, 116×10⁶, 117×10⁶, 118×10⁶, 119×10⁶, 120×10⁶, 121×10⁶, 122×10⁶, 123×10⁶, 124×10⁶, 125×10⁶, 126×10⁶, 127×10⁶, 128×10⁶, 129×10⁶, 130×10⁶, 131×10⁶, 132×10⁶, 133×10⁶, 134×10⁶, 135×10⁶, 136×10⁶, 137×10⁶, 138×10⁶, 139×10⁶, 140×10⁶, 141×10⁶, 142×10⁶, 143×10⁶, 144×10⁶, 145×10⁶, 146×10⁶, 147×10⁶, 148×10⁶, 149×10⁶, 150×10⁶, 151×10⁶, 152×10⁶, 153×10⁶, 154×10⁶, 155×10⁶, 156×10⁶, 157×10⁶, 158×10⁶, 159×10⁶, 160×10⁶, 161×10⁶, 162×10⁶, 163×10⁶, 164×10⁶, 165×10⁶, 166×10⁶, 167×10⁶, 168×10⁶, 169×10⁶, 170×10⁶, 171×10⁶, 172×10⁶, 173×10⁶, 174×10⁶, 175×10⁶, 176×10⁶, 177×10⁶, 178×10⁶, 179×10⁶, 180×10⁶, 181×10⁶, 182×10⁶, 183×10⁶, 184×10⁶, 185×10⁶, 186×10⁶, 187×10⁶, 188×10⁶, 189×10⁶, 190×10⁶, 191×10⁶, 192×10⁶, 193×10⁶, 194×10⁶, 195×10⁶, 196×10⁶, 197×10⁶, 198×10⁶, 199×10⁶, or 200×10⁶) viable CD19 specific CAR-T cells. In some embodiments, a dose is a therapeutically effective amount of viable CD19 specific CAR-T cells. In some embodiments, the dose is a clinically effective amount of viable CD19 specific CAR-T cells. In some embodiments, viable CD19 specific CAR-T cells include CD19 specific CAR expressing CD4+ T cells and CD19 specific CAR expressing CD8+ T cells at a ratio of about 1:1. In some embodiments, the CD19 specific CAR of the cells is lisocabtagene maraleucel (BREYANZI®), a structural equivalent thereof, or a functional equivalent thereof.

In some embodiments, a subject is administered about 50×10⁶ to about 200×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, 110×10⁶, 111×10⁶, 112×10⁶, 113×10⁶, 114×10⁶, 115×10⁶, 116×10⁶, 117×10⁶, 118×10⁶, 119×10⁶, 120×10⁶, 121×10⁶, 122×10⁶, 123×10⁶, 124×10⁶, 125×10⁶, 126×10⁶, 127×10⁶, 128×10⁶, 129×10⁶, 130×10⁶, 131×10⁶, 132×10⁶, 133×10⁶, 134×10⁶, 135×10⁶, 136×10⁶, 137×10⁶, 138×10⁶, 139×10⁶, 140×10⁶, 141×10⁶, 142×10⁶, 143×10⁶, 144×10⁶, 145×10⁶, 146×10⁶, 147×10⁶, 148×10⁶, 149×10⁶, 150×10⁶, 151×10⁶, 152×10⁶, 153×10⁶, 154×10⁶, 155×10⁶, 156×10⁶, 157×10⁶, 158×10⁶, 159×10⁶, 160×10⁶, 161×10⁶, 162×10⁶, 163×10⁶, 164×10⁶, 165×10⁶, 166×10⁶, 167×10⁶, 168×10⁶, 169×10⁶, 170×10⁶, 171×10⁶, 172×10⁶, 173×10⁶, 174×10⁶, 175×10⁶, 176×10⁶, 177×10⁶, 178×10⁶, 179×10⁶, 180×10⁶, 181×10⁶, 182×10⁶, 183×10⁶, 184×10⁶, 185×10⁶, 186×10⁶, 187×10⁶, 188×10⁶, 189×10⁶, 190×10⁶, 191×10⁶, 192×10⁶, 193×10⁶, 194×10⁶, 195×10⁶, 196×10⁶, 197×10⁶, 198×10⁶, 199×10⁶, or 200×10⁶) viable CD19 specific CAR-T cells described herein. In some embodiments, the dose is a therapeutically effective amount of viable CD19 specific CAR-T cells. In other embodiments, a dose is a clinically effective amount of viable CD19 specific CAR-T cells. In some instances, 50% of the viable CD19 specific CAR-T cells are CD19 specific CAR expressing CD4+ T cells and 50% of the viable CD19 specific CAR-T cells are CD19 specific CAR expressing CD8+ T cells. In some embodiments, the CD19 specific CAR of the cells is lisocabtagene maraleucel (BREYANZI®), a structural equivalent thereof, or a functional equivalent thereof.

In some embodiments, CD19 specific CAR-T cells as described herein are administered to a subject at a dose of about 2×10⁶ per kg of body weight. In some embodiments, a maximum dose administered is about 2×10⁸ viable CD19 specific CAR-T cells. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as axicabtagene ciloleucel (YESCARTA®), a structural equivalent thereof, or a functional equivalent thereof.

In some embodiments, the CD19 specific CAR-T cells described herein are administered to a subject at a dose of about 2×10⁶ per kg of body weight. In some embodiments, a maximum dose of about 2×10⁸ viable CD19 specific CAR-T cells is administered to a patient of about 100 kg of body weight and above. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as brexucabtagene autoleucel (TECARTUS®), a structural equivalent thereof, or a functional equivalent thereof.

In some embodiments, the CD19 specific CAR-T cells described herein are administered to a subject at a dose of up to about 2×10⁸ viable CD19 specific CAR-T cells. In some embodiments, a subject is administered from about 0.2×10⁶ to about 5.0×10⁶ (e.g., about 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, 2.5×10⁶, 2.6×10⁶, 2.8×10⁶, 2.9×10⁶, 3.0×10⁶, 3.2×10⁶, 3.4×10⁶, 3.5×10⁶, 3.6×10⁶, 3.8×10⁶, 3.9×10⁶, 4.0×10⁶, 4.2×10⁶, 4.4×10⁶, 4.5×10⁶, 4.6×10⁶, 4.8×10⁶, 4.9×10⁶, or 5.0×10⁶) viable CD19 specific CAR-T cells per kg of body weight for a subject with a body weight of about 50 kg or less. In some embodiments, a subject is administered from about 0.1×10⁸ to about 2.5×10⁸ (e.g., about 0.1×10⁶, 0.2×10⁶, 0.4×10⁶, 0.5×10⁶, 0.6×10⁶, 0.8×10⁶, 0.9×10⁶, 1.0×10⁶, 1.2×10⁶, 1.4×10⁶, 1.5×10⁶, 1.6×10⁶, 1.8×10⁶, 1.9×10⁶, 2.0×10⁶, 2.2×10⁶, 2.4×10⁶, or 2.5×10⁶) viable CD19 specific CAR-T cells for a subject with a body weight of greater than about 50 kg. In some embodiments, a subject is administered from about 0.6×10⁸ to about 6.0×10⁸ (e.g., about 0.6×10⁸, 0.8×10⁸, 0.9×10⁸, 1.0×10⁸, 1.2×10⁸, 1.4×10⁸, 1.5×10⁸, 1.6×10⁸, 1.8×10⁸, 1.9×10⁸, 2.0×10⁸, 2.2×10⁸, 2.4×10⁸, 2.5×10⁸, 2.6×10⁸, 2.8×10⁸, 2.9×10⁸, 3.0×10⁸, 3.2×10⁸, 3.4×10⁸, 3.5×10⁸, 3.6×10⁸, 3.8×10⁸, 3.9×10⁸, 4.0×10⁸, 4.2×10⁸, 4.4×10⁸, 4.5×10⁸, 4.6×10⁸, 4.8×10⁸, 4.9×10⁸, 5.0×10⁸, 5.2×10⁸, 5.4×10⁸, 5.5×10⁸, 5.6×10⁸, 5.8×10⁸, 5.9×10⁸, or 6.0×10⁸) viable CD19 specific CAR-T cells. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as tisagenlecleucel (KYMRIAH®), a structural equivalent thereof, or a functional equivalent thereof.

In some embodiments, a single dose of any of the CD19 specific CAR-T cells described herein includes about 50×10⁶ to about 200×10⁶ (e.g., 50×10⁶, 51×10⁶, 52×10⁶, 53×10⁶, 54×10⁶, 55×10⁶, 56×10⁶, 57×10⁶, 58×10⁶, 59×10⁶, 60×10⁶, 61×10⁶, 62×10⁶, 63×10⁶, 64×10⁶, 65×10⁶, 66×10⁶, 67×10⁶, 68×10⁶, 69×10⁶, 70×10⁶, 71×10⁶, 72×10⁶, 73×10⁶, 74×10⁶, 75×10⁶, 76×10⁶, 77×10⁶, 78×10⁶, 79×10⁶, 80×10⁶, 81×10⁶, 82×10⁶, 83×10⁶, 84×10⁶, 85×10⁶, 86×10⁶, 87×10⁶, 88×10⁶, 89×10⁶, 90×10⁶, 91×10⁶, 92×10⁶, 93×10⁶, 94×10⁶, 95×10⁶, 96×10⁶, 97×10⁶, 98×10⁶, 99×10⁶, 100×10⁶, 101×10⁶, 102×10⁶, 103×10⁶, 104×10⁶, 105×10⁶, 106×10⁶, 107×10⁶, 108×10⁶, 109×10⁶, 110×10⁶, 111×10⁶, 112×10⁶, 113×10⁶, 114×10⁶, 115×10⁶, 116×10⁶, 117×10⁶, 118×10⁶, 119×10⁶, 120×10⁶, 121×10⁶, 122×10⁶, 123×10⁶, 124×10⁶, 125×10⁶, 126×10⁶, 127×10⁶, 128×10⁶, 129×10⁶, 130×10⁶, 131×10⁶, 132×10⁶, 133×10⁶, 134×10⁶, 135×10⁶, 136×10⁶, 137×10⁶, 138×10⁶, 139×10⁶, 140×10⁶, 141×10⁶, 142×10⁶, 143×10⁶, 144×10⁶, 145×10⁶, 146×10⁶, 147×10⁶, 148×10⁶, 149×10⁶, 150×10⁶, 151×10⁶, 152×10⁶, 153×10⁶, 154×10⁶, 155×10⁶, 156×10⁶, 157×10⁶, 158×10⁶, 159×10⁶, 160×10⁶, 161×10⁶, 162×10⁶, 163×10⁶, 164×10⁶, 165×10⁶, 166×10⁶, 167×10⁶, 168×10⁶, 169×10⁶, 170×10⁶, 171×10⁶, 172×10⁶, 173×10⁶, 174×10⁶, 175×10⁶, 176×10⁶, 177×10⁶, 178×10⁶, 179×10⁶, 180×10⁶, 181×10⁶, 182×10⁶, 183×10⁶, 184×10⁶, 185×10⁶, 186×10⁶, 187×10⁶, 188×10⁶, 189×10⁶, 190×10⁶, 191×10⁶, 192×10⁶, 193×10⁶, 194×10⁶, 195×10⁶, 196×10⁶, 197×10⁶, 198×10⁶, 199×10⁶, or 200×10⁶) viable CD19 specific CAR-T cells. In some embodiments, the dose is a therapeutically effective amount of viable CD19 specific CAR-T cells. In other embodiments, the dose is a clinically effective amount of viable CD19 specific CAR-T cells. In some embodiments, the viable CD19 specific CAR-T cells include CD19 specific CAR expressing CD4+ T cells and CD19 specific CAR expressing CD8+ T cells at a ratio of about 1:1. In some embodiments, the CD19 specific CAR is the same CD19 specific CAR as lisocabtagene maraleucel (BREYANZI®), a structural equivalent thereof, or a functional equivalent thereof.

In some embodiments, a single dose of any of the CD19 specific CAR-T cells described herein includes about 2×10⁸ viable CD19 specific CAR-T cells. In some embodiments, a single infusion bag of any of the CD19 specific CAR-T cells described herein includes about 2×10⁸ viable CD19 specific CAR-T cells in a cell suspension of about 68 mL. In some embodiments, the CD19 specific CAR is the same CD19 specific CAR as axicabtagene ciloleucel (YESCARTA®), a structural equivalent thereof, or a functional equivalent thereof.

In some embodiments, a single dose of any of the CD19 specific CAR-T cells described herein includes about 2×10⁸ viable CD19 specific CAR-T cells. In some embodiments, a single infusion bag of any of the CD19 specific CAR-T cells described herein includes about 2×10⁸ viable CD19 specific CAR-T cells in a cell suspension of about 68 mL. In some embodiments, the CD19 specific CAR is the same CD19 specific CAR as brexucabtagene autoleucel (TECARTUS®), a structural equivalent thereof, or a functional equivalent thereof.

In some embodiments, a single dose of any of the CD19 specific CAR-T cells described herein includes about 0.2×10⁶ to about 5.0×10⁶ (e.g., about 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3.0×10⁶, 3.1×10⁶, 3.2×10⁶, 3.3×10⁶, 3.4×10⁶, 3.5×10⁶, 3.6×10⁶, 3.7×10⁶, 3.8×10⁶, 3.9×10⁶, 4.0×10⁶, 4.1×10⁶, 4.2×10⁶, 4.3×10⁶, 4.4×10⁶, 4.5×10⁶, 4.6×10⁶, 4.7×10⁶, 4.8×10⁶, 4.9×10⁶, or 5.0×10⁶) viable CD19 specific CAR-T cells per kg of body weight for a subject with a body weight of 50 kg or less. In some embodiments, a single dose of any of the CD19 specific CAR-T cells described herein includes about 0.1×10⁸ to about 2.5×10⁸ (e.g., about 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2.0×10⁶, 2.1×10⁶, 2.2×10⁶, 2.3×10⁶, 2.4×10⁶, or 2.5×10⁶) viable CD19 specific CAR-T cells per kg of body weight for a subject with a body weight of more than 50 kg. In some embodiments, a single dose of any of the CD19 specific CAR-T cells described herein includes about 0.6×10⁸ to about 6.0×10⁸ (e.g., about 0.6×10⁸, 0.7×10⁸, 0.8×10⁸, 0.9×10⁸, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD19 specific CAR-T cells. In some embodiments, a single infusion bag of any of the CD19 specific CAR-T cells described herein includes about 0.6×10⁸ to about 6.0×10⁸ (e.g., about 0.6×10⁸, 0.7×10⁸, 0.8×10⁸, 0.9×10⁸, 1.0×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2.0×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3.0×10⁸, 3.1×10⁸, 3.2×10⁸, 3.3×10⁸, 3.4×10⁸, 3.5×10⁸, 3.6×10⁸, 3.7×10⁸, 3.8×10⁸, 3.9×10⁸, 4.0×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5.0×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, or 6.0×10⁸) viable CD19 specific CAR-T cells in a cell suspension of from about 10 mL to about 50 mL. In some embodiments, the dose is a therapeutically effective amount of viable CD19 specific CAR-T cells. In other embodiments, the dose is a clinically effective amount of viable CD19 specific CAR-T cells. In some embodiments, the CD19 specific CAR of the cells is the same CD19 specific CAR as tisagenlecleucel (KYMRIAH®), a structural equivalent thereof, or a functional equivalent thereof.

### 2. Pharmaceutical Compositions

For therapeutic application, cells prepared according to the disclosed methods can typically be supplied in the form of a pharmaceutical composition comprising an isotonic excipient and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells can be packaged in a device or container suitable for distribution or clinical use.

In some embodiments, pharmaceutical compositions of the present disclosure comprise a population of engineered hypoimmunogenic T cells comprising: (a) reduced expression of Beta-2-Microglobulin (B2M), Class II Transactivator (CIITA), and T cell receptor alpha (TRAC) relative to a control T cell, (b) increased expression of CD47 encoded by a first exogenous polynucleotide relative to the control T cell, and (c) expression of a CD19-specific chimeric antigen receptor (CAR) encoded by a second exogenous polynucleotide, wherein the population of engineered hypoimmunogenic T cells are allogeneic to the subject receiving the pharmaceutical composition.

a. Pharmaceutically Acceptable Carriers

In some embodiments, a pharmaceutical composition provided herein further include a pharmaceutically acceptable carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); salts such as sodium chloride; and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline).

In some embodiments, a pharmaceutical composition includes one or more electrolyte base solutions selected from the group consisting of lactated CryoStor®, Ringer's solution, PlasmaLyte-A™, Iscove's Modified Dulbecco's Medium, Normosol-R™, Veen-D™, Polysal® and Hank's Balanced Salt Solution (containing no phenol red). These base solutions closely approximate the composition of extracellular mammalian physiological fluids.

In some embodiments, a pharmaceutical composition includes one or more cryoprotective agents selected from the group consisting of arabinogalactan, glycerol, polyvinylpyrrolidone (PVP), dextrose, dextran, trehalose, sucrose, raffinose, hydroxyethyl starch (HES), propylene glycol, human serum albumin (HSA), and dimethylsulfoxide (DMSO). In some embodiments, the pharmaceutically acceptable buffer is neutral buffer saline or phosphate buffered saline. In some embodiments, pharmaceutical compositions provided herein include one or more of CryoStor® CSB, Plasma-Lyte-A™, HSA, DMSO, and trehalose.

CryoStor® is an intracellular-like optimized solution containing osmotic/oncotic agents, free radical scavengers, and energy sources to minimize apoptosis, minimize ischemia/reperfusion injury and maximize the post-thaw recovery of the greatest numbers of viable, functional cells. CryoStor® is serum- and protein-free, and nonimmunogenic. CryoStor® is cGMP-manufactured from raw materials of USP grade or higher. CryoStor® is a family of solutions pre-formulated with 0%, 2%, 5% or 10% DMSO. CryoStor® CSB is a DMSO-free version of CryoStor®. In some embodiments, a pharmaceutical composition includes a base solution of CryoStor® CSB at a concentration of about 0-100%, 5-95%, 10-90%, 15-85%, 20-80%, 30-80%, 40-80%, 50-80%, 60-80%, 70-80%, 25-75%, 30-70%, 35-65%, 40-60%, or 45-55% w/w. In some embodiments, a pharmaceutical composition includes a base solution of CryoStor® CSB at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

PlasmaLyte-ATM is a non-polymeric plasma expander and contains essential salts and nutrients similar to those found in culture medium but does not contain additional constituents found in tissue culture medium which are not approved for human infusion, e.g., phenol red, or are unavailable in U.S.P. grade. PlasmaLyte-ATM contains about 140 mEq/liter of sodium (Na), about 5 mEq/liter of potassium (K), about 3 mEq/liter of magnesium (Mg), about 98 mEq/liter of chloride (Cl), about 27 mEq/liter of acetate, and about 23 mEq/liter of gluconate. (PlasmaLyte-ATM is commercially available from Baxter, Hyland Division, Glendale Calif., product No. 2B2543). In some embodiments, a pharmaceutical composition includes a base solution of PlasmaLyte-ATM at a concentration of about 0-100%, 5-95%, 10-90%, 15-85%, 15-80%, 15-75%, 15-70%, 15-65%, 15-60%, 15-55%, 15-50%, 15-45%, 15-40%, 15-35%, 15-30%, 15-25%, 20-80%, 20-75%, 20-70%, 20-65%, 20-60%, 20-55%, 20-50%, 20-45%, 20-40%, 20-35%, 20-30%, 25-75%, 30-70%, 35-65%, 40-60%, or 45-55% w/w. In some embodiments, the pharmaceutical composition includes a base solution of PlasmaLyte-A™ at a concentration of about 0%, 5%, 10%, 15%, 20%, 25%, 30%, 35%, 40%, 45%, 50%, 55%, 60%, 65%, 70%, 75%, 80%, 85%, 90%, 95%, or 100% w/w.

In some embodiments, a pharmaceutical composition includes a DMSO concentration (v/v) of at least about 1%, at least about 2%, at least about 3%, at least about 4%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%. In some embodiments, a pharmaceutical composition includes a DMSO concentration (v/v) of about 7.5%.

In some embodiments, a pharmaceutical composition includes human serum albumin (HSA) at a concentration of about 0-10%, 0.3-9.3%, 0.3-8.3%, 0.3-7.3%, 0.3-6.3%, 0.3-5.3%, 0.3-4.3%, 0.3-3.3%, 0.3-2.3%, 0.3-1.3%, 0.6-8.3%, 0.9-7.3%, 1.2-6.3%, 1.5-5.3%, 1.8-4.3%, or 2.1-3.3% w/v. In some embodiments, a pharmaceutical composition comprises human serum albumin (HSA) at a concentration (w/v) of at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%. In some embodiments, a pharmaceutical composition includes HSA at a concentration of about 0%, 0.3%, 0.6%, 0.9%, 1.2%, 1.5%, 1.8%, 2.1%, 2.4%, 2.7%, 3.0%, 3.3%, 3.6%, 3.9%, 4.3%, 4.6%, 4.9%, 5.3%, 5.6%, 5.9%, 6.3%, 6.6%, 6.9%, 7.3%, 7.6%, 7.9%, 8.3%, 8.6%, 8.9%, 9.3%, 9.6%, 9.9%, or 10% w/v. In some embodiments, a pharmaceutical composition comprises HSA at a concentration (w/v) of about 0.3%.

In some embodiments, a pharmaceutical composition includes dimethyl sulfoxide (DMSO) at a concentration of about 0-10%, 0.5-9.5%, 1-9%, 1.5-8.5%, 2-8%, 3-8%, 4-8%, 5-8%, 6-8%, 7-8%, 2.5-7.5%, 3-7%, 3.5-6.5%, 4-6%, or 4.5-5.5% v/v. In some embodiments, a pharmaceutical composition includes HSA at a concentration of about 0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, or 10.0% v/v.

In some embodiments, a pharmaceutical composition includes dimethyl sulfoxide (DMSO) at a concentration of about 0-10%, 0.5-9.5%, 1-9%, 1.5-8.5%, 2-8%, 3-8%, 4-8%, 5-8%, 6-8%, 7-8%, 2.5-7.5%, 3-7%, 3.5-6.5%, 4-6%, or 4.5-5.5% v/v. In some embodiments, a pharmaceutical composition includes HSA at a concentration of about 0%, 0.25%, 0.5%, 0.75%, 1.0%, 1.25%, 1.5%, 1.75%, 2.0%, 2.25%, 2.5%, 2.75%, 3.0%, 3.25%, 3.5%, 3.75%, 4.0%, 4.25%, 4.5%, 4.75%, 5.0%, 5.25%, 5.5%, 5.75%, 6.0%, 6.25%, 6.5%, 6.75%, 7.0%, 7.25%, 7.5%, 7.75%, 8.0%, 8.25%, 8.5%, 8.75%, 9.0%, 9.25%, 9.5%, 9.75%, or 10.0% w/v.

In some embodiments, a pharmaceutical composition includes trehalose at a concentration of about 0-500 mM, 50-450 mM, 100-400 mM, 150-350 mM, or 200-300 mM. In some embodiments, a pharmaceutical composition includes trehalose at a concentration of about 0 mM, 10 mM, 20 mM, 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, or 500 mM.

Exemplary pharmaceutical composition components are shown in Table 1.

TABLE 1
Exemplary pharmaceutical composition components

| | | | Additional | |
|---|---|---|---|---|
| Formulation | Base Solution | [DMSO] | [HSA]* | [trehalose] |
| A | 75% CroStor ® | 7.5% | 0.3% | |
| B | CSB + 25% | 3.75% | 0.3% | |
| C | PlasmaLyte A ™ + | | 5.3% | |
| D | 1.2% HSA | | 0.3% | 250 mM |
| E | 100% | 7.5% | 0.3% | |
| F | PlasmaLyte A ™ + | 7.5% | 5.3% | |
| G | 1.2% HSA | 7.5% | 5.3% | 250 mM |

*HSA in addition to PlasmaLyte.

In some embodiments, a pharmaceutical composition comprises hypoimmunogenic cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethyl sulfoxide (DMSO).

In some embodiments, a pharmaceutical composition comprises hypoimmunogenic cells described herein and a pharmaceutically acceptable carrier comprising 75% Cryostor CS10, 25% (v/v) Plasma-Lyte A, 7.5% (v/v) DMSO, and 1.2% (v/v) HSA.

In some embodiments, a pharmaceutical composition comprises hypoimmunogenic cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (w/v) of 25% human serum albumin (HSA), and 7.5% (v/v) dimethyl sulfoxide (DMSO).

In some embodiments, a pharmaceutical composition comprises hypoimmunogenic cells described herein and a pharmaceutically acceptable carrier comprising 75% Cryostor CS10, 25% (v/v) Plasma-Lyte A, 7.5% (v/v) DMSO, and 1.2% (w/v) HSA.

### 3. Diseases, Disorders and Conditions to be Treated

B cell lymphomas encompass a group of lymphoma subtypes categorized within 2 broad categories: non-Hodgkin lymphoma (NHL) and Hodgkin lymphoma (HL), with NHLs being the majority of diagnosed cases. The therapeutic landscape varies for each distinct tumor subtype, but generally there are limited effective therapies in relapsed and/or refractory (r/r) settings across B cell lymphomas (National Comprehensive Cancer Network [NCCN] 2021). Despite recent advancement, NHL remains an unmet medical need. In 2021 American Cancer Society estimated that 82,000 cases of NHL would be diagnosed, with approximately 20,000 attributed deaths in the US in 2021 (American Cancer Society 2021). In 2020, there were 123,000 new cases of NHL reported, with 50,000 attributed deaths in Europe (World Health Organization 2020). NHL is a heterogeneous group of cancers originating in B lymphocytes, T lymphocytes, or natural killer (NK) cells. In the US, B cell lymphomas represent approximately 85% of all NHL cases (American Cancer Society 2019). Subtypes of NHL include large B cell lymphomas (LBCL), such as diffuse LBCL (DLBCL), high-grade B cell lymphoma (HGBCL), and primary mediastinal B cell lymphoma (PMBCL), along with mantle lymphoma (MCL). Subtypes of indolent NHL (iNHL) include follicular lymphoma (FL) and marginal zone lymphoma (MZL).

DLBCL is the most aggressive NHL in adults, accounting for a third of all NHL cases, about 25,000 annually. In the US, the incidence of DLBCL is approximately 7 cases per 100,000 persons per year, with Caucasian-Americans having higher rates than other racial groups. There is a male predominance, and incidence increases with age with a median age of diagnosis of 64 years (Morton 2006, Shenoy 2011). Gene expression profiling has identified 2 distinct subtypes within DLBCL that are managed with similar standard of care—germinal center B cell (GCB) subtype and activated B cell (ABC) subtype. GCB subtype is associated with improved outcomes to therapy. MYC rearrangements are found in 5% to 8% of DLBCL patients and correlate with higher risk of disease progression (NCCN 2021).

HGBCL includes DLBCLs with MYC and B cell lymphoma 2 (BCL2) and/or B cell lymphoma 6 (BCL6) rearrangements (known as “double-hit” or “triple-hit lymphomas” based on the number of translocations). Generally, MYC rearrangements in DLBCLs are associated with a poor prognosis that is worsened in cases of concomitant BCL2 and/or BCL6 alterations. The majority of HGBCL are germinal center B cell like lymphomas and present with worse outcomes than DLBCL, though treatment algorithms are shared. Exceptions to HGBCL include cases of follicular or lymphoblastic lymphoma or HGBCL, not otherwise specified (NOS), which contains blastoid-appearing LBCLs or cases that lack MYC and BCL2 or BCL6 translocations (NCCN 2021, Swerdlow 2016).

PMBCL has distinct clinical, pathological, and molecular characteristics compared with DLBCL and represents approximately 2% to 4% of all patients with NHL (Bhatt 2015, Dabrowska-Iwanicka 2014). Initial treatment for PMBCL is similar to DLBCL (i.e., anthracycline-containing regimens with rituximab with or without involved-field radiotherapy), which is curative in the majority of cases. Chemosensitive patients who relapse may respond to further cytotoxic therapy followed by autologous stem cell transplant (ASCT); nonresponders have limited therapeutic options that lack durability (NCCN 2021).

Follicular lymphoma 3B (FL3B) is the most aggressive variant of FL, the most common subtype of indolent non-Hodgkin lymphoma. The majority of cases contain a t(14; 18) translocation, which juxtaposes BCL2 with the IGH locus. Pathologic grading of FL according to the number of centroblasts is a clinical predictor of outcome. Grade 3 follicular lymphoma is subclassified into FL3A (centrocytes still present) or FL3B (sheets of centroblasts), which have similar survival outcomes but may be managed differently. FL3B is commonly treated according to treatment recommendations for DLBCL (NCCN 2021).

Treatment options for LBCL include rituximab-based chemotherapy (R-CHOP) and ASCT. Most patients with DLBCL respond well to rituximab-based immunochemotherapy. However, outcome remains poor for patients with r/r DLBCL (1-year and 2-year overall survival [OS] rates of 23% and 16%, respectively). While ASCT confers durable responses, approximately half of DLBCL patients are ineligible for treatment and the majority of ASCT-treated patients will experience disease progression within 3 years (Sehn 2021, Crump 2014, Gisselbrecht 2010). Those patients who do not receive ASCT, either because they are not eligible even after showing response to the second-line chemotherapy or because they do not have responses to the second-line chemotherapy (refractory), have a very poor prognosis with a median OS of 4.4 months. For patients who have relapsed disease within 12 months after ASCT or primary refractory disease, only 26% of them respond to the next line of therapy (third line) with 7% of complete response and median OS of 6.2 months. Patients whose disease progresses after 2 or more prior lines of systemic therapy regimens are unlikely to benefit further from currently available systemic therapy options, unless they have experienced a long disease-free interval. Thus, there is considerable unmet medical need for DLBCL patients who do not respond to first-line immunochemotherapy or to subsequent courses of combination chemotherapy (NCCN 2021).

To assess the outcomes of patients with r/r LBCL, the multicenter retrospective study SCHOLAR-1 analyzed data from 2 large Phase 3 studies involving 636 patients. Results showed that refractory DLBCL patients have an objective response rate (ORR) of 26%, a complete response (CR) rate of 7%, and a median OS of 6.3 months (Crump 2017, Gisselbrecht 2012, Van Den Neste 2016). Poor outcomes were observed across all refractory subgroups (primary refractory, refractory to second-line or higher therapy, or relapse <1 year after ASCT) and regardless of disease stage, suggesting that these patients form a single population who lack effective treatment.

Recently, new therapeutic options have been approved for r/r LBCL. In 2020, FDA granted accelerated approval for tafasitamab in combination with lenalidomide for the treatment of adult patients with r/r DLBCL, NOS, including DLBCL arising from low-grade lymphoma, and who are not eligible for ASCT. Among 71 subjects with r/r DLBCL treated with tafasitamab plus lenalidomide, 37% achieved a CR and 18% achieved a partial response (PR) (MONJUVI Prescribing Information 2020). The estimated median duration of response (DOR) was 21.7 months. EMA granted approval for polatuzumab vedotin, a CD79b-directed antibody-drug conjugate, in combination with bendamustine and rituximab (BR) for treatment of r/r DLBCL patients who are not candidates for stem cell transplant (SCT). Polatuzumab vedotin plus BR resulted in an ORR of 45% (BR alone: 18%) and a CR rate of 40% (BR alone: 18%) (POLIVY Prescribing Information 2019, Sehn 2018). Pembrolizumab, a humanized monoclonal antibody (mAb) against programmed cell death protein 1 (PD-1), received accelerated approval by FDA for the treatment of PMBCL refractory to treatment or that has relapsed after 2 or more prior lines of therapy (Armand 2019, KEYTRUDA Prescribing Information 2019). The single-arm, multicenter KEYNOTE-170 study of pembrolizumab enrolled 53 patients with a median of 3 prior lines of therapy, and results showed an ORR of 45% (CR: 13%) with the median OS not reached.

Also recently, autologous CD19-directed chimeric antigen receptor (CAR) T cell therapies (tisagenlecleucel, axicabtagene ciloleucel, and lisocabtagene maraleucel) have been approved as a second-line and/or third-line therapy for patients with r/r LBCL. These therapies offer promising options for these patients, with CR rates ranging from 39% to 60% and median DOR from 9.2 months to 16.7 months. Key toxicities include cytokine release syndrome (CRS) and neurotoxicity with rates of Grade 3 and higher events ranging from 4% to 23% and 11% to 31%, respectively (KYMRIAH® Prescribing Information 2021; YESCARTA® Prescribing Information 2021; BREYANZI® Prescribing Information 2021). Three recent randomized trials evaluated CD19-directed CAR T cell therapies as second-line therapy for LBCL (Locke 2022, Bishop 2021, Kamdar 2022). The ZUMA-7 study demonstrated superiority of axicabtagene ciloleucel over standard of care (CR 65% vs 32%, event-free survival (EFS hazard ratio [HR] 0.40, p<0.001) and TRANSFORM demonstrated superiority of lisocabtagene maraleucel over standard of care (EFS 10.1 months vs 2.3 months, HR 0.35). However, the BELINDA study failed to demonstrate superiority of tisagenlecleucel versus standard of care (EFS HR 1.07, p=0.69) driven in part by differences in trial design and treatment delays associated with manufacturing. Despite their approvals, the manufacturing and logistical challenges of autologous CAR T cell therapies make it difficult for these therapies to be widely used (Gajra 2022). Therefore, considerable unmet medical need still exists for patients with r/r LBCL, and there is urgent need to develop innovative medicines including more readily available (i.e., off-the-shelf) CAR T cell therapies.

iNHLs include FL and MZL. FL is the most common indolent form of NHL, accounting for between 10% and 22% of all cases (NCCN 2021, Teras 2016). Although newly diagnosed, low-grade, limited-stage FL is often responsive to a variety of first-line treatments; the disease is characterized by a pattern of relapsing and remitting disease. Transformation from FL to DLBCL (transformed FL [tFL]) occurs at an annual rate of approximately 3% for 15 years and is associated with poor outcomes (Montoto 2007).

Outcomes in FL are mixed. High-risk patients with ≥3 Follicular Lymphoma International Prognostic Index (FLIPI) criteria have a 5-year OS rate of 53%, whereas low-risk patients with ≤1 FLIPI criterion have a 5-year survival rate of over 90%. Advanced-stage FL generally requires multiple successive lines of therapy leading to progressively shorter remission periods, chemorefractory disease, transformation to DLBCL, or death due to repeated treatment-related toxicities (Cheah 2018). Thus, regardless of the disease stage at diagnosis, most patients with FL ultimately experience disease relapse, even after a long-term response to first-line therapy, representing a significant unmet need.

First-line therapies for FL include R-CHOP, R-CVP, bendamustine+CD20 mAb, and lenalidomide+rituximab. High-dose (chemo) therapy/auto-SCT as a consolidative therapy for patients with FL who are in second or third remission is a potential therapeutic option (NCCN 2021). Allogeneic (allo)-SCT may offer improved relapse rates and disease control compared with auto-SCT; however, allo-SCT is associated with higher nonrelapse mortality rates and is not a viable treatment option for most patients with FL (Freedman 2018). New targeted agents have been developed to treat patients who progress following systemic chemotherapy and rituximab-containing regimens. The “enhancer of zeste homolog” (EZH)2 histone methyl transferase inhibitor tazemetostat was granted accelerated approval by the FDA for adult subjects with EZH2 mutant r/r FL who have received ≥2 prior therapies (or have no satisfactory alternative treatment options) (TAZVERIK 2020). Additionally, phosphatidylinositol 3-kinase inhibitors (i.e., idelalisib, copanlisib, and duvelisib) have been approved for r/r FL after 2 prior therapies.

CD19-directed CAR T cell therapy has also been utilized in the treatment of r/r FL. In a study of 21 subjects with r/r FL (8 with FL and 13 with tFL) treated with an investigational autologous CD19-directed CAR T therapy, CR rates were 88% and 46% for FL and tFL patients, respectively. All patients with FL who achieved CR remained in remission at a median follow-up of 24 months while the median duration of response for tFL was 10.2 months (Hirayama 2019). YESCARTA has received accelerated approval from the FDA for treatment of adult patients with r/r FL after 2 or more lines of systemic therapy. Approval was based on Study KTEC19105 (ZUMA5), where treatment with YESCARTA among 81 subjects with r/r FL resulted in an ORR of 91% and a CR rate of 60% (YESCARTA 2021). Subjects had at least 9 months of potential follow-up and a median of 3 prior lines of therapy. Among the 74 responding subjects, the median DOR was not estimable with a median follow-up time of 14.5 months.

These data highlight the need for novel treatment options for patients with r/r FL who have received 2 or more prior lines of therapy and demonstrate the promise of durable responses with CAR T cell therapy.

MZL is a rare form of NHL originating in the marginal zone of B cells (Kahl 2008). The low incidence rates and heterogeneous presentation render large-scale clinical studies of MZL challenging. First-line treatment options for MZL include R-CHOP, R-CVP, rituximab, and bendamustine+rituximab. Generally, patients respond to front-line therapy; however, patients with r/r MZL have few therapeutic options, including Bruton tyrosine kinase (BTK) inhibitors, phosphoinositide 3-kinase (PI3K) inhibitors, and lenalidomide plus rituximab (NCCN 2021).

Ibrutinib, a Bruton tyrosine kinase inhibitor (BTKi), is approved for patients with MZL who have received at least 1 prior anti-CD20-based therapy (IMBRUVICA 2022). Accelerated approval of ibrutinib was granted based on a prospective, multicenter, open-label Phase 2 study of 63 patients with MZL that reported an ORR of 46% and CR rate of 3.2%. After a median follow-up time of 19.4 months, the DOR was not reached. Lenalidomide, a thalidomide analogue, is approved for the treatment of previously treated MZL in combination with a rituximab product (REVLIMID 2019). In a pivotal study, among patients with MZL, the ORR by Independent Review Committee assessment was 65% (CR 29%) (Thieblemont 2019). After a median follow-up of 27.9 months, the median progression-free survival (PFS) was 20.2 months.

Preliminary results with CD19-directed CAR T cell therapy have also demonstrated promising results in r/r MZL. Treatment with YESCARTA among 7 subjects with r/r MZL yielded an ORR of 86% and a CR rate of 71% (Jacobson 2020). To this end, effective modalities to keep patients disease-free are warranted in patients relapsing after 2 lines of therapy and CAR T therapies demonstrate the promise of durable responses. MCL comprises approximately 6% of all newly diagnosed NHL cases.

MCL carries the unfavorable characteristics of both indolent and aggressive NHL subtypes due to the incurability of disease with conventional chemotherapy and a generally more aggressive disease course compared to indolent NHL. The MCL International Prognostic Index (MIPI) integrates clinical variables including age, performance status, lactate dehydrogenase, and white blood cell count to stratify patients into low, intermediate, and high-risk categories. High proliferation index (based on KI-67), blastoid/pleomorphic histology, complex karyotype, and TP53 mutations predict poor outcomes for patients (Romancik 2022).

Initial therapy for MCL often involves intensive, cytarabine-containing induction chemotherapy regimens such as RDHA, HyperCVAD, or NORDIC, followed by consolidation with an autologous stem cell transplant (ASCT) and 3 years of maintenance rituximab. Older patients and those with medical comorbidities may instead receive less intensive induction regimens such as bendamustine plus rituximab, R-CHOP, or lenalidomide plus rituximab. Regardless of initial therapy, most patients will eventually relapse and require multiple lines of subsequent therapies (NCCN 2021).

Targeted agents such as the BTK inhibitors, bortezomib, lenalidomide, and venetoclax are currently available to treat second-line r/r MCL. Three irreversible BTK inhibitors (ibrutinib, acalabrutinib, and zanubrutinib) are FDA approved for the treatment of r/r MCL. Ibrutinib was the first BTK inhibitor approval based on a pivotal Phase 2 in r/r MCL demonstrating 68% ORR, 21% CR, median DOR 17.5 months, and median 2-year OS of 47% (Wang 2013). Subsequently, more selective, second-generation BTK inhibitors (acalabrutinib and zanubrutinib) were approved. Acalabrutinib's approval was based on the Phase 2 ACE-LY-2004 trial with 124 r/r MCL patients demonstrating 81% ORR, 40% CR, DOR 26 months and median PFS 19.5 months (Wang 2018). Zanubrutinib's approval was based on the pivotal BGB-3111-206 and BGB3111-AU-003 trials demonstrating an 84% ORR and 25% to 69% CR (Tam 2021). Lenalidomide is also FDA approved for the treatment of r/r MCL. When sequenced after bortezomib, lenalidomide monotherapy is associated with an 28% ORR, 7.5% CR, and 16.6 month DOR (Goy 2013).

Brexucabtagene autoleucel (formerly KTE-X19) is a CD19 autologous CAR T product with a CD28 co-stimulatory domain that is now FDA approved for the treatment of r/r MCL based on the results of the ZUMA-2 trial (Wang 2020). In this study, the ORR was 85% (59% CR) in the intention-to-treat population, with 12-month PFS and OS rates of 61% and 83%, respectively. Notably, responses were observed in patients with high-risk disease characteristics, including pleomorphic/blastoid morphology, TP53 aberrations, and high Ki-67. Lisocabtagene maraleucel (liso-cel; formerly JCAR017) is an autologous CD19-directed CAR T product that contains a 4-1BB signaling domain and is administered by sequential infusions of the CD8+ and CD4+ CAR T cells. Liso-cel is currently FDA approved for the treatment of r/r large B cell lymphomas based on the results of the TRANSCEND NHL 001 trial. While this product is not yet approved for patients with MCL, the Phase 1 portion of the TRANSCEND trial included 32 patients with r/r MCL and reported an ORR of 84% (59% CR) in this population across all dose levels (Abramson 2020).

Despite the approval of brexucabtagene autoleucel, the product safety profile, manufacturing, and logistical challenges make it difficult for CD19-directed CAR T therapies to be widely used in MCL. Therefore, considerable unmet medical need still exists for patients with r/r MCL, and there is urgent need to develop innovative medicines including more readily available (i.e., off-the-shelf) CAR T cell therapies.

CLL is the most common leukemia in the USA and among the most common globally. CLL can vary from an indolent disease to very aggressive with potential for transformation to high-grade lymphoma (i.e., Richter's transformation; NCCN 2022). Previous treatments for CLL included alkylating agents such as chlorambucil and cyclophosphamide and purine nucleosides such as fludarabine, pentostatin, and cladribine. Historically, chemoimmunotherapy regimens like fludarabine, cyclophosphamide and rituximab (FCR), bendamustine plus rituximab (BR), chlorambucil, alemtuzumab, and lenalidomide were the mainstays of CLL treatment in both the front-line and relapsed settings. Over the past decade, BTK inhibitors (ibrutinib, acalabrutinib, zanubrutinib, and pirtobrutinib) BCL2 antagonists (venetoclax), and PI3K inhibitors (idelalisib and duvelisib), given as single agent or in combination with anti-CD20 antibodies (e.g., obinutuzumab), have emerged as new standards of care for front-line CLL. Front-line therapy often leads to durable responses, but despite significant progress, CLL remains incurable. Relapses frequently occur in groups with high-risk features, such as those with TP53 mutation, unmutated immunoglobulin heavy-chain variable (IGHV), deletion 11q or deletion 17p, or presence of complex karyotype. For this reason, chemoimmunotherapies are not recommended for patients with del(17p)/TP53 mutation due to low response rates (Rainone 2022).

Targeted therapies used in second-line and subsequent settings for CLL include BTK inhibitors, BCL2 antagonists, anti-CD20 antibodies, and PI3K inhibitors. The Phase 3 DUO study of duvelisib, a PI3K delta inhibitor, in 319 patients with r/r CLL demonstrated superior PFS to ofatumumab (13.3 months vs 9.9 months, HR=0.52, p<0.001) (Flinn 2018). The Phase 3 IDELA trial in r/r CLL evaluated the PI3K inhibitor idelalisib plus rituximab vs placebo plus rituximab and demonstrated superior efficacy in PFS and OS of the idelalisib-containing arm (Sharman 2019). Few treatment options exist for patients who progress on these targeted therapies and therefore a significant unmet medical need still exists for CLL. Allogeneic stem cell transplantation may provide a chance of cure for some patients but comes with high toxicities and may not be suitable for older CLL patients.

Recently, CAR T therapies have demonstrated encouraging durable responses for CLL patients. TRANSCEND CLL 004 is the first multicenter study of an CD19-directed CAR T cell therapy (lisocabtagene maraleucel [liso-cel]) for r/r CLL (Siddiqi 2022). Subjects had a median of 4 prior therapies and 83% had high-risk features, including mutated TP53 and del(17p). CRS was reported in 74% of subjects (Grade 3 in 9%), and neurological events in 39% of subjects (Grade 3/4 in 22%). Of 22 efficacy-evaluable subjects, 82% and 45% achieved overall and complete responses, respectively. Of 20 minimum residual disease (MRD)-evaluable subjects, 75% and 65% achieved undetectable MRD in blood and marrow, respectively. At a median follow-up of 24 months, the duration of response was not reached and median PFS was 18 months.

In some embodiments, a subject of the present disclosure has or is suspected of having a B cell malignancy. In some embodiments, a B cell malignancy is selected from the group consisting of: Non-Hodgkin's Lymphoma (NHL), Chronic Lymphocytic Leukemia (CLL), large B cell lymphoma (LBCL), diffuse LBCL (DLBCL), high-grade B cell lymphoma (HGBCL), primary mediastinal B cell lymphoma (PMBCL), mantle cell lymphoma (MCL), follicular lymphoma (FL), marginal zone lymphoma (MZL), or small lymphocytic lymphoma (SLL).

In some embodiments, a subject of the present disclosure has or is suspected of having a CLL with high-risk features, such as those with TP53 mutation, unmutated immunoglobulin heavy-chain variable (IGHV), deletion 11q or deletion 17p, or presence of complex karyotype. In some embodiments, a subject of the present disclosure who has or is suspected of having a CLL with high-risk features is treated with a composition of the present disclosure as a first-line therapy. In some embodiments, a method of the present disclosure comprises detecting one or more biomarkers in a sample from a subject of the present disclosure and identifying the subject as being a high risk CCL patient based on the one or more biomarkers. In some embodiments, the one or more biomarkers are selected from the group consisting of: del(17p), mutated TP53, unmutated immunoglobulin heavy-chain variable (IGHV) gene, and complex karyotype.

In some embodiments, a subject of the present disclosure who has or is suspected of having multiple myeloma is treated with a composition of the present disclosure as a first-line therapy.

B cells are thought to have multiple functions that include antigen presentation, cytokine secretion and autoantibody production. There is a plethora of autoantibodies associated with systemic lupus erythematosus (SLE), suggesting continued B cell stimulation and antigen processing in disease. Not all autoantibodies are known to be pathogenic, and autoantibodies are known to appear in advance of clinical disease (Arbuckle 2003). Several studies have highlighted B cell activating factor (BAFF) as having a crucial role in regulating B cell maturation, survival, and function (Vincent 2012). Elevated levels of BAFF have been reported in serum of patients suffering from autoimmune diseases, including SLE, suggesting a role in disease pathogenesis (Renaudineau 2004). Belimumab, a fully humanized mAb which neutralizes BAFF, led to impaired B cell survival and reduction in peripheral B cell numbers in SLE patients (Vincent 2012; Basta 2020). In the Belimumab International Study in Lupus Nephritis 2-year, randomized, placebo-controlled, Phase 3 trial, belimumab plus standard therapies (mycophenolate mofetil [MMF] or cyclophosphamide followed by azathioprine) were administered to adult patients with active lupus nephritis (LN). Compared with placebo, significantly more patients randomized to belimumab achieved a primary efficacy renal response (43% vs. 32%; p=0.03) based on reductions in urinary protein to creatine ratio (UPCR) and estimated glomerular filtration rate (eGFR), and a complete renal response (30% vs. 20%; p=0.02) using endpoints consistent with the proposed SC291-102 study. Renal-related events or death were also lower in the belimumab group compared with placebo, with almost 50% lower renal-related events among patients who received belimumab than those who received standard therapies alone (Furie 2020).

Rituximab, initially approved by the FDA in 1997 for the treatment of r/r NHL, is a mAb that selectively targets the B cell specific surface molecule CD20. The absence of CD20 antigen expression on the cell surface of hematopoietic stem cells, normal plasma cells, or other normal tissues, allows for selective depletion of B cells with rituximab (Fervenza 2008). Murine models have helped establish that CD20+ and circulating B cells are rapidly eliminated by CD20-targeting mAbs, through the reticuloendothelial system. However, B cells in some locations, such as marginal zones, are not depleted by mAbs targeting CD20, and the reliance on the reticuloendothelial system for B cell depletion is problematic in SLE where it is known to be compromised (Gong 2005).

Two large, randomized, placebo-controlled, Phase 2 studies in non-renal lupus (EXPLORER) and LN (LUNAR) both failed to meet their primary endpoints after treatment with rituximab, although both trials demonstrated partial responses in selected patients (Merrill 2010; Rovin 2012). Complete peripheral depletion of B cells with rituximab was not observed in all participants, and even in participants where complete peripheral depletion of B cells was observed, <50% achieved complete response (Gomez 2018).

As observed in murine models, the absence of a positive correlation between adequate peripheral depletion of B cells and complete response in the trial participants suggests that autoreactive B cells may persist in protected microenvironments like the lymphoid structures, kidney tubulointerstitial and bone marrow and therefore prolonged exposure to rituximab (>52 weeks) is needed (Gong 2005; Ahuja 2007; Ahuja 2011; Bekar 2010). Combination therapies involving rituximab and belimumab (a BAFF inhibitor) have been shown to reduce the re-emergence of autoreactive B cells following B cell depletion, enhance negative selection of autoreactive B cells, and lower anti-dsDNA antibody levels (Atisha-Fregoso 2021; van Schaik 2022). While combination treatments are effective in some patients, certain severe forms of SLE remain resistant to treatment.

Currently, there is no standard of care (SoC) treatment for achieving drug-free remission of SLE; therefore, patients often require life-long therapy. While a combination approach using antimalarials (hydroxychloroquine), systemic steroids, and conventional immunosuppressant medicines (azathioprine, MMF, and cyclophosphamide) are first-line options for SLE treatment, a significant proportion of patients continue to have high disease activity and recurrent relapses (Ginzler 2005; Rahman 2008; Appel 2009). Despite the apparent effectiveness of these regimens, complete renal remission in 30-50% of patients within the first year, between 20-35% of patients experience renal relapse on maintenance therapy over a 3-6-year follow-up period, with one study reporting that only 38% of patients were able to maintain a complete renal response over a 5-year period. A lack of response to therapy after 1 year of treatment was significantly associated with increased mortality and chronic kidney disease risk (p<0.005) (Anders 2015) and cyclophosphamide carries a secondary malignancy risk, in addition to increased risk of infertility with cumulative exposure (Ginzler 2005).

After the results of the LUNAR trial demonstrated no benefit of rituximab in complete renal response and a 15% improvement in partial renal response, a retrospective investigation was undertaken evaluating peripheral blood B cell depletion and occurrence of complete renal response. The data demonstrated that deeper B cell depletion was associated with improved complete renal response, and that poor peripheral blood B cell depletion was associated with non-response. This observation (Gomez 2018), coupled with data demonstrating that obinutuzumab (a type II mAb with greater antibody dependent cellular cytotoxicity through glycoengineering) in CLL was superior to rituximab in progression-free survival and minimal residual disease negativity in a head-to-head trial of CLL (Goede 2014) prompted the initiation of a Phase 2 trial of Obinutuzumab in LN. The hypothesis driving this trial was that deeper B cell depletion should increase complete renal response in LN patients. The Phase 2 results were published in 2021 (Furie 2022) and described a 12% improvement in complete renal response and 21% partial renal response at 52 weeks in the obinutuzumab-treated patients vs controls. The Phase 3 trial has completed enrolment and results are expected in 2024.

The current SoC treatment for anti-neutrophilic cytoplasmic autoantibody (ANCA) associated vasculitis (AAV) involves a combination of high dose glucocorticoids with either cyclophosphamide or rituximab, with evidence suggesting that the combination of rituximab and glucocorticoids is superior for remission induction in severe relapsing AAV compared with cyclophosphamide treatment alone (Stone 2010). Cyclophosphamide therapy is designed to target proliferating cells, resulting in blunted expansion of pathogenic clones, while memory B and T cells are unaffected. However, due to the dose-limiting AEs induced by cyclophosphamide, coupled with severe toxicity, complete depletion of pathogenic clones is rare, resulting in treatment failure and risk of chronic relapse. Cyclophosphamide is also associated with leukopenia, cancer, and ovarian failure (Mukhtyar 2008). Severe infections are also associated with prolonged cyclophosphamide exposure and are predominant causes of early death in patients (Jones 2010).

Results from the RAVE trial found that rituximab was non-inferior to cyclophosphamide for remission of disease defined as Birmingham Vasculitis Activity Scale (BVAS)/WG=0, without the use of prednisone, at 6 months (64% in rituximab group vs 53% in cyclophosphamide group) (Stone 2010). In addition, rituximab was superior to cyclophosphamide for induction of remission for relapsing disease (67% in rituximab group, 42% in cyclophosphamide group). Finally, there were no reported significant differences in rates of adverse events between the two treatments. Despite rituximab showing superiority against cyclophosphamide in reducing relapse rates, remission rates are still high in rituximab-treated patients, especially in patients with a history of relapse. The RITAZAREM trial demonstrated that repeated dosing of rituximab following remission (defined as BVAS/WG=0) in patients was superior compared to azathioprine at preventing relapse (HR 0.41; 95% CI 0.27 to 0.61, p<0.001) (Smith 2023). The MAINRITSAN trials demonstrated that prolonged rituximab treatment was an effective regimen in sustaining remission (defined as BVAS=0) in patients, however, for some patients after 2 years of maintenance, relapse risk remains high and extended therapy is advised (Tieu 2020; Charles 2018; Charles 2020; Guillevin 2014). Despite promising results with rituximab, there remains an unmet treatment need for this group of patients as highlighted in a recent study investigating the long-term outcomes and prognostic factors for survival of patients with AAV (Sanchez 2023). The study concluded that patients with AAV had an increased risk of mortality when compared to the general population, with treatment complications and organ damage being the main causes of limited survival and infection the leading cause of mortality.

Improved treatment options are needed as conventional therapies are not only associated with treatment failures and relapses, but also high levels of toxicity which result in severe morbidity and lethal adverse effects (Jayne 2003). A recent randomized controlled trial (ADVOCATE) investigated the C5a receptor inhibitor, avacopan, for the treatment of AAV (Jayne 2021). Avacopan is a C5a receptor antagonist that selectively blocks the effects of C5a preventing neutrophil chemoattraction and activation and has demonstrated beneficial effects on vasculitis in Phase 2 clinical trials (Jayne 2017; Merkel 2020). All patients were given either cyclophosphamide or rituximab and then assigned into two groups: one received avacopan and the other prednisone. Results showed avacopan was non-inferior but not superior to prednisone in inducing remission defined as BVAS-0 of vasculitis at 26 weeks and was superior to prednisone at 52 weeks in patients who received rituximab or cyclophosphamide.

In some embodiments, a subject of the present disclosure has or is suspected of having a B cell mediated autoimmune disorder. In some embodiments, a B cell mediated autoimmune disorder is selected from the group consisting of: systemic lupus erythematosus (SLE), extrarenal systemic lupus erythematosus (ERL), lupus nephritis (LN), CNS lupus, anti-neutrophilic cytoplasmic autoantibody (ANCA) associated vasculitis (AAV), granulomatous polyangiitis, microscopic polyangiitis, multiple sclerosis (relapsing and/or progressive), pemphigus vulgaris, autoimmune blistering skin diseases, membranous nephropathy (MN), anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, autoimmune hemolytic anemia, amyloidosis, scleroderma, idiopathic inflammatory myositis, and immune-mediated necrotizing myopathy. In some embodiments, a subject of the present disclosure is diagnosed to have systemic lupus crythematosus (SLE), extrarenal systemic lupus crythematosus (ERL), lupus nephritis (LN), or ANCA-associated vasculitis (AAV), or membranous nephropathy (MN). In some embodiments, a subject of the present disclosure has moderate to severe disease activity. In some embodiments, a subject of the present disclosure has a relapsed or refractory autoimmune condition. In some embodiments, a subject of the present disclosure has autoantibodies. In some embodiments, depth of B cell depletion in a subject of the present disclosure that has or is suspected of having a B cell mediated autoimmune disorder following administration of a drug product comprising hypoimmune allogenic CD19-directed CAR T cells of the present disclosure can inform treatment and may predict efficacy of the drug product. B cells drive disease pathology in many autoimmune diseases, so B cell depletion following administration of a drug product comprising hypoimmune allogenic CD19-directed CAR T cells of the present disclosure can be efficacious in many autoimmune diseases and deliver durable, long-term remission in a subject of the present disclosure that has or is suspected of having a B cell mediated autoimmune disorder.

In some embodiments, a subject of the present disclosure has adequate organ function. In some embodiments, a subject of the present disclosure has adequate hepatic function. In some embodiments, adequate hepatic function comprises: a total bilirubin level <1.5× upper limit of normal (ULN); and/or alanine aminotransferase (ALT) or aspartate aminotransferase (AST)<3×ULN. In some embodiments, a subject of the present disclosure has disease-related muscle involvement causing elevated ALT or AST. In some embodiments, adequate hepatic function comprises: a total bilirubin level <3× upper limit of normal (ULN) and/or alanine aminotransferase (ALT) or aspartate aminotransferase (AST)<5×ULN; wherein the subject has liver involvement; and wherein the total bilirubin level <3×ULN is not attributed to Gilbert's syndrome. In some embodiments, a subject of the present disclosure has adequate bone marrow function. In some embodiments, adequate bone marrow function comprises a hemoglobin level ≥8 g/da neutrophil count ≥1000/mm³ and/or a platelet count ≥50,000/mm³. In some embodiments, a subject of the present disclosure has adequate pulmonary function. In some embodiments, adequate pulmonary function comprises a baseline oxygen saturation >92% on room air; no clinically significant pleural effusion; and/or a forced expiratory volume in the first second/Forced Vital Capacity (FEV1/FVC)>70% as measured by spirometry. In some embodiments, a subject of the present disclosure is eligible for lymphodepleting chemotherapy. In some embodiments, a subject of the present disclosure is diagnosed to have SLE based on 2019 EULAR/ACR Classification Criteria. In some embodiments, a subject of the present disclosure is confirmed to have Class III (A or A/C) or IV (A or A/C) LN based on histological analysis using ISN/RPS 2018 criteria with active or active/chronic lupus nephritis performed within 6 months of screening. In some embodiments, a subject of the present disclosure is confirmed to have Class V LN. In some embodiments, a subject of the present disclosure is confirmed to have Class III or Class IV LN. In some embodiments, a subject of the present disclosure has at least one elevated autoantibody. In some embodiments, at least one elevated autoantibody comprises anti-dsDNA, anti-nuclear autoantibody (ANA) or anti-Sm (Smith) autoantibody. In some embodiments, an ANA comprises a titer of at least 1:80. In some embodiments, at least one elevated autoantibody comprises anit-PLA2R or anti-THSD7A. In some embodiments, a subject of the present disclosure has a prothrombin time-international normalized ratio ≤2.5 with no disease-related coagulopathy. In some embodiments, a subject of the present disclosure has an activated partial thromboplastin time ≤2.5×ULN. In some embodiments, a subject of the present disclosure has an estimated GFR of ≥45 mL/min/1.73m² as determined by a CKD-EPI equation. In some embodiments, a subject of the present disclosure has proteinuria with a UPCR determined by either spot urine collection or 24-hour urine collection of >1 g/dL and ≤7 g/dL. In some embodiments, a subject of the present disclosure has a SLEDAI-2K score ≥8. In some embodiments, a subject of the present disclosure is diagnosed to have granulomatous with polyangiitis (GPA) or microscopic polyangiitis (MPA) based on the Chapel Hill Consensus Conference criteria. In some embodiments, a subject of the present disclosure is diagnosed with a disease flare with a BVAS >2 following the achievement of remission induced by a regimen consisting of anti-CD20 B cell depletion (e.g., rituximab or obinutuzumab) and glucocorticoids; cyclophosphamide and glucocorticoids; or anti-CD20 B cell depletion, cyclophosphamide and glucocorticoids. In some embodiments, a disease flare is organ or life threatening. In some embodiments, a subject of the present disclosure has had at least one GPA or MPA flare, at least one prior induction regimen with rituximab, obinutuzumab or cyclophosphamide and a baseline BVAS of ≥1. In some embodiments, a subject of the present disclosure has severe disease or a history of severe disease as defined by one or more major BVAS items. In some embodiments, a subject of the present disclosure is positive for either PR3-ANCA or MPO-ANCA. In some embodiments, a subject of the present disclosure is refractory to rituximab or has relapsed after prior rituximab therapy with evidence of reduced eGFR.

In some embodiments, a subject of the present disclosure has or is suspected of having a disease, disorder, or condition that is relapsed and/or refractory. In some embodiments, a B cell malignancy is relapsed and/or refractory. In some embodiments, a B cell malignancy is relapsed and/or refractory NHL. In some embodiments, a B cell malignancy is relapsed and/or refractory CLL. In some embodiments, a B cell malignancy is relapsed and/or refractory LBCL. In some embodiments, the B cell malignancy is relapsed and/or refractory after at least 2 regimens of therapy. In some embodiments, a B cell malignancy is relapsed and/or refractory after autologous stem cell transplant (ASCT). In some embodiments, a B cell malignancy is relapsed and/or refractory, wherein relapsed and/or refractory comprises one or more of: no response to first line therapy, progressive disease as best response to most recent therapy regimen, stable disease as best response to most recent therapy with duration no longer than 6 months from last dose of therapy, disease progression or relapse within 12 months of ASCT, no response to or relapse after last line of therapy post-ASCT, and/or no response to or relapse after treatment with a first CD19 CAR T therapy. In some embodiments, a subject of the present disclosure has or is suspected of having relapsed and/or refractory CLL and has already been treated with at least 2 regimens of therapy. In some embodiments, a subject of the present disclosure has or is suspected of having relapsed and/or refractory multiple myeloma and has already been treated with at least 1 regimen of therapy. In some embodiments, a subject of the present disclosure has or is suspected of having relapsed and/or refractory LBCL and is transplant ineligible. In some embodiments, a subject of the present disclosure has already been treated with a bispecific antibody. In some embodiments, a bispecific antibody is selected from the group consisting of: mosunetuzumab (CD20×CD3 mAb), glofitamab (CD20⁽²⁾×CD3), odronextamab (CD20×CD3 mAb), and epcoritamab (CD20×CD3 mAb). In some embodiments, a B cell mediated disorder is relapsed and/or refractory. In some embodiments, a B cell mediated disorder is relapsed and/or refractory following a first line therapy comprising mycophenolate mofetil (MMF), cyclophosphamide, or a combination thereof. In some embodiments, a B cell mediated disorder is relapsed and/or refractory following a second line therapy comprising rituximab, belimumab, voclosporin, obinutuzumab, or a combination thereof. In some embodiments, a B cell mediated disorder is relapsed and/or refractory following at least two disease-modifying therapies selected from the group consisting of: MMF, azathioprine, cyclophosphamide, methotrexate, rituximab, belimumab, intravenous immunoglobulin (IVIg), and abatacept. In some embodiments, a subject does not achieve a complete renal response to the second line therapy within 6 months of initiation of the second line therapy. In some embodiments, a subject has disease activity despite maintenance on maximally tolerated doses of drugs that block the renin-angiotensin system (RAS).

In some embodiments, a subject of the present disclosure is 18-85 years of age. In some embodiments, a subject of the present disclosure is 18-80 years of age. In some embodiments, a subject of the present disclosure is 18-75 years of age.

### 4. Exclusion Criteria

In some embodiments, a subject of the present disclosure cannot or should not be treated with a pharmaceutical composition of the present disclosure if they meet certain criteria (exclusion criteria). In some embodiments, a subject of the present disclosure has not received a prior CD19-directed therapy. In some embodiments, a prior CD19-directed therapy is or comprises a CD19-directed CAR T cell treatment. In some embodiments, a prior CD19-directed therapy is or comprises a CD19-directed antibody treatment. In some embodiments, a prior CD19-directed therapy is or comprises a CD19-directed CAR NK cell treatment. In some embodiments, a subject of the present disclosure has not received a prior CD47-directed therapy. A CD47-directed therapy can include a binder of CD47 (e.g., an anti-CD47 antibody) that specifically binds to the CD47 and interferes with the interaction of CD47 and SIRPα. Examples of binders that specifically bind CD47 include magrolimab, Trillium (TTI)-621, ALX148, AO-176, lemzoparlimab, IBI188, SRF231, CC-90002, and AK117. A CD47-directed therapy can also include a protein and/or molecule that interferes with the interaction of CD47 and SIRPα by binding SIRPα, such as ADU-1805 and BI 765063. In some embodiments, a subject has not received a CD47-directed therapy within 1 year of being administered the composition comprising the population of engineered hypoimmunogenic T cells. In some embodiments, a subject has not received a CD47-directed therapy within about 1 year, about 11 months, about 10 months, about 9 months, about 8 months, about 7 months, about 6 months, about 5 months, about 4 months, about 3 months, about 2 months, or about 1 month of being administered the composition comprising the population of engineered hypoimmunogenic T cells. In some embodiments, a subject of the present disclosure having or suspected of having anti-CD47 antibodies cannot or should not be treated with a pharmaceutical composition of the present disclosure until anti-CD47 antibody levels are reduced. In some embodiments, a subject of the present disclosure having or suspected of having anti-CD47 antibodies cannot or should not be treated with a pharmaceutical composition of the present disclosure until a period of time known to be associated with reduced (i.e., cleared) anti-CD47 antibody levels has passed. For example, the period of time may be 1 year, about 11 months, about 10 months, about 9 months, about 8 months, about 7 months, about 6 months, about 5 months, about 4 months, about 3 months, about 2 months, or about 1 month. As another example, the period of time may be 5, 6, 7, 8, 9, or 10 half-lives of the CD47-directed therapy. In some embodiments, a subject of the present disclosure having or suspected of having anti-CD47 antibodies cannot or should not be treated with a pharmaceutical composition of the present disclosure until testing of anti-CD47 antibody levels indicates reduced levels. In some embodiments, a subject of the present disclosure having or suspected of having anti-CD47 antibodies cannot or should not be treated with a pharmaceutical composition of the present disclosure until anti-CD47 antibody levels are at undetectable levels. Any method known in the art for detecting antibodies in a sample from a patient can be used. For example, antibodies can be detected in a sample (e.g., serum or whole blood) using an enzyme-linked immunospot (ELISpot) assay, ELISA, immunofluorescence, flow cytometry, Western blot, or any other methodology of determining the antibody titer of a specific antibody. In some embodiments, a subject of the present disclosure subject does not have a history of primary central nervous system (CNS) lymphoma or CNS metastases. In some embodiments, a subject of the present disclosure does not have a history of Richter's transformation of chronic leukemic lymphoma, small lymphocytic lymphoma, or lymphoplasmacytic lymphoma. In some embodiments, a subject of the present disclosure does not have a fungal, bacterial, viral, or other infection that is uncontrolled or requiring IV antimicrobials for management. In some embodiments, a subject of the present disclosure has not been hospitalized for treatment of an infection within 28 days of baseline of >2 times within 1 year of administering the composition to the subject. In some embodiments, a subject of the present disclosure does not have acute symptoms of COVID-19 infection. In some embodiments, a subject of the present disclosure does not have a known history of infection with HIV and/or hepatitis B (HBsAg positive) and/or hepatitis C virus (anti-HCV positive). In some embodiments, a subject of the present disclosure does not have an active autoimmune disease and/or any other diseases requiring immunosuppressive therapy or corticosteroid therapy. In some embodiments, a subject of the present disclosure does not have primary immunodeficiency or history of concomitant genetic syndrome associated with bone marrow failure (e.g., Fanconi anemia, Kostmann syndrome, or Shwachman-Diamond). In some embodiments, a subject of the present disclosure has not been treated with a systemic cancer therapy within 14 days of administration of the composition. In some embodiments, a subject of the present disclosure has not been treated with any investigational product within 28 days of administration of the composition. In some embodiments, a subject of the present disclosure has not been treated with radiotherapy within 14 days of administration of the composition. In some embodiments, a subject of the present disclosure has not been treated with major surgery within 28 days of administration of the composition. In some embodiments, a subject of the present disclosure has not been treated with autologous hematopoietic stem cell transplant (HSCT) within 6 weeks of administration of the composition. In some embodiments, a subject of the present disclosure has not had myocardial infarction, cardiac angioplasty or stenting, cardiac arrhythmia requiring medication, unstable angina, New York Heart Association Class II or greater congestive heart failure, cardiac atrial or ventricular lymphoma involvement or clinically significant cardiac disease within one year of administration of the composition. In some embodiments, a subject of the present disclosure has not had an ejection fraction ≤50%, arrythmias and any structural heart disease (including clinically significant pericardial effusion), as assessed by echocardiogram or multiple gated acquisition scan performed within 1 month of administration of the composition. In some embodiments, a subject of the present disclosure does not have a history of non-line associated, clinically significant deep-vein thrombosis (i.e., proximal deep vein thrombosis [DVT]) or pulmonary embolism requiring therapeutic anticoagulation within 6 months of administration of the composition. In some embodiments, a subject of the present disclosure does not have a history or presence of CNS disorder (e.g., seizure disorder, cerebrovascular ischemia/hemorrhage, dementia, cerebellar disease, or any autoimmune disease with CNS involvement) within 12 months of administration of the composition. In some embodiments, a subject of the present disclosure does not have clinically significant ascites. In some embodiments, a subject of the present disclosure does not have a history of organ transplantation and/or is not awaiting organ transplantation. In some embodiments, a subject of the present disclosure does not have an indwelling line or drain. In some embodiments, a subject of the present disclosure has not received a live vaccine within 6 weeks of administration of the composition. In some embodiments, a subject of the present disclosure does not have any unresolved toxicity greater than Grade 1 from a previous anticancer therapy. In some embodiments, a subject of the present disclosure does not have a history of additional malignancy other than nonmelanoma skin cancer or carcinoma in situ. In some embodiments, a subject of the present disclosure does not have a severe immediate hypersensitivity to any of the components of a pharmaceutical composition of the present disclosure. In some embodiments, a subject of the present disclosure does not have an admission or evidence of illicit drug use, drug abuse, or alcohol abuse. In some embodiments, a subject of the present disclosure does not exhibit clinical evidence of significant unstable or uncontrolled acute or chronic disease not due to the B cell mediated disorder. In some embodiments, a significant unstable or uncontrolled acute or chronic disease comprises cardiovascular, pulmonary, hematologic, gastrointestinal, hepatic, renal, neurological, malignancy, or infectious disease. In some embodiments, a subject of the present disclosure does not have a history of an anaphylactic reaction to parenteral administration of contrast agents, human or murine proteins, or monoclonal antibodies. In some embodiments, a subject of the present disclosure does not have a history of cancer, or the subject has been disease-free from a basal carcinoma of the skin, cervix, bladder, prostate or breast for at least five years prior to administration of the composition. In some embodiments, a cancer comprises a hematologic malignancy, a solid tumor, or a carcinoma in situ. In some embodiments, a subject of the present disclosure is administered physiologic replacement glucocorticoids, topical glucocorticoids, or inhaled glucocorticoids. In some embodiments, a subject of the present disclosure is not administered glucocorticoids above physiologic replacement. In some embodiments, physiologic replacement glucocorticoids comprises 5 mg prednisone daily or equivalent. In some embodiments, a subject of the present disclosure has not received treatment with Leflunomide within 28 prior to administration of the composition. In some embodiments, a subject of the present disclosure does not have an immunoglobulin A (IgA) deficiency (IgA level <10 mg/dL). In some embodiments, a subject of the present disclosure does not have a grade ≥3 laboratory abnormality, except those associated with the B cell mediated disorder. In some embodiments, a subject of the present disclosure does not have a B cell mediated disorder related flare that needs immediate treatment. In some embodiments, a subject of the present disclosure does not have a CNS lupus manifestation, a history of a CNS disorder, or a presence of a CNS disorder. In some embodiments, a CNS disorder is or comprises primary seizure disorder, cerebrovascular ischemia/hemorrhage, dementia, or cerebellar disease. In some embodiments, a subject of the present disclosure has not been diagnosed with anti-phospholipid antibody syndrome that is actively treated. In some embodiments, a subject of the present disclosure has not been diagnosed with EGPA as defined by the Chapel Hill Consensus Conference criteria. In some embodiments, a subject of the present disclosure does not have an alveolar hemorrhage within 4 weeks prior to administration of the composition. In some embodiments, a subject of the present disclosure has not been diagnosed with a cerebrovascular accident caused by vasculitis or pachymeningitis.

In some embodiments, a subject of the present disclosure having or suspected of having a B cell mediated autoimmune disorder cannot or should not be treated with a pharmaceutical composition of the present disclosure if they have an active infection. In some embodiments, a subject of the present disclosure having or suspected of having a B cell mediated autoimmune disorder cannot or should not be treated with a pharmaceutical composition of the present disclosure if they are unable to receive a lymphodepletion regimen.

In some embodiments, a subject of the present disclosure should not take corticosteroid therapy at a pharmacologic dose (≥5 mg/day of prednisone or equivalent doses of other corticosteroids) and other immunosuppressive drugs within 5 days of administration of a lymphodepletion regimen of the present disclosure and/or administration of hypoimmune allogenic CD19-directed CAR T cells of the present disclosure.

In some embodiments, a subject of the present disclosure should not take corticosteroids and/or other immunosuppressive drugs in the 3 months after administration of hypoimmune allogenic CD19-directed CAR T cells of the present disclosure. In some embodiments, a subject of the present disclosure should not take nonsteroidal anti-inflammatory agents in the 3 months after administration of hypoimmune allogenic CD19-directed CAR T cells of the present disclosure. In some embodiments, a subject of the present disclosure should not take therapeutic doses of systemic anticoagulants, such as unfractionated heparin and low-molecular weight heparin after administration of hypoimmune allogenic CD19-directed CAR T cells of the present disclosure. In some embodiments, a subject of the present disclosure docs not receive treatments for underlying disease, such as surgery, chemotherapy, immunotherapy, targeted agents, radiation, and high-dose corticosteroids (other than those allowed herein) and other investigational agents after administration of hypoimmune allogenic CD19-directed CAR T cells of the present disclosure.

### 5. Lymphodepletion Regimen

Non-limiting examples of an immunosuppressive and/or immunomodulatory agent (such as, but not limited to a lymphodepletion agent) include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, or CD58, and antibodies binding to any of their ligands. In some embodiments, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, where an immunosuppressive and/or immunomodulatory agent is administered to the patient before or after the administration of the cells, the administration is at a lower dosage than would be required for cells with MHC I and/or MHC II expression, TCR expression and without exogenous expression of CD47. In some embodiments, where an immunosuppressive and/or immunomodulatory agent is administered to the patient before or after the first administration of the cells, the administration is at a lower dosage than would be required for cells with MHC I and MHC II expression, TCR expression and without exogenous expression of CD47.

In some embodiments, a subject of the present disclosure is administered a lymphodepleting chemotherapy regimen, wherein administering the lymphodepleting chemotherapy regimen occurs prior to administering a pharmaceutical composition of the present disclosure. In some embodiments, a lymphodepleting therapy is or comprises cyclophosphamide and fludarabine. In some embodiments, cyclophosphamide is administered at a dose of 500 mg/m². In some embodiments, cyclophosphamide is administered at a dose of 500 mg/m² daily for 3 days. In some embodiments, cyclophosphamide is administered at a dose of 1000 mg/m². In some embodiments, cyclophosphamide is administered at a dose of 1000 mg/m² one, 3 days prior to administration of a composition of the present disclosure. In some embodiments, fludarabine is administered at a dose of 30 mg/m². In some embodiments, fludarabine is administered at a dose of 25 mg/m². In some embodiments, a subject of the present disclosure has a moderate impairment of renal function, and fludarabine is administered at a dose of about 20 mg/m² with monitoring. In some embodiments, fludarabine is administered at a dose of 30 mg/m² daily for 3 days. In some embodiments, cyclophosphamide and fludarabine are administered concurrently. In some embodiments, a lymphodepleting chemotherapy regimen is administered intravenously. In some embodiments, a pharmaceutical composition of the present disclosure is administered to a subject 2-7 days after completion of the lymphodepleting chemotherapy regimen.

In some embodiments, a subject of the present disclosure has not received a live vaccine within 28 days of administration of the lymphodepleting chemotherapy.

In some embodiments, a subject of the present disclosure is weaned to discontinuation of a severe autoimmune therapy over the course of no more than 21 days prior to administration of a composition of the present disclosure to the subject. In some embodiments, a subject is weaned to 0 mg glucocorticoids. In some embodiments, a subject is weaned to a physiologic equivalent range of ≤5 mg/day of prednisone.

In some embodiments, a subject is not administered a lymphodepleting chemotherapy regimen prior to administration of a pharmaceutical composition of the present disclosure. In some embodiments, the following lymphodepletion protocol is followed for a subject of the present disclosure. On Days −5 through −3, before the infusion of hypoimmune allogenic CD19-directed CAR T cells of the present disclosure, subjects receive a non-myeloablative lymphodepletion (LD) regimen consisting of cyclophosphamide and fludarabine to induce lymphocyte depletion and create an optimal environment for the expansion of hypoimmune allogenic CD19-directed CAR T cells of the present disclosure in vivo. Hypoimmune allogenic CD19-directed CAR T cells of the present disclosure should be administered 3 days (range 2-7 days) after completion of LD chemotherapy.

In some embodiments, subjects of the present disclosure receive the following 3-day chemotherapy regimen per the order identified as follows: 1) IV hydration with 1 L of 0.9% sodium chloride (NaCl) given before cyclophosphamide on the day of infusion; 2) Cyclophosphamide 500 mg/m² IV over approximately 60 minutes on Days −5, −4, and −3; 3) Fludarabine 30 mg/m² IV over approximately 30 minutes on Days −5, −4, and −3 and subjects with moderate impairment of renal function (creatinine clearance 50-70 mL/min/1.73 m²) should have their fludarabine dose reduced by 20% and be monitored closely; 4) an additional 1 L of 0.9% NaCl at the completion of the fludarabine infusion; and 5) add Mesna (sodium 2-mercaptoethanesulfonate; a detoxifying agent used to inhibit the hemorrhagic cystitis induced by the chemotherapy) per institutional guidelines. Subjects at risk for tumor lysis syndrome (TLS) should receive TLS prophylaxis prior to LD chemotherapy according to institutional guidelines.

Administration of hypoimmune allogenic CD19-directed CAR T cells of the present disclosure can increase the risk of high-grade and fatal toxicities in subjects with ongoing infection and/or inflammation. In some embodiments, if a subject of the present disclosure has: a temperature >38° C. within 72 hours before LD chemotherapy; CRP ≥100 mg/L any time between enrollment and the start of LD chemotherapy; and/or a white blood cell (WBC) count or WBC differential concerning for infectious process between enrollment and the start of LD chemotherapy (e.g., WBC >20,000 cells/μL, rapidly increasing WBC, or WBC differential with a high percentage of segments/bands), the cause of the infection should be identified and treated before administration of an LD regimen of the present disclosure.

In some embodiments, if any screening assessments or procedures are repeated between confirmation of eligibility and the start of LD chemotherapy and the results are outside of the eligibility criteria provided herein, any suspected/identified infectious/inflammatory process condition must resolve before a subject of the present disclosure is administered an LD regimen of the present disclosure. In some embodiments, a complete medical history and physical examination of a subject of the present disclosure including the head, eyes, ears, nose, and throat (HEENT) and cardiac, vascular, respiratory, gastrointestinal, integumentary, and neurological systems does not reveal evidence of infection/inflammation before administration of an LD regimen of the present disclosure. In some embodiments, a subject of the present disclosure has not received systemic antimicrobials for the treatment of a known or suspected infection within the 48 hours before an LD regimen of the present disclosure (prophylactic use of antimicrobials is allowed). In some embodiments, a treatment course of any antimicrobials given for a known or suspected antecedent infection should be completed before a subject of the present disclosure is administered an LD regimen of the present disclosure. In some embodiments, if a subject of the present disclosure is confirmed to have an infectious process for which antimicrobials are not available (e.g., viral pneumonia), the infection must be clinically resolved before a subject of the present disclosure is administered an LD regimen of the present disclosure. In some embodiments, the most recently collected blood, urine, or other body fluid cultures must show no growth for at least 48 hours, and any other infectious workup performed (e.g., bacterial, PCR, or stool or imaging studies) is negative before a subject of the present disclosure is administered an LD regimen of the present disclosure. In some embodiments, if there is clinical suspicion of an infection for which cultures are unlikely to be positive within 48 hours (e.g., fungal infection), then adequate time is allowed to allow the cultures to become positive before a subject of the present disclosure is administered an LD regimen of the present disclosure.

### 6. Assessment of Subjects

In some embodiments, subjects of the present disclosure undergo safety assessments that include the collection of adverse events (AEs) and serious adverse events (SAEs), clinical laboratory tests, imaging, physical and neurologic examinations, vital signs, cardiac function (e.g., electrocardiogram [ECG]), and ECOG performance status, as described herein. In some embodiments, subjects of the present disclosure asked about any AEs that might have occurred since a previous evaluation. In some embodiments, serious adverse events (SAEs), treatment-related adverse events (TRAEs), treatment-emergent adverse events (TEAEs), targeted adverse events (TAEs), adverse events of special interest (AESIs), and/or dose-limiting toxicities (DLTs) are determined for a subject as part of an evaluation.

In some embodiments, subjects of the present disclosure undergo safety assessments that include clinical laboratory tests. Exemplary clinical laboratory tests include those found in Table 16 and Table 17 of the present disclosure. In some embodiments, subjects of the present disclosure undergo safety assessments that include physical examination. In some embodiments, a physical examination comprises measurement of, assessment of, or performance of one or more of: height, weight, vital signs (e.g., systolic/diastolic blood pressure, heart rate, respiratory rate, oxygen saturation, temperature), neurological status (e.g., immune effector cell-associated encephalopathy (ICE) score), Eastern Cooperative Oncology Group (ECOG) score, cardiac function, echocardiogram (ECHO), electrocardiogram (ECG), brain Magnetic Resonance Imaging (MRI), a lumbar puncture, and information about concomitant medications.

In some embodiments, subjects of the present disclosure are assessed for the presence of replication competent lentivirus (RCL) in peripheral blood mononuclear cells (PBMCs). In some embodiments, subjects of the present disclosure are assessed for abnormal T cell proliferation. In some embodiments, subjects of the present disclosure are assessed for the presence of anti-CD19-directed CAR antibodies in the subjects' serum. In some embodiments, serum from subjects of the present disclosure is assessed for cytokine profiling.

In some embodiments, subjects of the present disclosure are assessed for disease progression and/or disease response. In some embodiments, subjects of the present disclosure with NHL will be assessed based on Lugano classification criteria. In some embodiments, subjects of the present disclosure with CLL will be assessed based on the International Workshop on Chronic Lymphocytic Leukemia (iwCLL) criteria. In some embodiments, disease assessment in subjects of the present disclosure comprises imaging. In some embodiments, imaging comprises positron emission tomography (PET)-computed tomography (CT) and/or CT scans. In some embodiments, subjects of the present disclosure undergo PET-CT and/or CT scans within 24 months of administration of hypoimmune allogenic CD19-directed CAR T cells of the present disclosure. In some embodiments, disease assessment in subjects of the present disclosure comprises bone marrow aspirate and/or biopsy.

In some embodiments, PBMC samples from subjects of the present disclosure are assessed for cellular kinetics of hypoimmune allogenic CD19-directed CAR T cells of the present disclosure. In some embodiments, cellular kinetics are evaluated by droplet digital polymerase chain reaction (ddPCR) and reported as a level of hypoimmune allogenic CD19-directed CAR T cells per μg of genomic DNA (gDNA). In some embodiments, the derived parameters of Cₘₐₓ, Tₘₐₓ, t_{1/2}, Cₗₐₛₜ, Tₗₐₛₜ, and AUC are determined for hypoimmune allogenic CD19-directed CAR T cells of the present disclosure. In some embodiments, the number of hypoimmune allogenic CD19-directed CAR T cells of the present disclosure in a subject's blood will be assessed via flow cytometry. In some embodiments, cellular kinetics as described herein are used to determine dose-exposure relationships and/or correlation of persistence and expansion of hypoimmune allogenic CD19-directed CAR T cells of the present disclosure with safety and efficacy parameters.

In some embodiments, blood/serum samples from subjects of the present disclosure undergo pharmacodynamic assessments. In some embodiments, pharmacodynamic assessments comprise monitoring of levels of analytes in blood/serum over time. In some embodiments, analytes include one or more of: homeostatic/proliferative cytokines (e.g., interleukin (IL)-2, IL-7, IL-15, and granulocytecolony-stimulating factor (G-CSF)), inflammatory/immune-modulating cytokines (e.g., interferon-gamma (IFN-γ), IFN-α, IL-1β, IL-1Ra, IL-4, IL-6, IL-8, IL-10, IL-12p70, IL-13, IL-17A, granulocyte macrophage colony-stimulating factor (GM-CSF), tumor necrosis factor-alpha (TNF-α), macrophage inflammatory protein-1 Alpha (MIP-1α), and MIP-1β), correlates of acute phase response (e.g., C-reactive protein (CRP) and ferritin), chemokines (e.g., IL-8, C-X-C motif chemokine ligand-10 (CXCL-10), and monocyte chemotactic protein-1 (MCP-1)), and associated response related with CD19-directed CAR activity (e.g., circulating CD19+ B cell and lactate dehydrogenase (LDH)).

In some embodiments, subjects of the present disclosure are assessed for minimal residual disease (MRD). In some embodiments, MRD is determined using a next-generation sequencing (NGS) MRD assay to measure ctDNA levels before and after administration of hypoimmune allogenic CD19-directed CAR T cells of the present disclosure. In some embodiments, subjects of the present disclosure are assessed for immune responses against the administered hypoimmune allogenic CD19-directed CAR T cells of the present disclosure. In some embodiments, immune responses hypoimmune allogenic CD19-directed CAR T cells of the present disclosure are or comprise anti-hypoimmune allogenic CD19-directed CAR T cell antibodies, antibody-dependent cellular cytotoxicity (ADCC), complement-dependent cytotoxicity (CDC), immune activation against hypoimmune allogenic CD19-directed CAR T cells of the present disclosure, NK cell-mediated killing of hypoimmune allogenic CD19-directed CAR T cells of the present disclosure.

In some embodiments, methods of the present disclosure comprise measuring MRD in a subject to monitor treatment response or relapse of disease. In some embodiments, MRD refers to a small number of disease cells that remain in the subject during or after treatment when the subject is in remission. In remission, the subject may no longer display obvious symptoms or signs of disease, but MRD cells may remain and cause relapse of the disease. Testing for MRD may be useful for determining whether treatment has eradicated disease cells or whether traces remain, comparing efficacy of different treatments, monitoring patient remission status as well as detecting recurrence of the disease, and adjusting treatments accordingly. The number of MRD cells in a sample from the subject may be as low as one disease cell in a million normal cells.

In some embodiments, MRD can be measured using various methods as described in, but not limited to, US20220251654A1, US20170335391A1, US20210238694A1, and US20200370129A1.

In some embodiments, measuring MRD comprises collecting two or more biological samples from a subject at different time points (e.g. prior to, concurrent with, or after administration of hypoimmune allogenic CD19-directed CAR T cells). In some embodiments, a multiplex amplification reaction is performed on nucleic acids isolated from the biological samples to generate a set of amplicons, wherein each of the set of amplicons comprises recombined V(D)J gene segments at a gene locus of interest and/or wherein each of the set of amplicons comprises gene segments at one or more cancer specific gene loci of interest, and sequencing the set of amplicons, wherein sequences of the recombined V(D)J gene segments and/or cancer specific gene segments are indicative of presence of an immune cell and/or cancer in the biological sample.

In some embodiments, measuring MRD comprises assigning a pair of first and second polypeptides that form a TCR (TCRA or TCRB) or immunoglobulin (e.g., Ig heavy chain, or κ or λ light chain) heterodimer to a single biological source sample among the two or more biological samples. In some embodiments, the first rearranged nucleic acid sequences encoding the first polypeptides of the TCR or Ig heterodimers present in the source biological sample is determined for each of the plurality of biological samples and the first rearranged nucleic acid sequences are assigned to the source biological sample. The plurality of source biological samples are then pooled to form a combined population of cell and a plurality of cognate pairs of first and second rearranged nucleic acid sequences encoding first and second polypeptides of the TCR or Ig heterodimers is determined from the combined population of cells. The first rearranged nucleic acid sequences determined in each of the source samples are compared to the first rearranged nucleic acid sequences determined from the plurality of cognate pairs of rearranged nucleic acid sequences to assign each first rearranged nucleic acid sequence present in the combined population to a single biological source sample. For each first rearranged nucleic acid sequence assigned to a single biological source sample, the cognate second rearranged nucleic acid sequence of the cognate pair is assigned to the same single biological source sample.

In another embodiment, MRD is measured by determining a first rearranged nucleic acid sequence encoding the first polypeptide of the TCR or Ig heterodimer present in the biological source sample. In some embodiments, for each biological source sample, rearranged nucleic acid molecules extracted from the biological source sample are amplified in a single multiplex polymerase chain reaction (PCR) using a plurality of V-segment primers and a plurality of J-segment primers to produce a plurality of rearranged nucleic acid amplicons. The plurality of rearranged nucleic acid amplicons are sequenced to determine sequences of the first rearranged nucleic acid sequences in each biological source sample.

In some embodiments, MRD is measured by using a “bait set” of probes for hybridization capture. The bait set comprises a plurality of different oligonucleotide-containing probes, where each of the oligonucleotide-containing probes comprises a sequence of at least 30 bases in length that is complementary to either: (1) a sequence of a genomic region; or (2) a sequence that varies from the sequence of (1) only by one or more transitions.

In some embodiments, MRD is measured via immune repertoire sequencing. TCRs and immunoglobulins serve as a unique “barcode” for these lymphocytes. By sequencing the immune receptors, the immune response in patient samples and clonal expansion of specific lymphocyte subpopulations can be tracked and monitored.

In some embodiments, MRD is measured by amplifying and sequencing cell-free nucleic acids that are released into bodily fluid from cancer cells, e.g., circulating tumor DNA (ctDNA). In some embodiments, ctDNA can be non-encapsulated tumor-derived fragmented DNA and/or may have one or more epigenetic modifications, for example, the ctDNA may be acetylated, 5-methylated, ubiquitylated, phosphorylated, sumoylated, ribosylated, and/or citrullinated.

In some embodiments, subjects of the present disclosure undergo tumor biopsies or liquid biopsies and the samples are evaluated for CD19 expression. In some embodiments, subjects of the present disclosure undergo tumor biopsies or liquid biopsies before administration of hypoimmune allogenic CD19-directed CAR T cells of the present disclosure and biopsies are assessed to determine mechanisms of cancer resistance. In some embodiments, subjects of the present disclosure undergo tumor biopsies or liquid biopsies after administration of hypoimmune allogenic CD19-directed CAR T cells of the present disclosure and tumor cells are assessed to determine mechanisms of tumor resistance.

In some embodiments, PBMCs collected from subjects of the present disclosure before and after administration of hypoimmune allogenic CD19-directed CAR T cells of the present disclosure are assessed for immunophenotypic changes induced by the hypoimmune allogenic CD19-directed CAR T cells of the present disclosure, including expression level of CD47, broad immune cell phenotyping (e.g., lymphoid and myeloid cell phenotypes, e.g., T, B, NK and monocytes), and immune cell subsets (e.g., differentiation, activation, and exhaustion).

In some embodiments, subjects of the present disclosure are assessed for their survival after administration of hypoimmune allogenic CD19-directed CAR T cells of the present disclosure.

In some embodiments, subjects of the present disclosure are assessed for certain safety and efficacy criteria after administration of hypoimmune allogenic CD19-directed CAR T cells of the present disclosure. In some embodiments the certain safety criteria comprise CRS Grade 3/4 ≤10%, ICANS Grade 3/4 ≤15%, infections Grade 3/4 <15%, and other Grade 3/4 adverse events <5%. In some embodiments the certain safety criteria comprise hypoimmune allogenic CD19-directed CAR T cells of the present disclosure being no worse than autologous CAR T cells. In some embodiments, certain efficacy criteria comprise an event-free survival (EFS) of 10 months, complete response rate (CRR) of 65%, and a progression free survival (PFS) of 15 months for subjects with large B cell lymphoma (LBCL) administered hypoimmune allogenic CD19-directed CAR T cells of the present disclosure as a second-line treatment. In some embodiments, a complete response (CR) is defined as no detectable evidence of tumor. In some embodiments, certain efficacy criteria comprise an overall response rate (ORR) of 70%, CRR of 50%, durable CRR of ≥40%, and median duration of response (mDOR) ≥12 months for subjects with LBCL administered hypoimmune allogenic CD19-directed CAR T cells of the present disclosure as a third-line treatment. In some embodiments, certain efficacy criteria comprise an ORR of 70%, CRR of 50%, durable CRR of ≥40%, and mDOR ≥12 months for subjects with transplant ineligible LBCL administered hypoimmune allogenic CD19-directed CAR T cells of the present disclosure as a second-line treatment. In some embodiments, certain efficacy criteria comprise an ORR of 80%, CRR of 40%, durable CRR of 30-40%, mDOR ≥12 months, and 70% MRD negativity for subjects with CLL administered hypoimmune allogenic CD19-directed CAR T cells of the present disclosure as a third-line treatment. In some embodiments, certain efficacy criteria comprise ≥50% durable CRR and ≥60% best overall CRR in LBCL patients. In some embodiments, certain efficacy criteria comprise ≥30% best overall CRR and ≥3 months durability in CLL patients. In some embodiments, certain efficacy criteria comprise complete response with incomplete marrow recovery (CRi) and/or nodal partial response (nPR) in CLL patients. In some embodiments, CRi criteria comprise CR per iwCLL criteria along with any of the following: ANC <1.5×10⁹/L or requires exogenous growth factors (e.g., G-CSF)≤fourteen days (for at least seven days with “standard” growth factors) before the relevant blood count assessment to maintain an ANC ≥1.5×10⁹/L; platelet count <100×10⁹/L or requires exogenous growth factors or platelet transfusions ≤fourteen days before the relevant blood count assessment to maintain a platelet count >100×10⁹/L; and/or hemoglobin <110 g/L (11.0 g/dL) or requires exogenous growth factors or red blood cell transfusions ≤fourteen days before the relevant blood count assessment to maintain a hemoglobin ≥110 g/L (11.0 g/dL). In some embodiments, nPR criteria comprise all criteria for CR/CRi are met except for bone marrow that shows detectable lymphoid nodules. In some embodiments, hypoimmune allogenic CD19-directed CAR T cells of the present disclosure persist for at least 3 months and demonstrate HIP-mediated immune evasion in subjects of the present disclosure.

### 7. Procedures for Subjects

In some embodiments, subjects of the present disclosure undergo screening before administration of a lymphodepletion regiment as described herein and/or before administration of hypoimmune allogenic CD19-directed CAR T cells of the present disclosure. In some embodiments, screening comprises one or more of: informed consent, demographic data, medical history, previous cancer treatment history, concomitant medications documentation, clinical examination including height and weight, vital signs, neurologic examination (ICE Score), ECOG performance status, ECHO, ECG, brain MRI, lumbar puncture (if clinically indicated), disease assessment (including imaging and bone marrow biopsy/aspirate where applicable), tumor biopsy (if archival tissue not available), SAE reporting, local laboratory assessments (e.g., chemistry panel (including creatinine clearance, as estimated by MDRD cquation) (serum), CBC with differential (blood), beta-human chorionic gonadotropin (β-hCG) pregnancy test (serum or urine) for all females of childbearing potential, serologic tests (serum) (i.e., HIV, hepatitis B virus, and HCV), coagulation (plasma), urinalysis, LDH, CRP, Ferritin, beta-2 microglobulin, direct antiglobulin test, haptoglobin, COVID-19 test, serology (HIV, HBV, HCV), serum immunoglobulin (Ig) levels, immunohistochemistry analysis (biopsy), ctDNA, or MRD.

In some embodiments, the following lymphodepletion protocol is followed for a subject of the present disclosure. On Days −5 through −3, before the infusion of hypoimmune allogenic CD19-directed CAR T cells of the present disclosure, subjects receive a non-myeloablative lymphodepletion (LD) regimen consisting of cyclophosphamide and fludarabine to induce lymphocyte depletion and create an optimal environment for the expansion of hypoimmune allogenic CD19-directed CAR T cells of the present disclosure in vivo. Hypoimmune allogenic CD19-directed CAR T cells of the present disclosure should be administered 3 days (range 2-7 days) after completion of LD chemotherapy.

In some embodiments, subjects of the present disclosure receive the following 3-day chemotherapy regimen per the order identified as follows: 1) IV hydration with 1 L of 0.9% sodium chloride (NaCl) given before cyclophosphamide on the day of infusion; 2) Cyclophosphamide 500 mg/m² IV over approximately 60 minutes on Days −5, −4, and −3; 3) Fludarabine 30 mg/m² IV over approximately 30 minutes on Days −5, −4, and −3 and subjects with moderate impairment of renal function (creatinine clearance 50-70 mL/min/1.73 m²) should have their fludarabine dose reduced by 20% and be monitored closely; 4) an additional 1 L of 0.9% NaCl at the completion of the fludarabine infusion; and 5) add Mesna (sodium 2-mercaptoethanesulfonate; a detoxifying agent used to inhibit the hemorrhagic cystitis induced by the chemotherapy) per institutional guidelines. Subjects at risk for tumor lysis syndrome (TLS) should receive TLS prophylaxis prior to LD chemotherapy according to institutional guidelines.

Administration of hypoimmune allogenic CD19-directed CAR T cells of the present disclosure can increase the risk of high-grade and fatal toxicities in subjects with ongoing infection and/or inflammation. In some embodiments, if a subject of the present disclosure has: a temperature >38° C. within 72 hours before LD chemotherapy; CRP ≥100 mg/L any time between enrollment and the start of LD chemotherapy; and/or a white blood cell (WBC) count or WBC differential concerning for infectious process between enrollment and the start of LD chemotherapy (e.g., WBC >20,000 cells/μL, rapidly increasing WBC, or WBC differential with a high percentage of segments/bands), the cause of the infection should be identified and treated before administration of an LD regimen of the present disclosure.

In some embodiments, if any screening assessments or procedures are repeated between confirmation of eligibility and the start of LD chemotherapy and the results are outside of the eligibility criteria provided herein, any suspected/identified infectious/inflammatory process condition must resolve before a subject of the present disclosure is administered an LD regimen of the present disclosure. In some embodiments, a complete medical history and physical examination of a subject of the present disclosure including the head, eyes, ears, nose, and throat (HEENT) and cardiac, vascular, respiratory, gastrointestinal, integumentary, and neurological systems does not reveal evidence of infection/inflammation before administration of an LD regimen of the present disclosure. In some embodiments, a subject of the present disclosure has not received systemic antimicrobials for the treatment of a known or suspected infection within the 48 hours before an LD regimen of the present disclosure (prophylactic use of antimicrobials is allowed). In some embodiments, a treatment course of any antimicrobials given for a known or suspected antecedent infection should be completed before a subject of the present disclosure is administered an LD regimen of the present disclosure. In some embodiments, if a subject of the present disclosure is confirmed to have an infectious process for which antimicrobials are not available (e.g., viral pneumonia), the infection must be clinically resolved before a subject of the present disclosure is administered an LD regimen of the present disclosure. In some embodiments, the most recently collected blood, urine, or other body fluid cultures must show no growth for at least 48 hours, and any other infectious workup performed (e.g., bacterial, PCR, or stool or imaging studies) is negative before a subject of the present disclosure is administered an LD regimen of the present disclosure. In some embodiments, if there is clinical suspicion of an infection for which cultures are unlikely to be positive within 48 hours (e.g., fungal infection), then adequate time is allowed to allow the cultures to become positive before a subject of the present disclosure is administered an LD regimen of the present disclosure.

In some embodiments, subjects of the present disclosure are hospitalized during a treatment period from Day 0 before infusion with hypoimmune allogenic CD19-directed CAR T cells of the present disclosure until a minimum of 7 days after infusion. In some embodiments, the volume of hypoimmune allogenic CD19-directed CAR T cells of the present disclosure infused, thaw start/stop time, and the infusion start/stop time must all be noted in a subject's medical record. In some embodiments, hypoimmune allogenic CD19-directed CAR T cells of the present disclosure are not be thawed until the subject is ready for the infusion.

In some embodiments, subjects of the present disclosure remain hospitalized until all CAR-T related non-hematologic toxicities resolve to Grade 1 or return to baseline. In some embodiments, subjects may be discharged for a hospital with noncritical and clinically stable or improving toxicities (e.g., renal insufficiency) even if higher than Grade 1. In some embodiments, subjects should remain hospitalized for ongoing hypoimmune allogenic CD19-directed CAR T cells of the present disclosure-related fever, hypotension, hypoxia, or ongoing neurotoxicity of higher than Grade 1. In some embodiments, subjects of the present disclosure refrain from driving or hazardous activities for at least 8 weeks following infusion.

In some embodiments, after administration of hypoimmune allogenic CD19-directed CAR T cells of the present disclosure and being discharged from the hospital, a subject of the present disclosure will be evaluated at the following intervals: Day 8 (±1 day), Day 10 (±1 day), Day 13 (±1 day), Day 16 (±1 day), Day 21 (±4 days), Day 28 (±4 days), monthly in months 2-12 (±1 week) (or until start of a new systemic therapy). In some embodiments, subjects undergo the following assessments and procedures at the aforementioned intervals: clinical examination, vital signs, weight, ECOG assessment, concomitant medications documentation, neurologic examination to assess ICE score, ECG (if clinically indicated), brain MRI (if clinically indicated), lumbar puncture (if clinically indicated), tumor biopsy, disease/overall response assessments (including imaging and bone marrow biopsy/aspirate when applicable), AE/SAE/DLT reporting, chemistry panel (serum), CBC with differential (blood), coagulation (plasma), urinalysis (if clinically indicated), LDH, CRP, ferritin, beta-2 microglobulin, direct antiglobulin test, haptoglobin, serum Ig levels, Covid-19 and other viral serology testing (as clinically indicated), β-hCG pregnancy test (serum or urine) for all females of childbearing potential (as clinically indicated), analytes including cytokines (serum/plasma) and anti-CAR-T antibodies, i.e., donor specific antibody and CD19-directed CAR antibody (serum), ctDNA, MRD, CD19-directed CAR T cell kinetics (VCN and FACS), CD47 expression, RCL, T cell clonality, and exploratory analyses including immunophenotyping, T cell killing, NK cell killing, and scRNAseq (PBMCs), assessment of levels of analytes including cytokines (serum/plasma) and CD19-directed CAR T cells (PBMCs) should be drawn at the first onset and first reoccurrence of any Grade 2 or higher CAR-T-related toxicity, such as a Grade 2 CRS or neurologic event, and upon resolution of the event, if not already collected on the day (serum/plasma).

In some embodiments, a subject of the present disclosure does not receive subsequent anti-cancer therapy. In some embodiments, a subject of the present disclosure does receive subsequent anti-cancer therapy if their disease progression has been documented. In some embodiments, subsequent anti-cancer therapy is or comprises chemotherapy, immunotherapy, targeted agents, SCT, radiation therapy, corticosteroids (other than those used to manage CRS and ICANS), and other investigational agents.

### 8. Adverse Events and Toxicity Management

In some embodiments, a subject of the present disclosure experiences one or more adverse events (AEs) after administration of hypoimmune allogenic CD19-directed CAR T cells of the present disclosure. In some embodiments, an AE is or comprises any untoward medical occurrence in a subject of the present disclosure. In some embodiments, an AE is or comprises worsening of a pre-existing medical condition. Worsening indicates that the pre-existing medical condition has increased in severity, frequency, and/or duration or has an association with a worse outcome. In some embodiments, an AE does not include the following: a pre-existing condition that has not or involves an intervention such as elective cosmetic surgery or a medical procedure; interventions (and associated complications) for pre-treatment conditions (such as elective cosmetic surgery) or medical procedures that were planned before administration of hypoimmune allogenic CD19-directed CAR T cells of the present disclosure; hospitalization for infusions of hypoimmune allogenic CD19-directed CAR T cells or related procedures, and hospitalization as a precautionary measure. In some embodiments, “disease progression” as assessed by measurement of malignant lesions on radiographs or other methods is not considered to be an AE. In some embodiments, worsening of signs and symptoms of a B cell malignancy of the present disclosure are considered to be AEs.

In some embodiments, a subject of the present disclosure experiences one or more dose limiting toxicities (DLTs) after administration of hypoimmune allogenic CD19-directed CAR T cells of the present disclosure. In some embodiments, a DLT is or comprises one or more of the following events that are at least possibly related to treatment with hypoimmune allogenic CD19-directed CAR T cells of the present disclosure and that occur within 28 days of infusion with hypoimmune allogenic CD19-directed CAR T cells of the present disclosure: any Grade 5 event (death) without clinical or radiologic evidence of disease progression; CRS: all Grade 4 CRS, and Grade 3 CRS that fails to improve to ≤Grade 2 within 72 hours following adequate therapy; Neurotoxicity: All ≥Grade 3 neurotoxicity of any duration; Grade 3 or higher acute GvHD that requires oral or IV corticosteroids (>1 mg/kg) and does not resolve within 7 days. Grade 2 acute GvHD that is steroid refractory (i.e., progression after 3 days or no response after 7 days of systemic steroid treatment [≥1 mg/kg/day prednisone or equivalent]); Grade 4 neutropenia or thrombocytopenia, not attributable to underlying disease or lymphodepletion chemotherapy, that does not improve to ≤Grade 2 within 42 days. Grade 3 thrombocytopenia with clinically significant bleeding; ≥Grade 3 toxicity involving vital organs (e.g., cardiac, pulmonary) of any duration (Exceptions may be made for Grade 3 or 4 abnormal hepatic or renal function tests that improves to ≤Grade 2 within 7 days); all other Grade 3 toxicities, not attributable to underlying disease or lymphodepleting chemotherapy, that do not resolve to ≤Grade 2 within 72 hours. In some embodiments, the following events are not considered to be DLTs: Grade 3 cytopenia or Grade 4 lymphopenia/anemia, Grade 3 TLS for ≤2 weeks, and/or Grade 3 infections.

In some embodiments, a subject of the present disclosure experiences one or more serious adverse events (SAEs) after administration of hypoimmune allogenic CD19-directed CAR T cells of the present disclosure. In some embodiments, an SAE is defined as an AE that meets at least one of the following serious criteria: is fatal, is life-threatening (i.e., an event that places the subject at immediate risk of death; it does not refer to an event that hypothetically might have caused death if it were more severe), requires inpatient hospitalization or prolongation of existing or planned hospitalization, an AE would meet the criterion of “requires hospitalization” if the event necessitated an admission to a healthcare facility, events that require an escalation of care when the subject is already hospitalized should be recorded as an SAE (e.g., movement from routine care in the hospital to the intensive care unit or if that event resulted in a prolongation of the planned hospitalization, results in persistent or significant disability/incapacity, is a congenital anomaly/birth defect, and/or is another medically important serious event. The terms “severe” and “serious” are not synonymous. Severity refers to the intensity of an AE according to NCI Common Terminology Criteria for Adverse Events (CTCAE); the event itself may be of relatively minor medical significance and, therefore, may not meet the seriousness criteria listed above. Severity and seriousness need to be independently assessed for each AE. Progression of a malignancy is not considered to be a drug-related SAE; signs and symptoms of disease progression can be considered to be SAEs (and documented as being due to disease progression).

In some embodiments, an AE is or comprises a targeted AE. In some embodiments, an SAE is or comprises a targeted SAE. Targeted AEs/SAEs include neurologic, hematologic, infection, GvHD, autoimmune disorder, and second primary malignancy events. Adverse events of special interest (reflecting potential risks of CD19-directed CAR therapies) include: CRS, neurotoxicity (i.e., ICANS), Macrophage Activation Syndrome, TLS, Grade 3 cytopenia persisting through Day 28 post-treatment, Severe (Grade 3) infection through the first 28 days post-treatment.

In some embodiments, AEs are determined based on abnormal clinical laboratory findings. In some embodiments, abnormal laboratory findings without clinical significance are not AEs. In some embodiments, abnormal laboratory findings that result in new or worsening clinical sequelae or that require therapy or adjustment in current therapy are considered AEs. In some embodiments, an AE is an abnormal laboratory test result that results in a medical intervention (e.g., potassium supplementation for hypokalemia or iron replacement therapy for anemia) or a change in concomitant therapy. In some embodiments, an abnormal vital sign results is an AE if it is a change from baseline and is accompanied by clinical symptoms and/or results in a medical intervention or a change in concomitant therapy.

In some embodiments, risks associated with CD19-directed CAR T cell treatment are or comprise cytokine release syndrome (CRS), neurotoxicity, cytopenia, hypogammaglobulinemia and infections, and TLS. In some embodiments, risks associated with allogeneic CAR T cell therapy include GvHD and clonal expansion (e.g., second primary malignancy).

In some embodiments, if a subject of the disclosure experiences CRS, it is managed according to ASTCT guidelines (Lee 2019). In some embodiments, if a subject of the disclosure experiences neurotoxicity (i.e., ICANS) it is managed according to ASTCT guidelines (Lee 2019). In some embodiments, if a subject of the disclosure experiences GvHD it is managed according to Mount Sinai Acute GVHD International Consortium 2016 report (Harris 2016). In some embodiments, if a subject of the present disclosure experiences hemophagocytic lymphohistiocytosis (HLH), also known as Immune Effector Cell-Associated Hemophagocytic Lymphohistiocytosis-Like Syndrome (IEC-HS), it can be graded according to ASTCT IEC-HS 2023 guidelines.

Infections, hypogammaglobulinemia, and cytopenia are known side effects of CD19-directed CAR T cell therapy. Infections after CAR T cell therapy are common and have been reported in up to 70% of patients who received CAR T cell therapy in registrational clinical trials for approved agents (KYMRIAH® Prescribing Information, YESCARTA® Prescribing Information, BREYANZI® Prescribing Information). Bacterial, viral, and fungal infections have all been reported with use of CAR T cell therapy. Infections may occur for a number of concomitant reasons, including lymphodepleting (or antecedent) chemotherapy, CAR T cell-mediated B cell or plasma cell depletion, prolonged cytopenia, corticosteroid treatment, or as a consequence of the malignancy itself. The severity of CRS may also be associated with an increased risk of acute infections. Other potential risk factors for severe infections within the first 30 days of CAR-T treatment include ICANS, tocilizumab, and corticosteroid use. Subjects may remain at increased risk of complications for weeks to months after infusion (Logue 2021, Hill 2018). Infections are generally managed using agents that target the source of infection. Additionally, prophylaxis against vesicular stomatitis virus/herpes simplex virus reactivation and neumocystis jirovecii pneumonia infections is generally used for patients undergoing CAR T cell therapy and for several months after. The decision to administer antibacterial or antifungal prophylaxis should be risk-adjusted based on subject characteristics, such as prior lines of suppressive therapy and infection history (Thompson 2022).

Hypogammaglobulinemia is characterized by low antibody levels due to extremely low B cell or plasma cell counts, referred to as B cell or plasma cell aplasia, respectively. Hypogammaglobulinemia has been reported in up to 53% of patients who received CAR T cell therapy in registrational clinical trials and is associated with risk of infection (KYMRIAH® Prescribing Information, YESCARTA® Prescribing Information, BREYANZI® Prescribing Information). After lymphodepleting chemotherapy and treatment of the present disclosure, subjects can be monitored for serum immunoglobulin (Ig) levels and CBC with differential. Subjects with hypogammaglobulinemia (serum IgG levels <400-600 mg/dL) and serious or recurrent infections (particularly bacterial) should be managed with intravenous immunoglobulin (IVIG) replacement therapy (400-500 mg/kg IVIG given monthly). IVIG should be continued until serum IgG levels normalize and infections are resolved. The optimal IgG threshold to use may depend on subject characteristics and infection frequency or severity.

Subjects who receive CAR T cell therapy are also at risk for hematologic toxicities, including prolonged cytopenia, such as neutropenia, thrombocytopenia, anemia, and/or leukopenia (KYMRIAH® Prescribing Information, YESCARTA® Prescribing Information, BREYANZI® Prescribing Information). Cytopenia may occur in the weeks to months after lymphodepleting chemotherapy and CAR T cell therapy infusion. Factors that may contribute to prolonged cytopenia include CRS and ICANS severity, disease burden, the number of prior therapies, baseline blood cell counts, peak CRP and ferritin levels, and CAR construct. Although lymphodepletion may be a contributing factor, the pathophysiology of prolonged cytopenia after CAR T cell infusion remains unclear. Alternative causes of cytopenia in treated include myelodysplastic syndrome or second primary malignancy (Jain 2020, Fried 2019). Cytopenia are generally managed with transfusion or growth factor support, if the possibility of myelodysplastic syndrome has been ruled out. Growth factors may be considered for persistent cytopenia. GM-CSF is not recommended in the setting of CAR T cell therapy. An FDA-approved biosimilar is an appropriate substitute for filgrastim (Thompson 2022).

### B. Hypoimmunogenic Cells

In some embodiments, the present disclosure is directed primary cells (such as, but not limited to, primary T cells). In some embodiments, primary cells such as primary T cells, are engineered for reduced expression or lack of expression of MHC class I and/or MHC class II human leukocyte antigens, and in some instances, for reduced expression or lack of expression of a T-cell receptor (TCR) complex. In some embodiments, the hypoimmune (HIP) T cells and primary T cells overexpress CD47 and a chimeric antigen receptor (CAR) in addition to reduced expression or lack of expression of MHC class I and/or MHC class II human leukocyte antigens, and have reduced expression or lack expression of a T-cell receptor (TCR) complex. In some embodiments, the CAR is a CD19-specific CAR. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, engineered and/or hypoimmune (HIP) T cells and primary T cells overexpress CD47 and a chimeric antigen receptor (CAR), and include a genomic modification of the B2M gene. In some embodiments, engineered and/or hypoimmune (HIP) T cells and primary T cells overexpress CD47 and include a genomic modification of the CIITA gene. In some embodiments, engineered and/or hypoimmune (HIP) T cells and primary T cells overexpress CD47 and a CAR, and include a genomic modification of the TRAC gene. In some embodiments, engineered and/or hypoimmune (HIP) T cells and primary T cells overexpress CD47 and a CAR, and include one or more genomic modifications selected from the group consisting of the B2M, CIITA, and TRAC genes. In some embodiments, engineered and/or hypoimmune (HIP) T cells and primary T cells overexpress CD47 and a CAR, and include genomic modifications of the B2M, CIITA, and TRAC genes. In some embodiments, the cells are B2M−/−, CIITA−/−, TRAC−/−, CD47tg cells that also express CARs.

In certain embodiments, the cells are B2M^{indel/indel}, CIITA^{indel/indel}, TRAC^{indel/indel}, CD47tg cells that also express CARs. Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), γδ T cells, and any other subtype of T cells. In some embodiments, primary T cells are selected from a group that includes cytotoxic T-cells, helper T-cells, memory T-cells, regulatory T-cells, tumor infiltrating lymphocytes, and combinations thereof. In some embodiments, cells are modified or engineered as compared to a wild-type or control cell, including an unaltered or unmodified wild-type cell or control cell. In some embodiments, a wild-type cell or a control cell is a starting material. In some embodiments, starting material is a primary cell collected from a donor. In some embodiments, starting material is a primary blood cell collected from a donor, e.g., via a leukopak. In some embodiments, starting material is otherwise modified or engineered to have altered expression of one or more genes to generate an engineered cell.

In some embodiments, primary T cells are from a pool of primary T cells from one or more donor subjects that are different than the recipient subject (e.g., the patient administered the cells). Primary T cells can be obtained from 1, 2, 3, 4, 5, 6, 7, 8, 9, 10, 20, 50, 100 or more donor subjects and pooled together. Primary T cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, primary T cells are harvested from one or a plurality of individuals, and in some instances, primary T cells or a pool of primary T cells are cultured in vitro. In some embodiments, primary T cells or a pool of primary T cells are engineered to exogenously express CD47 and cultured in vitro.

In certain embodiments, primary T cells or a pool of primary T cells are engineered to express a chimeric antigen receptor (CAR). Useful CARs include those that bind a CD19 antigen. In some cases, a CAR is the same or equivalent to those used in FDA-approved CAR-T cell therapies such as, but not limited to, those used in tisagenlecleucel and axicabtagene ciloleucel, or others under investigation in clinical trials.

In some embodiments, the primary T cells or the pool of primary T cells are engineered to exhibit reduced expression of an endogenous T cell receptor compared to unmodified primary T cells. Methods of genetically modifying a cell including a T cell are described in detail, for example, in WO2020/018620 and WO2016/183041, the disclosures of which are herein incorporated by reference in their entireties, including the tables, appendices, sequence listing and figures.

In some embodiments, CAR-T cells comprise a second generation CAR comprising an antigen binding domain, a transmembrane domain, and at least two signaling domains.

In some embodiments, CAR-T cells comprise a CAR comprising an antigen binding domain, a transmembrane, and one or more signaling domains. In some embodiments, the CAR also comprises a linker. In some embodiments, the CAR comprises a CD19 antigen binding domain. In some embodiments, the CAR comprises a CD8α transmembrane domain. In some embodiments, the CAR comprises a CD8α signal peptide. In some embodiments, the CAR comprises a Whitlow linker GSTSGSGKPGSGEGSTKG (SEQ ID NO: 24). In some embodiments, the antigen binding domain of the CAR is selected from a group including, but not limited to, (a) an antigen binding domain targets an antigen characteristic of a neoplastic cell or an antigen binding domain targets an antigen characteristic of an autoimmune disorder.

In some embodiments, the CAR further comprises one or more linkers. The format of an scFv is generally two variable domains linked by a flexible peptide sequence, or a “linker,” either in the orientation VH-linker-VL or VL-linker-VH. Any suitable linker known to those in the art in view of the specification can be used in the CARs. Examples of suitable linkers include, but are not limited to, a GS based linker sequence, and a Whitlow linker GSTSGSGKPGSGEGSTKG (SEQ ID NO: 24).

In some embodiments, the antigen binding domain is selected from a group that includes an antibody, an antigen-binding portion or fragment thereof, an scFv, and a Fab. In some embodiments, the antigen binding domain binds to CD19. In some embodiments, the antigen binding domain is an anti-CD19 scFv such as but not limited to FMC63.

In some embodiments, the transmembrane domain comprises one selected from a group that includes a transmembrane region of TCRα, TCRβ, TCRζ, CD3ε, CD3γ, CD3δ, CD3ζ, CD4, CD5, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof. In some embodiments, the transmembrane domain comprises a CD8α transmembrane domain.

In some embodiments, the signaling domain(s) of the CAR comprises a costimulatory domain(s). For instance, a signaling domain can contain a costimulatory domain. Or, a signaling domain can contain one or more costimulatory domains. In certain embodiments, the signaling domain comprises a costimulatory domain. In other embodiments, the signaling domains comprise costimulatory domains. In some cases, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

In some embodiments, a CAR comprises a CD3 zeta (CD3ζ) intracellular domain or functional variant thereof. In some embodiments, a CAR comprises a 4-1BB co-stimulatory intracellular domain or functional variant thereof. In some embodiments, the CAR comprises (i) a CD3ζ domain or functional variant thereof; and (ii) a 4-1BB domain or functional variant thereof.

Methods for introducing a CAR construct or producing CAR-T cells are well known to those skilled in the art. Detailed descriptions are found, for example, in Vormittag et al., Curr Opin Biotechnol, 2018, 53, 162-181; and Eyquem et al., Nature, 2017, 543, 113-117.

In some embodiments, the cells derived from primary T cells comprise reduced expression of an endogenous T cell receptor, for example by disruption of an endogenous T cell receptor gene (e.g., T cell receptor alpha constant region (TRAC)).

In some embodiments, a CD47 transgene is inserted into a random locus of a cell. In some embodiments, a transgene encoding a CAR is inserted into a random locus of a cell. In some embodiments, a transgene encoding a CAR is inserted into a random locus of a cell via viral vector transduction/integration. In some embodiments, a CD47 transgene and a transgene encoding a CAR are inserted into a random locus of a cell via viral vector transduction/integration. In some embodiments, a vector is a self-inactivating lentiviral vector pseudotyped with a vesicular stomatitis VSV-G envelope. In some embodiments, a transgene encoding a CAR is inserted into at least one allele of a cell using viral transduction. In some embodiments, an exogenous polynucleotide is inserted into at least one allele of a cell using a lentivirus based viral vector.

In some embodiments, a CD47 transgene and a transgene encoding a CAR are inserted into the same locus.

In certain embodiments, a CD47 and a CAR are controlled by a single promoter and are encoded by a single transgene. In some instances, the promoter controlling expression of any transgene described is a constitutive promoter. In other instances, the promoter for any transgene described is an inducible promoter. In some embodiments, the promoter is an EF1α promoter. In some embodiments, the promoter is CAG promoter. In some embodiments, a CD47 transgene and a transgene encoding a CAR are both controlled by a constitutive promoter. In some embodiments, a CD47 transgene and a transgene encoding a CAR are both controlled by an inducible promoter.

Methods provided are useful for inactivation or ablation of MHC class I expression and/or MHC class II expression in cells such as primary T cells. In some embodiments, genome editing technologies utilizing rare-cutting endonucleases (e.g., CRISPR/Cas) are also used to reduce or eliminate expression of genes involved in an immune response (e.g., by deleting genomic DNA of genes involved in an immune response or by insertions of genomic DNA into such genes, such that gene expression is impacted) in cells. In certain embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an innate and/or an adaptive immune response) in a recipient subject.

In some embodiments, a cell includes a modification to increase expression of CD47.

In some embodiments, a cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including B2M and CIITA. In some embodiments, the cell comprises a genetic editing modification to the B2M gene. In some embodiments, the cell comprises a genetic editing modification to the CIITA gene. In some embodiments, the cell comprises genetic editing modifications to the B2M and CIITA genes. In certain embodiments, the genome of the cell has been altered to reduce or delete critical components of HLA expression. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the present disclosure provides a cell (e.g., primary T cell or CAR-T cell) or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell (e.g., primary T cell or CAR-T cell) or population thereof comprising a genome in which a gene has been edited to delete a contiguous stretch of genomic DNA, thereby reducing or eliminating surface expression of MHC class II molecules in the cell or population thereof. In numerous embodiments, the present disclosure provides a cell (e.g., primary T cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In certain embodiments, the expression of MHC I molecules and/or MHC II molecules is modulated by targeting and deleting a contiguous stretch of genomic DNA, thereby reducing or eliminating expression of a target gene selected from the group consisting of B2M and CIITA. In some embodiments, described herein are genetically edited cells (e.g., modified human cells) comprising exogenous CD47 proteins and inactivated or modified CIITA gene sequences, and in some instances, additional gene modifications that inactivate or modify B2M gene sequences.

Provided herein are cells exhibiting a modification of one or more targeted polynucleotide sequences that regulates the expression of any one of the following: (a) MHC I antigens, (b) MHC II antigens, (c) TCR complexes, (d) both MHC I and II antigens, and (e) MHC I and II antigens and TCR complexes. In certain embodiments, the modification includes increasing expression of CD47. In some embodiments, the cells include an exogenous or recombinant CD47 polypeptide. In certain embodiments, the modification includes expression of a chimeric antigen receptor. In some embodiments, the cells comprise an exogenous or recombinant chimeric antigen receptor polypeptide.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In certain embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In certain embodiments, the present disclosure provides a cell or population thereof comprising a genome in which a gene has been edited to delete a contiguous stretch of genomic DNA, thereby reducing or eliminating surface expression of TCR molecules in the cell or population thereof. In numerous embodiments, the present disclosure provides a cell or population thereof comprising a genome in which one or more genes has been edited to delete a contiguous stretch of genomic DNA, thereby reducing or eliminating surface expression of MHC class I and II molecules and TCR complex molecules in the cell or population thereof.

In some embodiments, the cells and methods described herein include genomically editing human cells to cleave CIITA gene sequences as well as editing the genome of such cells to alter one or more additional target polynucleotide sequences such as, but not limited to, B2M and TRAC. In some embodiments, the cells and methods described herein include genomically editing human cells to cleave B2M gene sequences as well as editing the genome of such cells to alter one or more additional target polynucleotide sequences such as, but not limited to, CIITA and TRAC. In some embodiments, the cells and methods described herein include genomically editing human cells to cleave TRAC gene sequences as well as editing the genome of such cells to alter one or more additional target polynucleotide sequences such as, but not limited to, B2M and CIITA.

In some embodiments, a population of engineered cells described evades NK cell mediated cytotoxicity upon administration to a recipient patient. In some embodiments, a population of engineered cells evades NK cell mediated cytotoxicity by one or more subpopulations of NK cells. In some embodiments, a population of engineered is protected from cell lysis by NK cells, including immature and/or mature NK cells upon administration to a recipient patient. In some embodiments, a population of engineered cells evades macrophage engulfment upon administration to a recipient patient. In some embodiments, a population of engineered cells does not induce an innate and/or an adaptive immune response to the cell upon administration to a recipient patient.

In some embodiments, cells described herein are controlled by a safety switch. The term “safety switch” used herein refers to a system for controlling the expression of a gene or protein of interest that, when downregulated or upregulated, leads to clearance or death of the cell, e.g., through recognition by the host's immune system. A safety switch can be designed to be triggered by an exogenous molecule in case of an adverse clinical event. A safety switch can be engineered by regulating the expression on the DNA, RNA and protein levels. A safety switch includes a protein or molecule that allows for the control of cellular activity in response to an adverse event. In some embodiments, a safety switch comprises a therapeutic agent that inhibits or blocks the interaction of CD47 and SIRPα. In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### 1. CIITA

In some embodiments, the technologies disclosed herein modulate (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present disclosure is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In certain cases, the amino acid sequence of CIITA is depicted as NCBI GenBank No. AAA88861.1. Additional descriptions of the CIITA protein and gene locus can be found in Uniprot No. P33076, HGNC Ref. No. 7067, and OMIM Ref. No. 600005.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS: 5184-36352 of Table 12 of WO2016183041, which is herein incorporated by reference. In some embodiments, the cell has a reduced ability to induce an innate and/or an adaptive immune response in a recipient subject. In some embodiments, an exogenous nucleic acid encoding a polypeptide as disclosed herein (e.g., a chimeric antigen receptor, CD47, or another tolerogenic factor disclosed herein) is inserted at the CIITA gene.

Assays to test whether the CIITA gene has been inactivated are known and described herein. In some embodiments, the resulting genetic modification of the CIITA gene by PCR and the reduction of HLA-II expression can be assays by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating genetic modification. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

### 2. B2M

In some embodiments, the technologies disclosed herein modulate (e.g., reduce or eliminate) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an innate and/or an adaptive immune response in a recipient subject.

In some embodiments, the target polynucleotide sequence of the present disclosure is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In certain cases, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref. No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS: 81240-85644 of Table 15 of WO2016183041, which is herein incorporated by reference. In some embodiments, an exogenous nucleic acid encoding a polypeptide as disclosed herein (e.g., a chimeric antigen receptor, CD47, or another tolerogenic factor disclosed herein) is inserted at the B2M gene. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

Assays to test whether the B2M gene has been inactivated are known and described herein. In some embodiments, the resulting genetic modification of the B2M gene by PCR and the reduction of HLA-I expression can be assays by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating genetic modification.

### 3. TRAC

In many embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some embodiments, the modulation occurs using a CRISPR/Cas system. By modulating (e.g., reducing or deleting) expression of TRAC, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in RefSeq. No. NG_001332.3 and NCBI Gene ID No. 28755. In certain cases, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS: 532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression can be assays by FACS analysis. In another embodiment, TRAC protein expression is detected using a Western blot of cells lysates probed with antibodies to the TRAC protein. In another embodiment, reverse transcriptase polymerase chain reactions (RT-PCR) are used to confirm the presence of the inactivating genetic modification.

### 4. TRB

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. P0DSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. P0DSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS: 610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, reverse transcriptase polymerase chain reactions (RT-PCR) are used to confirm the presence of the inactivating genetic modification.

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. P0DSE2. In some instances, the TRB gene locus is described in RefSeq. No. NG_001333.2 and NCBI Gene ID No. 6957. In certain cases, the amino acid sequence of TRB is depicted as Uniprot No. P01848. Additional descriptions of the TRB protein and gene locus can be found in GenBank No. L36092.2, Uniprot No. P0DSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS: 610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression can be assays by FACS analysis. In another embodiment, TRB protein expression is detected using a Western blot of cells lysates probed with antibodies to the TRB protein. In another embodiment, reverse transcriptase polymerase chain reactions (RT-PCR) are used to confirm the presence of the inactivating genetic modification.

### 5. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the cell is genetically modified to comprise an integrated exogenous polynucleotide encoding CD47 using homology-directed repair. In some instances, the cell expresses a nucleotide sequence encoding a human CD47 polypeptide such that the nucleotide sequence is inserted into at least one allele of a safe harbor or target locus. In some instances, the cell expresses a nucleotide sequence encoding a human CD47 polypeptide wherein the nucleotide sequence is inserted into at least one allele of an AAVS1 locus. In some instances, the cell expresses a nucleotide sequence encoding a human CD47 polypeptide wherein the nucleotide sequence is inserted into at least one allele of an CCR5 locus. In some instances, the cell expresses a nucleotide sequence encoding a human CD47 polypeptide wherein the nucleotide sequence is inserted into at least one allele of a safe harbor or target gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a human CD47 polypeptide wherein the nucleotide sequence is inserted into at least one allele of a TRAC locus.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 2.

TABLE 2
Amino acid sequences of human CD47

| | SEQ | | Amino |
|---|---|---|---|
| | ID | | acid |
| Protein | NO: | Sequence | residues |
| Human | 37 | QLLFNKTKSVEFTFCNDTVVIPCFVTNMEAQNTTEV | aa 19-323 |
| CD47 | | YVKWKFKGRDIYTFDGALNKSTVPTDESSAKIEVS | |
| (without | | QLLKGDASLKMDKSDAVSHTGNYTCEVTELTREG | |
| signal | | ETIIELKYRVVSWFSPNENILIVIFPIFAILLFWGQFGI | |
| sequence) | | KTLKYRSGGMDEKTIALLVAGLVITVIVIVGAILFVP | |
| | | GEYSLKNATGLGLIVTSTGILILLHYYVFSTAIGLTSF | |
| | | VIAILVIQVIAYILAVVGLSLCIAACIPMHGPLLISGL | |
| | | SILALAQLLGLVYMKFVASNQKTIQPPRKAVEEPLN | |
| | | AFKESKGMMNDE | |
| Human | 38 | MWPLVAALLLGSACCGSAQLLENKTKSVEFTFCND | aa 1-323 |
| CD47 (with | | TVVIPCFVTNMEAQNTTEVYVKWKFKGRDIYTFDG | |
| signal | | ALNKSTVPTDESSAKIEVSQLLKGDASLKMDKSDA | |
| sequence) | | VSHTGNYTCEVTELTREGETIIELKYRVVSWFSPNE | |
| | | NILIVIFPIFAILLFWGQFGIKTLKYRSGGMDEKTIAL | |
| | | LVAGLVITVIVIVGAILFVPGEYSLKNATGLGLIVTS | |
| | | TGILILLHYYVFSTAIGLTSFVIAILVIQVIAYILAVVG | |
| | | LSLCIAACIPMHGPLLISGLSILALAQLLGLVYMKFV | |
| | | ASNQKTIQPPRKAVEEPLNAFKESKGMMNDE | |

In some embodiments, the cell comprises a CD47 polypeptide having at least 95% sequence identity (e.g., 95%, 96%, 97%, 98%, 99%, or more) to the amino acid sequence of SEQ ID NO: 37. In some embodiments, the cell comprises a CD47 polypeptide having the amino acid sequence of SEQ ID NO: 37. In some embodiments, the cell comprises a CD47 polypeptide having at least 95% sequence identity (e.g., 95%, 96%, 97%, 98%, 99%, or more) to the amino acid sequence of SEQ ID NO: 38. In some embodiments, the cell comprises a CD47 polypeptide having the amino acid sequence of SEQ ID NO: 38.

In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO: 37. In some embodiments, the cell comprises a nucleotide sequence encoding a CD47 polypeptide having the amino acid sequence of SEQ ID NO: 37. In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO: 38. In some embodiments, the cell comprises a nucleotide sequence encoding a CD47 polypeptide having the amino acid sequence of SEQ ID NO: 38. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some cases, the polynucleotide encoding CD47 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is inserted into any one of the gene loci provided herein. In certain embodiments, the polynucleotide encoding CD47 is operably linked to a promoter.

In some embodiments, the polynucleotide encoding CD47 is inserted into at least one allele of the T cell using viral transduction. In some embodiments, the polynucleotide encoding CD47 is inserted into at least one allele of the T cell using a lentivirus based viral vector. In some embodiments, the lentivirus based viral vector is a pseudotyped, self-inactivating lentiviral vector that carries the polynucleotide encoding CD47. In some embodiments, the lentivirus based viral vector is a self-inactivating lentiviral vector pseudotyped with a vesicular stomatitis VSV-G envelope, and which carries the polynucleotide encoding CD47.

In another embodiment, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

### C. Chimeric Antigen Receptors

Provided herein are hypoimmunogenic cells comprising a chimeric antigen receptor (CAR). CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. In some embodiments, a CAR may comprise an extracellular binding domain (also referred to as a “binder”) that specifically binds a target antigen, a transmembrane domain, and an intracellular signaling domain. In some embodiments, a CAR may further comprise one or more additional elements, including one or more signal peptides, one or more extracellular hinge domains, and/or one or more intracellular costimulatory domains. Domains may be directly adjacent to one another, or there may be one or more amino acids linking the domains.

A nucleotide sequence encoding a CAR may be derived from a mammalian sequence, for example, a mouse sequence, a primate sequence, a human sequence, or combinations thereof. In the cases where a nucleotide sequence encoding a CAR is non-human, the sequence of the CAR may be humanized. A nucleotide sequence encoding a CAR may also be codon-optimized for expression in a mammalian cell, for example, a human cell. In any of these embodiments, a nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. Sequence variations may be due to codon-optimalization, humanization, restriction enzyme-based cloning scars, and/or additional amino acid residues linking the functional domains, etc.

In some embodiments, a CAR binds to CD19. In some embodiments, the CAR is a second generation CAR. In some embodiments, the CAR includes a single binding domain that binds to a single target antigen.

In some embodiments, a CD19 specific CAR includes an anti-CD19 single-chain antibody fragment (scFv), a transmembrane domain such as one derived from human CD8α, a 4-1BB (CD137) co-stimulatory signaling domain, and a CD35 signaling domain.

In some embodiments, a CAR comprises a commercial CAR construct carried by a T cell. Non-limiting examples of commercial CAR-T cell based therapies include brexucabtagene autoleucel (TECARTUS®), axicabtagene ciloleucel (YESCARTA®), idecabtagene vicleucel (ABECMA®), lisocabtagene maraleucel (BREYANZI®), tisagenlecleucel (KYMRIAH®), Descartes-08 and Descartes-11 from Cartesian Therapeutics, CTL110 from Novartis, P-BMCA-101 from Poseida Therapeutics, AUTO4 from Autolus Limited, UCARTCS from Cellectis, PBCAR19B and PBCAR269A from Precision Biosciences, FT819 from Fate Therapeutics, and CYAD-211 from Clyad Oncology.

In some embodiments, a hypoimmunogenic cell described herein comprises a polynucleotide encoding a chimeric antigen receptor (CAR) comprising an antigen binding domain. In some embodiments, a hypoimmunogenic cell described herein comprises a chimeric antigen receptor (CAR) comprising an antigen binding domain. In some embodiments, the polynucleotide is or comprises a chimeric antigen receptor (CAR) comprising an antigen binding domain. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

### 1. Antigen Binding Domain (ABD)

In some embodiments, an extracellular binding domain of a CAR may comprise one or more antibodies specific to one target antigen or multiple target antigens. An antibody may be an antibody fragment, for example, an scFv, or a single-domain antibody fragment, for example, a VHH. In some embodiments, an antigen binding domain comprises a light chain amino acid sequence and a heavy chain amino acid sequence. In some embodiments, a light chain amino acid sequence is not a full-length light chain sequence, but does comprise a light chain variable domain sequence. In some embodiments, a heavy chain amino acid sequence is not a full-length heavy chain sequence, but does comprise a light chain variable domain sequence. In certain embodiments, the scFv may comprise a heavy chain variable region (VH) and a light chain variable region (VL) of an antibody connected by a linker. The VH and the VL may be connected in either order, i.e., VH-linker-VL or VL-linker-VH. Non-limiting examples of linkers include Whitlow linker, (G4S)n (n can be a positive integer, e.g., 1, 2, 3, 4, 5, 6, etc.) linker, and variants thereof. In any of these embodiments, the extracellular binding domain of the CAR can be codon-optimized for expression in a host cell or have variant sequences to increase functions of the extracellular binding domain.

In some embodiments, the antigen binding domain (ABD) targets an antigen characteristic of a neoplastic cell. In other words, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is CD19 or an antigenic fragment or antigenic portion thereof.

In some embodiments, the antigen binding domain targets an antigen characteristic of a Bell cell mediated autoimmune disorder. In some embodiments, the ABD binds an antigen associated with a Bell cell mediated autoimmune disorder. In some instances, the antigen is expressed by a cell associated with a Bell cell mediated autoimmune disorder. In some embodiments, the a Bell cell mediated autoimmune disorder is selected from the group consisting of: systemic lupus erythematosus (SLE), lupus nephritis, CNS lupus, anti-neutrophilic cytoplasmic autoantibody (ANCA) associated vasculitis, granulomatous polyangiitis, microscopic polyangiitis, multiple sclerosis, pemphigus vulgaris, autoimmune blistering skin diseases, membranous nephropathy, anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, and autoimmune hemolytic anemia.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, e.g., US 2003/0077249; WO 2017/058753; WO 2017/058850, the contents of which are herein incorporated by reference.

In some embodiments, a CAR antigen binding domain is or comprises an antibody or an antigen-binding portion thereof. In some embodiments, a CAR antigen binding domain is or comprises an scFv or Fab. In some embodiments, a CAR antigen binding domain comprises an scFv or Fab fragment of a CD19 antibody.

### 2. Transmembrane Domain

In some embodiments, a CAR of the present disclosure comprises a transmembrane domain comprising a CD8α transmembrane domain or functional variant thereof. Table 3 provides the amino acid sequences of exemplary transmembrane domains.

TABLE 3
Exemplary sequences of transmembrane domains

| SEQ ID | | |
|---|---|---|
| NO: | Sequence | Description |
| 14 | IYIWAPLAGTCGVLLLSLVITLYC | CD8α transmembrane domain |
| 15 | FWVLVVVGGVLACYSLLVTVAFIIFW | CD28 transmembrane domain |
| | V | |
| 2 | MFWVLVVVGGVLACYSLLVTVAFIIF | CD28 transmembrane domain |
| | WV | |

### 3. Intracellular Signaling Domain

In some embodiments, a CAR described herein comprises two intracellular signaling domains, wherein the two intracellular signaling domains are or comprise a CD32 signaling domain (or a functional fragment thereof) and 4-1BB co-stimulatory domain (or a functional fragment thereof). Table 4 provides amino acid sequences few exemplary intracellular costimulatory and/or signaling domains. In certain embodiments, as in the case of tisagenlecleucel as described below, the CD3ζ signaling domain of SEQ ID NO:18 may have a mutation, e.g., a glutamine (Q) to lysine (K) mutation, at amino acid position 14 (see SEQ ID NO: 3).

TABLE 4
Exemplary sequences of intracellular costimulatory and/or signaling domains

| SEQ ID | | |
|---|---|---|
| NO: | Sequence | Description |
| 16 | KRGRKKLLYIFKQPFMRPVQTTQEEDGC | 4-1BB costimulatory domain |
| | SCRFPEEEEGGCEL | |
| 17 | RSKRSRLLHSDYMNMTPRRPGPTRKHYQ | CD28 costimulatory domain |
| | PYAPPRDFAAYRS | |
| 18 | RVKFSRSADAPAYQQGQNQLYNELNLGR | CD3ζ signaling domain |
| | REEYDVLDKRRGRDPEMGGKPRRKNPQ | |
| | EGLYNELQKDKMAEAYSEIGMKGERRR | |
| | GKGHDGLYQGLSTATKDTYDALHMQAL | |
| | PPR | |
| 3 | RVKFSRSADAPAYKQGQNQLYNELNLGR | CD3ζ signaling domain (with |
| | REEYDVLDKRRGRDPEMGGKPRRKNPQ | Q to K mutation at position |
| | EGLYNELQKDKMAEAYSEIGMKGERRR | 14) |
| | GKGHDGLYQGLSTATKDTYDALHMQAL | |
| | PPR | |

### 4. Additional CAR Elements

In some embodiments, a CAR comprises a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 5 below.

TABLE 5
Exemplary sequences of signal peptides

| SEQ ID | | |
|---|---|---|
| NO: | Sequence | Description |
| 6 | MALPVTALLLPLALLLHAARP | CD8α signal peptide |
| 7 | METDTLLLWVLLLWVPGSTG | IgK signal peptide |
| 8 | MLLLVTSLLLCELPHPAFLLIP | GMCSFR-α (CSF2RA) signal peptide |

In certain embodiments, a CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 6 below.

TABLE 6
Exemplary sequences of hinge domains

| SEQ ID | | |
|---|---|---|
| NO: | Sequence | Description |
| 9 | TTTPAPRPPTPAPTIASQPLSLRPEACRPAAGG | CD8α hinge domain |
| | AVHTRGLDFACD | |
| 10 | IEVMYPPPYLDNEKSNGTIIHVKGKHLCPSPLF | CD28 hinge domain |
| | PGPSKP | |
| 1 | AAAIEVMYPPPYLDNEKSNGTIIHVKGKHLCP | CD28 hinge domain |
| | SPLFPGPSKP | |
| 11 | ESKYGPPCPPCP | IgG4 hinge domain |
| 12 | ESKYGPPCPSCP | IgG4 hinge domain |
| 13 | ESKYGPPCPPCPAPEFLGGPSVFLFPPKPKDTL | IgG4 hinge-CH2-CH3 |
| | MISRTPEVTCVVVDVSQEDPEVQFNWYVDGV | domain |
| | EVHNAKTKPREEQFNSTYRVVSVLTVLHQDW | |
| | LNGKEYKCKVSNKGLPSSIEKTISKAKGQPREP | |
| | QVYTLPPSQEEMTKNQVSLTCLVKGFYPSDIA | |
| | VEWESNGQPENNYKTTPPVLDSDGSFFLYSRL | |
| | TVDKSRWQEGNVFSCSVMHEALHNHYTQKSL | |
| | SLSLGK | |

### 5. CD19 CARS

In some embodiments, a CAR is a CD19 CAR (“CD19-CAR”). In some embodiments, a CD19 CAR comprises a signal peptide, an extracellular binding domain that specifically binds CD19, a hinge domain, a transmembrane domain, an intracellular costimulatory domain, and/or an intracellular signaling domain in tandem.

In some embodiments, a CD19 CAR signal peptide comprises a CD8α signal peptide. In some embodiments, a CD8α signal peptide comprises or consists of an amino acid sequence set forth in SEQ ID NO:6 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:6. In some embodiments, a signal peptide comprises an IgK signal peptide. In some embodiments, an IgK signal peptide comprises or consists of an amino acid sequence set forth in SEQ ID NO:7 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:7. In some embodiments, a signal peptide comprises a GMCSFR-α or CSF2RA signal peptide. In some embodiments, a GMCSFR-α or CSF2RA signal peptide comprises or consists of an amino acid sequence set forth in SEQ ID NO: 8 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:8.

In some embodiments, a CD19 CAR extracellular binding domain is specific to CD19, for example, human CD19. A CD19 CAR extracellular binding domain can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of a CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34 (16-17): 1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 7 below. In some embodiments, a CD19-specific scFv comprises or consists of an amino acid sequence set forth in SEQ ID NO:19, 20, or 25, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:19, 20, or 25. In some embodiments, a CD19-specific scFv may comprise one or more CDRs having amino acid sequences set forth in SEQ ID NOs: 21-23 and 26-28. In some embodiments, a CD19-specific scFv may comprise a light chain with one or more CDRs having amino acid sequences set forth in SEQ ID NOs: 21-23. In some embodiments, a CD19-specific scFv may comprise a heavy chain with one or more CDRs having amino acid sequences set forth in SEQ ID NOs: 26-28. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of a CD19 CAR comprises or consists of the one or more CDRs as described herein. In some embodiments, a CD19-specific scFv comprises CDRs defined by Kabat numbering. In some embodiments, a CD19-specific scFv comprises CDRs defined by Chothia numbering.

In some embodiments, a linker linking the V_{H} and the V_{L} portions of an scFv is a Whitlow linker having an amino acid sequence set forth in SEQ ID NO:24. In some embodiments, a Whitlow linker may be replaced by a different linker, for example, a 3xG₄S linker having an amino acid sequence set forth in SEQ ID NO:30, which gives rise to a different FMC63-derived scFv having an amino acid sequence set forth in SEQ ID NO:29. In certain of these embodiments, the CD19-specific scFv comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29.

TABLE 7
Exemplary sequences of anti-CD19 scFv and components

| SEQ ID NO: | Amino Acid Sequence | Description |
|---|---|---|
| 19 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | Whitlow linker |
| | LEQEDIATYFCQQGNTLPYTFGGGT | |
| | KLEITGSTSGSGKPGSGEGSTKGEVK | |
| | LQESGPGLVAPSQSLSVTCTVSGVSL | |
| | PDYGVSWIRQPPRKGLEWLGVIWGS | |
| | ETTYYNSALKSRLTIIKDNSKSQVFL | |
| | KMNSLQTDDTAIYYCAKHYYYGGS | |
| | YAMDYWGQGTSVTVSS | |
| 20 | DIQMTQTTSSLSASLGDRVTISCRAS | Anti-CD19 FMC63 scFv |
| | QDISKYLNWYQQKPDGTVKLLIYHT | light chain variable region |
| | SRLHSGVPSRFSGSGSGTDYSLTISN | |
| | LEQEDIATYFCQQGNTLPYTFGGGT | |
| | KLEIT | |
| 21 | QDISKY | Anti-CD19 FMC63 scFv |
| | | light chain CDR1 |
| 22 | HTS | Anti-CD19 FMC63 scFv |
| | | light chain CDR2 |
| 23 | QQGNTLPYT | Anti-CD19 FMC63 scFv |
| | | light chain CDR3 |
| 24 | GSTSGSGKPGSGEGSTKG | Whitlow linker |
| 25 | EVKLQESGPGLVAPSQSLSVTCTVS | Anti-CD19 FMC63 scFv |
| | GVSLPDYGVSWIRQPPRKGLEWLG | heavy chain variable |
| | VIWGSETTYYNSALKSRLTIIKDNSK | region |
| | SQVFLKMNSLQTDDTAIYYCAKHY | |
| | YYGGSYAMDYWGQGTSVTVSS | |
| 26 | GVSLPDYG | Anti-CD19 FMC63 scFv |
| | | heavy chain CDR1 |
| 27 | IWGSETT | Anti-CD19 FMC63 scFv |
| | | heavy chain CDR2 |
| 28 | AKHYYYGGSYAMDY | Anti-CD19 FMC63 scFv |
| | | heavy chain CDR3 |
| 29 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | 3xG₄S linker |
| | LEQEDIATYFCQQGNTLPYTFGGGT | |
| | KLEITGGGGSGGGGSGGGGSEVKLQ | |
| | ESGPGLVAPSQSLSVTCTVSGVSLPD | |
| | YGVSWIRQPPRKGLEWLGVIWGSET | |
| | TYYNSALKSRLTIIKDNSKSQVFLK | |
| | MNSLQTDDTAIYYCAKHYYYGGSY | |
| | AMDYWGQGTSVTVSS | |
| 30 | GGGGSGGGGSGGGGS | 3xG4S linker |

In some embodiments, the extracellular binding domain of a CD19 CAR is derived from an antibody specific to CD19, including, for example, SJ25C1 (Bejcek et al., Cancer Res. 55:2346-2351 (1995)), HD37 (Pezutto et al., J. Immunol. 138 (9): 2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB 12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148 (10): 2983-2987 (1992)), and CLB-CD19 (De Rie Cell. Immunol. 118:368-381 (1989)). In any of these embodiments, the extracellular binding domain of the CD19 CAR can comprise or consist of the V_{H}, the V_{L}, and/or one or more CDRs of any of the antibodies.

In some embodiments, the hinge domain of a CD19 CAR comprises a CD8α hinge domain, for example, a human CD8α hinge domain. In some embodiments, the CD8α hinge domain comprises or consists of an amino acid sequence set forth in SEQ ID NO:9 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:9. In some embodiments, the hinge domain comprises a CD28 hinge domain, for example, a human CD28 hinge domain. In some embodiments, the CD28 hinge domain comprises or consists of an amino acid sequence set forth in SEQ ID NO:10 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:10. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:11 or SEQ ID NO:12, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:11 or SEQ ID NO:12. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:13 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:13.

In some embodiments, the transmembrane domain of a CD19 CAR comprises a CD8α transmembrane domain, for example, a human CD8α transmembrane domain. In some embodiments, the CD8α transmembrane domain comprises or consists of an amino acid sequence set forth in SEQ ID NO:14 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:14. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 15 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:15.

In some embodiments, the intracellular costimulatory domain of a CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:16 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:16. In some embodiments, the intracellular costimulatory domain comprises a CD28 costimulatory domain. CD28 is another co-stimulatory molecule on T cells. In some embodiments, the CD28 costimulatory domain is human. In some embodiments, the CD28 costimulatory domain comprises or consists of an amino acid sequence set forth in SEQ ID NO:17 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:17. In some embodiments, the intracellular costimulatory domain of the CD19 CAR comprises a 4-1BB costimulatory domain and a CD28 costimulatory domain as described.

In some embodiments, the intracellular signaling domain of a CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD35 signaling domain is human. In some embodiments, the CD35 signaling domain comprises or consists of an amino acid sequence set forth in SEQ ID NO:18 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:18.

In some embodiments, a polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO: 29, the CD8α hinge domain of SEQ ID NO:9, the CD8α transmembrane domain of SEQ ID NO: 14, the 4-1BB costimulatory domain of SEQ ID NO: 16, the CD35 signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO: 29, the IgG4 hinge domain of SEQ ID NO:11 or SEQ ID NO:12, the CD28 transmembrane domain of SEQ ID NO:15, the 4-1BB costimulatory domain of SEQ ID NO:16, the CD35 signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, a polycistronic vector comprises an expression cassette that contains a nucleotide sequence encoding a CD19 CAR, including, for example, a CD19 CAR comprising the CD19-specific scFv having sequences set forth in SEQ ID NO:19 or SEQ ID NO: 29, the CD28 hinge domain of SEQ ID NO:10, the CD28 transmembrane domain of SEQ ID NO: 15, the CD28 costimulatory domain of SEQ ID NO:17, the CD35 signaling domain of SEQ ID NO:18, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:4 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:4 (see Table 8). The encoded CD19 CAR has a corresponding amino acid sequence set forth in SEQ ID NO:5 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:5, with the following components: CD8α signal peptide, FMC63 scFv (V_{L}-Whitlow linker-V_{H}), CD8α hinge domain, CD8α transmembrane domain, 4-1BB costimulatory domain, and CD35 signaling domain.

In some embodiments, a polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, a polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3xG₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 8, with annotations of the sequences provided in Table 9.

In some embodiments, a polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}-Whitlow linker-V_{H}), IgG4 hinge domain, CD28 transmembrane domain, 4-1BB costimulatory domain, and CD3 signaling domain. The nucleotide and amino acid sequence of the CD19 CAR in lisocabtagene maraleucel are provided in Table 8, with annotations of the sequences provided in Table 10.

In some embodiments, a polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}-Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 8, with annotations of the sequences provided in Table 11.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, a polycistronic vector comprises an expression cassette that contains a nucleotide sequence encoding a CD19 CAR as set forth in SEQ ID NO: 31, 33, or 35, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

TABLE 8
Exemplary sequences of CD19 CARS

| SEQ ID NO: | Sequence | Description |
|---|---|---|
| 4 | atggccttaccagtgaccgccttgctcctgccgctggccttgctgct | Exemplary CD19 |
| | ccacgccgccaggccggacatccagatgacacagactacatcctc | CAR nucleotide |
| | cctgtctgcctctctgggagacagagtcaccatcagttgcagggca | sequence |
| | agtcaggacattagtaaatatttaaattggtatcagcagaaaccagat | |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggaccaagc | |
| | tggagatcacaggctccacctctggatccggcaagcccggatctg | |
| | gcgagggatccaccaagggcgaggtgaaactgcaggagtcagg | |
| | acctggcctggtggcgccctcacagagcctgtccgtcacatgcact | |
| | gtctcaggggtctcattacccgactatggtgtaagctggattcgcca | |
| | gcctccacgaaagggtctggagtggctgggagtaatatggggtag | |
| | tgaaaccacatactataattcagctctcaaatccagactgaccatcat | |
| | caaggacaactccaagagccaagttttcttaaaaatgaacagtctgc | |
| | aaactgatgacacagccatttactactgtgccaaacattattactacg | |
| | gtggtagctatgctatggactactggggccaaggaacctcagtcac | |
| | cgtctcctcaaccacgacgccagcgccgcgaccaccaacaccgg | |
| | cgcccaccatcgcgtcgcagcccctgtccctgcgcccagaggcgt | |
| | gccggccagcggcggggggcgcagtgcacacgagggggctgg | |
| | acttcgcctgtgatatctacatctgggcgcccttggccgggacttgt | |
| | ggggtccttctcctgtcactggttatcaccctttactgcaaacggggc | |
| | agaaagaaactcctgtatatattcaaacaaccatttatgagaccagta | |
| | caaactactcaagaggaagatggctgtagctgccgatttccagaag | |
| | aagaagaaggaggatgtgaactgagagtgaagttcagcaggagc | |
| | gcagacgcccccgcgtaccagcagggccagaaccagctctataa | |
| | cgagctcaatctaggacgaagagaggagtacgatgttttggacaa | |
| | gagacgtggccgggaccctgagatggggggaaagccgagaag | |
| | gaagaaccctcaggaaggcctgtacaatgaactgcagaaagataa | |
| | gatggcggaggcctacagtgagattgggatgaaaggcgagcgcc | |
| | ggaggggcaaggggcacgatggcctttaccagggtctcagtaca | |
| | gccaccaaggacacctacgacgcccttcacatgcaggccctgccc | |
| | cctcgc | |
| 5 | MALPVTALLLPLALLLHAARPDIQMTQTTS | Exemplary CD19 |
| | SLSASLGDRVTISCRASQDISKYLNWYQQK | CAR amino acid |
| | PDGTVKLLIYHTSRLHSGVPSRFSGSGSGT | sequence |
| | DYSLTISNLEQEDIATYFCQQGNTLPYTFG | |
| | GGTKLEITGSTSGSGKPGSGEGSTKGEVKL | |
| | QESGPGLVAPSQSLSVTCTVSGVSLPDYGV | |
| | SWIRQPPRKGLEWLGVIWGSETTYYNSAL | |
| | KSRLTIIKDNSKSQVFLKMNSLQTDDTAIY | |
| | YCAKHYYYGGSYAMDYWGQGTSVTVSST | |
| | TTPAPRPPTPAPTIASQPLSLRPEACRPAAG | |
| | GAVHTRGLDFACDIYIWAPLAGTCGVLLLS | |
| | LVITLYCKRGRKKLLYIFKQPFMRPVQTTQ | |
| | EEDGCSCRFPEEEEGGCELRVKESRSADAP | |
| | AYQQGQNQLYNELNLGRREEYDVLDKRR | |
| | GRDPEMGGKPRRKNPQEGLYNELQKDKM | |
| | AEAYSEIGMKGERRRGKGHDGLYQGLSTA | |
| | TKDTYDALHMQALPPR | |
| 31 | atggccttaccagtgaccgccttgctcctgccgctggccttgctgct | Tisagenlecleucel |
| | ccacgccgccaggccggacatccagatgacacagactacatcctc | CD19 CAR |
| | cctgtctgcctctctgggagacagagtcaccatcagttgcagggca | nucleotide |
| | agtcaggacattagtaaatatttaaattggtatcagcagaaaccagat | sequence |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggaccaagc | |
| | tggagatcacaggtggcggtggctcgggcggtggtgggtcgggt | |
| | ggcggcggatctgaggtgaaactgcaggagtcaggacctggcct | |
| | ggtggcgccctcacagagcctgtccgtcacatgcactgtctcagg | |
| | ggtctcattacccgactatggtgtaagctggattcgccagcctccac | |
| | gaaagggtctggagtggctgggagtaatatggggtagtgaaacca | |
| | catactataattcagctctcaaatccagactgaccatcatcaaggac | |
| | aactccaagagccaagttttcttaaaaatgaacagtctgcaaactga | |
| | tgacacagccatttactactgtgccaaacattattactacggtggtag | |
| | ctatgctatggactactggggccaaggaacctcagtcaccgtctcct | |
| | caaccacgacgccagcgccgcgaccaccaacaccggcgcccac | |
| | catcgcgtcgcagcccctgtccctgcgcccagaggcgtgccggc | |
| | cagcggcggggggcgcagtgcacacgagggggctggacttcgc | |
| | ctgtgatatctacatctgggcgcccttggccgggacttgtggggtcc | |
| | ttctcctgtcactggttatcaccctttactgcaaacggggcagaaag | |
| | aaactcctgtatatattcaaacaaccatttatgagaccagtacaaact | |
| | actcaagaggaagatggctgtagctgccgatttccagaagaagaa | |
| | gaaggaggatgigaactgagagtgaagttcagcaggagcgcaga | |
| | cgcccccgcgtacaagcagggccagaaccagctctataacgagc | |
| | tcaatctaggacgaagagaggagtacgatgttttggacaagagac | |
| | gtggccgggaccctgagatggggggaaagccgagaaggaaga | |
| | accctcaggaaggcctgtacaatgaactgcagaaagataagatgg | |
| | cggaggcctacagtgagattgggatgaaaggcgagcgccggag | |
| | gggcaaggggcacgatggcctttaccagggtctcagtacagccac | |
| | caaggacacctacgacgcccttcacatgcaggccctgccccctcg | |
| | c | |
| 32 | MALPVTALLLPLALLLHAARPDIQMTQTTS | Tisagenlecleucel |
| | SLSASLGDRVTISCRASQDISKYLNWYQQK | CD19 CAR amino |
| | PDGTVKLLIYHTSRLHSGVPSRFSGSGSGT | acid sequence |
| | DYSLTISNLEQEDIATYFCQQGNTLPYTFG | |
| | GGTKLEITGGGGSGGGGSGGGGSEVKLQE | |
| | SGPGLVAPSQSLSVTCTVSGVSLPDYGVSW | |
| | IRQPPRKGLEWLGVIWGSETTYYNSALKSR | |
| | LTIIKDNSKSQVFLKMNSLQTDDTAIYYCA | |
| | KHYYYGGSYAMDYWGQGTSVTVSSTTTP | |
| | APRPPTPAPTIASQPLSLRPEACRPAAGGAV | |
| | HTRGLDFACDIYIWAPLAGTCGVLLLSLVI | |
| | TLYCKRGRKKLLYIFKQPFMRPVQTTQEED | |
| | GCSCRFPEEEEGGCELRVKFSRSADAPAYK | |
| | QGQNQLYNELNLGRREEYDVLDKRRGRD | |
| | PEMGGKPRRKNPQEGLYNELQKDKMAEA | |
| | YSEIGMKGERRRGKGHDGLYQGLSTATKD | |
| | TYDALHMQALPPR | |
| 33 | atgctgctgctggtgaccagcctgctgctgtgcgagctgccccacc | Lisocabtagene |
| | ccgcctttctgctgatccccgacatccagatgacccagaccacctc | maraleucel CD19 |
| | cagcctgagcgccagcctgggcgaccgggtgaccatcagctgcc | CAR nucleotide |
| | gggccagccaggacatcagcaagtacctgaactggtatcagcag | sequence |
| | aagcccgacggcaccgtcaagctgctgatctaccacaccagccg | |
| | gctgcacagcggcgtgcccagccggtttagcggcagcggctccg | |
| | gcaccgactacagcctgaccatctccaacctggaacaggaagata | |
| | tcgccacctacttttgccagcagggcaacacactgccctacaccttt | |
| | ggcggcggaacaaagctggaaatcaccggcagcacctccggca | |
| | gcggcaagcctggcagcggcgagggcagcaccaagggcgagg | |
| | tgaagctgcaggaaagcggccctggcctggtggcccccagccag | |
| | agcctgagcgtgacctgcaccgtgagcggcgtgagcctgcccga | |
| | ctacggcgtgagctggatccggcagccccccaggaagggcctgg | |
| | aatggctgggcgtgatctggggcagcgagaccacctactacaaca | |
| | gcgccctgaagagccggctgaccatcatcaaggacaacagcaag | |
| | agccaggtgttcctgaagatgaacagcctgcagaccgacgacacc | |
| | gccatctactactgcgccaagcactactactacggcggcagctacg | |
| | ccatggactactggggccagggcaccagcgtgaccgtgagcagc | |
| | gaatctaagtacggaccgccctgcccccctigccctatgticigggt | |
| | gctggtggtggtcggaggcgtgctggcctgctacagcctgctggt | |
| | caccgtggccttcatcatcttttgggtgaaacggggcagaaagaaa | |
| | ctcctgtatatattcaaacaaccatttatgagaccagtacaaactactc | |
| | aagaggaagatggctgtagctgccgatttccagaagaagaagaag | |
| | gaggatgtgaactgcgggtgaagttcagcagaagcgccgacgcc | |
| | cctgcctaccagcagggccagaatcagctgtacaacgagctgaac | |
| | ctgggcagaagggaagagtacgacgtcctggataagcggagag | |
| | gccgggaccctgagatgggcggcaagcctcggcggaagaaccc | |
| | ccaggaaggcctgtataacgaactgcagaaagacaagatggccg | |
| | aggcctacagcgagatcggcatgaagggcgagcggaggcggg | |
| | gcaagggccacgacggcctgtatcagggcctgtccaccgccacc | |
| | aaggatacctacgacgccctgcacatgcaggccctgcccccaag | |
| | g | |
| 34 | MLLLVTSLLLCELPHPAFLLIPDIQMTQTTS | Lisocabtagene |
| | SLSASLGDRVTISCRASQDISKYLNWYQQK | maraleucel CD19 |
| | PDGTVKLLIYHTSRLHSGVPSRFSGSGSGT | CAR amino acid |
| | DYSLTISNLEQEDIATYFCQQGNTLPYTFG | sequence |
| | GGTKLEITGSTSGSGKPGSGEGSTKGEVKL | |
| | QESGPGLVAPSQSLSVTCTVSGVSLPDYGV | |
| | SWIRQPPRKGLEWLGVIWGSETTYYNSAL | |
| | KSRLTIIKDNSKSQVFLKMNSLQTDDTAIY | |
| | YCAKHYYYGGSYAMDYWGQGTSVTVSSE | |
| | SKYGPPCPPCPMFWVLVVVGGVLACYSLL | |
| | VTVAFIIFWVKRGRKKLLYIFKQPFMRPVQ | |
| | TTQEEDGCSCRFPEEEEGGCELRVKFSRSA | |
| | DAPAYQQGQNQLYNELNLGRREEYDVLD | |
| | KRRGRDPEMGGKPRRKNPQEGLYNELQK | |
| | DKMAEAYSEIGMKGERRRGKGHDGLYQG | |
| | LSTATKDTYDALHMQALPPR | |
| 35 | atgcttctcctggtgacaagccttctgctctgtgagttaccacaccca | Axicabtagene |
| | gcattcctcctgatcccagacatccagatgacacagactacatcctc | ciloleucel CD19 |
| | cctgtctgcctctctgggagacagagtcaccatcagttgcagggca | CAR nucleotide |
| | agtcaggacattagtaaatatttaaattggtatcagcagaaaccagat | sequence |
| | ggaactgttaaactcctgatctaccatacatcaagattacactcagg | |
| | agtcccatcaaggttcagtggcagtgggtctggaacagattattctc | |
| | tcaccattagcaacctggagcaagaagatattgccacttacttttgcc | |
| | aacagggtaatacgcttccgtacacgttcggaggggggactaagtt | |
| | ggaaataacaggctccacctctggatccggcaagcccggatctgg | |
| | cgagggatccaccaagggcgaggtgaaactgcaggagtcagga | |
| | cctggcctggtggcgccctcacagagcctgtccgtcacatgcactg | |
| | tctcaggggtctcattacccgactatggtgtaagctggattcgccag | |
| | cctccacgaaagggtctggagtggctgggagtaatatggggtagt | |
| | gaaaccacatactataattcagctctcaaatccagactgaccatcatc | |
| | aaggacaactccaagagccaagtittcttaaaaatgaacagtctgca | |
| | aactgatgacacagccatttactactgtgccaaacattattactacgg | |
| | tggtagctatgctatggactactggggtcaaggaacctcagtcacc | |
| | gtctcctcagcggccgcaattgaagttatgtatcctcctccttaccta | |
| | gacaatgagaagagcaatggaaccattatccatgtgaaagggaaa | |
| | cacctttgtccaagtcccctatttcccggaccttctaagcccttttggg | |
| | tgctggtggtggttgggggagtcctggcttgctatagcttgctagta | |
| | acagtggcctttattattttctgggtgaggagtaagaggagcaggct | |
| | cctgcacagtgactacatgaacatgactccccgccgccccgggcc | |
| | cacccgcaagcattaccagccctatgccccaccacgcgacttcgc | |
| | agcctatcgctccagagtgaagttcagcaggagcgcagacgccc | |
| | ccgcgtaccagcagggccagaaccagctctataacgagctcaatc | |
| | taggacgaagagaggagtacgatgttttggacaagagacgtggcc | |
| | gggaccctgagatggggggaaagccgagaaggaagaaccctca | |
| | ggaaggcctgtacaatgaactgcagaaagataagatggcggagg | |
| | cctacagtgagattgggatgaaaggcgagcgccggaggggcaa | |
| | ggggcacgatggcctttaccagggtctcagtacagccaccaagga | |
| | cacctacgacgcccttcacatgcaggccctgccccctcgc | |
| 36 | MLLLVTSLLLCELPHPAFLLIPDIQMTQTTS | Axicabtagene |
| | SLSASLGDRVTISCRASQDISKYLNWYQQK | ciloleucel CD19 |
| | PDGTVKLLIYHTSRLHSGVPSRFSGSGSGT | CAR amino acid |
| | DYSLTISNLEQEDIATYFCQQGNTLPYTFG | sequence |
| | GGTKLEITGSTSGSGKPGSGEGSTKGEVKL | |
| | QESGPGLVAPSQSLSVTCTVSGVSLPDYGV | |
| | SWIRQPPRKGLEWLGVIWGSETTYYNSAL | |
| | KSRLTIIKDNSKSQVFLKMNSLQTDDTAIY | |
| | YCAKHYYYGGSYAMDYWGQGTSVTVSSA | |
| | AAIEVMYPPPYLDNEKSNGTIIHVKGKHLC | |
| | PSPLFPGPSKPFWVLVVVGGVLACYSLLVT | |
| | VAFIIFWVRSKRSRLLHSDYMNMTPRRPGP | |
| | TRKHYQPYAPPRDFAAYRSRVKFSRSADA | |
| | PAYQQGQNQLYNELNLGRREEYDVLDKR | |
| | RGRDPEMGGKPRRKNPQEGLYNELQKDK | |
| | MAEAYSEIGMKGERRRGKGHDGLYQGLS | |
| | TATKDTYDALHMQALPPR | |

TABLE 9
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 10
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 11
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, a polycistronic vector comprises an expression cassette that contains a nucleotide sequence encoding CD19 CAR as set forth in SEQ ID NO: 31, 33, or 35, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 31, 33, or 35. The encoded CD19 CAR has a corresponding amino acid sequence set forth in SEQ ID NO: 32, 34, or 36, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 32, 34, or 36, respectively.

### D. Characteristics of Hypoimmunogenic Cells

In some embodiments, a administered population of hypoimmunogenic cells such as hypoimmunogenic CAR-T cells elicits a decreased or lower level of immune activation in the subject or patient. In some instances, a level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, an administered population of hypoimmunogenic cells fails to elicit immune activation in the subject or patient.

In some embodiments, an administered population of hypoimmunogenic cells such as hypoimmunogenic CAR-T cells elicits a decreased or lower level of T cell response in the subject or patient. In some instances, a level of T cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of T cell response produced by the administration of immunogenic cells. In some embodiments, an administered population of hypoimmunogenic cells fails to elicit a T cell response to the cells in the subject or patient.

In some embodiments, an administered population of hypoimmunogenic cells such as hypoimmunogenic CAR-T cells elicits a decreased or lower level of NK cell response in the subject or patient. In some instances, a level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell response produced by the administration of immunogenic cells. In some embodiments, an administered population of hypoimmunogenic cells fails to elicit an NK cell response to the cells in the subject or patient.

In some embodiments, an administered population of hypoimmunogenic cells such as hypoimmunogenic CAR-T cells elicits a decreased or lower level of macrophage engulfment in the subject or patient. In some instances, a level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of macrophage engulfment produced by the administration of immunogenic cells. In some embodiments, an administered population of hypoimmunogenic cells fails to elicit macrophage engulfment of the cells in the subject or patient.

In some embodiments, an administered population of hypoimmunogenic cells such as hypoimmunogenic CAR-T cells elicits a decreased or lower level of systemic TH1 activation in the subject or patient. In some instances, a level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, an administered population of hypoimmunogenic cells fails to elicit systemic TH1 activation in the subject or patient.

In some embodiments, an administered population of hypoimmunogenic cells such as hypoimmunogenic CAR-T cells elicits a decreased or lower level of NK cell killing in the subject or patient. In some instances, a level of NK cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell killing produced by the administration of immunogenic cells. In some embodiments, an administered population of hypoimmunogenic cells fails to elicit NK cell killing in the subject or patient.

In some embodiments, an administered population of hypoimmunogenic cells such as hypoimmunogenic CAR-T cells elicits a decreased or lower level of immune activation of peripheral blood mononuclear cells (PBMCs) in the subject or patient. In some instances, a level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, an administered population of hypoimmunogenic cells fails to elicit immune activation of PBMCs in the subject or patient.

In some embodiments, an administered population of hypoimmunogenic cells such as hypoimmunogenic CAR-T cells elicits a decreased or lower level of donor-specific IgG antibodies in the subject or patient. In some instances, a level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, an administered population of hypoimmunogenic cells fails to elicit donor-specific IgG antibodies in the subject or patient.

In some embodiments, an administered population of hypoimmunogenic cells such as hypoimmunogenic CAR-T cells elicits a decreased or lower level of donor-specific IgM antibodies in the subject or patient. In some instances, a level of donor-specific IgM antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgM antibodies produced by the administration of immunogenic cells. In some embodiments, an administered population of hypoimmunogenic cells fails to elicit donor-specific IgM antibodies in the subject or patient.

In some embodiments, an administered population of hypoimmunogenic cells such as hypoimmunogenic CAR-T cells elicits a decreased or lower level of IgM and IgG antibody production in the subject or patient. In some instances, a level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, an administered population of hypoimmunogenic cells fails to elicit IgM and IgG antibody production in the subject or patient.

In some embodiments, an administered population of hypoimmunogenic cells such as hypoimmunogenic CAR-T cells elicits a decreased or lower level of cytotoxic T cell killing in the subject or patient. In some instances, a level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, an administered population of hypoimmunogenic cells fails to elicit cytotoxic T cell killing in the subject or patient.

In some embodiments, an administered population of hypoimmunogenic cells such as hypoimmunogenic CAR-T cells elicits a decreased or lower level of complement-dependent cytotoxicity (CDC) in the subject or patient. In some instances, a level of CDC elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of CDC produced by the administration of immunogenic cells. In some embodiments, an administered population of hypoimmunogenic cells fails to elicit CDC in the subject or patient.

E. Therapeutic Cells from Primary T Cells

Provided herein are hypoimmunogenic cells including, but not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, a pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, a donor subject is different from a patient (e.g., the recipient that is administered the therapeutic cells). In some embodiments, a pool of T cells do not include cells from the patient. In some embodiments, one or more of the donor subjects from which the pool of T cells is obtained are different from the patient.

In some embodiments, hypoimmunogenic cells do not activate an innate and/or an adaptive immune response in the patient (e.g., recipient upon administration). Provided are methods of treating a disorder by administering a population of hypoimmunogenic cells to a subject (e.g., recipient) or patient in need thereof. In some embodiments, hypoimmunogenic cells described herein comprise T cells engineered (e.g., are modified) to express a chimeric antigen receptor including but not limited to a chimeric antigen receptor described herein. In some instances, T cells are populations or subpopulations of primary T cells from one or more individuals. In some embodiments, T cells described herein such as the engineered or modified T cells comprise reduced expression of an endogenous T cell receptor.

In some embodiments, the present disclosure is directed to hypoimmunogenic primary T cells that overexpress CD47 and CARs, and have reduced expression or lack expression of MHC class I and/or MHC class II human leukocyte antigens and have reduced expression or lack expression of TCR complex molecules. The cells outlined herein overexpress CD47 and CARs and evade immune recognition. In some embodiments, the primary T cells display reduced levels or activity of MHC class I antigens, MHC class II antigens, and/or TCR complex molecules. In certain embodiments, primary T cells overexpress CD47 and CARs and harbor a genomic modification in the B2M gene. In some embodiments, T cells overexpress CD47 and CARs and harbor a genomic modification in the CIITA gene. In some embodiments, primary T cells overexpress CD47 and CARs and harbor a genomic modification in the TRAC gene. In some embodiments, T cells overexpress CD47 and CARs and harbor genomic modifications in one or more of the following genes: the B2M, CIITA, and TRAC genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In certain embodiments, T cells express CCR7, CD27, CD28, and CD45RA. In some embodiments, central T cells express CCR7, CD27, CD28, and CD45RO. In other embodiments, effector memory T cells express PD-1, CD27, CD28, and CD45RO. In other embodiments, effector memory RA T cells express PD-1, CD57, and CD45RA.

In some embodiments, a T cell is a modified (e.g., an engineered) T cell. In some cases, a modified T cell comprises a modification causing the cell to express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, a modified T cell comprise a modification causing the cell to express at least one protein that modulates a biological effect of interest in an adjacent cell, tissue, or organ when the cell is in proximity to the adjacent cell, tissue, or organ. Useful modifications to primary T cells are described in detail in US2016/0348073 and WO2020/018620, the disclosures of which are incorporated herein in their entireties.

In some embodiments, a hypoimmunogenic T cell includes a polynucleotide encoding a CAR, wherein the polynucleotide is inserted in a genomic locus. In some embodiments, a polynucleotide encoding a CAR is randomly integrated into the genome of a cell. In some embodiments, a polynucleotide encoding a CAR is randomly integrated into the genome of a cell via viral vector transduction. In some embodiments, a polynucleotide encoding a CAR is randomly integrated into the genome of a cell via lentiviral vector transduction.

In some embodiments, a hypoimmunogenic T cell includes a polynucleotide encoding a CAR that is expressed in a cell using an expression vector. In some embodiments, a CAR is introduced to a cell using a viral expression vector that mediates integration of the CAR sequence into the genome of the cell. For example, the expression vector for expressing the CAR in a cell comprises a polynucleotide sequence encoding the CAR. The expression vector can be an inducible expression vector. The expression vector can be a viral vector, such as but not limited to, a lentiviral vector.

In some embodiments, hypoimmunogenic T cells provided herein are useful for the treatment of diseases, disorders, or conditions associated with CD19 expression. In some embodiments, hypoimmunogenic T cells provided herein are useful for treatment of B cell malignancies including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In some embodiments, hypoimmunogenic T cells provided herein are useful for the treatment of a relapsed and/or refractory B cell malignancy as described herein. In some embodiments, hypoimmunogenic T cells provided herein are useful for the treatment of relapsed and/or refractory NHL. In some embodiments, hypoimmunogenic T cells provided herein are useful for the treatment of relapsed and/or refractory CLL.

### F. Assays for Hypoimmunogenicity Phenotypes and Retention of Pluripotency

Once the hypoimmunogenic cells have been generated, they may be assayed for their hypoimmunogenicity and/or retention of pluripotency as is described in WO2016183041 and WO2018132783.

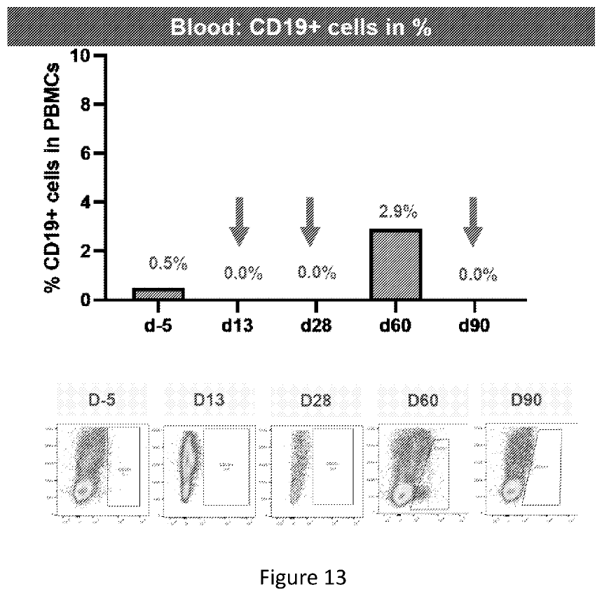

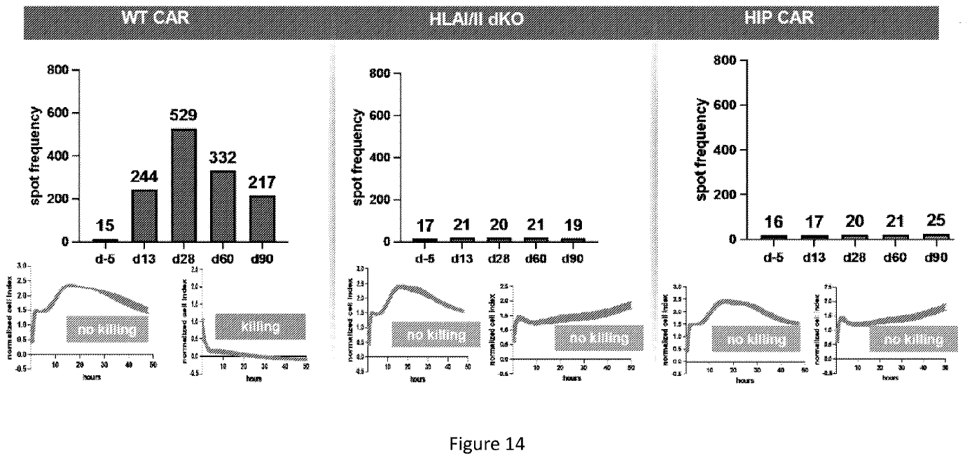

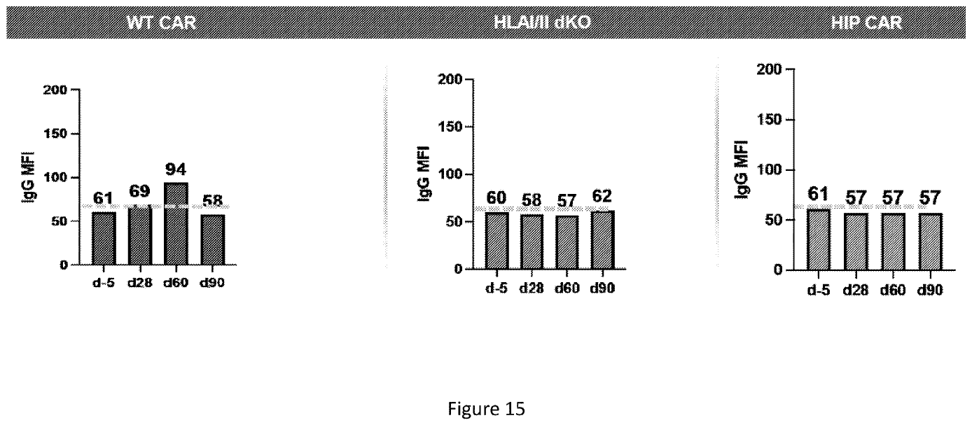

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogenic humanized immunodeficient mouse model. In some instances, the hypoimmunogenic pluripotent stem cells are transplanted into an allogenic humanized NSG-SGM3 mouse and assayed for cell rejection, cell survival, and teratoma formation. In some instances, grafted hypoimmunogenic pluripotent stem cells or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116 (21), 10441-10446, the disclosures including the figures, figure legends, and description of methods are incorporated herein by reference in their entirety.

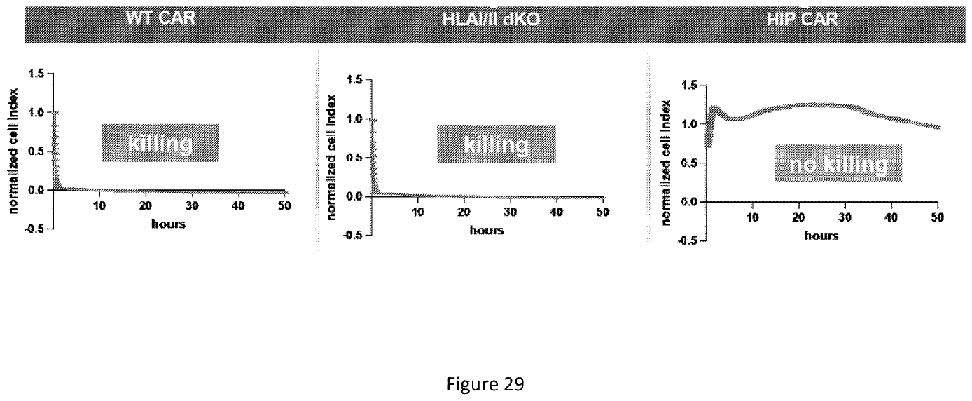

Similarly, the retention of pluripotency is tested in a number of ways. In some embodiments, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, HLA-B, and HLA-C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the MHC II function (HLA II when the cells are derived from human cells) in the pluripotent cells or their derivatives can be measured using techniques known in the art such as Western blotting using antibodies to the protein, FACS techniques, RT-PCR techniques, etc.

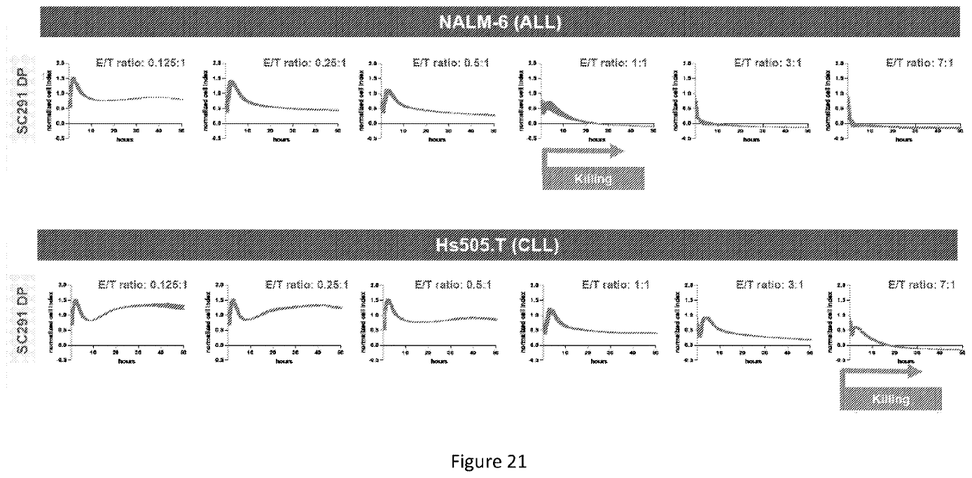

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II HLA-DR, DP and most DQ antigens.

In addition to the reduction of HLA I and II (or MHC I and II), the hypoimmunogenic cells of the technology have a reduced susceptibility to macrophage phagocytosis and NK cell killing. The resulting hypoimmunogenic cells “escape” the immune macrophage and innate pathways due to reduction or lack of the TCR complex and the expression of one or more CD47 transgenes.

### EXAMPLES

### Example 1: Administration of Hypoimmune Allogeneic CD19-Directed CAR T Cells to Subjects with Relapsed and/or Refractory B Cell Malignancies

In CLL, patients progressing on BTK and BCL2 inhibitors have significant unmet medical need with no curative therapies, and hypoimmune allogenic CD19-directed CAR T cells of the present disclosure may represent a novel therapeutic option. In DLBCL, while the durable clinical efficacy of autologous CAR T cell therapies has been established, limitations of the technology include long manufacturing and “vein-to-vein” times (i.e., time from leukapheresis to CAR T cell treatment) lasting 2 to 4 weeks. This may lead to treatment delays and disease progression in subjects with advanced disease. Further, the reliance on the patient's T cells, which may be dysfunctional or limited in numbers, contributes to treatment variability and failure and may be associated with contamination with malignant tumor cells. The complex logistical and manufacturing process incurs high cost for patients and payers, thereby limiting subject access.

Off-the-shelf, allogeneic CAR T cell therapies may offer advantages over autologous strategies, including batch manufacturing from healthy donors, which allows immediate access to therapy and the option of retreatment. Allogeneic CAR T cells derived from healthy donors may overcome the challenges of T cell dysfunction in malignancy, providing a more consistently potent product with avoidance of malignant contamination. Further, a batch of allogeneic CAR T cells can be administered to multiple subjects, allowing a standardized treatment to be manufactured at reduced cost and reducing the barriers to patient access that currently exist.

A primary limitation of allogeneic CAR T cell approaches is limited CAR T cell expansion and persistence due, in part, to adaptive and/or innate immune recognition, leading to host-mediated CAR T cell destruction and poor patient responses. Additionally, the recipient's cells may appear “foreign” to the TCRs of the allogeneic CAR T cells, which may induce their activation and result in acute graft-versus-host disease (GvHD). Thus, there remains a significant need to develop allogeneic CAR T cell therapies with enhanced immune evasion and improved expansion, persistence, and response durability.

Therapeutic compositions comprising hypoimmune allogenic CD19-directed CAR T cells will be administered to adult subjects with relapsed or refractory (r/r) Non-Hodgkin's Lymphoma (NHL) and Chronic Lymphocytic Leukemia (CLL). The hypoimmune technology disclosed herein enables engineered hypoimmunogenic T cells of the present disclosure to evade both the innate and adaptive immune system, thus potentially enabling enhanced cellular expansion and persistence with standard lymphodepletion. The studies described in this example evaluate the safety and tolerability of hypoimmunogenic T cells of the present disclosure. Phase 1a dose escalation will evaluate 3 dose levels of hypoimmunogenic T cells in subjects with NHL or CLL using a 3+3 design and identify a recommended phase 2 dose (RP2D). Phase 1b dose expansion will evaluate safety and efficacy of hypoimmunogenic T cells at the RP2D in subjects with large B cell lymphoma (LBCL) and CLL.

### Objectives and Endpoints

In this example, safety and tolerability of hypoimmunogenic T cells of the present disclosure, will be evaluated by monitoring: dose-limited toxicities (DLTs), treatment-emergent adverse events (TEAEs), treatment-related adverse events (TRAEs), targeted adverse events (TAEs), and/or adverse events of special interest (AESIs).

Secondary objectives of the studies of this example include evaluating the anti-tumor activity of hypoimmunogenic T cells of the present disclosure. Anti-tumor activity of hypoimmunogenic T cells of the present disclosure, will be evaluated by monitoring: objective responses, minimal residual disease (MRD) undetectable response (for CLL subjects), duration of responses (DOR), time to next treatment, progression free survival (PFS), event free survival (EFS), overall survival (OS) and/or MRD negativity.

Secondary objectives of the studies of this example also include evaluating the cellular kinetics and persistence of hypoimmunogenic T cells of the present disclosure. In this example, the cellular kinetics (CK)-related parameters of hypoimmunogenic T cells of the present disclosure, will be evaluated by CAR copy number and includes: peak observed in peripheral blood after administration (Cₘₐₓ), time of first occurrence of maximum-observed concentration (Tₘₐₓ), terminal disposition phase half-life (t_{1/2}), last observed quantifiable concentration in peripheral blood (Cₗₐₛₜ), time of last observed quantifiable concentration in peripheral blood (days) (Tₗₐₛₜ), and area under the concentration-time curves (AUCs).

Secondary objectives of the studies of this example also include evaluating the host immunogenicity of hypoimmunogenic T cells of the present disclosure. In this example, the host immunogenicity of hypoimmunogenic T cells of the present disclosure will be evaluated by monitoring host humoral immune responses.

Exploratory objectives of this study include evaluating additional immunogenicity assessments of hypoimmune allogenic CD19-directed CAR T cells of the present disclosure administered to subjects with relapsed and/or refractory B cell malignancies. In this example, additional immunogenicity assessments of hypoimmunogenic T cells of the present disclosure will be evaluated by monitoring host cellular immune responses (ELISpot/T cell killing/NK cell killing) and donor-specific antibody production (donor-specific antibody [DSA]/antibody-dependent cellular cytotoxicity [ADCC]/complement-dependent cytotoxicity [CDC]).

Exploratory objectives of this study also include evaluating the impact on baseline donor characteristics and attributes of hypoimmune allogenic CD19-directed CAR T cells of the present disclosure on subject safety, outcomes, and cellular kinetics. In this example, the impact of administration of hypoimmune allogenic CD19-directed CAR T cells of the present disclosure on subject safety, outcomes, and cellular kinetics will be evaluated by monitoring subject: T cells, B cells and other immune cell phenotypes (CAR T enumeration and immunophenotyping); T cell activation, expansion, effector function, and exhaustion (immunophenotyping); expression levels of CD 19-directed CAR, CD47, TCR, and HLA I/II (CAR T enumeration and CD47 expression); and molecular immune landscape.

Exploratory objectives of this study also include evaluating markers that are indicative of a durable response in subjects to hypoimmune allogenic CD19-directed CAR T cells of the present disclosure. In this example, markers indicating a durable response in subjects to hypoimmune allogenic CD19-directed CAR T cells of the present disclosure will be evaluated by longitudinal circulating tumor DNA (ctDNA) assessment (minimum residual disease (MRD) negativity).

Exploratory objectives of this study also include evaluating the time to treatment of hypoimmune allogenic CD19-directed CAR T cells of the present disclosure. In this example, the time to treatment of hypoimmune allogenic CD19-directed CAR T cells of the present disclosure will be evaluated by monitoring: the time from study enrollment to treatment with hypoimmune allogenic CD19-directed CAR T cells; the time from study enrollment to lymphodepleting (LD) chemotherapy; and the time from start of LD chemotherapy to treatment with hypoimmune allogenic CD19-directed CAR T cells.

### Study Design

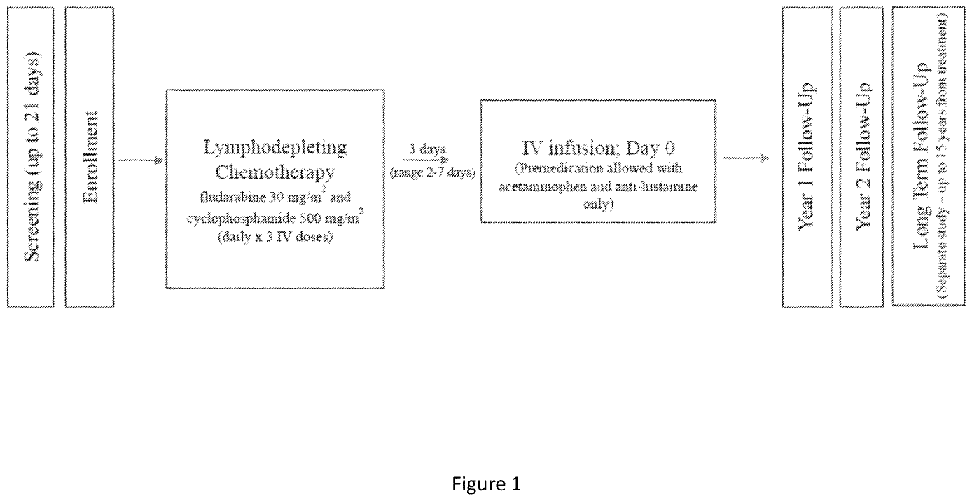

As shown in FIG. 1, hypoimmune allogenic CD19-directed CAR T cells of the present disclosure will be administered intravenously (IV) in a single infusion following standard lymphodepletion in subjects with NHL or CLL who have received two or more prior systemic treatments per standard of care (or after autologous stem cell transplant [ASCT] for NHL).

### Screening

Screening begins when a subject signs an institutional review board/independent ethics committee (IRB/IEC)-approved informed consent form (ICF) and is provided with a study-specific subject number. Screening begins up to 21 days before enrollment and ends with confirmation of eligibility and enrollment.

### Inclusion Criteria

Subjects considered for the safety and tolerability of the present example have the following inclusion criteria:
(i) male or female aged 18 to 80 years;
(ii) diagnosed NHL (WHO 2016 criteria) or CLL (iwCLL criteria), including
   a. large B cell lymphoma, (e.g., diffuse large B cell lymphoma (DLBCL) not otherwise specified, for example, arising from indolent lymphoma), primary mediastinal large B cell lymphoma, high-grade B cell lymphoma, follicular lymphoma Grade 3B;
   b. follicular lymphoma (Phase 1a dose escalation only, except for follicular lymphoma Grade 3B);
   c. marginal zone lymphoma (Phase 1a dose escalation only);
   d. mantle cell lymphoma (Phase 1a dose escalation only); and
   e. small lymphocytic lymphoma (SLL),
where histological confirmation of NHL can be by archived tumor biopsy tissue from last relapse and corresponding pathology report, pre-treatment biopsy at time of diagnosis, or recent biopsy taken within 6 months in subjects with treatment failure;
(iii) relapsed/refractory disease after at least 2 regimens per standard of care or after autologous stem cell transplant (ASCT), where refractory disease is defined as no response to first line therapy, progressive disease (PD) as best response to most recent therapy regime, or stable disease (SD) as best response to most recent therapy with duration no longer than 6 months from last dose of therapy;
(iv) CLL subjects will have received a Bruton tyrosine kinase (BTK) inhibitor or a B cell lymphoma 2 (BCL2) inhibitor (with no more than 5 lines of therapy in dose escalation);
(v) subjects with bulky disease will be allowed in Phase 1b dose expansion but not Phase 1a dose escalation. (Bulky disease is defined as: a. CLL subjects with any lymph node ≥10 cm OR any lymph node ≥5 cm and absolute lymphocyte count (ALC) ≥25×10⁹/L; or NHL subjects with a single lesion diameter >7.5 cm or sum of the product of lymph node diameters (SPD) >5000 mm³;
(vi) Eastern Cooperative Oncology Group (ECOG) performance status of 0 or 1;
(vii) NHL subjects will have at least one measurable lesion per Lugano Classification criteria;
(viii) CLL subjects will have met all iwCLL treatment criteria;
(ix) adequate organ function as confirmed by clinical laboratory values including,
   a. total bilirubin <1.5×upper limit of normal (ULN) (or <3×ULN for subjects with liver involvement),
   b. Alanine aminotransferase (ALT) or aspartate aminotransferase (AST)<3×ULN (<5×ULN for subjects with liver involvement),
   c. calculated creatinine clearance (as estimated by MDRD equation) ≥50 mL/min/1.73 m²,
   d. hemoglobin ≥8 g/dL (without blood transfusion within 7 days from screening assessment),
   e. neutrophil count >1000/mm³ (without growth factor support within 7 days from screening assessment),
   f. platelet count >50,000/mm³ (without platelet transfusion within 7 days from screening assessment),
   g. prothrombin time-international normalized ratio (INR)≤1×ULN,
   h. activated partial thromboplastin time (APTT)≤1.5×ULN;
   i. cardiac ejection fraction >40%, without evidence of clinically significant pericardial effusion, as assessed by echocardiogram (ECHO) scan performed within 1 month prior to hypoimmune allogenic CD19-directed CAR T cells treatment;
   j. baseline oxygen saturation >92% on room air, and no clinically significant pleural effusion.
(x) ≥12 week life expectancy;
(xi) eligibility for LD chemotherapy;
(xii) willingness to practice birth control and refrain from donating sperm or oocytes from the time of enrollment in the study through 12 months after receiving the study treatment; and
(xiii) negative pregnancy test for women of childbearing potential.

### Exclusion Criteria

Subjects considered for the safety and tolerability of the present example will not have any of the following exclusion criteria:
(i) prior CD19-directed therapy including CD19-directed CAR T cell therapy or other CD19-directed antibody or cell therapy (e.g., natural killer cell [NK cell]) (Phase 1b dose expansion only-prior approved CD19-directed CAR T cell therapy required);
(ii) history of primary central nervous system (CNS) lymphoma or presence of CNS metastases based on brain magnetic resonance imaging (MRI) performed within 3 months of study treatment (subjects with CNS symptoms will have lumbar puncture during screening to rule out CNS metastases);
(iii) history of Richter's transformation of chronic leukemic lymphoma, small lymphocytic lymphoma, or lymphoplasmacytic lymphoma;
(iv) presence of fungal, bacterial, viral, or other infection that is uncontrolled or requiring IV antimicrobials for management (simple urinary tract infection (UTI) and uncomplicated bacterial pharyngitis are permitted if responding to active treatment);
(v) acute symptoms of COVID-19 infection within 10 days prior to LD chemotherapy;
(vi) history of infection with HIV or hepatitis B (HbsAg positive) or hepatitis C virus (anti-HCV positive) (history of hepatitis B or hepatitis C is permitted if the viral load is undetectable per quantitative polymerase chain reaction (PCR) and/or nucleic acid testing);
(vii) active autoimmune disease or any other diseases requiring immunosuppressive therapy or corticosteroid therapy (defined as >10 mg/day prednisone or equivalent) (physiologic replacement, topical, and inhaled steroids are permitted);
(viii) primary immunodeficiency or history of concomitant genetic syndrome associated with bone marrow failure such as Fanconi anemia, Kostmann syndrome, or Shwachman-Diamond syndrome;
(ix) systemic anticancer therapy (including platinum-based chemotherapies and other small molecules and biologics) within 14 days (in some embodiments, 5 half-lives or whichever is shorter) before treatment with hypoimmune allogenic CD19-directed CAR T cells (28 days for biologics);
(x) anti-CD47 therapies within 1 year from last dose before treatment with hypoimmune allogenic CD19-directed CAR T cells;
(xi) treatment with any investigational products within 28 days before treatment with hypoimmune allogenic CD19-directed CAR T cells;
(xii) treatment with radiotherapy within 14 days before treatment with hypoimmune allogenic CD19-directed CAR T cells;
(xiii) treatment with major surgery within 28 days before treatment with hypoimmune allogenic CD19-directed CAR T cells (minor surgical procedures such as lymph node biopsy/excision or catheter placement are permitted);
(xiv) autologous hematopoietic stem cell transplantation (HSCT) within 6 weeks of treatment with hypoimmune allogenic CD19-directed CAR T cells or allogeneic HSCT at any time;
(xv) myocardial infarction, cardiac angioplasty or stenting, cardiac arrhythmia requiring medication, unstable angina, or other clinically significant cardiac disease within 6 months of enrollment;
(xvi) history of non-line associated, clinically significant deep-vein thrombosis (i.e., proximal DVT) or pulmonary embolism requiring therapeutic anticoagulation within the 6 months before enrollment;
(xvii) history or presence of CNS disorder, such as seizure disorder, cerebrovascular ischemia/hemorrhage, dementia, cerebellar disease, or any autoimmune disease with CNS involvement, within 12 months of enrollment;
(xviii) clinically significant ascites, defined as ascites that are physically positive or require intervention (e.g., puncture or medication) for control (subject with imaging result showing ascites requiring no intervention may be included);
(xix) history of organ transplantation or awaiting organ transplantation;
(xx) presence of any indwelling line or drain (e.g., percutaneous nephrostomy tube, indwelling Foley catheter, biliary drain, G/J-tube, or pleural/peritoneal/pericardial catheter; Ommaya reservoirs or other dedicated central venous access catheters such as a Port-a-Cath or Hickman catheter are permitted);
(xxi) administered live vaccine ≤6 weeks before the planned start date of the LD chemotherapy regimen;
(xxii) any unresolved toxicity greater than Grade 1 from previous anticancer therapy (except for cytopenia and clinically non-significant toxicities such as alopecia);
(xxiii) history of additional malignancy other than nonmelanoma skin cancer or carcinoma in situ (e.g., cervix, bladder, breast) unless disease-free for at least 2 years;
(xxiv) subjects with severe immediate hypersensitivity to any of the components of hypoimmune allogenic CD19-directed CAR T cells drug product; and/or
(xxv) prior treatment with a CD47-directed therapy and/or detectable levels of anti-CD47 antibodies.

### Treatment

The treatment and Year 1 follow-up phase begins with initiation of LD chemotherapy followed by hypoimmune allogenic CD19-directed CAR T cells administration and ends at the end of Year 1 (or until subject death, consent withdrawal, start of new anti-cancer therapy, or other predefined situations). Safety, efficacy, and biomarker assessments will be collected according to the schedule of assessments (“SOA”; Table 14 and Table 15).

Prior to hypoimmune allogenic CD19-directed CAR T cells infusion, subjects will receive LD chemotherapy (fludarabine [30 mg/m² IV daily for 3 doses] and cyclophosphamide [500 mg/m² IV daily for 3 doses] given concurrently). Subjects with moderate impairment of renal function (creatinine clearance 50-70 mL/min/1.73 m²) should have their fludarabine dose reduced by 20% and be monitored closely. Cyclophosphamide (500 mg/m²/day IV) can be infused over approximately 60 minutes on Days −5, −4, and −3 and fludarabine (30 mg/m²/day IV) can be infused over approximately 30 minutes on Days −5, −4, and −3.

Hypoimmune allogenic CD19-directed CAR T cells should be administered 3 days (range 2-7 days) after completion of LD chemotherapy. Hypoimmune allogenic CD19-directed CAR T cells can be administered via central venous access, such as a port or peripherally inserted central catheter.

Each subject will receive a single IV infusion of hypoimmune allogenic CD19-directed CAR T cells. Hypoimmune allogenic CD19-directed CAR T cells will be administered on treatment Day 0. Subjects will be pre-medicated with acetaminophen (500 to 1,000 mg orally or equivalent) and/or H1 or H2 antihistamine (diphenhydramine or famotidine) prior to hypoimmune allogenic CD19-directed CAR T cells infusion. Pre-medication with corticosteroids or tocilizumab is not permitted. Antimicrobial prophylaxis may be permitted.

Hypoimmune allogenic CD19-directed CAR T cells manufactured from at least one a single donor will be used for dose escalation. Hypoimmune allogenic CD19-directed CAR T cells derived from approximately 3 donors (including the donor used for dose escalation) will be utilized in dose expansion.

In the present example, corticosteroid therapy at a pharmacologic dose (≥5 mg/day of prednisone or equivalent doses of other corticosteroids) and other immunosuppressive drugs should be avoided for 5 days prior to hypoimmune allogenic CD19-directed CAR T cells infusion.

In the present example, systemic corticosteroids should also be avoided as a premedication in subjects for whom computed tomography (CT) scans with contrast are contraindicated (e.g., subjects with contrast allergy or impaired renal clearance) if the administration is within 5 days before the hypoimmune allogenic CD19-directed CAR T cell treatment. Such subjects should undergo MRI with contrast and noncontrast CT scans instead during the washout period specified above. In such cases, alternative dyes such as gadolinium could be used.

In the present example, corticosteroids and other immunosuppressive drugs should also be avoided for 3 months after the hypoimmune allogenic CD19-directed CAR T cells infusion unless used to manage infusion-related toxicities. Other immunomodulatory medications that might interfere with the evaluation of hypoimmune allogenic CD19-directed CAR T cells, such as nonsteroidal anti-inflammatory agents, should also be avoided for the same period unless medically necessary.

In the present example, therapeutic doses of systemic anticoagulants, such as unfractionated heparin and low-molecular weight heparin, should also be avoided whenever possible for subjects who are at risk of bleeding due to thrombocytopenia.

In the present example, treatments for underlying disease, such as surgery, chemotherapy, immunotherapy, targeted agents, radiation, and high-dose corticosteroids (other than those defined/allowed in this protocol) and other investigational agents should be prohibited, except as needed for treatment of disease progression after the hypoimmune allogenic CD19-directed CAR T cells infusion.

In the present example, live vaccines should not be administered within 6 weeks before the planned start date of the LD regimen or within 3 months after hypoimmune allogenic CD19-directed CAR T cells treatment.

### Phase 1a: Dose Escalation

Subjects in Phase 1 will not have received prior CD19-directed CAR T therapy (autologous or allogeneic).

The Phase 1a dose escalation will identify the recommended phase 2 dose (RP2D) of hypoimmune allogenic CD19-directed CAR T cells of the present disclosure in subjects with NHL or CLL using a 3+3 design. An RP2D will be identified based on an assessment of the totality of data from emerging safety, anti-tumor activity, and cellular kinetics.

Three hypoimmune allogenic CD19-directed CAR T cell dose levels (DL) will be explored in 3 separate cohorts in dose escalation. If DL1 is not tolerated due to occurrence of DLT(s), the dose will be de-escalated to DL-1 (Table 12).

TABLE 12
Dose Escalation Cohorts

| | | Number of |
|---|---|---|
| Dose Level | Dose (Viable CAR⁺ Cells) | Subjects* |
| −1* | 30 × 10⁶ | Up to 10 |
| *DL −1 will be opened if 2 | | |
| DLTs occur at DL1. | | |
| 1 | 60 × 10⁶ | Up to 10 |
| 2 | 120 × 10⁶ | Up to 10 |
| 3 | 200 × 10⁶ | Up to 10 |
| 4 | 400 × 10⁶ | Up to 10 |

*Number of subjects can be changed upon review and approval based on clinical data during the study.

The Phase 1a dose escalation employs a gradual dose-escalation strategy, starting at the fixed cell dose of 60×10⁶ CD19-directed CAR T cells and increasing the cell dose by ~2-fold for each successive dose cohort. The initial starting dose along with the gradual dose-escalation approach will provide adequate margin of safety for subjects enrolled in the study.

Assessment and recommendations for dose escalation/de-escalation will be made at the end of each cohort in accordance with the rules described in Table 13.

TABLE 13
Rules for Dose Escalation

| Number of Subjects with a | |
|---|---|
| DLT at a Given Dose Level | Escalation Decision Rule |
| 0/3 | Escalate to the next dose level. |
| 1/3 | Enroll 3 additional subjects at the current |
| | dose level for a total of 6 subjects. |
| 1/6 | Escalate to the next dose level. |
| 2 or more subjects in a | The maximum tolerated dose (MTD) has |
| dose level (up to 6 | been exceeded. De-escalate to the next |
| subjects) | lower dose level or declare MTD at the |
| | next lower dose level if 6 subjects are |
| | already tested. |

### Phase 1b: Dose Expansion

Phase 1b dose expansion will further evaluate safety and efficacy at the RP2D in subjects with LBCL and CLL. Subjects in Phase 1b dose expansion studies will be a separate cohort of CAR-naïve subject and subjects who have received prior approved CD19 CAR T therapy.

Approximately 20 subjects with LBCL who have not received prior CD19-directed CAR T cell therapy (autologous or allogeneic) will be enrolled into the Phase 1b dose expansion part of the study to further characterize the safety and clinical activity of hypoimmune allogenic CD19-directed CAR T cells dosed at the RP2D. Hypoimmune allogenic CD19-directed CAR T cells derived from approximately 3 donors (including the donor(s) used for Phase 1a dose escalation) will be utilized in dose expansion. Separate Phase 1b cohorts in other indications (e.g., mantle lymphoma [MCL]) may be added based on emerging data.

### Dose Limiting Toxicities (DLT)

A DLT is defined as the occurrence of any one of the following events that is at least possibly related to hypoimmune allogenic CD19-directed CAR T cell infusion within 28 days of administration:
any Grade 5 event (death) without clinical or radiologic evidence of disease progression;
all Grade 4 CRS, and Grade 3 CRS that fail to improve to ≤Grade 2 within 72 hours following adequate therapy;
all ≥Grade 3 neurotoxicities of any duration;
all ≥Grade 3 acute graft-versus-host disease (GvHD) events that require oral or IV corticosteroids (>1 mg/kg) and does not resolve within 7 days (as described in Harris et al., Biol Blood Marrow Transplant., 2016).
all Grade 2 acute GvHD events that are steroid refractory (i.e., progression after 3 days or no response after 7 days of systemic steroid treatment [≥1 mg/kg/day prednisone of equivalent])
Grade 4 neutropenia or thrombocytopenia, not attributable to underlying disease or lymphodepletion chemotherapy, that does not improve to ≤Grade 2 within 42 days;
Grade 3 thrombocytopenia with clinically significant bleeding;
all ≥Grade 3 toxicities involving vital organs (e.g., cardiac, pulmonary) of any duration (Exceptions may be made for Grade 3 or 4 abnormal hepatic or renal function tests that improve to ≤Grade 2 within 7 days); and
all other Grade 3 toxicities, not attributable to underlying disease or lymphodepleting chemotherapy, that do not resolve to ≤Grade 2 within 72 hours.

The following events may be expected from CD19-directed CAR T cell therapy and will not be considered DLTs:
Grade 3 cytopenia or Grade 4 lymphopenia/anemia;
Grade 3 tumor lysis syndrome (TLS) for ≤2 weeks (as described in Coiffier et al, J Clin Oncol., 2008); and
Grade 3 infections.

### Study Assessments

### Demographic Data

Demographic data will be collected at screening for each subject including sex, year of birth, race, ethnicity, to assess the possible association of demographics with subject safety and treatment effectiveness. Known baseline genetic disease mutations will be recorded for all subjects, where applicable. Additionally, for female subjects the number of previous births (along with gender of births) will be collected to assess for potential Rh alloimmunization.

### Medical and Treatment History

Relevant medical history from before enrollment will be collected during screening. Relevant medical history is defined as information about the subject's medical condition(s) that would be typically shared in a referral letter.

In addition to the medical history, all history related to the subject's cancer profile and status, treatment(s), and response(s) to treatment(s) dating back to the original diagnosis will be collected.

### Safety Assessments

Safety assessments will include the collection of adverse events (AEs) and serious adverse events (SAEs), clinical laboratory tests, imaging, physical and neurologic examinations, vital signs, cardiac function (e.g., electrocardiogram [ECG]), and ECOG performance status.

### Adverse Events

During clinic visits, subjects will be asked about any AEs that might have occurred since the last study visit. SAEs, TRAEs, TEAE and/or TAEs, AESIs, and DLTs will be collected at each study visit.

### Clinical Laboratory Tests

Blood, serum, or urine samples will be collected for clinical laboratory tests for safety assessments at the time points indicated in the SOA (Table 14 and Table 15). Additional samples may be collected as needed for further safety testing.

TABLE 14
Schedule of Assessments for Month 1 (Table 14A) and Months 2-24 (Table 14B)
A. Schedule of Assessments for Month 1

| | | | | | Hypoimmune | | | | | | | | | | | | | |
|---|---|---|---|---|---|---|---|---|---|---|---|---|---|---|---|---|---|---|
| | | | | | allogenic | | | | | | | | | | | | | |
| | | | | | CD19-directed | | | | | | | | | | | | | |
| | | | | | CAR T cell | | | | | | | | | | | | | |
| | | LD | | | Day | | | | | | | | | | | | | |
| | Screening | Chemo | | | 0 | 0 | Month 1 Follow-Up | | | | | | | | | | | |
| | (Up to 21 | Day | | | (Pre- | (Post- | Day | | | | | | | | | | | |
| Procedure | Days) | −5 | −4 | −3 | Dose) | Dose) | 1 | 2 | 3 | 4 | 5 | 6 | 8 | 10 | 13 | 16 | 21 | 28 |
| Demographics and | X | | | | | | | | | | | | | | | | | |
| Medical History | | | | | | | | | | | | | | | | | | |
| Height and Weight | X | X | X | X | X | | | | | | | | | | | | | X |
| Concurrent | Continuously | | | | | | | | | | | | | | | | | |
| Medications | | | | | | | | | | | | | | | | | | |
| Clinical Exam | X | X | X | X | X | X | X | X | X | X | X | X | X | X | X | X | X | X |
| Neurologic Exam | X | | | | X | | X | X | X | X | X | X | X | X | X | X | X | X |
| ECOG Assessment | X | X | | | X | | As clinically indicated | | | | | | | | | | | X |
| Vital Signs | X | X | X | X | X | Every | Twice daily (every 12 hours) | | | | | | X | X | X | As clinically | | |
| | | | | | | 6 hr | | | | | | | | | | indicated | | |
| | | | | | | (±1 hr) | | | | | | | | | | | | |
| Brain MRI | X | | | | As clinically indicated | | | | | | | | | | | | | |
| Lumbar Puncture | X | | | | As clinically indicated | | | | | | | | | | | | | |
| ECHO | X | | | | | | | | | | | | | | | | | |
| Bone Marrow | X | | | | | | | | | | | | | | | | | X |
| Aspiration/Biopsy | | | | | | | | | | | | | | | | | | |
| Tumor Biopsy | X | | | | | | | | | | | | | | | | | X |
| Disease Assessment | X | X | | | | | | | | | | | | | | | | |
| (e.g., Lugano or | | X | | | | | | | | | | | | | | | | |
| iwCLL) | | | | | | | | | | | | | | | | | | |
| Telemetry Cardiac | | | | | | At the onset of Grade 2 CRS until resolution of Grade ≤1, and additional as | | | | | | | | | | | | |
| Monitoring | | | | | | clinically indicated | | | | | | | | | | | | |
| 12-Lead ECG | X | As clinically indicated | | | | | | | | | | | | | | | | |
| SAE Assessment | Continuously | | | | | | | | | | | | | | | | | |
| AE Assessment | | Continuously | | | | | | | | | | | | | | | | |
| DLT Assessment | | | | | | Continuously | | | | | | | | | | | | |
| Chemistry Panel | X | X | X | X | X | | X | X | X | | X | | X | | X | | X | X |
| (Serum) | | | | | | | | | | | | | | | | | | |
| CBC with Diff. | X | X | X | X | X | | X | X | X | | X | | X | | X | | X | X |
| (Blood) | | | | | | | | | | | | | | | | | | |
| Coagulation | X | X | | X | | | | | | | | | X | | X | | X | X |
| Urinalysis | X | As clinically indicated | | | | | | | | | | | | | | | | |
| LDH/CRP/Ferritin | X | X | | | X | | X | X | X | | X | | X | | X | | X | X |
| Serum Ig Levels | X | | | | | | | | | | | | | | X | | | X |

| | | |
|---|---|---|
| Covid-19 Test | X | As clinically indicated |
| Serology | X | As clinically indicated |
| Pregnancy Test | X | As clinically indicated |

| | |
|---|---|
| Biomarker | See Table 15 |

B. Schedule of Assessments for Months 2-12, Year 2, and Final Follow-Up

| | Months 2-12 Follow-Up (±7 days) | | | | | | | | | | | Year 2 | Final |
|---|---|---|---|---|---|---|---|---|---|---|---|---|---|
| Procedure | M2 | M3 | M4 | M5 | M6 | M7 | M8 | M9 | M10 | M11 | M12 | Follow-Up | Follow-Up |
| Height and Weight | | X | | | X | | | | | | X | | X |
| Concurrent | Continuously | | | | | | | | | | | Quarterly | X |
| Medications | | | | | | | | | | | | | |
| Clinical Exam | X | X | X | X | X | X | X | X | X | X | X | Quarterly | X |
| Neurologic Exam | As clinically indicated | | | | | | | | | | | | X |
| ECOG Assessment | X | X | | | X | | | X | | | X | | X |
| Bone Marrow | | X | | | X | | | X | | | X | M18 | X |
| Aspiration/Biopsy | | | | | | | | | | | | | |
| Tumor Biopsy | | | | | | | | | | | | | X |
| Disease Assessment | | X | | | X | | | X | | | X | M18 | X |
| (e.g., Lugano or | | | | | | | | | | | | | |
| iwCLL) | | | | | | | | | | | | | |
| SAE Assessment | Continuously | | | | | | | | | | | Quarterly | X |
| AE Assessment | Continuously | | | | | | | | | | | Quarterly | X |
| DLT Assessment | | | | | | | | | | | | | |
| Chemistry Panel | X | X | | | X | | | X | | | X | | X |
| (Serum) | | | | | | | | | | | | | |
| CBC with Diff. | X | X | X | X | X | | | X | | | X | Quarterly | X |
| (Blood) | | | | | | | | | | | | | |
| Coagulation | | | | | | | | | | | | | X |
| LDH/CRP/Ferritin | X | X | | | X | | | X | | | X | | X |
| Serum Ig Levels | X | X | X | X | X | | | X | | | X | | X |
| Pregnancy Test | As clinically indicated | | | | | | | | | | | | X |
| Biomarker | See Table 15 | | | | | | | | | | | | |
| Assessments | | | | | | | | | | | | | |
| Survival Status | | | | | | | | | | | | Quarterly | X |

TABLE 15
Biomarker Schedule of Assessments

| | | | Hypoimmune | | | | | | | | | | | | | | |
|---|---|---|---|---|---|---|---|---|---|---|---|---|---|---|---|---|---|
| | | | allogenic | | | | | | | | | | | | | | |
| | | | CD19-directed | | | | | | | | | | | | | | |
| | | | CAR T cell | | | | | | | | | | | | | | |
| | Screening | LD | Day | | Year 1 Follow-Up | | | | | | | | | | Month | | |
| | (Up to | Chemo | 0 | 0 | | | | | | Day (±2 | | Day/Month | | | (±7 Days) | Year 2 | Final |
| | 21 | Day | (Pre- | (Post- | Day (±1 Day) | | | | | Days) | | (±4 Days) | | | M 7-12 | Follow- | Follow- |
| Assessment | Days) | −5 | Dose) | Dose) | 1 | 3 | 6 | 8 | 10 | 13 | 16 | 21 | 28 | M 2-6 | Days) | Up | Up |
| Immunohistoche | X | | | | | | | | | | | | X | | | | X |
| mistry analysis | | | | | | | | | | | | | | | | | |
| (biopsy) | | | | | | | | | | | | | | | | | |
| CAR T Cellular | | X | | X | X | X | X | X | X | X | X | X | X | X | M9, M12 | X | X |
| Kinetics (CAR | | | | | | | | | | | | | | | | | |
| VCN) | | | | | | | | | | | | | | | | | |
| CAR T Cell | | X | | X | X | X | X | X | X | X | X | X | X | X | M9, M12 | X | X |
| Enumeration | | | | | | | | | | | | | | | | | |
| (FACS) | | | | | | | | | | | | | | | | | |
| CD47 Expression | | X | | | X | | X | | | X | | X | X | X | M9, M12 | X | X |
| (FACS) | | | | | | | | | | | | | | | | | |
| Cytokine | | X | X | | X | X | X | | | X | | X | X | M3, | | | X |
| Profiling | | | | | | | | | | | | | | M6 | | | |
| Replication | | X | | | | | | | | | | | | M3, | M12 | M18 | X |
| Competent | | | | | | | | | | | | | | M6 | | | |
| Lentivirus | | | | | | | | | | | | | | | | | |
| T Cell Clonality | | X | | | | | | | | X | | | X | M3, | M12 | M18 | X |
| (Clonal | | | | | | | | | | | | | | M6 | | | |
| Expansion) | | | | | | | | | | | | | | | | | |
| Immunophenoty | | X | | | | | X | | | X | | X | X | M3, | M9, M12 | X | X |
| ping | | | | | | | | | | | | | | M6 | | | |
| MRD/ctDNA | X | X | X | | | | X | | | | | | X | M3, | | | |
| | | | | | | | | | | | | | | M6 | | | |
| Humoral | | X | | | | | X | | | X | | | X | M3, | M12 | M18 | X |
| Immunogenicity | | | | | | | | | | | | | | M6 | | | |
| (anti-CD19- | | | | | | | | | | | | | | | | | |
| directed CAR) | | | | | | | | | | | | | | | | | |
| Humoral | | X | | | | | | | | | | | X | M2, | M7, M9, | | X |
| Response | | | | | | | | | | | | | | M3, | M12 | | |
| (DSA/ADCC/ | | | | | | | | | | | | | | M5 | | | |
| CDC) | | | | | | | | | | | | | | | | | |
| T Cell Response | | X | | | | | | | | | | | X | M2, | M7, M9, | | X |
| (ELISpot/T Cell | | | | | | | | | | | | | | M3, | M12 | | |
| killing) | | | | | | | | | | | | | | M5 | | | |
| NK Cell | | X | | | | | | | | | | | X | M2, | M7, M9, | | X |
| Response (NK | | | | | | | | | | | | | | M3, | M12 | | |
| Cell killing) | | | | | | | | | | | | | | M5 | | | |
| Single Cell | | X | | | X | | | | | X | | | X | M3, | | | X |
| RNAseq | | | | | | | | | | | | | | M6 | | | |
| (scRNAseq) | | | | | | | | | | | | | | | | | |
| Bulk RNAseq | | X | | | | | | | | X | | | X | M2, | M12 | M18 | × |
| | | | | | | | | | | | | | | M3, | | | |
| | | | | | | | | | | | | | | M6 | | | |
| Cellular | | X | | | | | | | | X | | | X | M3 | | | X |
| Immunogenicity | | | | | | | | | | | | | | | | | |
| (CD19-direted | | | | | | | | | | | | | | | | | |
| CAR peptides) | | | | | | | | | | | | | | | | | |
| Serum protein | | X | X | | | | | | | X | | | X | | | | X |
| quantification | | | | | | | | | | | | | | | | | |
| (e.g., soluble | | | | | | | | | | | | | | | | | |
| SIRPa) | | | | | | | | | | | | | | | | | |

All clinical laboratory test assessments listed in Table 16 will be performed at the local laboratory, unless otherwise noted.

TABLE 16
Local Laboratory Tests for Safety Assessments

| Serum | | | | |
|---|---|---|---|---|
| Chemistry | Hematology | Coagulation | Urinalysis | Other |
| Albumin | Absolute | Activated | Specific gravity | Pregnancy test |
| | lymphocyte | thromboplastin | | |
| | count | time | | |
| Alanine | Absolute | Prothrombin | pH | Serologic testing |
| aminotransferase | neutrophil count | time; | | (e.g., HIV, |
| (Serum | | International | | HepB, HepC) |
| glutamic- | | normalized | | |
| pyruvic | | Ratio | | |
| transaminase) | | | | |
| Alkaline | Hemoglobin | Fibrinogen | Blood | COVID-19 test |
| phosphatase | | | | |
| Aspartate | Red blood cell | D-dimer | Protein | Serum |
| aminotransferase | count | | | immunoglobulin |
| (Serum | | | | levels |
| glutamic- | | | | |
| oxaloacetic | | | | |
| transaminase) | | | | |
| Total | Hematocrit | | Glucose | Beta-2 |
| bicarbonate | | | | microglobulin |
| | | | | (CLL only) |
| Total bilirubin/ | Reticulocytes | | Bilirubin | Direct |
| direct bilirubin | | | | Antiglobulin test |
| | | | | (CLL only) |
| Blood urea | Platelets | | White blood cell | Haptoglobin |
| nitrogen or urea | | | count | (CLL only) |
| Total calcium | White blood cell | | Red blood cell | |
| | count | | count | |
| Chloride | Differential: | | Epithelial cells | |
| Creatinine | Neutrophils | | Bacteria | |
| Creatinine | Eosinophils | | Casts | |
| clearance (as | | | | |
| estimated by | | | | |
| MDRD | | | | |
| equation) | | | | |
| (screening only) | | | | |
| Standard c- | Basophils | | Crystals | |
| reactive protein | | | | |
| Ferritin | Lymphocytes | | | |
| Glucose | Monocytes | | | |
| Inorganic | | | | |
| phosphorus | | | | |
| Lactate | | | | |
| dehydrogenase | | | | |
| Magnesium | | | | |
| Potassium | | | | |
| Sodium | | | | |
| Uric acid | | | | |
| Phosphorus | | | | |
| Total Protein | | | | |

Central laboratory test assessments intended for assessment of safety and other endpoints (e.g., immunogenicity and efficacy) are listed in Table 17.

TABLE 17
Central Laboratory Assessments

| Use | Primary Specimen | Analytical Method |
|---|---|---|
| Determine | CAR T enumeration | Flow cytometry (FACS) |
| cellular kinetics | CAR T cellular kinetics | ddPCR, CAR, vector copy number |
| parameters and | CD47 expression | FACS |
| safety | Replication competent lentivirus | RCL |
| assessments | T cell clonality | Clonal expansion |
| | Immunophenotyping | Flow cytometry |
| | Cytokine profiling | Meso-scale discovery |
| Evaluate | NK response | NK cell killing |
| hypoimmune | T cell response | ELISpot/T cell killing |
| allogenic | Humoral response | Donor specific antibody/antibody- |
| CD19-directed | Humoral immunogenicity | dependent cell-mediated |
| CAR T cell | | cytotoxicity/complement dependent |
| immune | | cytotoxicity |
| response | | Anti-CD19-directed CAR |
| Exploratory | Single cell RNA | Single cell RNA sequencing |
| analyses | Minimum residual | ClonoSeq (Phase 1b dose expansion |
| | disease/circulating tumor DNA | only) |
| | Bulk RNA | Bulk RNA sequencing |
| Evaluate | Cellular immunogenicity | CD19-directed CAR peptide |
| hypoimmune | Immunohistochemistry analysis | Biopsy staining for CD19 (optional) |
| allogenic | | |
| CD19-directed | | |
| CAR T cell- | | |
| derived | | |
| immunogenicity | | |

### Clinical Examination

Clinical examinations will be performed during screening and at times noted in the SOA (Table 14). At subsequent examinations, clinically significant adverse changes when compared with the baseline examination will be reported as AEs. Any clinically relevant findings will be documented.

Height will be measured at screening only. Weight will be collected during screening and at times noted in the SOA (Table 14).

Vital signs include systolic/diastolic blood pressure, heart rate, respiratory rate, oxygen saturation, and temperature. Vital signs will be monitored as indicated in the SOA (Table 14). During hypoimmune allogenic CD19-directed CAR T cell treatment, vital signs will be monitored before and after infusion and then at regular intervals described in Table 14. Subject will be in a supine position and in a rested and calm state for at least 5 minutes before blood pressure assessments are conducted. Oxygen saturation will be measured using a standard pulse oximeter. The subject will be in a rested and calm state for at least 5 minutes before pulse oximetry assessments are completed.

### Neurologic Examination

Neurologic examination will be performed to assess the immune effector cell-associated encephalopathy (ICE) score, according to the time points specified in the SOA (Table 14). The subject's neurological status will be evaluated at screening to establish a baseline. Changes in neurological status will be reported as an AE if considered clinically significant.

### Eastern Cooperative Oncology Group Assessment (ECOG)

Performance status as measured by the ECOG scale (Oken 1982) will be performed at the time points specified in the SOA (Table 14) to quantify the subject's general well-being and ability to perform activities of daily life.

### Cardiac Function Assessment

Each subject's cardiac function including ejection fraction, as measured by an ECHO scan, will be assessed during the screening period to confirm study eligibility. An ejection fraction >40% with no evidence of clinically significant pericardial effusion is required for study eligibility. An ECHO scan performed within 1 month prior to hypoimmune allogenic CD19-directed CAR T cell treatment may be used for confirmation of eligibility.

To establish a baseline, a 12-lead ECG will be performed during the screening period. ECG assessments will include PR duration, QRS duration, QT duration, RR duration, and corrected QT interval. The corrected QT interval will be calculated using Fridericia's formula (Fridericia 2003). Subjects with cardiac arrhythmias requiring medication will be excluded from the study.

### Brain Magnetic Resonance Imaging

To confirm study eligibility, a brain MRI will be performed with contrast whenever possible or without contrast in case of contraindication. If the subject cannot undergo MRI, a head CT may be done, preferably with IV contrast if not contraindicated.

Each subject will undergo screening brain MRI to rule out CNS metastasis during the screening period of the study. An MRI performed after the subject's last chemotherapy treatment and within 3 months before enrollment may be used for eligibility. An MRI collected as standard of care within this timeframe is acceptable.

An MRI can be performed post-treatment for subjects with new onset of clinically significant neurologic events of severity Grade 2 or higher after the hypoimmune allogenic CD19-directed CAR T cell infusion.

### Lumbar Puncture

Lumbar puncture will be performed under certain circumstances during screening or post-treatment with hypoimmune allogenic CD19-directed CAR T cell at the time points specified in the SOA (Table 14). Opening pressures should be measured and recorded in the subject's site chart with each lumbar puncture whenever possible.

Subjects with a history of CNS malignancy or leptomeningeal carcinomatosis, symptoms of CNS malignancy, such as new onset of severe headaches, neck stiffness, or any focal neurologic findings on physical examination, will undergo lumbar puncture at the screening visit for examination of cerebrospinal fluid (CSF) to determine the presence of CNS malignancy.

Lumbar puncture is also recommended for subjects with new onset of clinically significant neurologic events of severity Grade 2 or higher after the hypoimmune allogenic CD19-directed CAR T cell infusion.

### Replication Competent Lentivirus (RCL)

As hypoimmune allogenic CD19-directed CAR T cells comprise lentiviral vector-transduced T cells, the presence of RCL in the blood of treated subjects will be monitored per the schedule presented in the SOA (Table 15).

Peripheral blood mononuclear cells (PBMCs) for RCL testing will be obtained at baseline (before LD chemotherapy), and at months 3, 6, 12, 18, and 24. If a positive RCL assay result is obtained from a subject blood specimen, the investigator(s) will be informed, and the subject will be rescheduled for RCL retest. Thereafter, samples will only be collected if an RCL event is clinically suspected and/or a subject's PBMC sample tests positive for RCL at any time point within the first year. If the latter, samples will continue to be collected and tested annually for up to 15 years or as clinically indicated. This RCL testing will end after 2 consecutive negative results. If a subject dies or develops a second primary malignancy during the study or follow-up and RCL is suspected, every effort should be made to obtain a blood sample to assay for RCL and vector elements. In the case of a solid tumor second primary malignancy, every effort should be made to obtain a biopsy sample of the neoplastic tissue or the pertinent autopsy tissue to assay for the presence of vector elements.

### T Cell Clonality Assays

Samples will be collected for monitoring of abnormal T cell proliferation by flow cytometry for CAR⁺ T cells over the first 3 months. In addition, samples will also be collected for storage and will be analyzed if abnormal CAR T cell expansion is observed. T cell clonal expansion will be monitored by flow and vector copy number (VCN) for the CD19-directed CAR transgene, and by complete blood count (CBC). If more than 1% of the T cells are positive for CD19-directed CAR (by flow cytometry) after 3 months on treatment or if T cell expansion occurs after contraction (by VCN) during the first 3 months on treatment, further studies to assess T cell clonality (including but not limited to vector integration sites) will be performed.

Additionally, fresh tumor samples may be collected at the time of suspected second primary malignancy and suspected RCL for additional characterization if necessary. A portion of the tumor tissue may be subjected to exploratory biomarker analysis.

### Immunogenicity Assays

The presence of Anti-CD19-directed CAR antibodies in the subject's serum will be monitored at time points identified in the SOA (Table 14).

The hypoimmune allogenic CD19-directed CAR T cells induced immunogenicity will be evaluated by detecting the following analytes.
(i) Total anti-chimeric antigen receptor (CAR) transgene protein specific antibody response (anti-CAR Tab) in human serum.
(ii) Assessment of neutralizing anti-CAR transgene protein humoral responses will be considered based on the overall data obtained during clinical study conduct. Appropriate samples will be collected and stored for later analysis.
(iii) Cellular immunogenicity will be tested using ELISpot for CD19-directed CAR peptides. Samples will be saved and analysed if abnormal CAR T proliferation/kinetic was observed.

### Cytokine Profiling

Samples will be collected for cytokine profiling at time points described in the SOA (Table 14). A multiplexed MSD platform method will be applied to detect and quantify specific analytes.

### Disease Assessment

Disease response and progression will be assessed in NHL subjects based on the Lugano classification criteria and in CLL subjects based on the International Workshop on Chronic Lymphocytic Leukemia (iwCLL) criteria.

Disease assessments will be performed at months 1, 3, 6, 9, 12, 18, and 24 (±2 weeks). For dose escalation, determination of disease status may be performed in concordance with central imaging assessments.

For all positron emission tomography (PET)-CT scans (NHL) or CT scans (CLL):
(i) the CT must be of diagnostic quality with IV-iodinated contrast, performed by either of the following modalities, depending on the site's capability: as part of a combined PET-diagnostic CT; or as a separate diagnostic quality CT (with IV-iodinated contrast);
(ii) the PET-CT or CT scan must include the neck, chest, abdomen, and pelvis, along with the appropriate imaging of all other sites of disease. For CLL subjects, the liver and spleen size should be recorded.
(iii) if IV-iodinated contrast is contraindicated, then the diagnostic CT scan can use non-iodinated contrast. If non-iodinated contrast is not available or not recommended, then the diagnostic CT can be non-enhanced.

To confirm eligibility and/or to establish a baseline, PET-CT or CT scans of the neck, chest, abdomen, and pelvis, along with the appropriate imaging of all other sites of disease, will be required at screening. PET-CT or CT scans should be performed as close to enrollment as possible and within 21 days before LD chemotherapy for eligibility. A PET-CT or CT scan performed after the subject's last line of therapy may be used for confirmation of eligibility if within 21 days before enrollment/LD chemotherapy and no other anticancer treatment has been administered. If a PET-CT or CT scan is performed >21 days before the initiation of LD chemotherapy or if the subject receives any anticancer therapy (e.g., radiation, supraphysiologic doses of steroids, chemotherapy; including bridging therapy) between the last PET-diagnostic CT scan and the initiation of LD chemotherapy, the PET-CT or CT scan will be repeated before LD chemotherapy to establish a new baseline. Lesions that have been previously irradiated will be considered measurable only if progression has been documented following completion of radiation therapy.

The PET-CT or CT scans will be performed at the time points outlined in the SOA (Table 14) through Month 24 or until disease progression, whichever occurs first. A 2-week window is allowed for each time point. If the subject's disease has not progressed by month 24, disease assessments will continue to be performed per standard of care. A PET-CT or CT scan can be performed at any time disease progression is suspected.

For NHL subjects, the PET portion of the PET-diagnostic CT scan will be scored per the Deauville-5 point scale. Both the PET and CT portions of the scan will be interpreted according to the Lugano criteria (Cheson et al., J Clin Oncol., 2014). For CLL, disease assessment will be performed according to iwCLL criteria.

For dose escalation, determination of disease status will be assessed and central imaging assessments may be performed for concordance analysis. For dose expansion, determination of disease status will be based on central assessment. Imaging scans may be collected retrospectively and submitted for review and/or concordance analysis.

For fluorodeoxyglucose (FDG)-avid lymphoma, a subject's bone marrow involvement should be assessed by PET-diagnostic CT. If not available, bone marrow involvement should be assessed by bone marrow aspirate and biopsy before enrollment. For CLL subjects, bone marrow aspirate/biopsy should be performed.

For lymphoma, if there is evidence of baseline bone marrow involvement and if PET-diagnostic CT is not available (or if the lymphoma is not FDG avid), a bone marrow aspirate and biopsy should be performed (Cheson et al., J Clin Oncol., 2014). For CLL subjects, bone marrow aspirate/biopsy should be performed on Day 28, Month 3, Month 6, Month 9, Month 12, Month 18, and Month 24 for disease assessment according to iwCLL criteria (Hallek et al., Blood, 2018).

A bone marrow aspirate and/or biopsy should be considered for any subject with unexplained cytopenia or where there is suspicion of hemophagocytic lymphohistiocytosis (HLH) or any potential cell therapy-related phenomenon after the hypoimmune allogenic CD19-directed CAR T cell infusion.

### Cellular Kinetics Assessments

PBMC samples for cellular kinetics assessments will be drawn according to the schedule presented in the SOA (Table 15) as CAR VCN (droplet digital polymerase chain reaction [ddPCR]) and CAR T cell enumeration (flow cytometry).

Hypoimmune allogenic CD19-directed CAR T cells cellular kinetics (CK) will be evaluated by a validated ddPCR method and reported as level of anti-CD19 CAR transgene copies/μg of genomic DNA (gDNA). For all subjects treated with hypoimmune allogenic CD19-directed CAR T cells, the CK profile and the following derived parameters will be reported: Cₘₐₓ, Tₘₐₓ, t_{1/2}, Cₗₐₛₜ, Tₗₐₛₜ, AUCs.

In addition, the number of CD19-directed CAR expressing cells in the blood will also be tracked by flow cytometry. The assessment of hypoimmune allogenic CD19-directed CAR T cells CK will be used to examine the dose-exposure relationship, and possible correlation of persistence and expansion of hypoimmune allogenic CD19-directed CAR T cells with safety and efficacy endpoints.

The timing, but not the total number of samples, may be modified during the study based on emerging CK data if a change in sampling scheme is considered necessary to better characterize the CK.

### Pharmacodynamic Assessments

Blood/serum samples for pharmacodynamic assessments will be drawn according to the schedule presented in the SOA (Table 15).

Pharmacodynamic assessments will include monitoring of levels of analytes in blood/serum over time. Analytes can include, but are not limited to, the following:
(i) Homeostatic/proliferative cytokines: interleukin (IL)-2, IL-7, IL-15, and granulocytecolony-stimulating factor (G-CSF);
(ii) Inflammatory/immune-modulating cytokines: interferon-gamma (IFN-γ), IFN-α, IL-1β, IL-1Ra, IL-4, IL-6, IL-8, IL-10, IL-12p70, IL-13, IL-17A, granulocyte macrophage colony-stimulating factor (GM-CSF), and tumor necrosis factor-alpha (TNF-α), macrophage inflammatory protein-1 Alpha (MIP-1α), and MIP-1β;
(iii) Correlates of an acute phase response: C-reactive protein (CRP), and ferritin;
(iv) Chemokines: IL-8, C-X-C motif chemokine ligand-10 (CXCL-10), and monocyte chemotactic protein-1 (MCP-1); and
(v) Associated responses related with CD19-directed CAR activity: circulating CD19⁺ B cells, and lactate dehydrogenase (LDH).

### Minimal Residual Disease (MRD) Assessments

A next-generation sequencing (NGS) MRD assay will be used to measure ctDNA MRD levels before and after the hypoimmune allogenic CD19-directed CAR T cells infusion (Frank 2019). Tumor DNA will be extracted from archival or fresh tissue biopsy to establish tumor clonotypes(s). Subsequent plasma samples will be collected to assess the MRD-negative response rate as an exploratory endpoint.

During dose escalation, MRD assessment of CLL subjects using peripheral blood will be performed using a validated institutional assay per NCCN 2022 guidelines (e.g., flow cytometry, NGS, or polymerase chain reaction using allele-specific oligonucleotides [ASO-PCR]).

### Hypoimmune Allogeneic CAR T Cell-Mediated Immune Recognition Assessments

The immune responses against hypoimmune allogenic CD19-directed CAR T cells will be evaluated at time points identified in the SOA (Table 15) by detecting the following analytes:
(i) Humoral response: anti-hypoimmune allogenic CD19-directed CAR T cell antibodies, e.g., donor specific antibody (DSA), may have an impact on CK and efficacy. Assays of antibody-dependent cellular cytotoxicity (ADCC) and complement-dependent cytotoxicity (CDC) will be conducted and evaluated as an exploratory endpoint.
(ii) T cell response: the ELISpot assay will be utilized to assess host immune activation against hypoimmune allogenic CD19-directed CAR T cells, and cellular anti-CAR T cytotoxicity assay will be conducted and evaluated for exploratory analyses.
(iii) NK cell response: an NK cell-mediated killing study will be utilized to explore NK cell activation against hypoimmune allogenic CD19-directed CAR T cells.

### Tumor Biopsy Assessments

The most recently available archived or newly collected/on-study pre-hypoimmune allogenic CD19-directed CAR T cells infusion tumor biopsy samples will be collected and submitted to the central laboratory for evaluation of CD19 (or CD20/CD22) expression and prognostic variables, including MRD. Archived tumor biopsy samples will consist of either a formalin-fixed paraffin-embedded tumor block or 20 unstained slides (10 slides are acceptable for dose escalation). If the most recent archived biopsy is older than 6 months from the time of screening, a fresh tumor sample should be obtained before initiation of LD chemotherapy to continue on-study. Slides that have been previously stained with antibodies or other reagents are not acceptable.

On-study biopsies of the tumor can be performed at screening, at Month 1 after hypoimmune allogenic CD19-directed CAR T cells treatment, and upon disease progression. Biopsies are not required in subjects without evidence of disease. The tumor biopsy taken at disease progression will help determine mechanisms of tumor resistance.

Exploratory analysis of tumor or immune cell markers that correlate with response to hypoimmune allogenic CD19-directed CAR T cells or disease prognosis will be executed. Tumor biopsies are not required for CLL subjects who have provided a bone marrow biopsy.

### Other Exploratory Assessments

PBMCs will be collected for assessment of immunophenotypic changes induced by hypoimmune allogenic CD19-directed CAR T cells. These blood samples as well as final product samples will be analyzed at time points identified in the SOA (Table 15) for the presence and changes in immune cell state by flow cytometry; including, but not limited to:
(i) Expression level of CD47 on hypoimmune allogenic CD19-directed CAR T cells;
(ii) Broad immune cell phenotyping: lymphoid and myeloid cell phenotypes, e.g., T cells, B cells, NK cells and monocytes; and
(iii) Immune cell subsets: differentiation, activation, and exhaustion.

These PBMCs and final product samples may be assessed by gene expression profiling through single cell RNA sequencing for broad molecular landscape analyses. Remaining samples collected as part of the study will be stored for up to 15 years for future exploratory analysis related to abnormal clonal expansion. Additional studies involving, but not limited to, NGS may be performed.

Tumor biopsies provided at baseline or post-infusion will primarily be evaluated for disease assessments and diagnostic confirmation. In addition, tumor biopsies may also be utilized for exploratory mutational analysis, tumor microenvironment assessments, and or gene/protein expression profiling.

In addition to the bone marrow aspirate and biopsies to be performed as part of disease assessment, if a subject experiences unexplained cytopenia or suspicion of hemophagocytic lymphohistiocytosis (HLH) or any potential cell therapy-related phenomenon after the hypoimmune allogenic CD19-directed CAR T cells infusion and a bone marrow biopsy/aspirate is obtained and sent to the local laboratory for assessment, a portion of the sample can also be subjected to exploratory analysis.

### Post-Treatment Follow-Up

After completing the hypoimmune allogenic CD19-directed CAR T cells infusion, all subjects will be followed during the post-treatment follow-up period. Counting from Day 0 (the day of infusion), subjects will be evaluated at the following intervals: Day 8 (±1 day), Day 10 (±1 day), Day 13 (±1 day), Day 16 (±1 day), Day 21 (±4 days), Day 28 (±4 days), monthly in Months 2-12 (±1 week) (or until withdrawal of consent and/or start of new systemic therapy).

The following assessments and procedures will be completed for subjects according to the schedule presented in the SOA (Table 14): clinical examination; vital signs; weight; ECOG assessment; concomitant medications documentation; neurologic examination to assess Immune Effector Cell Encephalopathy (ICE score; as described in Lee et al. Biol Blood Marrow Tranplant., 2019); ECG (if clinically indicated); brain MRI if clinically indicated; Lumbar puncture if clinically indicated; tumor biopsy; disease/overall response assessments, including imaging and bone marrow biopsy/aspirate when applicable; AE/SAE/DLT reporting; laboratory assessments for chemistry panel (serum), CBC with differential (blood), coagulation (plasma), urinalysis (if clinically indicated), LDH, CRP, ferritin, Beta-2 microglobulin (CLL only), direct antiglobulin test (CLL only), haptoglobin (CLL only), serum Ig levels, Covid-19 and other viral serology testing (as clinically indicated), β-hCG pregnancy test (serum or urine) for all females of childbearing potential (as clinically indicated); Analytes including cytokines (serum/plasma) and anti-hypoimmune allogenic CD19-directed CAR T cells antibodies, i.e., donor specific antibody and CD19-directed CAR antibody (serum); ctDNA/MRD (dose expansion only; during dose escalation, MRD assessment of CLL subjects is allowed using an institutional assay); CD19-directed CAR T cell kinetics (VCN and FACS), CD47 expression, RCL, T cell clonality, and exploratory analyses including immunophenotyping, T cell killing, NK cell killing, and scRNAseq (PBMCs); and as applicable, an additional sample for the assessment of levels of analytes including cytokines (serum/plasma) and CD19-directed CAR T cells (PBMCs) should be drawn at the first onset and first reoccurrence of any Grade 2 or higher infusion-related toxicity, such as a Grade 2 cytokine release syndrome (CRS; as described in Lee et al. Biol Blood Marrow Tranplant., 2019; Hayden et al., Ann Oncol., 2021; and Thompson et al., J Natl Compr Canc Netw., 2022) or neurologic event, and upon resolution of the event, if not already collected on the day (serum/plasma).

If a subject is readmitted to the hospital after the initial hospitalization observation period with any hypoimmune allogenic CD19-directed CAR T cells related AE, the following samples will be collected on the day of hospital readmission and then weekly through, and including, the day of discharge: analytes including cytokines (serum/plasma); and CD19-directed CAR T cells (PBMCs).

Subjects will begin the Year 2 follow-up period after they have completed the Month 12 visit of the Year 1 follow-up period. The Year 2 follow-up period will assess subjects every 3 months (±2 weeks) starting after Month 12 through Month 24 after the infusion of hypoimmune allogenic CD19-directed CAR T cells.

If a subject experiences disease progression, a final follow-up visit should be completed prior to start of new systemic therapy.

Assessments will include the following: disease/overall response assessments, including imaging and bone marrow biopsy/aspirate when applicable (if the subject's disease has not progressed by Month 24, the disease/overall response assessments will be continue to be performed per standard of care); clinical examination; concomitant medications documentation; CBC; AE/SAE reporting; survival status; CD19-directed CAR antibody (serum); CD19-directed CAR T cell kinetics (VCN and FACS); CD47 expression; RCL; T cell clonality; and exploratory analyses including immunophenotyping (PBMCs); reporting of all deaths; reporting of pregnancies in female subjects or female partners of male subjects; subsequent anticancer therapy for the treatment of B cell cancer; and subjects with suspected second primary malignancy should undergo workup, including collection of tumor biopsy and blood samples, for central lab analysis.

After completion of the Year 2 follow-up period (or at the start of new anticancer therapy or withdrawal of consent prior to end of Year 2), subjects will be enrolled in a separate LTFU protocol for continued safety and survival monitoring for up to 15 years from hypoimmune allogenic CD19-directed CAR T cells treatment

### Results

Subjects as described above were treated with either a Level 1 dose or a Level 2 dose of a drug product comprising hypoimmune allogenic CD19-directed CAR T cells of the present disclosure. The Level 2 dose contained twice as many CAR+ T cells as the Level 1 dose. Subjects 1, 2, and 3 received the Level 1 dose and Subject 4 received the Level 2 dose. Subject 1 had been diagnosed with chronic lymphocytic leukemia (CLL), Subject 2 had been diagnosed with Mantle Cell Lymphoma (MCL), Subject 3 had been diagnosed with Diffuse Large B-Cell Lymphoma (DLBCL), and Subject 4 had been diagnosed with Transformed DLBCL. Subject 2 received a lymphodepletion regimen comprising cyclophosphamide at a dose of 500 mg/m²/day for three days and fludarabine at a dose of 30 mg/m²/day for three days. Subject 1 received a reduced lymphodepletion regimen due to renal insufficiency and received a lymphodepletion regimen comprising cyclophosphamide at a dose of 500 mg/m²/day for three days and fludarabine at a dose of 24 mg/m²/day for three days. Subject 3 and Subject 4 also received lymphodepletion regimens as described herein.

Peripheral blood mononuclear cell (PBMC) samples were taken from subjects at various time points and were characterized via flow cytometry to identify CD19+ cells.

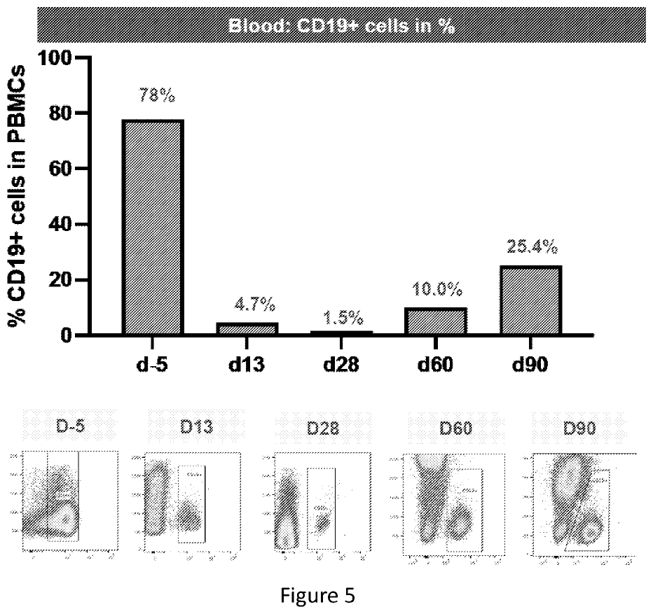

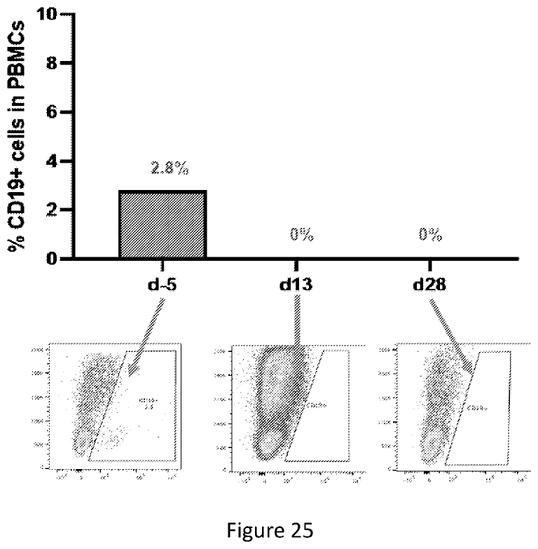

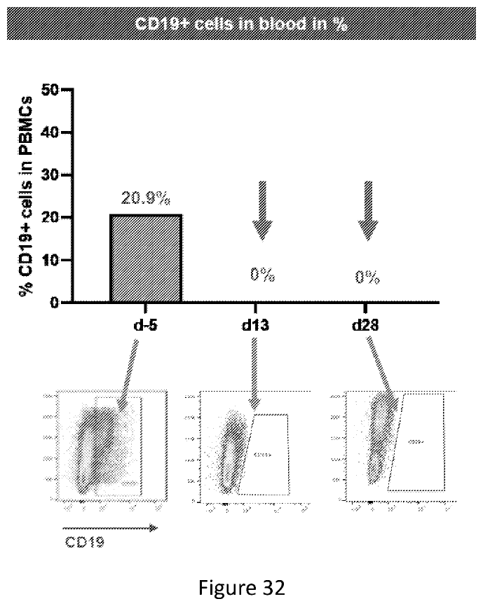

As shown in FIG. 5, Subject 1 exhibited depleted levels of CD19+ cancer cells in PBMCs until day 28, but the percentage of CD19+ cells started to increase at day 60. For Subject 2, as shown in FIG. 13, CD19+ cancer cells were fully depleted at day 13 and day 28, then the percentage of CD19+ started to increase on day 60, but then were fully depleted again at day 90. As shown in FIG. 25, Subject 3 exhibited depleted levels of CD19+ cancer cells in PBMCs until day 28. As shown in FIG. 32, Subject 4 also exhibited depleted levels of CD19+ cancer cells in PBMCs until day 28.

In addition to monitoring levels of CD19+ cells, the effect of the subject's immune system on the administered drug product was determined. The administered drug product comprised subgroups of cells including 1) T cells partially edited to express an anti-CD19 CAR, exogenous CD47, and knock-out TCR (designated herein “WT CAR”; 2) T cells partially edited to knock-out HLA I and HLA II, and knock-out TCR (designated herein “HLAI/II dKO” or “dKO”); and 3) T cells fully edited to express an anti-CD19 CAR and exogenous CD47, knock-out HLA I and HLA II, and knock-out TCR (designated herein “HIP CAR”). Flow sorting methods were used for sorting HLA-I and HLA-II, and CD47 populations in the administered drug product. Various flow sorting methods were also used to create populations of T cells and NK cells from subjects' PBMC samples. A donor specific antibody (DSA) assay was also used to measure the presence and level of IgM and IgG antibodies in subjects' serum samples against donor HLA from the drug product. Serum from the subjects was incubated with the drug product to assess antibody reactivity with drug product antigens and DSAs were evaluated using a flow-based measurement. A time lapse NK cell killing assay was performed using sorted drug product T cells as target cells and sorted NK cells from the subjects' serum were used as effector cells. The cells were imaged using a viability fluorescent stain (target cells) and phase contrast (effector cells) to show survival or non-survival of target cells.

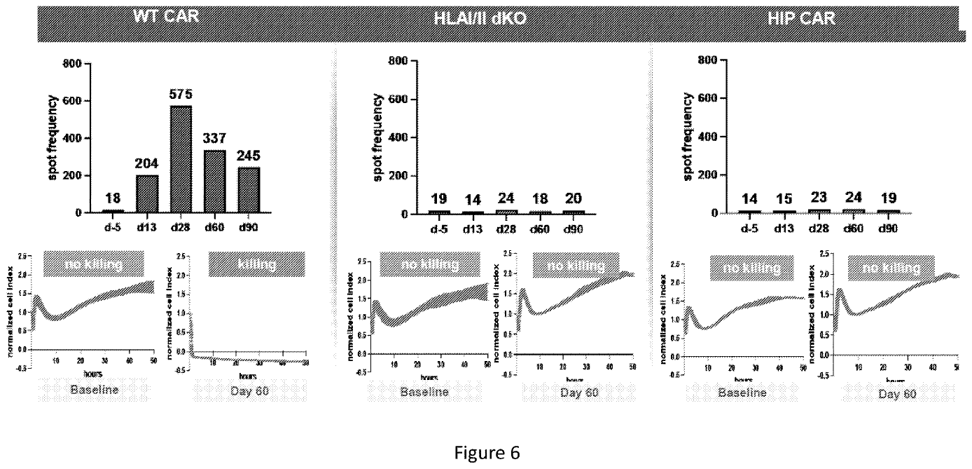

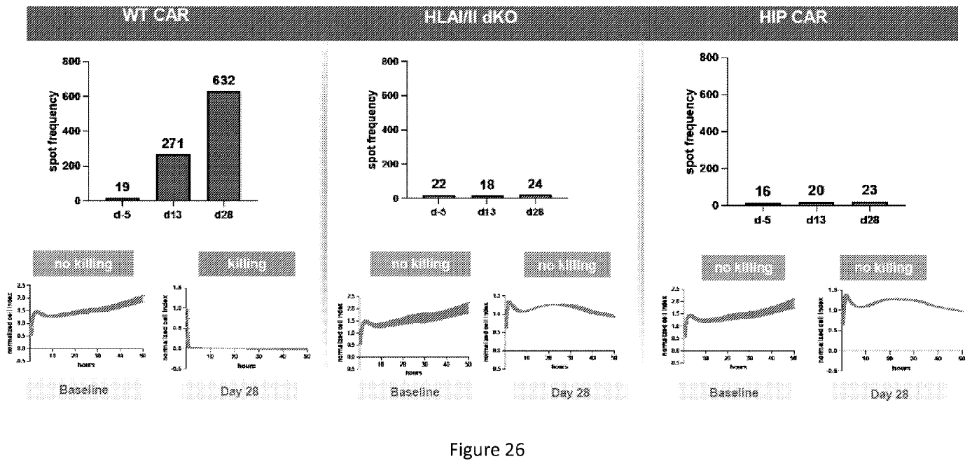

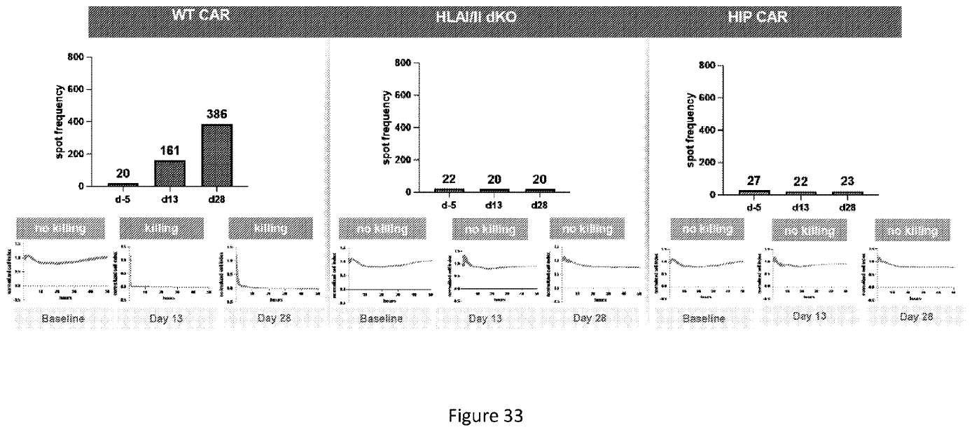

As shown in FIG. 6 (data from Subject 1), FIG. 14 (data from Subject 2), FIG. 26 (data from Subject 3), and FIG. 33 (data from Subject 4), T cell activation was seen against “WT CAR” cells from the subjects (first column), due to the presence of donor HLA on the WT CAR T cells. No T cell activation was seen against “HLAI/II dKO” cells (middle column) or “HIP CAR” cells (third column), demonstrating that HLA knock-out allowed for evasion of T cell recognition.

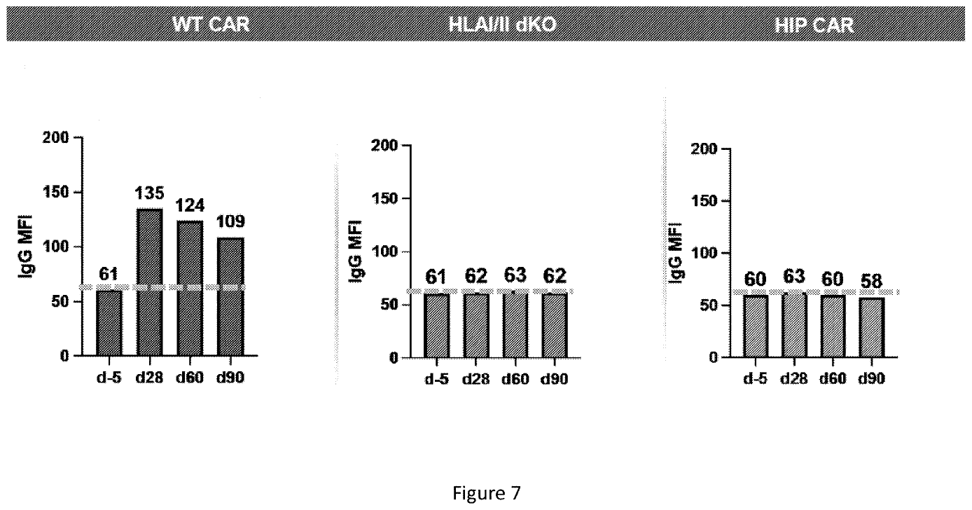

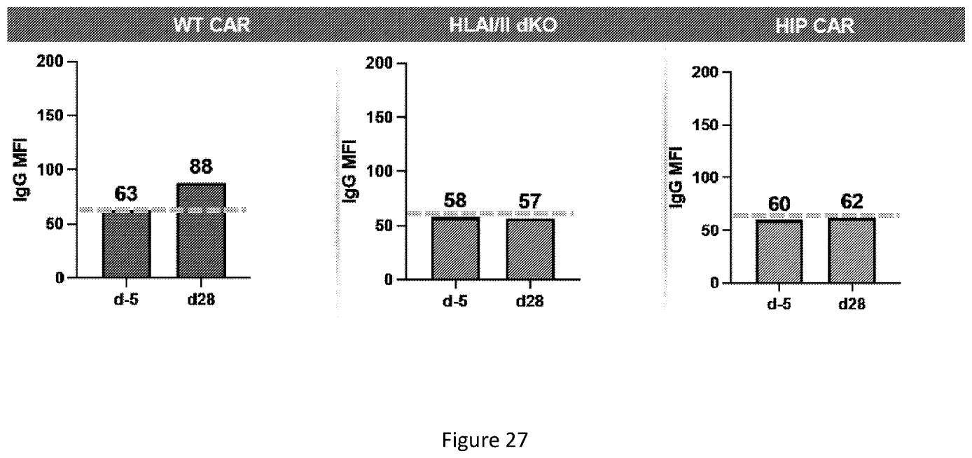

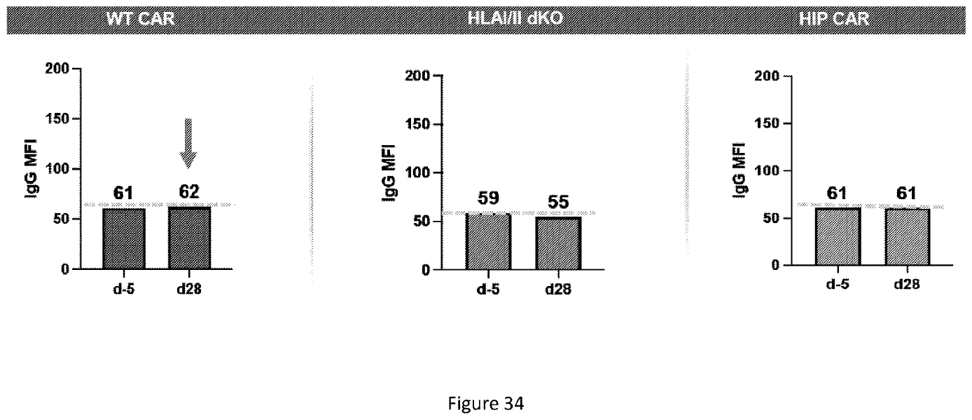

As shown in FIG. 7, baseline samples from Subject 1 (d-5) showed no sensitization (IgG-DSA binding) before the subject was administered the drug product. On days 28, 60, and 90, donor-specific antibodies were present and bound HLA-expressing drug product cells (WT CAR cells). However, no DSA occurred against HLAI/II dKO cells or against HIP CAR cells at day 28, day 60, or day 90. Anti-HLA antibody production in Subject 1 indicates that B cells were not fully depleted in Subject 1, potentially in part due to the reduced lymphodepletion regimen the subject received. For Subject 2, no donor-specific antibody binding against HLA was observed against any of the drug product cellular subpopulations (see FIG. 15), which indicates successful deep tissue B cell depletion in Subject 2. As shown in FIG. 27, for Subject 3, baseline samples (d-5) showed no sensitization (IgG-DSA binding) before the subject was administered the drug product. On day 28, donor-specific antibodies were present and bound HLA-expressing drug product cells (WT CAR cells). However, no DSA occurred against HLAI/II dKO cells or against HIP CAR cells at day 28. Anti-HLA antibody production in Subject 3 indicates that B cells were not fully depleted in Subject 3. For Subject 4, no donor-specific antibody binding against HLA was observed against any of the drug product cellular subpopulations (see FIG. 34), which indicates successful deep tissue B cell depletion in Subject 4.

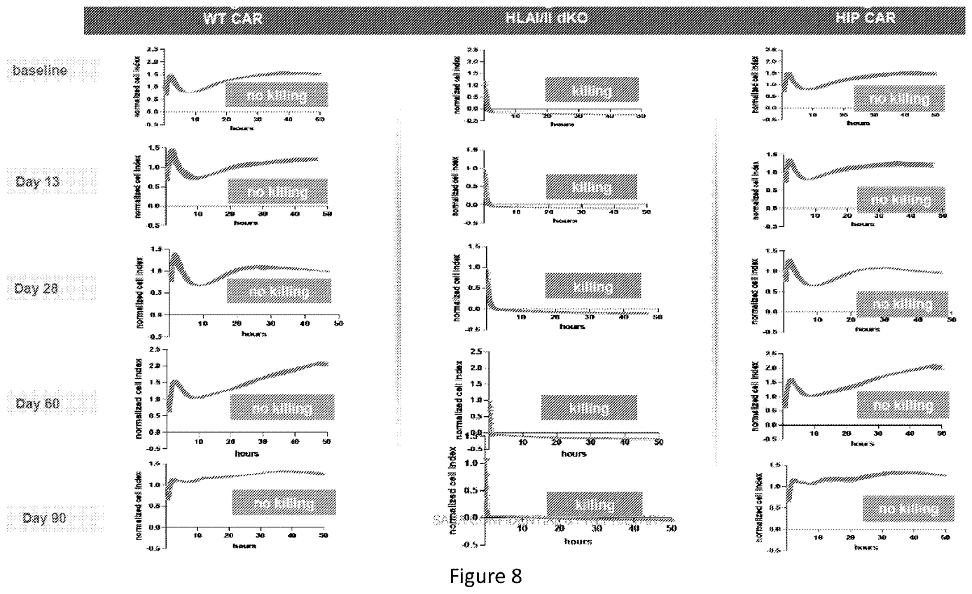

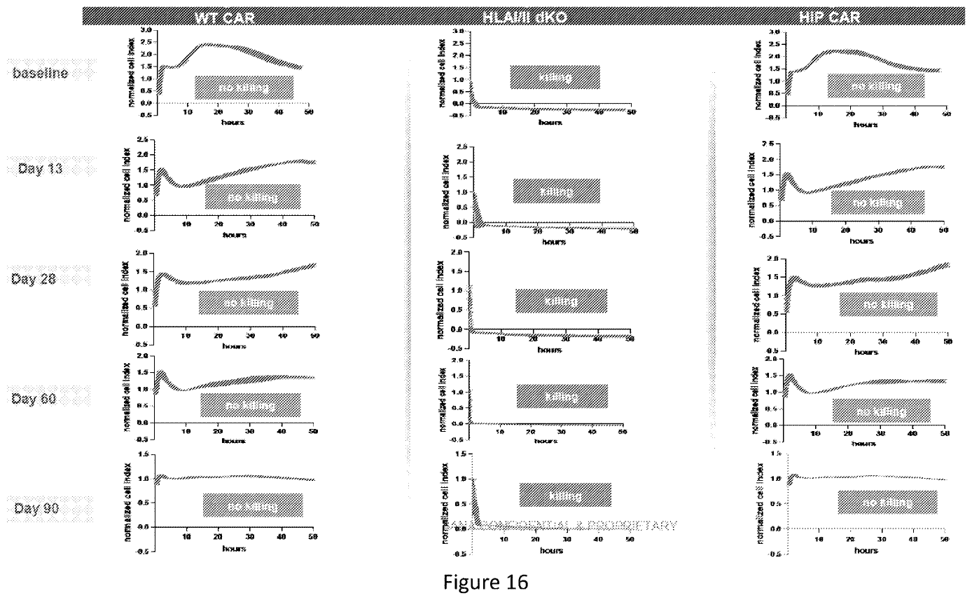

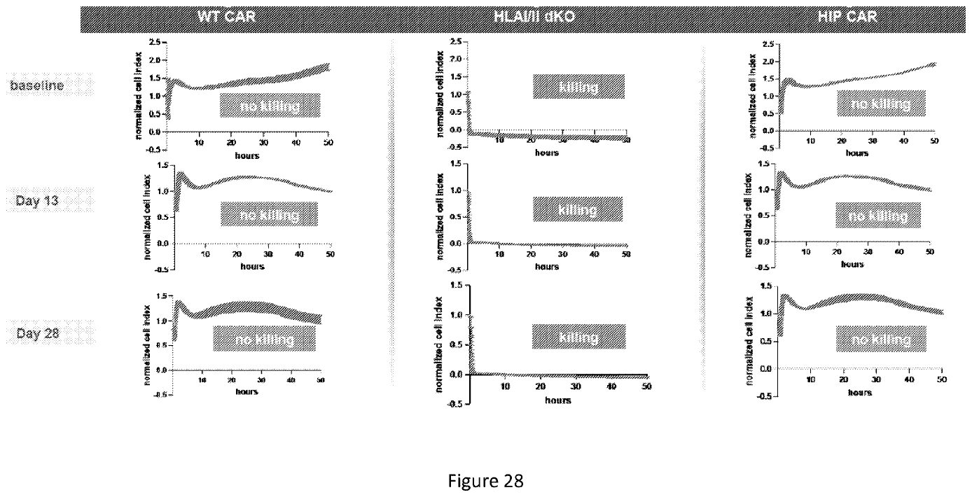

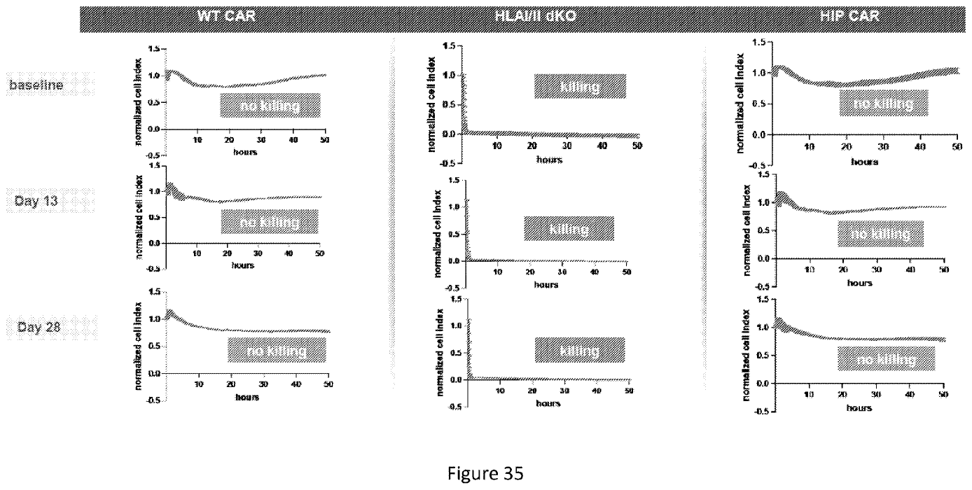

As shown in FIG. 8, at all timepoints, “HLAI/II dKO” cells from the drug product were killed by NK cells from Subject 1. Conversely, “WT CAR” cells and “HIP CAR” cells from the drug product were not killed by Subject 1's NK cells due to CD47 expression. Similarly, as shown in FIG. 16, FIG. 28, and FIG. 35, at all timepoints, “HLAI/II dKO” cells from the drug product were killed by NK cells from Subject 2, Subject 3, and Subject 4 (respectively) while “WT CAR” cells and “HIP CAR” cells from the drug product were not killed by Subject 2's, Subject 3's, and Subject 4's NK cells due to CD47 expression.

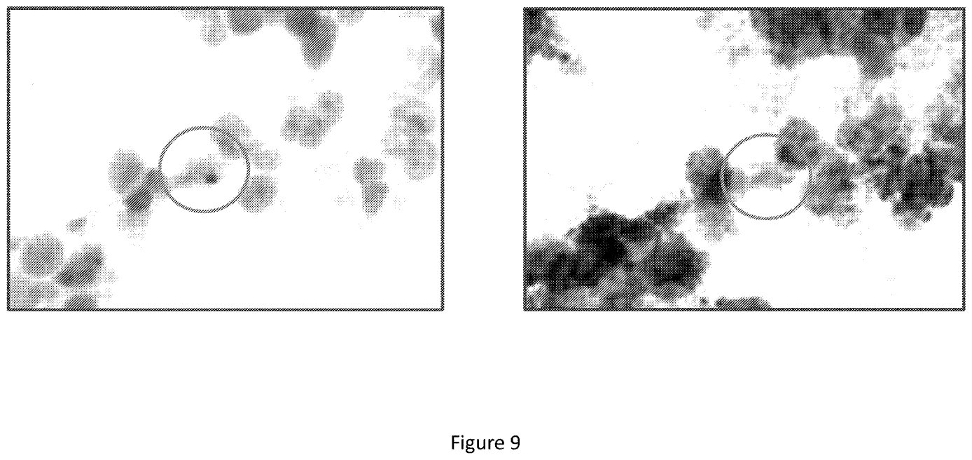

Additionally, as shown in FIG. 9, ISH staining for WPRE (left image) identified CAR+ cells from the drug product in bone marrow from Subject 1 four weeks after dosing. Immunohistochemistry for B2M (right image) identified CAR+ cells as “HIP CAR” cells.

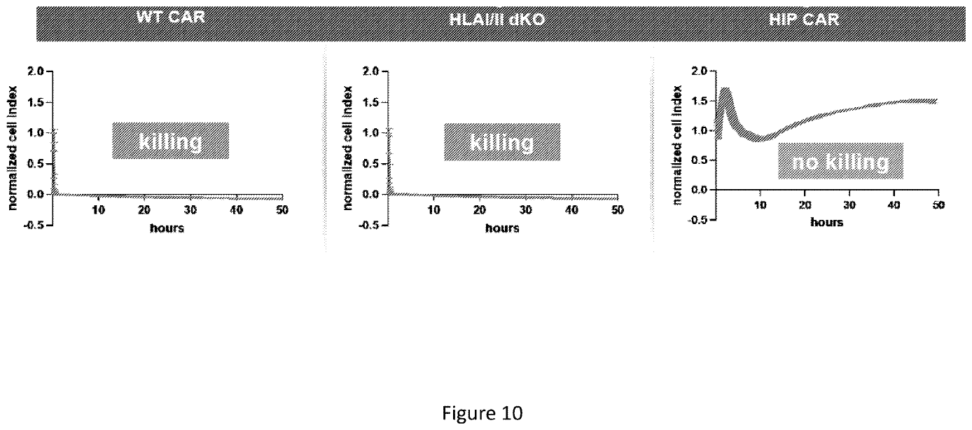

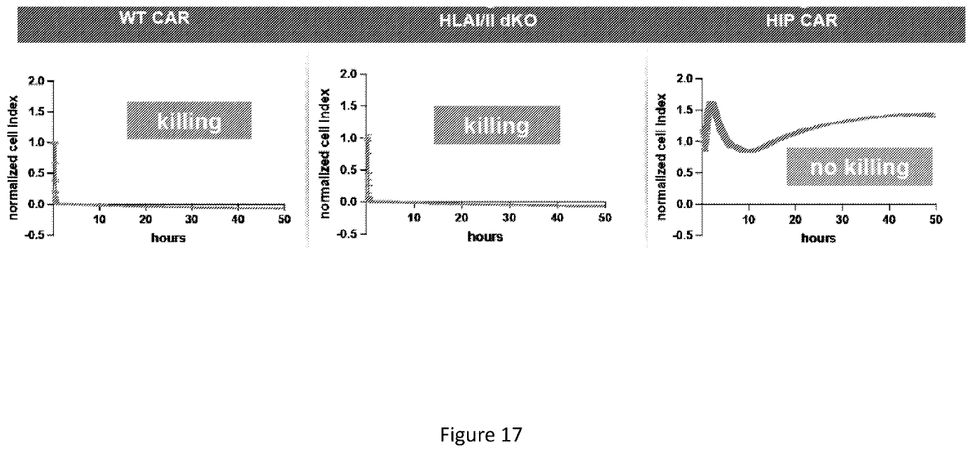

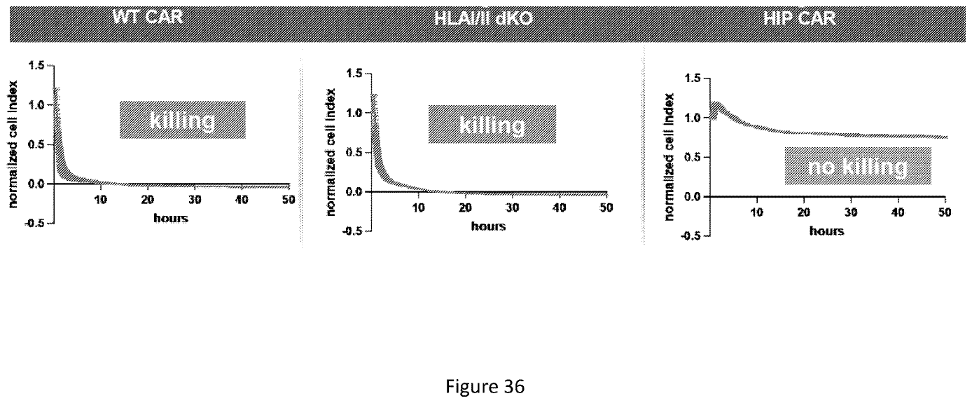

XCelligence assays were performed using day 28 samples from Subject 1, Subject 2, Subject 3, and Subject 4 and, as shown in FIG. 10, FIG. 17, FIG. 29, and FIG. 36 (respectively). While “WT CAR” and “HLAI/II dKO” cells from the drug product were killed by immune cells from the subjects, the “HIP CAR” cells were not.

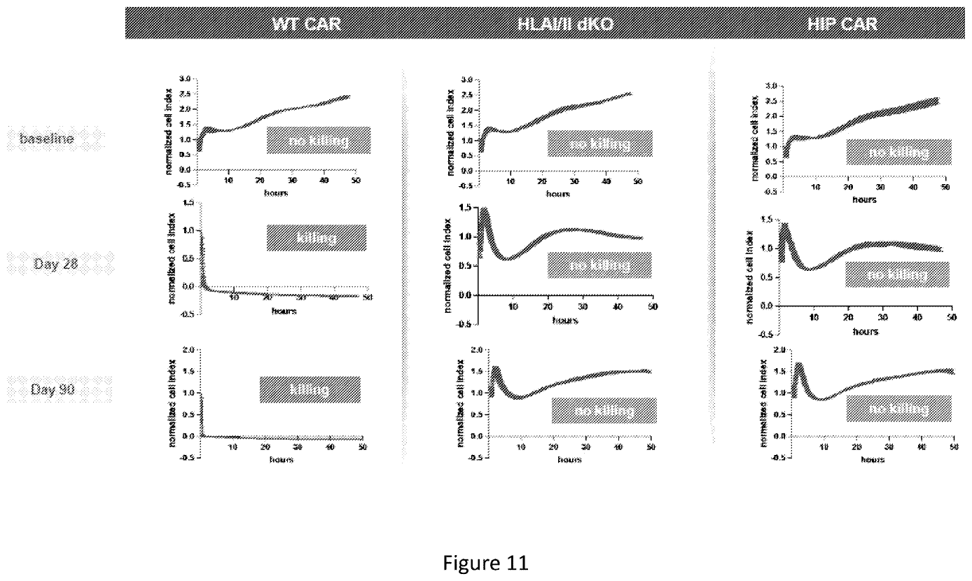

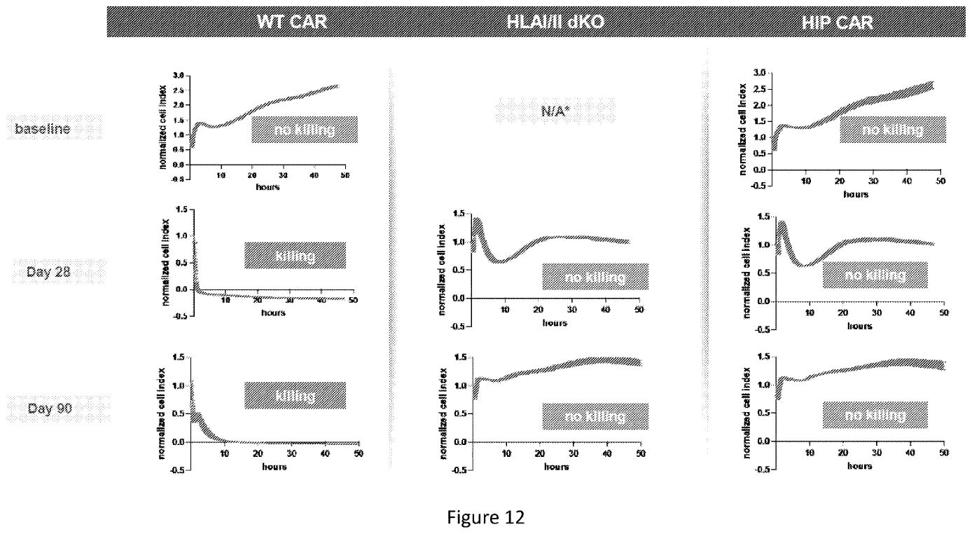

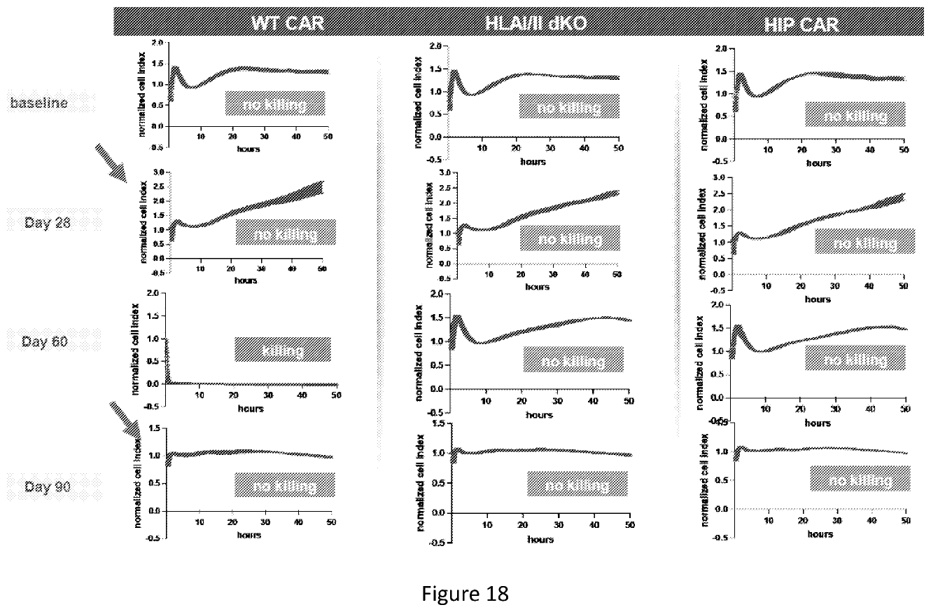

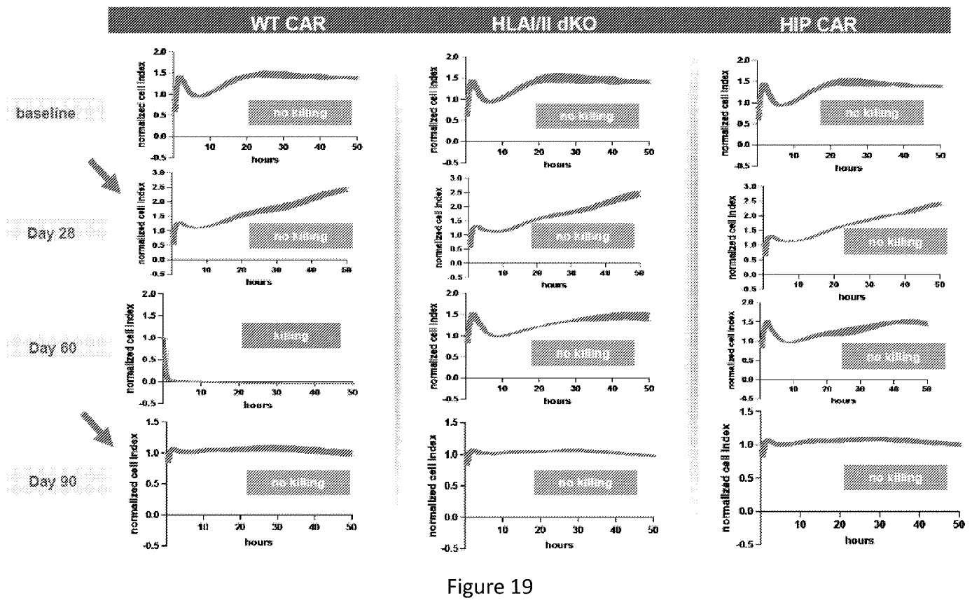

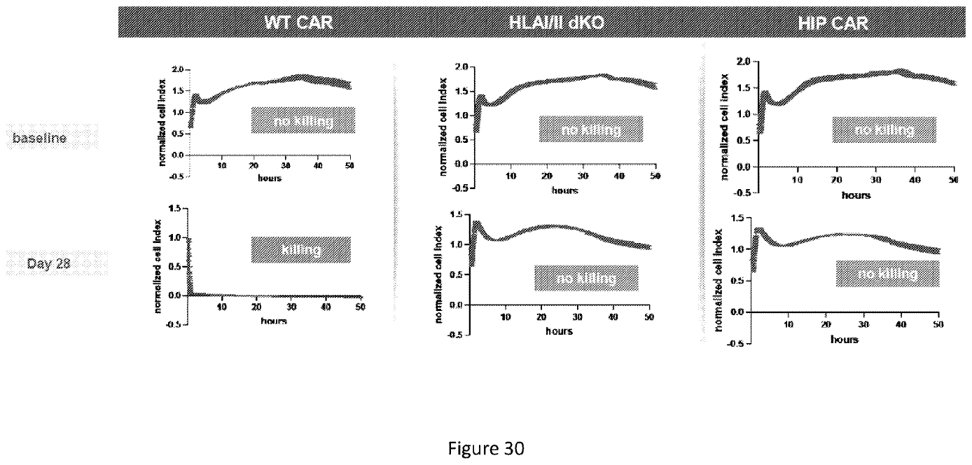

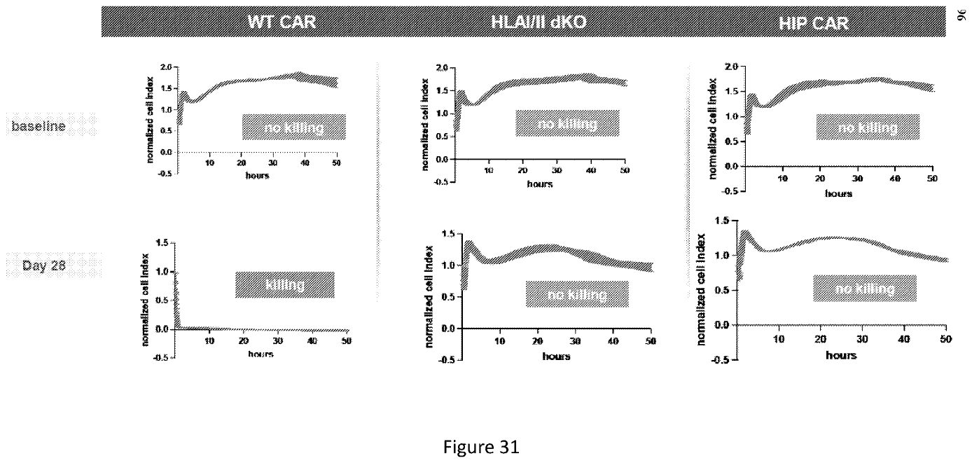

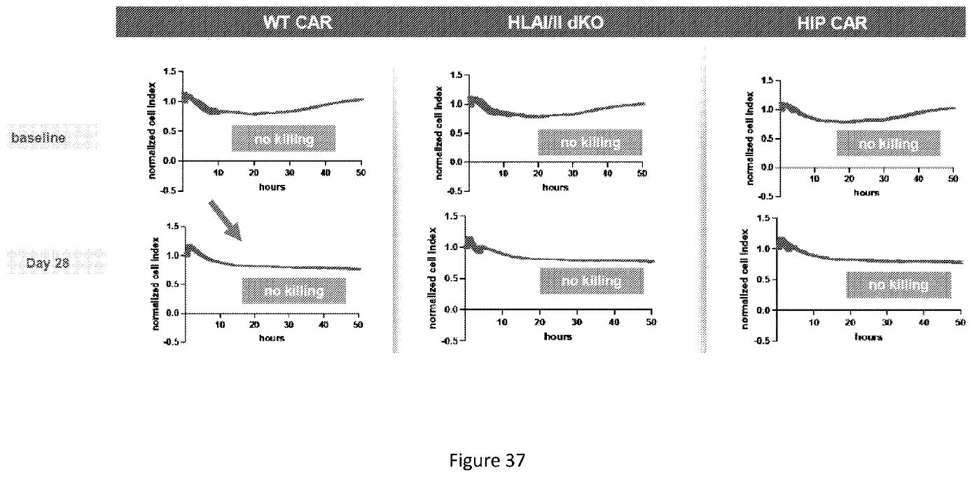

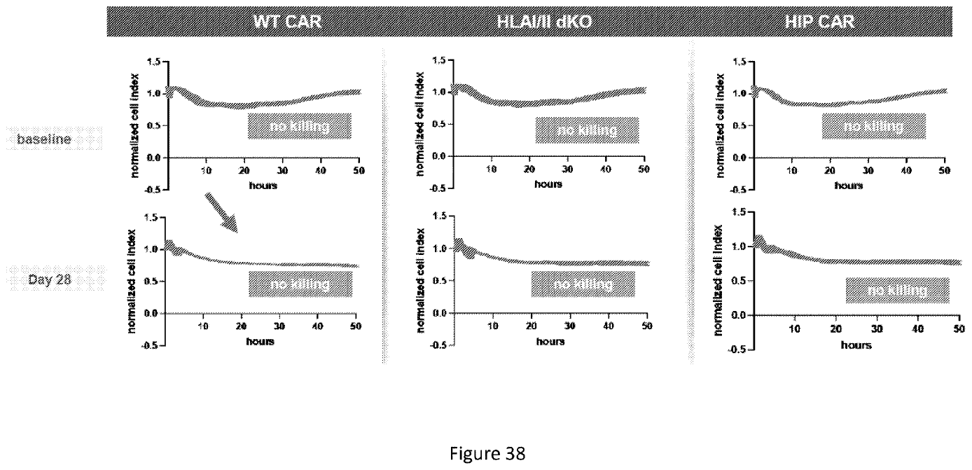

Finally, as shown in FIG. 11 and FIG. 12, no complement-dependent cytotoxicity (CDC) or antibody-dependent cellular cytotoxicity (ADCC) (respectively) were observed against “HIP CAR” cells by day 90 for Subject 1. Conversely, antibodies generated against HLA in “WT CAR” cells resulted in CDC and ADCC at days 28 and 90. Interestingly, for Subject 2, as shown in FIG. 18 and FIG. 19, no CDC or ADCC against “HIP CAR” cells was observed up to day 90, and no antibodies to HLA in “WT CAR” cells was observed on day 28, confirming full B cell depletion. However, antibodies against “WT CAR” cells on day 60 resulted in CDC and ADCC, but on day 90, when CD19+ cells were fully depleted again, no CDC or ADCC against “WT CAR” cells was observed. For Subject 3, as shown in FIG. 30 and FIG. 31, respectively, no CDC or ADCC was observed against “HIP CAR” cells by day 28, while antibodies generated against “WT CAR” cells resulted in CDC and ADCC. For Subject 4, as shown in FIG. 37 and FIG. 38, respectively, no CDC or ADCC was observed against “HIP CAR” cells by day 28, and no CDC or ADCC was observed against “WT CAR” cells at day 28, confirming full B cell depletion.

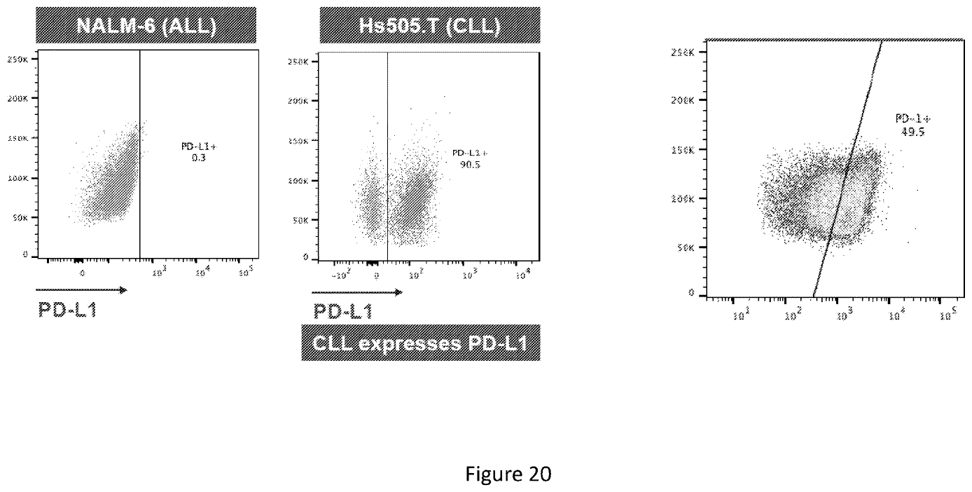

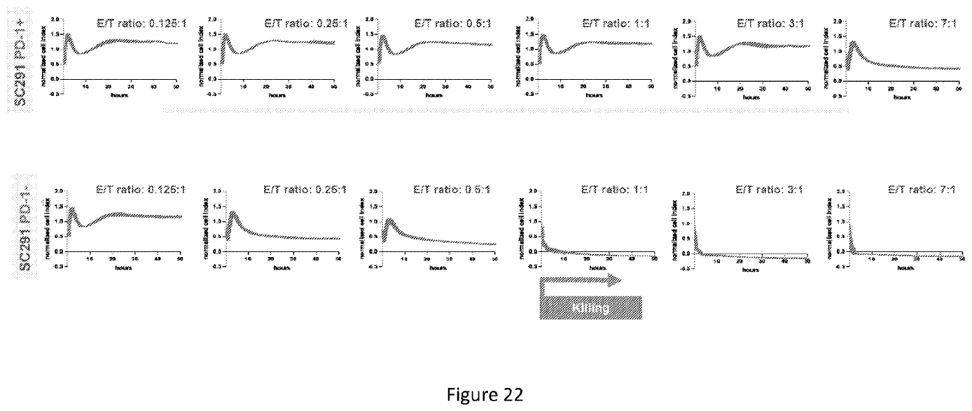

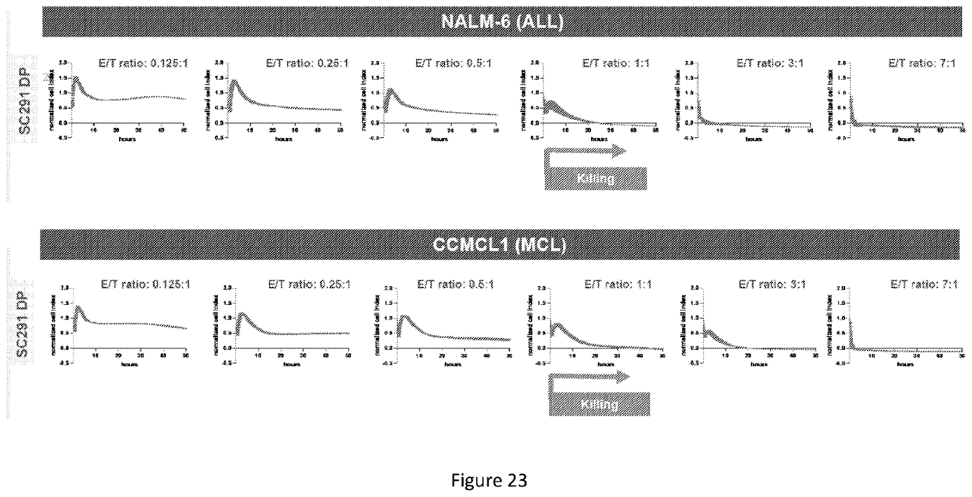

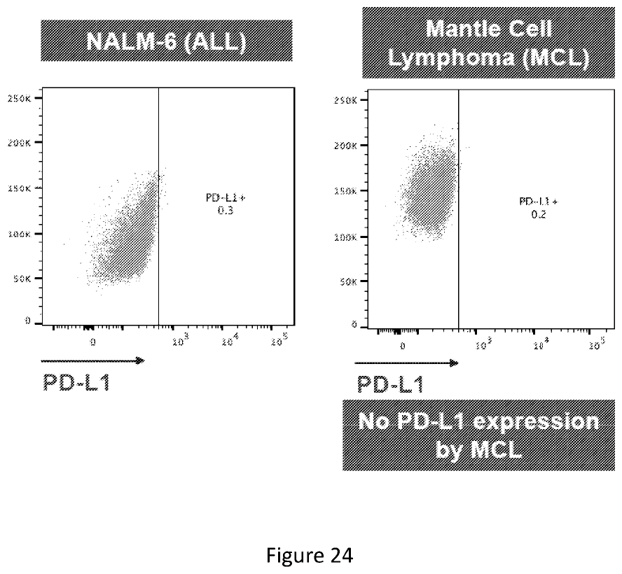

In addition to the above, it was observed that a higher percentage of CLL cells (for example, Hs505.T cells) express PD-L1 relative to acute lymphocytic leukemia (ALL) cells (for example, NALM-6 cells), as illustrated in FIG. 20. Also, as shown in the image on the right in FIG. 20, about half of the cells in the drug product described herein express PD-1. As shown in FIG. 21, when NALM-6 ALL cells were treated with the drug product, killing was observed at E/T ratios of 1:1, 3:1, and 7:1, whereas Hs505.T CLL cells weren't killed by the drug product until the E/T ratio was 7:1. The relationship between killing ability of drug product cells and PD-L1/PD-1 expression was elucidated by sorting the drug product cells into PD-1 positive and PD-1 negative populations and treating the CLL cells accordingly. As shown in FIG. 22, when only PD-1 positive cells from the drug product were used, no killing of CLL cells was observed. Conversely, when only PD-1 negative cells from the drug product were used, the killing of CLL cells was comparable to the above described killing of ALL cells (i.e., at an E/T ratio of 1:1). Interestingly, as shown in FIG. 23, when CCMCL1 mantle cell lymphoma (MCL) cells were treated with the drug product, killing was observed at the same E/T ratios observed in NALM-6 ALL cells, and MCL cells also did not express PD-L1 (see FIG. 24).

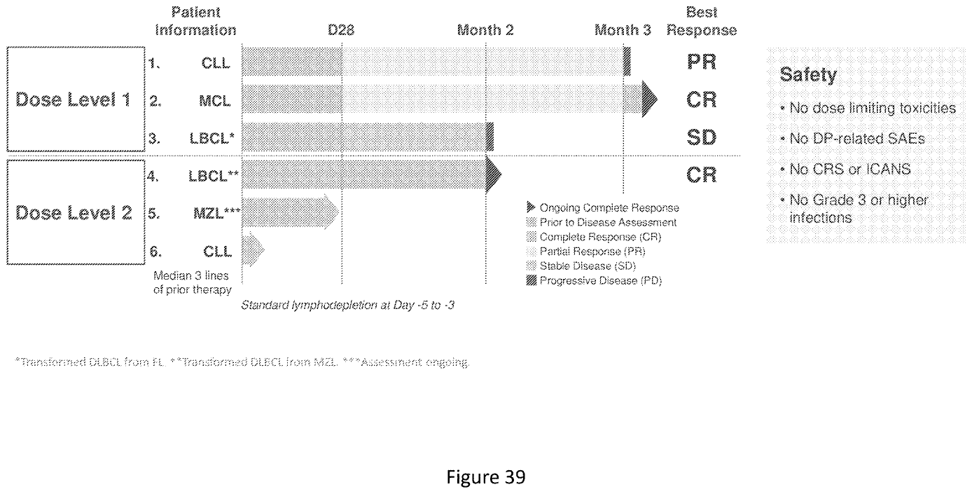

FIG. 39 shows a graph illustrating the efficacy of a Level 1 dose or a Level 2 dose of a drug product comprising hypoimmune allogenic CD19-directed CAR T cells of the present disclosure in Subjects 1, 2, 3, and 4 as described above. Between day 28 and month 3, Subject 1 exhibited a partial response (PR), and then progressive disease (PD) at month 3. Subject 2 exhibited a partial response at the day 28 assessment, but achieved a complete response (CR) at the month 3 assessment. Subject 3 exhibited stable disease at the day 28 assessment, and exhibited progressive disease at the month 2 assessment. Subject 4 exhibited an ongoing complete response, starting at the day 28 assessment.

### Example 2: Administration of Hypoimmune Allogeneic CD19-Directed CAR T Cells to Subjects with Autoimmune Disease

In B cell mediated autoimmune conditions (e.g., systemic lupus erythematosus (SLE), lupus nephritis (LN), extrarenal systemic lupus crythematosus (ERL), CNS lupus, anti-neutrophilic cytoplasmic autoantibody (ANCA) associated vasculitis, granulomatous polyangiitis, microscopic polyangiitis, multiple sclerosis, pemphigus vulgaris, autoimmune blistering skin diseases, membranous nephropathy, anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, and autoimmune hemolytic anemia), there is a significant unmet medical need and hypoimmune allogenic CD19-directed CAR T cells of the present disclosure may represent a novel therapeutic option.

Therapeutic compositions comprising hypoimmune allogenic CD19-directed CAR T cells will be administered to adult subjects with autoimmune conditions (e.g., systemic lupus erythematosus (SLE), lupus nephritis (LN), extrarenal systemic lupus erythematosus (ERL), CNS lupus, anti-neutrophilic cytoplasmic autoantibody (ANCA) associated vasculitis, granulomatous polyangiitis, microscopic polyangiitis, multiple sclerosis, pemphigus vulgaris, autoimmune blistering skin diseases, membranous nephropathy, anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, and autoimmune hemolytic anemia). The hypoimmune technology disclosed herein enables engineered hypoimmunogenic T cells of the present disclosure to evade both the innate and adaptive immune system, thus potentially enabling enhanced cellular expansion and persistence. The studies described in this example evaluate the safety and tolerability of hypoimmunogenic T cells of the present disclosure.

### Objectives and Endpoints

In this example, safety and tolerability of hypoimmunogenic T cells of the present disclosure, will be evaluated by monitoring: dose-limited toxicities (DLTs), treatment-emergent adverse events (TEAEs), treatment-related adverse events (TRAEs), targeted adverse events (TAEs), and/or adverse events of special interest (AESIs).

Secondary objectives of the studies of this example include evaluating the activity of hypoimmunogenic T cells of the present disclosure and their ability to treat autoimmune conditions (e.g., systemic lupus erythematosus (SLE), lupus nephritis (LN), extrarenal systemic lupus erythematosus (ERL), CNS lupus, anti-neutrophilic cytoplasmic autoantibody (ANCA) associated vasculitis, granulomatous polyangiitis, microscopic polyangiitis, multiple sclerosis, pemphigus vulgaris, autoimmune blistering skin diseases, membranous nephropathy, anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, and autoimmune hemolytic anemia). For all subjects, secondary objectives include determining: change in disease activity, change in renal function and proteinuria, change in patient-reported outcomes, change in physician global assessment, change in autoantibody profiles, change in serum complement levels, duration of drug free remission, and time to relapse. For Cohort 1 (subjects with LN), secondary objectives include: proportion of subjects achieving complete renal response or partial renal response, and change from baseline in Systemic Lupus Erythematosus Disease Activity Index (SLEDAI) 2000 (2K) response score. For Cohort 2 (subjects with ERL), secondary objectives include: Change from baseline of SLEDAI-2K and proportion of subjects achieving modified DORIS remission. For Cohort 3 (subjects with AAV), secondary objectives include: proportion of subjects achieving remission (Birmingham Vasculitis Activity Score version 3 [BVAS v3] of 0) and change from baseline in BVAS v3.

Secondary objectives of the studies of this example also include evaluating the cellular kinetics and persistence of hypoimmunogenic T cells of the present disclosure. In this example, the cellular kinetics (CK)-related parameters of hypoimmunogenic T cells of the present disclosure, will be evaluated by CAR copy number and includes: peak observed in peripheral blood after administration (Cₘₐₓ), time of first occurrence of maximum-observed concentration (Tₘₐₓ), terminal disposition phase half-life (t_{1/2}), last observed quantifiable concentration in peripheral blood (Cₗₐₛₜ), time of last observed quantifiable concentration in peripheral blood (days) (Tₗₐₛₜ), and area under the concentration-time curves (AUCs).

Secondary objectives of the studies of this example also include evaluating the host immunogenicity of hypoimmunogenic T cells of the present disclosure. In this example, the host immunogenicity of hypoimmunogenic T cells of the present disclosure will be evaluated by monitoring host humoral immune responses.

Exploratory objectives of this study include evaluating additional immunogenicity assessments of hypoimmune allogenic CD19-directed CAR T cells of the present disclosure administered to subjects with autoimmune conditions (e.g., systemic lupus erythematosus (SLE), lupus nephritis, CNS lupus, anti-neutrophilic cytoplasmic autoantibody (ANCA) associated vasculitis, granulomatous polyangiitis, microscopic polyangiitis, multiple sclerosis, pemphigus vulgaris, autoimmune blistering skin diseases, membranous nephropathy, anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, and autoimmune hemolytic anemia). In this example, additional immunogenicity assessments of hypoimmunogenic T cells of the present disclosure will be evaluated by monitoring host cellular immune responses (ELISpot/T cell killing/NK cell killing) and donor-specific antibody production (donor-specific antibody [DSA]/antibody-dependent cellular cytotoxicity [ADCC]/complement-dependent cytotoxicity [CDC]). CK-related parameters evaluated by CAR transgene copy number include: peak observed in peripheral blood after administration (Cmax), time of first occurrence of maximum-observed plasma concentration (Tmax), terminal disposition phase half-life (t½) and other cellular kinetics (CK) parameters. Evaluations of subjects' immunogenicity to hypoimmune allogenic CD19-directed CAR T cells of the present disclosure include: cellular and humoral immunogenicity assessment, donor-specific antibody evaluation, and subject HLA typing. Evaluations exploring a correlation of pharmacodynamic (PD) biomarkers with subject safety and outcomes may include but is not limited to: B cell depletion (circulating CD19+ B cells), an autoantibody panel, soluble markers of inflammation, soluble markers of B cell activation, including B cell activating factor (BAFF), serum immunoglobulins, immunophenotyping by flow cytometry, cytokines and other cytokine-release syndrome (CRS)-related PD biomarkers, frequency and/or counts of CD19 CAR, CD47, T cell receptor (TCR), and major histocompatibility complex (MHC) I/II, immunogenicity and immune evasion, gene expression profiling via RNA sequencing, pre-study and on-study review of kidney biopsies or other biopsy material as clinically indicated and available, and protective immunoglobulins such as tetanus, influenza, and others. Evaluations exploring the impact of baseline donor blood compositions on subject safety, outcomes, and CK include product characteristics parameters including: T cell and other immune cell phenotypes, T cell activation, expansion, effector function and exhaustion, frequency and/or counts of CD19-CAR, CD47, TCR, MHC I/II, and molecular immune landscape analysis. Evaluations exploring markers of a durable response to hypoimmune allogenic CD19-directed CAR T cells of the present disclosure include (for all subjects): proteinuria level at 3, 6, 9, and 12 months, serum creatinine and estimated glomerular filtration rate (eGFR) at 3, 6, 9 and 12 months, and autoantibody status over time. Evaluations exploring markers of a durable response to hypoimmune allogenic CD19-directed CAR T cells of the present disclosure include (for Cohort 1—subjects with LN): pre-study ISN/RPS LN class on available biopsy material or report.

Exploratory objectives of this study also include evaluating the impact on baseline donor characteristics and attributes of hypoimmune allogenic CD19-directed CAR T cells of the present disclosure on subject safety, outcomes, and cellular kinetics. In this example, the impact of administration of hypoimmune allogenic CD19-directed CAR T cells of the present disclosure on subject safety, outcomes, and cellular kinetics will be evaluated by monitoring subject: T cells, B cells and other immune cell phenotypes (CAR T enumeration and immunophenotyping); T cell activation, expansion, effector function, and exhaustion (immunophenotyping); expression levels of CD 19-directed CAR, CD47, TCR, and HLA I/II (CAR T enumeration and CD47 expression); and molecular immune landscape.

Exploratory objectives of this study also include evaluating markers that are indicative of a durable response in subjects to hypoimmune allogenic CD19-directed CAR T cells of the present disclosure.

### Study Design

Hypoimmune allogenic CD19-directed CAR T cells of the present disclosure will be administered intravenously (IV) in a single infusion in subjects with autoimmune conditions (e.g., systemic lupus erythematosus (SLE), lupus nephritis, CNS lupus, anti-neutrophilic cytoplasmic autoantibody (ANCA) associated vasculitis, granulomatous polyangiitis, microscopic polyangiitis, multiple sclerosis, pemphigus vulgaris, autoimmune blistering skin diseases, membranous nephropathy, anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, and autoimmune hemolytic anemia).

### Screening

Screening begins when a subject signs an institutional review board/independent ethics committee (IRB/IEC)-approved informed consent form (ICF) and is provided with a study-specific subject number. Screening begins up to 21 days before enrollment and ends with confirmation of eligibility and enrollment.
Subjects included in this study are diagnosed with lupus nephritis (LN), extrarenal lupus (ERL), or anti-neutrophil cytoplasmic antibody (ANCA)-associated vasculitis (AAV) who have refractory disease or have relapsed or have not shown appropriate clinical responses following prior systemic treatments. Subjects must not have received prior autologous CD19-directed CAR T cell therapy.

### Inclusion Criteria

Subjects considered for the safety and tolerability of the present example have the following inclusion criteria:
Male or female subjects aged 18-75 years.
Adequate organ function as confirmed by clinical laboratory values as specified below:

### Hepatic Function:

Total bilirubin <1.5×upper limit of normal (ULN) (or <3×ULN for subjects with liver involvement) (except when attributed to Gilbert's syndrome)
Alanine aminotransferase (ALT) or aspartate aminotransferase (AST)<3×ULN (<5×ULN for subjects with liver involvement). Subjects with disease-related muscle involvement causing elevated ALT or AST may be included.

### Bone Marrow Function:

Hemoglobin ≥8 g/d.
Neutrophil count³ 1000/mm³
Platelet count³ 50,000/mm³

### Pulmonary Function:

Baseline oxygen saturation >92% on room air, and no clinically significant pleural effusion
Forced expiratory volume in the first second/Forced Vital Capacity (FEV₁/FVC) >70% by spirometry
Subjects must be eligible for LD chemotherapy.
All subjects must be willing to practice birth control and refrain from donating sperm or oocytes from the time of enrollment in this study through 12 months after receiving hypoimmune allogeneic CD19-directed T cells of the present disclosure.
Women of child bearing potential (WOCBP) must test negative for pregnancy at screening. WOCBP are defined as any women who are not postmenopausal or who have not had a hysterectomy, have functional ovaries and no documented impairment of oviductal or uterine function that would cause sterility. This category includes women with oligomenorrhoea [even severe], women who are perimenopausal, or have just begun to menstruate. Postmenopausal is defined as women over the age of 55 who have not had a menstrual period for ≥1 year.
Contraception: WOCBP will practice a nonhormonal method of contraception and one additional effective (barrier) method at the same time, from the time of signing the ICF through at least 12 months following infusion. Male subjects, even if surgically sterilized (i.e., post vasectomy), agree to practice effective barrier contraception from the time of signing the ICF through at least 12 months following infusion.
Activated partial thromboplastin time ≤1.2×ULN.
Prothrombin time-international normalized ratio (INR)≤1.2 with no disease-related coagulopathy.
Estimated GFR of ≥45 mL/min/1.73m² as determined by the CKD-EPI equation.
   Cohort 1 Only (Subjects with LN)
Diagnosis of SLE based on 2019 EULAR/ACR Classification Criteria for SLE.
Histological confirmation of Class III (A or A/C) or IV (A or A/C) LN based on ISN/RPS 2018 criteria with active or active/chronic lupus nephritis performed within 6 months of screening. Subjects may have Class V lupus nephritis in addition to either Class III or IV disease.
Refractory disease defined as an inadequate response to ≥2 prior treatment regimens (mycophenolate mofetil (MMF) or cyclophosphamide as first-line therapy for LN, followed by either rituximab, belimumab, voclosporin, or obinutuzumab as second-line therapy). Refractory disease is defined by the inability to achieve a complete renal response to second-line therapy within 6 months of initiation of the therapy at target doses specified in the protocol. Refractory disease is also defined by the inability to achieve an improvement in proteinuria (with eGFR normalization/stabilization) within 4 months, or a partial renal response (50% reduction in proteinuria) within 6-12 months to a stable dose of second-line therapy at target doses (refer to Table 6) following EULAR/ERA-EDTA guidelines. Patients that have not achieved a complete renal response (<0.5-0.7 g/24 hours) after 2 years of treatment will also be eligible.
Disease progression despite maintenance on maximally tolerated doses of drugs that block the renin-angiotensin system (RAS).
Presence of at least 1 elevated autoantibody, including anti-dsDNA, anti-nuclear autoantibody (ANA) titer ≥1:80 (Hep2) or anti-Sm (Smith) autoantibody or other autoantibodies.
Prothrombin time-international normalized ratio ≤2.5 with no disease-related coagulopathy.
Activated partial thromboplastin time ≤2.5×ULN
Estimated GFR of ≥45 mL/min/1.73m² as determined by the CKD-EPI equation.
Proteinuria with a UPCR determined by either spot urine collection >1 g/g and ≤7 g/g or 24-hour urine collection of >1 g/dL and ≤7 g/dL.
Systemic Lupus Erythematosus Disease Activity Index 2000 (SLEDAI-2K) score ≥8, designating severe lupus activity.
   Cohort 2 Only (Subjects with ERL)
Diagnosis of SLE based on 2019 EULAR/ACR Classification Criteria for SLE.
Severe or relapsing disease not responding to at least 2 prior recent disease-modifying therapies such as MMF, azathioprine, cyclophosphamide, methotrexate, rituximab, belimumab, anifrolumab, abatacept or other therapies, excluding glucocorticoids.
Presence of at least 1 elevated autoantibody, including anti-dsDNA autoantibody ANA titer ≥1:80 or anti-Sm (Smith) autoantibody.
Prothrombin time-international normalized ratio ≤2.5 with no disease-related coagulopathy.
Activated partial thromboplastin time ≤2.5×ULN.
Estimated GFR of ≥45 mL/min/1.73m² as determined by the CKD-EPI equation.
SLEDAI-2K score >8, designating severe lupus activity.
   Cohort 3 Only (Subjects with AAV)
Diagnosed with GPA or MPA based on the Chapel Hill Consensus Conference criteria.
Diagnosed with a disease flare with a BVAS >2 following the achievement of remission induced by a regimen consisting of anti-CD20 B cell depletion (e.g., rituximab or obinutuzumab) and glucocorticoids; cyclophosphamide and glucocorticoids; or anti-CD20 B cell depletion, cyclophosphamide and glucocorticoids. The flare must be considered organ or life threatening by the Investigator. Subjects with more than 1 GPA or MPA flare and at least 1 prior induction regimen with rituximab, obinutuzumab or cyclophosphamide may be enrolled if baseline BVAS is ≥1.
Must have severe disease or a history of severe disease as defined by one or more of the major BVAS items
Must be positive for either PR3-ANCA or MPO-ANCA at Screening.
Estimated GFR of ≥45 mL/min/1.73m² as determined by the CKD-EPI equation.

### Exclusion Criteria

Subjects considered for the safety and tolerability of the present example will not have any of the following exclusion criteria:

All subjects (Cohorts 1-3)
Clinical evidence of significant unstable or uncontrolled acute or chronic diseases not due to their autoimmune disease (i.e., cardiovascular, pulmonary, hematologic, gastrointestinal, hepatic, renal, neurological, malignancy, or infectious diseases).
Prior anti-CD19 cell therapy including CAR-T treatment or other genetically modified cell therapy (e.g., NK cell).
History of an anaphylactic reaction to parenteral administration of contrast agents, human or murine proteins, or monoclonal antibodies or severe immediate hypersensitivity to any of the components of hypoimmune allogeneic CD19-directed T cell drug product.
History of cancer including hematologic malignancies, solid tumors, or carcinoma in situ (except basal carcinomas of the skin, cervix, bladder, prostate or breast which have been treated or excised and have resolved >5 years from start of participation in this trial).
Presence of any ongoing infection.
Known history of infection with human immunodeficiency virus or hepatitis B virus (HBV; hepatitis B surface antigen positive) or hepatitis C virus (HCV; anti-HCV positive). Prior HCV with documented receipt of a direct-acting antiviral and undetectable viral load 3 months after completion of therapy will be allowed.
Glucocorticoids anticipated to be dosed above physiologic replacement during the protocol are excluded. Physiologic replacement glucocorticoids (e.g. ≤5-7.5 mg prednisone daily or equivalent) and topical and inhaled glucocorticoids are permitted.
Treatment with any investigational products within 28 days before infusion.
Treatment with Leflunomide within 28 days prior to Screening.
Treatment with major surgery within 28 days before infusion (minor surgical procedures such as catheter placement are permitted).
Ejection fraction ≤50%, arrythmias and any structural heart disease (including clinically significant pericardial effusion), as assessed by echocardiogram or multiple gated acquisition scan performed within 1 month prior to infusion.
Myocardial infarction, cardiac angioplasty or stenting, cardiac arrythmia requiring medication, unstable angina, or other clinically significant cardiac disease within 12 months of enrollment.
Receipt of a live vaccine within 28 days of initiation of LD chemotherapy.
Subjects with a history of organ transplantation or awaiting organ transplantation including history of major organ transplant (e.g., heart, lung, kidney, liver) or hematopoietic stem cell transplantation/marrow transplant or are due to receive transplantation.
Immunoglobulin A (IgA) deficient (IgA level <10 mg/dL).
Immunoglobulin G (IgG) deficient (IgG level <600 mg/dL).
Receipt of immunosuppressant therapy (except corticosteroids in Screening) within 5 half-lives of the start of LD chemotherapy.
Hospitalization for treatment of infection within 28 days of baseline of >2 times over the past year.
Grade≥3 laboratory abnormality.
A serious medical or psychiatric illness that could potentially interfere with the completion of treatment.
Any disease related flare during Screening that needs immediate treatment other than corticosteroids and makes the immunosuppressive washout impossible.
Prior treatment with a CD47-directed therapy and/or detectable levels of anti-CD47 antibodies.
Women who are breastfeeding.
Presence of a concomitant autoimmune disease or a history of autoimmune disease requiring immunosuppressive therapy for ongoing symptoms.
Subjects with coagulation abnormalities.
History of chronic pulmonary disease (e.g., asthma, COPD) unless well controlled without evidence of an exacerbation in the prior 6 months requiring oral or iv corticosteroids or hospitalization.
History of tobacco exposure (>10 pack-years).
   Cohort 1 (Subjects with LN)
Have a CTCAE version 5.0 Grade≥3 laboratory abnormality, except for Grade 3 proteinuria (≥3.5 g/24 hours and ≤7 g/24 hours).
Central nervous system lupus manifestations or history or presence of CNS disorder, such as primary seizure disorder, cerebrovascular ischemia/hemorrhage, dementia, or cerebellar disease.
Diagnosis of anti-phospholipid antibody syndrome with a positive beta-2 glycoprotein-1 antibody test or a positive lupus anticoagulant assay.
Any pulmonary manifestations of SLE (e.g., interstitial lung disease), excluding pleurisy which is allowed.
   Cohort 2 (Subjects with ERL)
Have a CTCAE version 5.0≥3 laboratory abnormality.
Central nervous system lupus manifestations or history or presence of CNS disorder, such as primary seizure disorder, cerebrovascular ischemia/hemorrhage, dementia, or cerebellar disease.
Diagnosis of anti-phospholipid antibody syndrome with a positive beta-2 glycoprotein-1 antibody test or a positive lupus anticoagulant assay.
Any pulmonary manifestations of SLE (e.g., interstitial lung disease), excluding pleurisy which is allowed.
   Cohort 3 only (Subjects with AAV)
Have a CTCAE version 5.0≥3 laboratory abnormality.
Diagnosis of EGPA as defined by the Chapel Hill Consensus Conference criteria.
Presence of alveolar hemorrhage within the 4 weeks prior to baseline treatment with hypoimmune allogeneic CD19-directed T cells.
Diagnosis of cerebrovascular accident caused by vasculitis or pachymeningitis.
Presence of severe items of massive hemoptysis/alveolar hemorrhage, respiratory failure, (pachy)meningitis, cerebrovascular accident, spinal cord lesions, cranial nerve palsy, sensorineural hearing loss, sensory peripheral neuropathy, and mononeuritis multiplex as defined by BVAS v3.

Demographic data is collected at screening for each subject. Where applicable, demographic data will include sex, year of birth, race, ethnicity, and country of enrollment with the intent to assess the possible association of demographics with subject safety and treatment effectiveness. Additionally, for female subjects the number of previous births (along with gender of births) are collected to assess for potential Rh alloimmunization.
Relevant medical history (i.e., information about the subject's medical condition(s) that would be typically shared in a referral letter) from before enrollment is collected during screening. All history related to the subject's autoimmune disease and status, treatment(s), and response(s) to treatment(s) dating back to an original diagnosis is collected.

Subjects receiving infusion of hypoimmune allogeneic CD19-directed T cells should not have:
a body temperature >38° C. within the 72 hours before infusion;
C-reactive protein (CRP) levels ≥100 mg/L any time between enrollment and the start of infusion;
a white blood cell (WBC) count or WBC differential concerning for infectious process between enrollment and the start of infusion (e.g., WBC >20,000 cells/μL, rapidly increasing WBC, or WBC differential with a high percentage of segments/bands);
received systemic antimicrobials for the treatment of a known or suspected infection within the 48 hours before infusion (prophylactic use of antimicrobials is allowed); or
had a positive result for microbial infection (e.g., bacterial, fungal, or viral) in a collected blood, urine, or other body fluid culture within 48 hours of infusion.

To confirm the absence of infectious and/or inflammatory states, the following work-up may be performed and must not suggest the presence of an active infection:
perform CT imaging of the chest with IV contrast;
perform CT imaging of the abdomen and pelvis with IV contrast if clinically indicated;
perform blood cultures (aerobic and anaerobic×2 bottles each), urinalysis, and urine culture;
perform deep/induced sputum culture if clinically indicated;
perform nasopharyngeal-throat swab or equivalent assay for viral infection such as Covid-19, influenza A/B (including H1N1), parainfluenza 1/2/3, adenovirus, respiratory syncytial virus, and metapneumovirus;
collect fungal cultures and markers as appropriate (e.g., galactomannan or Fungitell®);
collect appropriate serum viral studies (e.g., cytomegalovirus); and
if a CNS process is suspected, then perform the appropriate brain imaging and subsequent lumbar puncture with cytology, culture, Gram stain, and viral PCR.

### Excluded Medications

Corticosteroid therapy at a pharmacologic dose (≥5 mg/day of prednisone or equivalent doses of other corticosteroids) and other immunosuppressive drugs are avoided for 5 days prior to hypoimmune allogeneic CD19-directed T cell infusion. Pre-medication with corticosteroids, tocilizumab, or anakinra is not permitted.
Immunosuppressant therapies commonly used to treat SLE must not have been administered within at least 5 half lives before the start of LD chemotherapy.

TABLE 18
Target doses and half lives of immunosuppressant
therapies commonly used in the treatment of SLE

| Drug | MMF | Cyclophosphamide | Voclosporin | Azathioprine |
|---|---|---|---|---|
| Target dose | Induction 2-3 | Low dose: 500 mg | Starting dose: | 100 mg/day |
| | g/day; dose may | i.v. every 2 weeks | 23.7 mg twice | |
| | be lowered | for 6 doses | daily. Dose may | |
| | | High dose: 500- | be reduced | |
| | | 1000 mg/m² i.v. | stopped or re- | |
| | | monthly for 6 doses | initiated based | |
| | | | on eGFR | |
| t_{1/2} × 5 | 5 days | 3 days | 7 days | 1 day |

| Drug | Rituximab | Belimumab | Obinutuzumab |
|---|---|---|---|
| Target dose | Initial dose: | 10 mg/kg (i.v.) at 2 | Clinical trial 1000 mg (i.v.) Day 1 |
| | 1000 mg and at | week intervals for | and Weeks 2, 24 and 26 |
| | 2 weeks then | first 3 doses and at | |
| | every 16-20 | 4 week intervals | |
| | weeks | thereafter | |
| | | 400 mg (s.c) once | |
| | | weekly for 4 doses | |
| | | and 200 mg once | |
| | | weekly thereafter | |
| t_{1/2} × 5 | 95 days | 97 days | 142 day (based on patients with CLL) |

Corticosteroids and other immunosuppressive drugs are avoided for 3 months after the hypoimmune allogeneic CD19-directed T cell infusion unless used to manage hypoimmune allogeneic CD19-directed T cell-related toxicities. Other immunomodulatory medications that might interfere with the evaluation of hypoimmune allogeneic CD19-directed T cells of the present disclosure, such as nonsteroidal anti-inflammatory agents, should also be avoided for the same period unless medically necessary.
Therapeutic doses of systemic anticoagulants, such as unfractionated heparin and low-molecular weight heparin, are avoided whenever possible for subjects who are at risk of bleeding due to thrombocytopenia when possible.

Treatments for underlying disease, such as surgery, immunotherapy, targeted agents, and high-dose corticosteroids (other than those defined/allowed in this protocol) and other investigational agents are prohibited, except as needed for treatment of disease worsening after the hypoimmune allogeneic CD19-directed T cell infusion upon consultation with the medical monitor.
Vaccinations must not have been received within 6 weeks of screening visit. Live vaccines should not be administered within 3 months after hypoimmune allogeneic CD19-directed T cell treatment.
Exposure to leflunomide is not permitted within 28 days of screening due to the drug's extended half-life.

### Treatment

The treatment and Year 1 follow-up phase begins with initiation of LD chemotherapy followed by hypoimmune allogenic CD19-directed CAR T cells administration and ends at the end of Year 1 (or until withdrawal of consent, subject death, or other predefined situations). Safety, efficacy, and biomarker assessments will be collected according to the schedule of assessments (“SOA”; Table 14 and Table 15).
Prior to infusion with hypoimmune allogeneic CD19-directed T cells, subjects receive lymphodepleting (LD) chemotherapy. The LD chemotherapy consists of fludarabine 25 mg/m² IV daily for 3 days between Days −5, and −3 and cyclophosphamide 1000 mg/m² IV once on Day −3 prior to infusion with hypoimmune allogeneic CD19-directed T cells on Day 0.
Three subjects per disease cohort are administered a LD chemotherapy of fludarabine 25 mg/m² IV daily for 3 days on Days −5, −4, −3 and cyclophosphamide 1000 mg/m² IV once on Day −3.
Subjects receive the following 3-day pretreatment chemotherapy regimen per the order identified below:
Hydration (IV) with 1 L of 0.9% sodium chloride (NaCl) given before cyclophosphamide on the day of infusion.
Cyclophosphamide 1000 mg/m² IV over approximately 60 minutes on Day −3.
Fludarabine 25 mg/m² IV over approximately 30 minutes on Days −5, −4, and −3. Subjects with moderate impairment of renal function have their fludarabine dose reduced by 20% and are monitored closely.
An additional 1 L of 0.9% NaCl at the completion of the fludarabine infusion.
Add Mesna (sodium 2-mercaptoethanesulfonate; a detoxifying agent used to inhibit the hemorrhagic cystitis induced by the chemotherapy).

Hypoimmune allogeneic CD19-directed T cells are administered IV once 3 days (range 2-7 days) after completion of the LD chemotherapy. Subjects with moderate impairment of renal function may have their fludarabine dose reduced by 20% and be monitored closely. Fludarabine 25 mg/m²/day IV can be infused over approximately 30 minutes on Days −5, −4, and −3.
In the present example, Pneumocystis jiroveci prophylaxis is administered in screening prior to hypoimmune allogeneic CD19-directed T cell dosing. Atovaquone (Mepron) dosed at 750 mg BID p.o is given to subjects with extrarenal lupus (ERL) and lupus nephritis (LN). Single or double strength trimethoprim/sulfamethoxazole (Bactrim) is given p.o. to subjects with AAV and MN. Subjects with an allergy to Bactrim may be given Atovaquone doses at 750 mg BID p.o. or Dapsone (100 mg/day) after measurement of G6-PD level. Pneumocystis prophylaxis is given to subjects for 6 months after hypoimmune allogenic CD19-directed T cell dosing.

In the present example, additional antimicrobial, antiviral and antifungal prophylaxis is administered as clinically indicated. Pre-medication with corticosteroids or tocilizumab or anakinra is not permitted.
In the present example, A single IV infusion of hypoimmune allogeneic CD19-directed T cells of the present disclosure are administered to each enrolled subject at Day 0 at one of the dose levels presented in Table 19. The initial dose level in the study across all indication cohorts will be 90×10⁶ CAR+ T cells. Subjects are pre-medicated with acetaminophen and antihistamine prior to infusion with hypoimmune allogeneic CD19-directed T cells.

TABLE 19
Dose Exploration Cohorts

| Dose | Dose (viable | | |
|---|---|---|---|
| Level | CAR⁺ T cells) | Number of Subjects^{a} | Comments |
| −1 | 60 × 10⁶ | Will vary based on | If de-escalation recommendation is |
| | | decisions to reduce dose. | made by the iDMC or decision is |
| | | | made by the Sponsor |
| 1 | 90 × 10⁶ | 9-36 | Starting dose |
| 2 | 120 × 10⁶ | Will vary, based on cohort | Increased dose if significant benefit |
| | | specific decision to | not observed with starting dose and |
| | | increase dose | safety deemed acceptable in |
| | | | consultation with the iDMC |

Abbreviations: CAR, chimeric antigen receptor; iDMC, independent Data Monitoring Committee.
^{a}Pending safety and efficacy data, additional subjects (e.g., up to 10 total) may be enrolled to a specific dose level deemed safe according to 3 + 3 design to further characterize the safety and efficacy profile in consultation with the iDMC.

The following pre-infusion medications are administered approximately 1 hour before the infusion of hypoimmune allogeneic CD19-directed T cells
Acetaminophen 500 to 1,000 mg orally (or equivalent); and/or
H1 or H2 antihistamine (e.g., diphenhydramine or famotidine).

Subjects with autoimmune disease are at higher risk of seizure than normal patients. Anti-seizure prophylaxis medication with Keppra (levetiracetam) (100 mg p.o./day) is administered on day 0 and continued for at least 1 month after dosing of hypoimmune allogeneic CD19-directed T cell. Dose reductions are considered in subjects with mild or moderate renal impairment.
Central venous access, such as a port or peripherally inserted central catheter, may be used for the infusion of hypoimmune allogeneic CD19-directed T cell.

Dose escalation will be conducted using a 3+3 design with dose escalation rules defined in Table 19. Subjects will be enrolled and treated at sequential dose-escalation levels and evaluated for dose limiting toxicities (DLTs). Dose escalation will begin with Dose Level 1 (DL1) and there will be 2 planned dose levels based on fixed numbers of CAR T cells.

In the present example, after a dose is deemed safe and tolerable based on 3+3 design, i.e., 0 (0 or 1) DLT out of 3 (6) subjects, a higher dose may be tested or the current dose may be expanded by enrolling an additional 3 subjects.

In the present example, for subjects whose condition worsens or who experience a severe disease flare during the 2 years of study, conventional therapies can be given as clinically indicated at the Investigator discretion.

In the present example, Hypoimmune allogeneic CD19-directed T cells derived from up to 5 donors are used.

### Dose Limiting Toxicities

Dose limiting toxicities (DLTs) are graded using the ASTCT Consensus Grading for CRS and ICANS, Mount Sinai Acute GvHD International Consortium 2016 report for GvHD and CTCAE version 5 for all others. A DLT is defined as the following events that are at least possibly related to hypoimmune allogeneic CD19-directed T cell events occurring within 28 days of infusion:
Death
Grade≥3 CRS of any duration
Grade 2 CRS that does not improve to a Grade≤1 within 7 days following adequate therapy
Grade≥3 neurotoxicity of any duration and any Grade 2 neurotoxicity that fails to improve to Grade≤1 within 7 days
Grade≥2 acute GvHD requiring systemic therapy (oral or IV glucocorticoids (≥1 mg/kg/day)) and without resolution to Grade≤1 within 7 days or any Grade≥3 acute GvHD of any duration
Grade 1 acute GvHD that is glucocorticoid-refractory (i.e., progression after 3 days or no response after 7 days of systemic glucocorticoid treatment [≥1 mg/kg/day prednisone or equivalent])
Any Grade≥4 toxicity (including hematologic)
Prolonged Grade 3 hematologic toxicity (cytopenia) that dose not resolve within 28 days of nadir
Grade≥3 systemic hypersensitivity infusion reaction of any duration
Grade≥3 organ toxicity (cardia, dermatologic, gastrointestinal, hepatic, pulmonary, renal/genitourinary, or neurologic)
All other Grade≥3 toxicities that do not resolve to Grade≤2 or Baseline within 14 days

The following events may be expected from the LD chemotherapy or CD19-directed CAR T cell therapy and will not be considered DLTs:
Grade 3 or 4 laboratory values that are not considered clinically significant by the investigator
Myelosuppression, defined as lymphopenia, anemia, neutropenia, and thrombocytopenia unless meeting the DLT definition described above
Grade 3 or 4 fever or chills lasting ≤1 week
Grade 3 transaminases for ≤2 weeks
Grade 3 bone pain due to T cell expansion in marrow compartments for ≤2 weeks
Grade 3 or 4 lymphopenia or leukopenia
Grade 3 or 4 infections, B-cell aplasia, and hypogammaglobulinemia
Grade 3 or 4 renal function tests that are considered TRAEs and worsen from baseline and do not improve to Grade≤2 within 14 days

Events occurring beyond 28 days after the hypoimmune allogeneic CD19-directed T cell infusion identified as clinically significant may be considered as an additional DLT. The period of DLT evaluation may be extended based on emerging data.

### Study Assessments

### Demographic Data

Demographic data will be collected at screening for each subject including sex, year of birth, race, ethnicity, to assess the possible association of demographics with subject safety and treatment effectiveness. Known baseline genetic disease mutations will be recorded for all subjects, where applicable. Additionally, for female subjects the number of previous births (along with gender of births) will be collected to assess for potential Rh alloimmunization.

### Medical and Treatment History

Relevant medical history from before enrollment will be collected during screening. Relevant medical history is defined as information about the subject's medical condition(s) that would be typically shared in a referral letter.

In addition to the medical history, all history related to the subject's cancer profile and status, treatment(s), and response(s) to treatment(s) dating back to the original diagnosis will be collected.

### Safety Assessments

Safety assessments include the collection of AEs and serious adverse events (SAEs), clinical laboratory tests, physical and neurologic examinations, vital signs, and cardiac function (e.g., electrocardiogram [ECG]). Safety assessments are conducted according to the schedule presented in the SOA (Table).

### Adverse Events

During clinic visits, subjects are asked about any adverse events (AEs) that might have occurred since the last study visit. SAEs, treatment-related AEs, treatment emergent AEs, adverse event of special interest, and DLTs are collected as indicated in the SOA (Table).

### Clinical Laboratory Tests

Blood, serum, or urine samples are collected for clinical laboratory tests for safety assessments at the time points indicated in the SOA (Table and Table 24). Additional samples are collected as needed for further safety testing. All clinical laboratory test assessments listed in Table are performed at a local laboratory, unless otherwise noted.

TABLE 20
Local Laboratory Tests for Safety Assessments

| Serum Chemistries | Hematology | | | |
|---|---|---|---|---|
| (Serum) | (Blood) | Coagulation | Urinalysis | Other |
| Albumin | Absolute lymphocyte | APTT | Specific gravity | Pregnancy test |
| | count | | | |
| ALT (SGPT) | Absolute neutrophil | PT and INR | pH | Serologic testing |
| | count | | | |
| ALP | Hemoglobin | Fibrinogen | Blood | Covid-19 test |
| AST (SGOT) | RBC | D-dimer | Protein | Serum Ig levels |
| Bicarbonate (CO₂) total | Hematocrit | | Glucose | |
| (as clinically indicated) | | | | |
| Bilirubin, total and | Reticulocytes | | Bilirubin | |
| direct | | | | |
| BUN or urea | Platelets | | WBC | |
| Calcium total | WBC | | RBC | |
| Chloride | Differential: | | Epithelial cells | |
| Creatinine | Neutrophils | | Bacteria | |
| Creatinine clearance | Eosinophils | | Casts | |
| (eGFR as estimated by | Basophils | | Crystals | |
| CKD-EPI equation) | Lymphocytes | | | |
| (screening only)^{e} | Monocytes | | | |
| Standard C-reactive | | | Spot UPCR | |
| protein | | | | |
| Ferritin | | | | |
| Glucose | | | | |
| Inorganic phosphorus | | | | |
| LDH | | | | |
| Magnesium | | | | |
| Potassium | | | | |
| Sodium | | | | |
| Uric Acid | | | | |
| Phosphorus | | | | |
| Total protein | | | | |

Abbreviations: ALP, alkaline phosphatase; ALT, alanine aminotransferase; APTT, activated thromboplastin time; AST, aspartate aminotransferase; BUN, Blood urea nitrogen; CKD-EPI, chronic kidney disease epidemiology; eGFR, estimated glomerular filtration rate; Ig, immunoglobin; INR, international normalized ratio; LDH, lactate dehydrogenase; PT, prothrombin time; RT-PCR, reverse transcriptase polymerase chain reaction; SGOT, serum glutamic-oxaloacetic transaminase; SGPT, serum glutamic-pyruvic transaminase; RBC, red blood cell; UPCR, urine protein creatinine ratio; WBC, white blood cell.

Central laboratory test assessments intended for assessment of safety and other endpoints (e.g., immunogenicity and efficacy) are listed in Table.

TABLE 21
Central Laboratory Assessments
Assessment

CAR VCN (ddPCR)
CAR T cell enumeration/CD 47 expression (flow cytometry)
Cytokine profiling/soluble markers of inflammation
Replication competent lentivirus (PCR)
T cell clonality (clonal expansion)
Immunophenotyping (flow cytometry)
Humoral immunogenicity (anti-CD19 CAR)
Cellular immunogenicity (anti-CD19 CAR peptides)
Autoantibody profile
Complement profile
Quantitative Immunoglobulins
Protective autoantibody profile
Protective antibodies include assessments of anti-tetanus, anti-
pneumococcal, and other antibodies induced by prior immunization
Soluble markers of B cell activation including BAFF, APRIL, and
others
Urine biomarkers
Abbreviations: APRIL, A proliferation-inducing ligand; BAFF, B-cell activating factor; CAR, chimeric antigen receptor; ddPCR, droplet digital polymerase chain reaction; DSA, donor-specific antibody; HIP, hypoimmune; PCR, polymerase chain reaction; scRNAseq, single cell RNA (ribonucleic acid) sequencing; VCN, vector copy number.

The lupus anticoagulant autoantibody can interfere with the assays for measurement of activated thromboplastin time (aPTT) and international normalized ratio (INR) in subjects with SLE. This may cause an artificial elevation of these lab tests and could exclude an eligible patient from participation. If a subject with SLE has an elevated PTT or INR above normal range and not associated with anticoagulation, the cause is documented and reviewed in screening to determine eligibility.

### Clinical Examination

Clinical examinations are performed during screening and at times noted in the SOA (Table). At subsequent examinations, clinically significant adverse changes are compared with the baseline examination are reported as AEs. Any clinically relevant findings are documented. Height is measured at screening only. Weight is collected during screening and at times noted in the SOA (Table).
Vital signs include systolic/diastolic blood pressure, heart rate, respiratory rate, oxygen saturation, and temperature. Vital signs are monitored as indicated in the SOA (Table). During hypoimmune allogeneic CD19-directed T cell treatment, vital signs are monitored before and after the infusion and then at regular intervals described in (Table).
Oxygen saturation is measured using a standard pulse oximeter.
Neurologic examination is performed to assess the immune effector cell-associated encephalopathy (ICE) score, according to the time points specified in the SOA (Table). The subject's neurological status is evaluated at screening to establish a baseline. Changes in neurological status is reported as an AE if considered clinically significant.

Subjects with CNS lupus manifestations or neurologic involvement in AAV (e.g., pachymeningitis, cerebrovascular accident) will be excluded from the study. Since subjects in each cohort are at risk for both immune effector cell-associated neurotoxicity syndrome (ICANS) as well as a potential flare of neurologic symptoms of their underlying disease, careful assessment of neurologic symptoms is undertaken to determine the appropriate diagnosis and treatment.
Estimated glomerular filtration rates using the CKD-EPI equation is assessed at the time points specified in the SOA to inform subject eligibility for the study, fludarabine dose used in LD chemotherapy and to assess disease status at post-dose time points (Table).
Each subject's cardiac function including ejection fraction, as measured by an ECHO/MUGA scan, is assessed during the screening period to confirm study eligibility. Subjects with an ejection fraction ≤50%, arrythmias and any structural heart disease (including clinically significant pericardial effusion), as assessed by echocardiogram or multiple gated acquisition scan performed within 1 month prior to hypoimmune allogeneic CD19-directed T cell treatment are excluded from the study. An ECHO/MUGA scan performed within 1 month prior to hypoimmune allogeneic CD19-directed T cell treatment may be used for confirmation of eligibility.
To establish a baseline, a 12-lead ECG is performed during the screening period. ECG assessments include PR duration, QRS duration, QT duration, RR duration, and corrected QT interval. The corrected QT interval is calculated using Fridericia's formula. Subjects with cardiac arrhythmias requiring medication are excluded from the study. 12-lead ECGs are also be performed at the time points specified in the SOA (Table).

At the onset of Grade 2 cardio-renal syndrome (CRS), or if clinically indicated, telemetry cardiac monitoring is performed at the time points specified in the SOA (Table).
Spirometry is performed at Screening. A Forced Expiratory Volume in the first second/Forced Vital Capacity (FEV1/FVC)>70% by spirometry is required for study eligibility. Spirometry is included to evaluate any underlying pulmonary disorder potentially impacted by CRS or other drug related toxicities.

Baseline oxygen saturation >92% on room air, and no clinically significant pleural effusion evaluated at screening is required for study eligibility.
A chest X-ray is performed at screening for all subjects and as specified in the SOA for subjects with AAV (Table).

### Disease Assessment

Disease assessments are performed according to the schedule presented in the SOA (Table). Subjects with symptoms suggestive of disease progression are evaluated for progression at the time symptoms occur.
A kidney biopsy within 6 months of screening or during the screening interval is required for inclusion.
   Renal Response in Subjects with LN

Renal response is assessed according to the schedule presented in the SOA (Table). A complete renal response is defined as the proportion of subjects achieving urine protein creatinine ratio (UPCR) of <0.5, and eGFR ≥60 mL/min/1.73 m² or no confirmed decrease from baseline in eGFR of >20% or no treatment- or disease-related eGFR-associated event. A partial renal response is defined as a ≥50% reduction in UPCR from baseline to a value <1 (to <3 if baseline UPCR was ≥3), or no confirmed decrease from baseline in eGFR of >20% or no treatment- or disease-related eGFR-associated event. eGFR is calculated using the CKD-EPI equation.

Remission in Subjects with ERL

A modified DORIS remission definition is evaluated with remission defined as SLEDAI-2K of 0 and Evaluator's Global Assessment score of <0.5 (scale 0-3). The subject may be on anti-malarials, low-dose glucocorticoids (prednisolone <5 mg/day), and/or stable immunosuppressives including biologics.
Remission in subjects with ERL is also assessed based on the SLEDAI-2K score with disease remission defined as a score of 0. Partial response is defined as a 50% reduction in SLEDAI-2K score without the use of immunosuppressives or biologics from baseline.
   Remission in Subjects with AAV

The Birmingham Vasculitis Activity Score (BVAS) version 3 (v3) is a comprehensive multisystem assessment of systemic vasculitis. The BVAS v3 is a clinician-reported outcome to assess persistent and new or worsening symptoms and signs of systemic vasculitis. The score consists of 56 items grouped into 9 sections (general, cutaneous, mucous membrane/eyes, ENT, chest, cardiovascular, abdominal, renal, and nervous system). Persistent scores can range from 0-33 whilst new/worsening scores can range from 0-63. Higher scores reflect more severe disease. Disease remission in AAV subjects is defined as a BVAS v3 of 0.

Disease Status in Subjects with LN and ERL

Disease status for subjects with LN and ERL is evaluated at the time points indicated in the SOA using the SLEDAI-2K tool, a clinician-reported outcome. The SLEDAI-2K is also assessed for the modified DORIS remission in ERL.
The SLEDAI index was introduced as a measure of the assessment of SLE disease activity based on a series of 24 clinical and laboratory variables with the most current iteration being the SLEDAI 2000 (2K) index. Disease activity may range on a point scale from 0-105. SLEDAI scores for the presence of autoantibodies (anti-dsDNA antibody titers), low complement, and other hematological and renal parameters. SLEDAI scores >5 are usually associated with a >50% probability of initiating therapy. Activity categories have been defined on the basis of SLEDAI scores: no activity (SLEDAI=0), mild activity (SLEDAI=1 to 5), moderate activity (SLEDAI=6 to 10), high activity (SLEDAI=11 to 19), and very high activity (SLEDAI ≥20) (101). As in the original SLEDAI, all the descriptors in the SLEDAI-2K must be attributable to lupus activity. SLEDAI-2K describes disease activity at varying activity levels and describes changes in disease activity over time.

### Damage Index

Subjects previously diagnosed and treated for disease can have suffered from either disease-related or treatment-related damage. Patients with autoimmune disease can suffer from active disease for a prolonged amount of time prior to their initial diagnosis and the start of the present study. Evidence of damage at baseline and throughout the study is collected to determine if damage is worsened in any organ system.
The Systemic Lupus International Collaborating Clinics/American College of Rheumatology (SLICC/ACR) is implemented for ERL and LN cohorts. SLICC/ACR is a clinician-reported outcome developed to assess ongoing disease activity in SLE patients and to assess irreversible damage resulting from SLE disease activity and treatment. Damage is defined for 12 organ systems: ocular (range 0-2), neuropsychiatric (0-6), renal (0-3), pulmonary (0-5), cardiovascular (0-6), peripheral vascular (0-5), gastrointestinal (0-6), musculoskeletal (0-7), skin (0-3), endocrine (diabetes) (0-1), gonadal (0-1) and malignancies (0-2). Items should be present for at least 6 months with the exception that manifestations such as myocardial infarction and stroke are recorded once they occur. Damage over time can only be stable or increase to a maximum score of 47 points.

Subjects with AAV damage are assessed using the vasculitis damage index (VDI). The VDI is a clinician-reported outcome developed to measure damage from vasculitis or treatment and has been validated and used in AAV clinical trials. Irreversible disease manifestations must be attributed to vasculitis or its therapy and present for at least 3 months before being scored as damage rather than ongoing activity. The cause of damage (i.e., vasculitis versus treatment) is not considered in scoring. The VDI comprises 64 items that are categorized into 11 groups by organ system (musculoskeletal, skin/mucous membranes, ocular, ENT, pulmonary, cardiovascular, peripheral vascular disease, gastrointestinal, renal, neuropsychiatric, other).

### Patient-Reported Outcomes

Subjects complete the following outcomes at the timepoints indicated in the SOA to evaluate potential changes in quality of life and disease status (Table).
SF36 v2 (all subjects): the SF36 is a 36-item self-report questionnaire that measures health related quality of life (QOL). It has 8 subscales measuring different domains of health related QOL: physical functioning, role-physical, bodily pain, general health, vitality, social functioning, role emotional, and mental health. Two component scores are derived from the eight subscales: a physical health component score and a mental health component score. The SF-36 also includes a single item that assesses perceived change in health status over the past year.
LupusQOL (subjects with LN and ERL): the LupusQOL is a 34-item self-report questionnaire that measures disease specific health related QOL in adult SLE. It uses a 5 point Likert scale (0=all the time to 4=never) and covers 8 domains: physical health, emotional health, body image, pain, planning, fatigue, intimate relationships, and burden to others.
AVV-PRO (subjects with AAV): the AAV-PRO is a 29-item self-report questionnaire that measures disease specific health related QOL. It uses a 5 point Likert scale (0=never to 4=all the time), with higher scores denoting greater severity or impact. It comprises 6 domains: organ-specific symptoms, systemic symptoms, treatment side effects, social and emotional impact, concerns about the future, and physical function.

### Physician Global Assessment

The physician global assessment (PGA) is completed at the timepoints indicated in the SOA (Table) to assess the overall response to treatment as assessed by a physician. The PGA for this study is a 0-3 visual analogue scale for measuring disease activity in patients with SLE in the preceding month. The PGA is intended to rate the overall disease activity, taking into account the severity of active manifestations and clinical laboratory results, but excluding organ damage, serology, and subjective findings unrelated to disease activity. The PGA scale ranges from “no disease activity” (0) to the “most severe disease activity” (3) and incorporates the values 1 and 2 as inner markers to categorize disease activity as mild (≥0.5 to 1), moderate (>1 and ≤2) and severe (>2 to 3). Investigators will be trained to rate the study PGA, and it should be scored by the same rater at each visit. This PGA is used for all disease cohorts.

### Cellular Kinetics Assessments

Samples for cellular kinetics CK assessments are drawn according to the schedule presented in the SOA (Table). CAR vector copy number (VCN) is determined by droplet digital polymerase chain reaction (ddPCR) and CAR T cell enumeration will be determined by flow cytometry. Whole blood is used for CAR T cell enumeration by flow cytometry. Hypoimmune allogeneic CD19-directed T cell CK is evaluated by a validated ddPCR method and reported as level of hypoimmune allogeneic CD19-directed T cell transgene copies/μg of genomic DNA. For all subjects treated with hypoimmune allogeneic CD19-directed T cell, the CK profile and the following derived parameters are reported: Cₘₐₓ, Tₘₐₓ, t_{1/2}, Cₗₐₛₜ, Tₗₐₛₜ, AUCs.

In addition, the number of CD19-directed CAR expressing cells in the blood are tracked by flow cytometry. The assessment of hypoimmune allogeneic CD19-directed T cell CK are used to examine the dose-exposure relationship, and possible correlation of persistence and expansion of hypoimmune allogeneic CD19-directed T cell with safety and efficacy endpoints. The timing, but not the total number of samples, may be modified during the study based on emerging CK data if a change in sampling scheme is considered necessary to better characterize the CK.

### Pharmacodynamic Assessments

Blood/serum samples for pharmacodynamic assessments are drawn according to the schedule presented in Table. Pharmacodynamic assessments include monitoring of levels of analytes in blood/serum over time. Analytes include, but are not limited to, the following:
Circulating CD19+ B cells and their subsets (BRC isotypes and bulk B cell receptor repertoire);
Anti-dsDNA, anti-MPO, anti-PR3, anti-PLA2R, and others
Serum complement
Soluble markers of B cell activation including BAFF, APRIL, and others
Inflammatory/immune-modulating cytokines: interferon-gamma (IFN-γ), IFN-α, IL-1β, IL-1Ra, IL-4, IL-6, IL-8, IL-10, IL-12p70, IL-13, IL-17A, granulocyte macrophage-colony stimulating factor (GM-CSF), macrophage inflammatory protein (MIP)-1α, MIP 1β, ferritin, and sIL2R
Correlates of acute phase response: C-reactive protein (CRP) and ferritin
Chemokines: IL-8, C-X-C motif chemokine ligand-10, and monocyte chemotactic protein-1 (MCP-1)
CD19-CAR, CD47, TCR, MHC I/II

### Immunogenicity Assays

The presence of CD19-directed CAR antibodies in the subject's serum is monitored at time points identified in the Table 24. The hypoimmune allogeneic CD19-directed T cell-induced immunogenicity is evaluated by detecting the following analytes:
Total anti-CAR transgene protein specific antibody response (anti-CAR TAb) in human serum
Assessment of neutralizing anti-CAR transgene protein humoral responses will be considered based on the overall data obtained during clinical study conduct. Appropriate samples will be collected and stored for later analysis
Cellular immunogenicity: Study will be ELISpot using anti-CD19 CAR peptides. Sample is saved and analysed if abnormal CAR T proliferation/kinetic was observed.

### Cytokine Profiling/Soluble Markers of Inflammation and Immunophenotyping

Sample is collected for cytokine profiling and soluble markers of inflammation at time points described in Table. A multiplexed platform method is applied to detect and quantify analytes. PBMCs are collected for assessment of immunophenotypic changes induced by hypoimmune allogeneic CD19-directed T cells. These blood samples as well as final product samples are analyzed at time points identified in Table 24 for the presence and changes in immune cell state by flow cytometry; including, but not limited to:
Expression level of CD47 on hypoimmune allogeneic CD19-directed T cells
Broad immune cell phenotyping: lymphoid and myeloid cell phenotypes, e.g., T, B, NK, and monocytes
Immune cell subsets: differentiation, activation, and exhaustion

### RNA Sequencing

PBMCs and final product samples are assessed by gene expression profiling through single cell RNA sequencing for broad molecular landscape analyses. Remaining samples collected as part of the study are stored for up to 15 years for future exploratory analysis related to abnormal clonal expansion. Additional studies involving, but not limited to, next generation sequencing may be performed.

### Autoantibody Profile

Autoantibody levels are determined at the time point indicated in Table 24. The planned autoantibody panel by indication is provided in Table 22.

TABLE 22
Autoantibody Panels by Indication

| Extra Renal Lupus/ | Antibody-associated |
|---|---|
| Lupus nephritis | vasculitis |
| ANA (Hep2) | ANCA |
| Anti-dsDNA | Anti-PR3 |
| Anti-Sm | Anti-MPO |
| Anti-RNP | |
| Anti-Ro52 | |
| Anti-Ro60 | |
| Anti-La | |
| Anti-histone | |
| Anti-cardiolipin IgG, IgM, | |
| IgA | |
| Anti-B2GPI IgG, IgM, IgA | |
| Subject unique autoantibodies | |
| as previously (if historically | |
| positive) | |

### Complement Profile

Multiple abnormalities of the complement cascade present in different autoimmune diseases. Complement levels may be reduced at baseline in patients with severely active disease. Samples are collected for complement profiling at baseline and throughout the conduct of the study and may include C3, C4, CH50 and other complement proteins at time points described in Table.

### Soluble Markers of B Cell Activation

Autoimmune disease may be characterized by B cell hyperactivity and with elevated levels of soluble markers of B cell activation including BAFF, APRIL, and others. B cell depletion therapy may also increase these cytokines, making them a potential pharmacodynamic marker. These B cell specific cytokines are evaluated at the time points indicated in the Table.

### Urine Biomarkers

With the regulatory approval of new therapies in ERL, LN and AAV, scientific knowledge of urinary biomarkers in these diseases has advanced. Urinary biomarkers of disease activity remain a potentially non-invasive assessment of renal inflammation and function. Specific urinary biomarkers are collected and analyzed as outlined in Table.

Management of Toxicities Associated with CAR T Cell Treatment

CRS and its manifestations are managed according to the ASTCT guidelines (Lee 2019, incorporated herein by reference in its entirety). Neurologic events (i.e., ICANS) are managed according to ASTCT guidelines (Lee 2019, incorporated herein by reference in its entirety). GvHD are managed according to the Mount Sinai Acute GVHD International Consortium 2016 report (Harris 2016, incorporated herein by reference in its entirety). IEC-HS are managed according to ASTCT guidelines.

### Analysis Conventions

The following analysis sets are used to analyze the study data:
Full analysis set (FAS): This data set consists of all enrolled subjects, which is the intention-to-treat (ITT) population. The full analysis set will be used for summaries of subject disposition.
Modified Intention-To-Treat (mITT) analysis set: This data set consists of all subjects enrolled and treated with hypoimmune allogeneic CD19-directed T cells. The mITT analysis set is used for all efficacy analyses unless otherwise specified.
DLT-evaluable set (DES): This data set consists of all subjects treated with hypoimmune allogeneic CD19-directed T cell who:
   are followed for at least 28 days after the infusion of hypoimmune allogeneic CD19-directed T cell; or
   experience a DLT during the 28-day period after infusion.
      This analysis set will be used for DLT analysis in support of dose finding decisions.
Safety analysis set (SAS): This data set consists of all subjects treated with any dose of hypoimmune allogeneic CD19-directed T cell. This analysis set will be used for safety analysis.
Safety analysis set LD (SASLD): This data set consists of all subjects treated with any dose of LD chemotherapy, irrespective of hypoimmune allogeneic CD19-directed T cell treatment. This analysis set is used for safety analysis.
   Cellular kinetics analysis set (CAS): This set consists of consists of subjects who are treated with hypoimmune allogeneic CD19-directed T cell and have at least 1 post-dose cellular kinetic sample. This analysis set is used for evaluation of CAR-T cell distribution and expansion.

Demographic and baseline measurements are summarized using standard descriptive methods. Demographic summaries will include sex, race/ethnicity, and age. The efficacy of hypoimmune allogeneic CD19-directed T cell in subjects in ERL will be summarized as the estimated proportion of subjects achieving modified DORIS remission with an exact 2-sided 95% Cis. The efficacy of hypoimmune allogeneic CD19-directed T cell in subjects with LN is summarized as the estimated proportion of subjects achieving a complete renal response or partial renal response with an exact 2-sided 95% CI. The efficacy of hypoimmune allogeneic CD19-directed T cell in subjects in AAV is summarized as the estimated proportion of subjects achieving a complete remission (i.e., a BVAS v3 score of 0) is evaluated. SLEDAI-2K scores, change from baseline and percent change from baseline of the scores will be summarized at each visit for each cohort using descriptive statistics including the number of subjects, mean, standard deviation (SD), median, minimum, and maximum and 2-sided 95% CI.

BVAS v3 scores, change from baseline and percent change from baseline of the scores will be summarized at each visit for each cohort using descriptive statistics including the number of subjects, mean, SD, median, minimum, and maximum and 2-sided 95% CI. The duration of response (DOR) in subjects with LN, ERL and AAV is defined as the time from the first observed response (complete response/remission or partial response/remission) to disease worsening or death due to any cause, whichever occurs first. If worsening or death is not observed, the DOR will be censored at the last evaluable assessment date on or prior to the censoring events defined as:
Ongoing without event
Lost to follow-up
Withdrew consent
New systemic treatment for the indication under study
Adequate assessments no longer available

Kaplan-Meier estimates and 2-sides 95% CIs will be generated for the DOR.

For SF-36, physical component score (PCS) and mental component score (MCS), change from baseline and percent change from baseline of the corresponding scores are summarized at each visit for each cohort using descriptive statistics including the number of subjects, mean, SD, median, minimum, and maximum and 2-sided 95% CI.

For LupusQOL and AAV-PRO, total scores of each domain, change from baseline and percent change from baseline of the corresponding scores will be summarized by domain at each visit for each cohort using descriptive statistics including the number of subjects, mean, SD, median, minimum, and maximum and 2-sided 95% CI.

PGA scores will be summarized at each visit for each cohort using descriptive statistics including proportion of each PGA score and 2-sided exact confidence interval.

Incidence rates of TEAEs, defined as AEs with an onset during the LD chemotherapy period and on or after the hypoimmune allogenic CD19-directed T cell infusion, will be summarized. TEAEs including all, serious, fatal, Grade≥3, TRAEs, TAEs, and AESI will be tabulated by preferred term and/or system organ class. CTCAE v5.0 grade changes in safety laboratory values will be summarized with descriptive statistics. The incidence of concomitant medications will be summarized. Clinical and laboratory AEs will be coded using the Medical Dictionary for Regulatory Activities.

For Year 2 follow-up period, tables and/or narratives of deaths and treatment-related SAEs will be provided.

The subject incidence of CD19-directed CAR antibodies is using descriptive statistics.

Hypoimmune allogeneic CD19-directed T cell cellular kinetics (CK) parameters are estimated using a non-compartmental analysis approach when the data is sufficient. The CK parameters (Cmax, Tmax, t½, Clast, Tlast, and AUCs) are estimated from PCR based hypoimmune allogeneic CD19-directed T cell-concentration (copies/microgram gDNA)-time profiles for the CK analysis set as well as flow cytometry-based hypoimmune allogeneic CD19-directed T cell concentration. The CK parameters are determined, as permitted by data, by DL, indication, and responder/non responder.

For each pharmacodynamic and biomarker time point, peak and time to peak values are determined for pro inflammatory and immune-modulating cytokines, chemokines, and effector molecules in serum/plasma over time. Descriptive statistics are provided for all PD and biomarker parameters.

### Replication Competent Lentivirus

As hypoimmune allogeneic CD19-directed T cells comprise lentiviral vector-transduced T cells, the presence of replication competent lentivirus (RCL) in the blood of treated subjects is monitored according to the schedule presented in the SOA (Table 23).

Peripheral blood mononuclear cells (PBMCs) for RCL testing are obtained at baseline (before the LD chemotherapy), and at Months 2, 3, 6, 12, and 24. If a positive RCL assay result is obtained from a subject blood specimen, the subject is rescheduled for RCL retest. Thereafter, samples are only collected if an RCL event is clinically suspected and/or a subject's PBMC sample tests positive for RCL at any time point within the first year. If the latter, samples continue to be collected and tested annually for up to 15 years or as clinically indicated. RCL testing ends after 2 consecutive negative results.

### T Cell Clonality Assays

A sample is collected for monitoring of abnormal T cell proliferation by flow cytometry for CAR+ T cells over the first 3 months. In addition, samples are also collected for storage and analyzed if abnormal CAR T cell expansion is observed. T cell clonal expansion is monitored by flow and vector copy number (VCN) for the anti-CD19 CAR transgene, and by complete blood count (CBC). If more than 1% of the T cells are positive for anti-CD19 CAR after 3 months on treatment or if T cell expansion occurs after contraction during the first 3 months on treatment, further studies to assess T cell clonality (including but not limited to vector integration sites) is performed.

### Post-Treatment Follow-Up

After completing the hypoimmune allogenic CD19-directed CAR T cells infusion, all subjects will be followed during the post-treatment follow-up period.

Subjects remain hospitalized for at least 7 days after the hypoimmune allogeneic CD19-directed T cell infusion for daily monitoring of signs and symptoms of CRS, neurologic events, and other anticipated complications. In addition, the assessments described in the SOA (Table 23 and Table 24) is performed during this period.

Subjects are monitored for one year following administration with hypoimmune allogeneic CD19-directed T cells. Adverse Events (AEs) are collected, and disease status is assessed according to the Schedule of Assessments (SOA; Table 23 and Table 24). An additional year of follow up assesses disease status and AEs quarterly. Subjects are monitored for an additional 13 years for continued safety and survival monitoring.

In the post-treatment period, subjects within each cohort are at risk of disease flares. Disease flares in subjects with extrarenal lupus (ERL) are assessed and recorded using the SLEDAI-2K disease assessment scale. In subjects with AAV, the BVAS v3 is used for disease assessment and recording.

If a subject's condition worsens or if they experience a severe disease flare during the 2 years of study, conventional therapies are given, as clinically indicated.

TABLE 23
Schedule of Assessments (SOA)
A. Screening, Immunosuppression Taper, LD chemotherapy, Hypoimmune Allogeneic CD19-directed T cell Treatment, and Month 1

| | |
|---|---|
| | Hypoimmune |
| | Allogeneic |
| | CD19- |
| Screening | directed T cell |

| | |
|---|---|
| and IS | Treatment |

| taper | LD | D0 | | Month 1 FU Phase^{b} | | | | | |
|---|---|---|---|---|---|---|---|---|---|
| Up to 21 | chemotherapy^{a} | Pre- | Post- | | D10 | D13 | D16 | D21 | D28 |

| Procedure | days | D-5^{c} | D-4 | D-3 | dose | dose | D1 | D2 | D3 | D4 | D5 | D6 | D8 | ±1 D | ±1 D | ±1 D | ±4 D | ±4 D |
|---|---|---|---|---|---|---|---|---|---|---|---|---|---|---|---|---|---|---|
| Informed consent | X | | | | | | | | | | | | | | | | | |
| Demographics and | X | | | | | | | | | | | | | | | | | |
| medical history | | | | | | | | | | | | | | | | | | |
| Height and weight^{c} | X | X | X | X | X | | | | | | | | | | | | | X |
| Clinical examination^{d} | X | X | X | X | X | X | X | X | X | X | X | X | X | X | X | X | X | X |
| Neurologic exam | X | | | | X | | X | X | X | X | X | X | X | X | X | X | X | X |

| | | | | | | | | | | | |
|---|---|---|---|---|---|---|---|---|---|---|---|
| Vital signs | X | X | X | X | X | q6 h | q12 hr | X | X | X | As |

| | | | | | | (±1 h) | | | | | | | | | clinically |
|---|---|---|---|---|---|---|---|---|---|---|---|---|---|---|---|
| | | | | | | | | | | | | | | | indicated |
| eGFR | X | | | | X | | | | | | | X | X | | X |
| ECHO/MUGA^{e} | X | | | | | | | | | | | | | | |
| Brain MRI^{f} | X | | | | | | | | | | | | | | |
| Chest X-ray | X | | | | | | | | | | | | | | X |
| Spirometry | X | | | | | | | | | | | | | | |
| Urinalysis for spot | X | | | | | | | | | | | X | X | X | X |
| UPCR | | | | | | | | | | | | | | | |
| 24-hour urine collection | X | | | | | | | | | | | | | | X |
| Immunosuppression | X | | | | | | | | | | | | | | |
| taper^{g} | | | | | | | | | | | | | | | |
| Telemetry cardiac | | | | | | At the onset of Grade 2 CRS until resolution to Grade ≤1, and | | | | | | | | | |
| monitoring | | | | | | additionally as clinically indicated | | | | | | | | | |
| 12-lead ECG | X | X | X | X | X | As clinically indicated | | | | | X | | | | |
| SAE assessment^{h} | Continuously | | | | | | | | | | | | | | |
| AE assessment | Continuously | | | | | | | | | | | | | | |
| DLT assessment | | | | | | Continuously | | | | | | | | | |
| Concomitant | Continuously | | | | | | | | | | | | | | |
| medications | | | | | | | | | | | | | | | |
| CBC, chemistry, | X | X | X | X | X | | X | X | X | X | | X | X | X | X |
| coagulation, urinalysis | | | | | | | | | | | | | | | |
| testⁱ | | | | | | | | | | | | | | | |
| LDH/CRP/Ferritin | X | X | | | X | | X | X | X | X | | X | X | X | X |
| Virus tests | X | | | | | | As clinically indicated | | | | | | | | |
| (COVID-19, HIV, HBV, | | | | | | | | | | | | | | | |
| HCV, HTLV, RSV) | | | | | | | | | | | | | | | |
| CK/biomarker | See CK/Biomarker table (Table 24) | | | | | | | | | | | | | | |
| assessment | | | | | | | | | | | | | | | |
| Pregnancy | X | | | | | | | | | | | | | | X |
| test/Gynecology consult^{j} | (twice) | | | | | | | | | | | | | | |
| Hospitalization^{k} | | | | | | | X | | | | | | | | |
| Cyclophosphamide | | | | X | | | | | | | | | | | |
| Fludarabine | | X | X | X | | | | | | | | | | | |
| Pre-medication and | | | | | X | | | | | | | | | | |
| prophylaxis^{l} | | | | | | | | | | | | | | | |
| Hypoimmune | | | | | | X | | | | | | | | | |
| Allogeneic CD19- | | | | | | | | | | | | | | | |
| Directed T cell | | | | | | | | | | | | | | | |
| Treatment | | | | | | | | | | | | | | | |

Efficacy assessment for all subjects (Cohorts 1-3)

| | | | |
|---|---|---|---|
| SF36 v2 | X | | X |
| PGA | X | X | X |

Cohort 1 and 2-specific assessments (subjects with LN and ERL)

| | | |
|---|---|---|
| SLEDAI-2K | X | X |
| LupusQOL | X | X |
| SLICC/ACR Damage | X | X |
| Index | | |

Cohort 3-specific assessments (subjects with AAV only)

| | | |
|---|---|---|
| BVAS | X | X |
| VDI | X | X |
| AAV-PRO | X | X |

Abbreviations: AAV-PRO, anti-neutrophil cytoplasmic antibody (ANCA)-associated vasculitis-patient reported outcome; AE, adverse event; BVASv3, Birmingham Vasculitis Activity Score;
CBC, complete blood count; CK, cellular kinetics; CRP, C-reactive protcin; D, day(s); DLT, dose-limiting toxicity; ECG, electrocardiogram; ECHO, echocardiogram; cGFR, estimated
glomerular filtration rate; FU, follow-up; HBV, hepatitis B virus; HCV, hepatitis C virus; HIV, human immunodeficiency virus; hr, hour; HTLV, Human T-lymphotropic virus 1; IS,
immunosuppression; LD, lymphodepleting; LDH, Lactate dehydrogenase; LTFU, long-term follow-up; LN, lupus nephritis; MN, membranous nephropathy; M, month;
MUGA, multigated acquisition; PGA, Physician Global Assessment; PRO, patient-reported outcome; QOL, Quality of Life; q6hr, every 6 hours; q12 hr, every 12 hours; RSV, respiratory
syncytial virus; SAE, serious adverse event; SF36, 36-Item Short Form Survey; SLEDAI-2K, Systemic Lupus Erythematosus Disease Activity Index 2000; SLICC/ACR, Systemic Lupus
International Collaborating Clinics/American College of Rheumatology; UPCR, urine protein creatinine ratio; VDI, Vasculitis Damage Index.
Footnotes:
^{a}Assessments to be performed after completion of the LD chemotherapy and prior to hypoimmune allogeneic CD19-directed T cell infusion.
^{b}Assessments to be performed for up to 12 months following hypoimmune allogeneic CD19-directed T cell infusion (or until withdrawal of consent and/or start of new systemic therapy).
^{c}Height will be measured at screening only.
^{d}After subject discharge from initial hospitalization, clinical examination may occur as clinic visit, telemedicine visit, or home health visit (per investigator discretion).
^{e}Ejection fraction at screening must be > 50%, without evidence of pericardial effusion, as assessed by ECHO/MUGA scan performed within 1 month prior to hypoimmune allogeneic
CD19-directed T cell treatment.
^{f}Brain MRI is conducted to rule out seizure risk.
^{g}Tapering of immunosuppressive medications will occur at the Investigator's discretion during the Screening period with the aim of discontinuation before LD chemotherapy initiation
at −Day 5. Antimalarial therapy can be continued during this immunosuppression tapering period if the subject was receiving them at the time of consent. Antimalarials should be
stopped before LD chemotherapy.
^{h}During LTFU, TRAEs will be assessed quarterly (or until subject begins next new systemic treatment).
ⁱPre-dose hematology and serum chemistry samples may be taken up to 3 days before the visit, and coagulation testing and urinalysis may be conducted 24 hours before the visit.
Subjects with MN and low albumin should be evaluated for initiation of anticoagulation in the enrollment phase, and therapeutic anticoagulation should be reached prior to initiation of
lymphodepletion. On Day 0, all results must be evaluated before hypoimmune allogeneic CD19-directed T cell dosing. Microscopic analysis of urine sediment should be performed if
significant abnormalities are detected in proteins, leukocytes, or blood. Serum creatinine clearance is to be calculated using the CKD-EPI equation.
^{j}For women of childbearing potential, pregnancy tests will be performed at Screening and within 7 days before starting LD. Pregnancy testing will occur at Day 28 and monthly thereafter,
coinciding with clinic visits. At the Screening visit, a gynecology consult must be conducted to review infertility risks and recommendations prior to administration of the LD chemotherapy.
^{k}Reduced hospitalization or outpatient administration may be permitted within a cohort after review of data on at least 6 subjects with a minimum of 28 days of data and in consultation
with the iDMC.
^{l}All subjects will be pre-medicated with acetaminophen and antihistamine prior to hypoimmune allogeneic CD19-directed T cell infusion. Pre-medication with glucocorticoids,
tocilizumab, or anakinra is not permitted.
B. Screening, Immunosuppression Taper, LD chemotherapy, Hypoimmune Allogeneic CD19-directed T cell Treatment, and
Month 1

| | Months 2-12 FU^{a} | | | | | | | | | | | Year 2 FU^{b} | |
|---|---|---|---|---|---|---|---|---|---|---|---|---|---|
| | M2 | M3 | M4 | M5 | M6 | M7 | M8 | M9 | M10 | M11 | M12 | M15, M18, | Unscheduled |
| | D60 | D90 | D120 | D150 | D180 | D210 | D240 | D270 | D300 | D330 | D360 | M21, M24 | and Final FU |
| Procedure | ±7 D | ±7 D | ±7 D | ±7 D | ±7 D | ±7 D | ±7 D | ±10 D | ±10 D | ±10 D | ±10 D | ±14 D | visit^{e} |
| Weight^{d} | X | X | X | X | X | X | X | X | X | X | X | | X |
| Clinical | X | X | X | X | X | X | X | X | X | X | X | Quarterly | X |
| examination^{e} | | | | | | | | | | | | | |
| Neurologic exam | As clinically indicated | | | | | | | | | | | | X |
| Vital signs | As clinically indicated | | | | | | | | | | | | X |
| eGFR | X | X | | | X | | | X | | | X | Quarterly | X |
| Chest X-ray (or | X | X | | | X | | | X | | | X | Quarterly | X |
| Chest CT for AAV | | | | | | | | | | | | | |
| subjects only or as | | | | | | | | | | | | | |
| clinically | | | | | | | | | | | | | |
| indicated)^{f} | | | | | | | | | | | | | |
| Urinalysis for spot | X | X | | | X | | | X | | | X | Quarterly | X |
| UPCR | | | | | | | | | | | | | |
| Telemetry cardiac | At the onset of Grade 2 CRS until resolution to Grade ≤ 1, and | | | | | | | | | | | | X |
| monitoring | additionally as clinically indicated | | | | | | | | | | | | |
| 12-lead ECG | As clinically indicated | | | | | | | | | | | | |
| SAE assessment^{g} | Continuously | | | | | | | | | | | Quarterly | X |
| AE assessment | Continuously | | | | | | | | | | | Quarterly | X |
| Concomitant | Continuously | | | | | | | | | | | | X |
| medications | | | | | | | | | | | | | |
| CBC, chemistry, | X | X | X | X | X | X | X | X | X | X | X | Quarterly | X |
| coagulation, | | | | | | | | | | | | | |
| urinalysis test | | | | | | | | | | | | | |
| LDH/CRP/Ferritin | X | X | | | X | | | X | | | X | | X |
| Virus tests | As clinically indicated | | | | | | | | | | | | X |
| (COVID-19, HIV, | | | | | | | | | | | | | |
| HBV, HCV, | | | | | | | | | | | | | |
| HTLV, RSV) | | | | | | | | | | | | | |
| CK/biomarker | See CK/Biomarker table (Table ) | | | | | | | | | | | | |
| assessment | | | | | | | | | | | | | |
| Pregnancy test^{h} | X | X | X | X | X | X | X | X | X | X | X | | X |

Efficacy assessment for all subjects (Cohorts 1-3)

| | | | | | | | |
|---|---|---|---|---|---|---|---|
| SF36 v2 | X | X | X | X | X | Quarterly | X |

| | | | | | | | |
|---|---|---|---|---|---|---|---|
| PGA | X | X | X | X | X | Quarterly | X |

Cohort 1 and 2-specific assessments (subjects with LN and ERL)

| | | | | | | | |
|---|---|---|---|---|---|---|---|
| SLEDAI-2K | X | X | X | X | X | Quarterly | X |
| LupusQOL | X | X | X | X | X | Quarterly | X |
| SLICC/ACR | X | X | X | X | X | Quarterly | X |
| Damage Index | | | | | | | |

Cohort 3-specific assessments (subjects with AAV only)

| | | | | | | | |
|---|---|---|---|---|---|---|---|
| BVAS | X | X | X | X | X | Quarterly | X |
| VDI | X | X | X | X | X | Quarterly | X |
| AAV-PRO | X | X | X | X | X | Quarterly | X |

Abbreviations: AAV-PRO, anti-neutrophil cytoplasmic antibody (ANCA)-associated vasculitis-patient reported outcome; AE, adverse event; BVASv3, Birmingham Vasculitis Activity
Score; CBC, complete blood count; CK, cellular kinetics; CRP, C-reactiv eprotein; CT, computed tomography; D, day(s); DLT, dose-limiting toxicity; ECG, electrocardiogram;
ECHO, echocardiogram; eGFR, estimated glomerular filtration rate; HBV, hepatitis B virus; HCV, hepatitis C virus; HIV, human immunodeficiency virus; hr, hour; HTLV, Human
T-lymphotropic virus 1; LD, lymphodepleting; LDH, lactate dehydrogenase; LTFU, long-term follow-up; LN, lupus nephritis; MN, membranous nephropathy; M, month; MUGA,
multigated acquisition; PGA, Physician Global Assessment; PRO, patient-reported outcome; QOL, Quality of Life; q6hr, every 6 hours; q12hr, every 12 hours; RSV, respiratory
syncytial virus; SAE, serious adverse event; SF36, 36-Item Short Form Survey; SLEDAI-2K, Systemic Lupus Erythematosus Disease Activity Index 2000; UPCR, urine protein
creatinine ratio; VDI, Vasculitis Damage Index.
Footnotes:
^{a}Assessments to be performed during the first week of the indicated months after Day 0 (e.g., M2 = one calendar month after Day 0 of treatment). Monthly assessments have a +/-7 day
time window (relative to the monthly anniversary of Day 0). Assessments to be performed for up to 12 months following hypoimmune allogeneic CD19-directed T cell treatment (or until
withdrawal of consent and/or start of new systemic therapy).
^{b}Assessments to be performed during Year 2 follow-up. ″Quarterly″ refers to assessments that should be performed every 3 months (i.e., in M15, M18, M21, and M24 relative to Day 0).
^{c}Assessments to be performed at the final follow-up visit (e.g., M24 visit or prior to start of new systemic therapy, withdrawal of consent, or other circumstances prior to M24).
^{d}Height to be measured at screening only; weight to be measured only at the indicated scheduled visits.
^{e}After the Day 28 clinic visit, clinical examination beginning with Month 2 may occur as clinic visit or home health visit (per investigator discretion). Home health visits may occur during the
M4, M5, M7, M8, M10, M11, M15, and M21 visits only (investigators may use telemedicine in conjunction with home health visits).
^{f}Chest X-ray performed for all subjects at Screening and as specified in SOA for subjects with AAV. A chest x-ray may be performed as clinically indicated for any subject during the study.
^{g}During LTFU, TRAEs will be assessed quarterly (or until subject begins next new systemic treatment).
^{h}For women of childbearing potential, pregnancy tests will be performed at Screening and monthly thereafter that coincide with clinic visits.

TABLE 24
Cellular Kinetics/Biomarker Schedule of Assessmentsª

| | | | Hypoimmune | | | | | | | | | | | | | | |
|---|---|---|---|---|---|---|---|---|---|---|---|---|---|---|---|---|---|
| | | | Allogeneic | | | | | | | | | | | | | | |
| | | | CD19- | | | | | | | | | | | | | | |
| | | | Directed T | | | | | | | | | | | | | | |
| | | LD | cell | | | | | | | | | | | | | | 2 |
| | | chemo- | treatment | | 1 Year FU | | | | | | | | | | | | Year |
| | Screening | therapy | DO | | | | | | | | | | | | | M2 | FU |
| | Up to 21 | −5 to −3 | Pre- | Post- | | | | | | | D10 | D13 | D16 | D21 | D28 | −12 | Year |
| Assessment | days | Days | dose | dose^{a} | D1 | D2 | D3 | D4 | D5 | D6 | ±1D | ±1D | ±1D | ±4D | ±4D | ±7D | 2 |
| VCN | | X | | X | X | | X | | | X | X | X | X | X | X | M1, | X |
| (CAR-TPK) | | | | | | | | | | | | | | | | 2, | |
| | | | | | | | | | | | | | | | | 3, | |
| | | | | | | | | | | | | | | | | 4,5 | |
| | | | | | | | | | | | | | | | | 6, | |
| | | | | | | | | | | | | | | | | 9, | |
| | | | | | | | | | | | | | | | | 12 | |
| CAR-T | | X | | X | X | | X | | | X | X | X | X | X | X | M2, | X |
| enumeration/CD4 | | | | | | | | | | | | | | | | 3, | |
| 7 expression | | | | | | | | | | | | | | | | 6, | |
| (flow cytometry) | | | | | | | | | | | | | | | | 12 | |
| Cytokine | X | X | X | X | X | | X | | | X | X | X | | X | X | M2, | X |
| profiling/soluble | | | | | | | | | | | | | | | | 3, | |
| markers of | | | | | | | | | | | | | | | | 6. | |
| inflammation | | | | | | | | | | | | | | | | 12 | |
| RCL | | X | | | | | | | | | | | | | X | M2, | X |
| | | | | | | | | | | | | | | | | 3, | |
| | | | | | | | | | | | | | | | | 6, | |
| | | | | | | | | | | | | | | | | 12 | |
| T cell clonality | | | | | | | | | | | | X | | | X | | |
| (clonal | | | | | | | | | | | | | | | | | |
| expansion) | | | | | | | | | | | | | | | | | |
| Immunopheno- | X | X | X | | X | | | | | X | | X | | X | X | M2, | X |
| typing (flow | | | | | | | | | | | | | | | | 3, | |
| cytometry)^{b} | | | | | | | | | | | | | | | | 6, | |
| | | | | | | | | | | | | | | | | 12 | |
| Immunogenicity | | | X | | X | | | | | X | | X | | X | X | M2, | X |
| assessments^{c} | | | | | | | | | | | | | | | | 3, | |
| | | | | | | | | | | | | | | | | 6, | |
| | | | | | | | | | | | | | | | | 12 | |
| Autoantibody | X | | X | | X | | | | | X | | | | X | X | M2, | X |
| profile | | | | | | | | | | | | | | | | 3, | |
| | | | | | | | | | | | | | | | | 6, | |
| | | | | | | | | | | | | | | | | 12 | |
| Complement | X | | X | | X | | | | | X | | | | X | X | M2, | X |
| profile | | | | | | | | | | | | | | | | 3, | |
| | | | | | | | | | | | | | | | | 6, | |
| | | | | | | | | | | | | | | | | 12 | |
| Quantitative | X | | X | | X | | | | | X | | | | X | X | M2, | X |
| Immunoglobulins | | | | | | | | | | | | | | | | 3, | |
| | | | | | | | | | | | | | | | | 6, | |
| | | | | | | | | | | | | | | | | 12 | |
| Protective | X | | X | | X | | | | | X | | | | X | X | M2, | X |
| antibody profilo^{d} | | | | | | | | | | | | | | | | 3, | |
| | | | | | | | | | | | | | | | | 6, | |
| | | | | | | | | | | | | | | | | 12 | |
| RNA sequencing | X | | X | | X | | | | | X | | | | X | X | M2, | X |
| | | | | | | | | | | | | | | | | 3, | |
| | | | | | | | | | | | | | | | | 6, | |
| | | | | | | | | | | | | | | | | 12 | |
| Urine biomarkers | X | | | | | | | | | | | | | | X | M2, | X |
| | | | | | | | | | | | | | | | | 3, | |
| | | | | | | | | | | | | | | | | 6, | |
| | | | | | | | | | | | | | | | | 12 | |

Abbreviations:
APRIL, A proliferation-inducing ligand; BAFF, B-cell activating factor; CAR T, chimeric antigen receptor-T cell; CD, cluster of differentiation; D, day; FU, follow-up; LD, lymphodepleting; M, month; PK, pharmacokinetics; RCL, replication competent lentivirus; RNA, ribonucleic acid; VCN, vector copy number.
Footnotes:
^{a}Assessments to be performed at the end of the indicated months after Day 0 (e.g., M1 = approximately 30 days after Day 0 and M2 = approximately 60 days after Day 0). Monthly assessments have a ±7-day time window (relative to the monthly anniversary of Day 0).
^{b}Includes B and T cell subsets impacted by CAR-T cell therapy and CD47 expression
^{c}Humoral and cellular immunogenicity will be assessed.
^{d}Protective antibodies include assessments of anti-tetanus, anti-pneumococcal and other antibodies induced by prior immunization.

### Certain Embodiments I

1. A method of treating a subject having or suspected of having a B cell malignancy or a B cell mediated disorder, the method comprising:
administering to the subject a composition comprising a population of engineered hypoimmunogenic T cells, wherein the hypoimmunogenic T cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules,
(b) increase expression of CD47 encoded by a first exogenous polynucleotide, wherein the increased expression of (b) is relative to a control or wild-type T cell that does not comprise the modification, and
(c) express a CD19-specific chimeric antigen receptor (CAR) encoded by a second exogenous polynucleotide.

2. The method of embodiment 1, wherein the one or more modifications reduce expression of the one or more MHC class I molecules, the one or more MHC class II molecules, and/or the one or more TCR molecules in the hypoimmunogenic T cells, relative to control or wild-type T cells.

3. The method of embodiment 1 or embodiment 2, wherein expression of the one or more MHC class I molecules, the one or more MHC class II molecules, and the one or more TCR molecules is reduced in the hypoimmunogenic T cells relative to the control or wild-type T cell.

4. The method of any one of embodiments 1-3, wherein the control or wild-type T cells do not comprise the modifications.

5. The method of any one of embodiments 1-4, wherein the one or more modifications reduce cell surface expression of the one or more MHC class I molecules relative to the control or wild-type T cell.

6. The method of any one of embodiments 1-5, wherein the one or more modifications reduce cell surface trafficking of the one or more MHC class I molecules relative to the control or wild-type T cell.

7. The method of any one of embodiments 1-6, wherein the one or more modifications reduce a function of the one or more MHC class I molecules.

8. The method of any one of embodiments 1-7, wherein the one or more MHC class I molecules is one or more human leukocyte antigen (HLA) class I molecules.

9. The method of embodiment 8, wherein the one or more HLA class I molecules is HLA-A, HLA-B, HLA-C, or a combination thereof.

10. The method of any one of embodiments 1-9, wherein the one or more molecules that regulate cell surface expression of the one or more MHC class I molecules, regulate cell surface trafficking of the one or more MHC class I molecules, and/or reduce a function of the one or more MHC class I molecules are beta-2 microglobulin (B2M).

11. The method of any one of embodiments 1-10, wherein the one or more modifications comprise a modification that inactivates or disrupts one or more alleles of B2M.

12. The method of embodiment 11, wherein the modification that inactivates or disrupts one or more alleles of B2M reduces mRNA expression of the B2M gene.

13. The method of embodiment 11 or embodiment 12, wherein the modification that inactivates or disrupts one or more alleles of B2M reduces protein expression of B2M.

14. The method of any one of embodiments 11-13, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the hypoimmunogenic T cell.

15. The method of any one of embodiments 11-14, wherein the modification that inactivates or disrupts one or more alleles of B2M comprises an indel in the B2M gene.

16. The method of any one of embodiments 11-15, wherein the modification that inactivates or disrupts one or more alleles of B2M comprises a frameshift mutation or a deletion of a contiguous stretch of genomic DNA of the B2M gene.

17. The method of any one of embodiments 1-16, wherein the one or more modifications reduce cell surface expression of the one or more MHC class II molecules relative to the control or wild-type cell.

18. The method of any one of embodiments 1-17, wherein the one or more modifications reduce cell surface trafficking of the one or more MHC class II molecules relative to the control or wild-type cell.

19. The method of any one of embodiments 1-18, wherein the one or more modifications reduce a function of the one or more MHC class II molecules.

20. The method of embodiment 19, wherein the function of the one more MHC class II molecules is antigen presentation.

21. The method of any one of embodiments 1-20, wherein the one or more MHC class II molecules is one or more human leukocyte antigen (HLA) class II molecules.

22. The method of embodiment 21, wherein the one or more HLA class II molecules is HLA-DP, HLA-DQ, HLA-DR, or a combination thereof.

23. The method of any one of embodiments 1-22, wherein the one or more molecules that regulate expression of the one or more MHC class II molecules are Class II Major Histocompatibility Complex Transactivator (CIITA).

24. The method of any one of embodiments 1-23, wherein the one or more modifications comprise a modification that inactivates or disrupts one or more alleles of CIITA.

25. The method of embodiment 24, wherein the modification that inactivates or disrupts one or more alleles of CIITA reduces mRNA expression of the CIITA gene.

26. The method of embodiment 24 or embodiment 25, wherein the modification that inactivates or disrupts one or more alleles of CIITA reduces protein expression of CIITA.

27. The method of any one of embodiments 24-26, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the hypoimmunogenic T cell.

28. The method of any one of embodiments 24-27, wherein the modification that inactivates or disrupts one or more alleles of CIITA comprises an indel in the CIITA gene.

29. The method of any one of embodiments 24-28, wherein the modification that inactivates or disrupts one or more alleles of CIITA comprises a frameshift mutation or a deletion of a contiguous stretch of genomic DNA of the CIITA gene.

30. The method of any one of embodiments 1-29, wherein expression of HLA-A, HLA-B, HLA-C, HLA-DP, HLA-DQ, and HLA-DR are reduced in the hypoimmunogenic T cell.

31. The method of any of embodiments 1-30, wherein the one or more modifications reduce cell surface protein expression of the one or more TCR molecules relative to the control or wild-type cell.

32. The method of any of embodiments 1-31, wherein the one or more modifications reduce cell surface trafficking of the one or more TCR molecules relative to the control or wild-type cell.

33. The method of any of embodiments 1-32, wherein the one or more modifications reduce a function of the one or more TCR molecules.

34. The method of embodiment 33, wherein the function of the one or more TCR molecules is antigen recognition.

35. The method of any of embodiments 1-34, wherein the one or more TCR molecules is T cell receptor alpha constant (TRAC), T cell receptor beta constant (TRBC), or a combination thereof.

36. The method of any of embodiments 1-35, wherein the one or more TCR molecules comprise TRAC.

37. The method of any of embodiments 1-36, wherein the one or more modifications comprise a modification that inactivates or disrupts one or more alleles of TRAC.

38. The method of embodiment 37, wherein the modification that inactivates or disrupts one or more alleles of TRAC reduces mRNA expression of the TRAC gene.

39. The method of embodiment 37 or 38, wherein the modification that inactivates or disrupts one or more alleles of TRAC reduces protein expression of TRAC.

40. The method of any of embodiments 37-39, wherein the modification that inactivates or disrupts one or more alleles of TRAC comprises:
inactivation or disruption of one allele of the TRAC gene;
inactivation or disruption of both alleles of the TRAC gene; or
inactivation or disruption of all TRAC coding alleles in the hypoimmunogenic T cell.

41. The method of any of embodiments 37-40, wherein the modification inactivation or disruption that inactivates or disrupts one or more alleles of TRAC comprises an indel in the TRAC gene.

42. The method of any of embodiments 37-41, wherein the inactivation or disruption comprises a frameshift mutation or a deletion of a contiguous stretch of genomic DNA of the TRAC gene.

43. The method of any one of embodiments 1-42, wherein the one or more modifications increase expression of one or more tolerogenic factors encoded by a third exogenous polynucleotide, wherein the increased expression of the one or more tolerogenic factors is relative to a control or wild-type T cell, and wherein the one or more tolerogenic factors comprise CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or a combination thereof.

44. A method of treating a subject having or suspected of having a B cell malignancy or a B cell mediated disorder, the method comprising:
administering to the subject a composition comprising a population of engineered hypoimmunogenic T cells comprising,
(a) reduced expression of beta-2 microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide, wherein the increased expression of (b) is relative to the control or wild-type T cells, and
(c) expression of a CD19-specific chimeric antigen receptor (CAR) encoded by a second exogenous polynucleotide.

45. The method of any one of embodiments 1-44, wherein the composition is administered to the subject at a dosage of about 5×10⁶ cells to about 400×10⁶ cells.

46. The method of any one of embodiments 1-45, wherein the composition is administered to the subject at a dosage of about 20×10⁶ cells to about 400×10⁶ cells.

47. The method of any one of the embodiments 1-46, wherein the composition is administered to the subject at a dosage of at least about 30×10⁶ cells, at least about 60×10⁶ cells, at least about 90×10⁶ cells, at least about 120×10⁶ cells, or at least about 200×10⁶ cells.

48. The method of any one of the embodiments 1-46, wherein the composition is administered to the subject at a dosage of at least about 30×10⁶ cells.

49. The method of any one of the embodiments 1-46, wherein the composition is administered to the subject at a dosage of at least about 60×10⁶ cells.

50. The method of any one of the embodiments 1-46, wherein the composition is administered to the subject at a dosage of at least about 90×10⁶ cells.

51. The method of any one of the embodiments 1-46, wherein the composition is administered to the subject at a dosage of at least about 120×10⁶ cells.

52. The method of any one of the embodiments 1-46, wherein the composition is administered to the subject at a dosage of at least about 200×10⁶ cells.

53. The method of any one of embodiments 1-46, wherein the composition is administered to the subject at a dosage of no more than about 200×10⁶ cells.

54. The method of any one of embodiments 1-46, wherein the composition is administered to the subject at a dosage of no more than about 400×10⁶ cells.

55. The method of any one of embodiments 1-54, wherein the composition is administered to the subject at a dosage of about 5×10⁶ CAR+ cells to about 400×10⁶ CAR+ cells.

56. The method of any one of embodiments 1-55, wherein the composition is administered to the subject at a dosage of about 20×10⁶ CAR+ cells to about 400×10⁶ CAR+ cells.

57. The method of any one of embodiments 1-56, wherein the composition is administered to the subject at a dosage of at least about 30×10⁶ CAR+ cells, at least about 60×10⁶ CAR+ cells, at least about 90×10⁶ CAR+ cells, at least about 120×10⁶ CAR+ cells, or at least about 200×10⁶ CAR+ cells.

58. The method of any one of the embodiments 1-46, wherein the composition is administered to the subject at a dosage of at least about 30×10⁶ CAR+ cells.

59. The method of any one of the embodiments 1-46, wherein the composition is administered to the subject at a dosage of at least about 60×10⁶ CAR+ cells.

60. The method of any one of the embodiments 1-46, wherein the composition is administered to the subject at a dosage of at least about 90×10⁶ CAR+ cells.

61. The method of any one of the embodiments 1-46, wherein the composition is administered to the subject at a dosage of at least about 120×10⁶ CAR+ cells.

62. The method of any one of the embodiments 1-46, wherein the composition is administered to the subject at a dosage of at least about 200×10⁶ CAR+ cells.

63. The method of any one of embodiments 1-56, wherein the composition is administered to the subject at a dosage of no more than about 200×10⁶ CAR+ cells.

64. The method of any one of embodiments 1-56, wherein the composition is administered to the subject at a dosage of no more than about 400×10⁶ CAR+ cells.

65. The method of any one of embodiments 1-64, wherein a total volume of about 1.25 mL to about 100 mL of the composition is administered.

66. The method of any one of embodiments 1-65, wherein a total volume of about 5 mL to about 100 mL of the composition is administered.

67. The method of any one of embodiments 1-66, wherein a total volume of at least about 7.5 mL, at least about 15 mL, at least about 30 mL, or at least about 50 mL of the composition is administered.

68. The method of any one of embodiments 1-67, wherein the composition comprises a concentration of engineered hypoimmunogenic T cells of at least 1.25×10⁶ cells/mL.

69. The method of any one of embodiments 1-68, wherein the composition comprises a concentration of engineered hypoimmunogenic T cells of at least 4.0×10⁶ cells/mL.

70. The method of any one of embodiments 1-69, wherein the composition comprises a concentration of engineered hypoimmunogenic T cells of at least 1.2×10⁶ CAR+ cells/mL.

71. The method of any one of embodiments 1-70, wherein the composition comprises a concentration of engineered hypoimmunogenic T cells of at least 4.0×10⁶ CAR+ cells/mL.

72. The method of any one of embodiments 1-71, wherein the method comprises administering the composition in at least one dose, at least two doses, at least three doses, at least four doses, at least five doses, at least six doses, at least seven doses, at least eight doses, at least ten doses, at least eleven doses, or at least twelve doses.

73. The method of any one of embodiments 1-72, wherein the method comprises administering the composition in a single dose.

74. The method of any one of embodiments 1-72, wherein the method comprises administering the composition in two doses.

75. The method of any one of embodiments 1-74, wherein the engineered hypoimmunogenic T cells comprise CD8+ and/or CD4+ T cells.

76. The method of any one of embodiments 1-75, wherein the engineered hypoimmunogenic T cells comprise allogeneic T cells.

77. The method of any one of embodiments 1-76, wherein the engineered hypoimmunogenic T cells comprise primary T cells.

78. The method of any one of embodiments 1-77, wherein the engineered hypoimmunogenic T cells comprise donor-derived T cells.

79. The method of embodiment 78, wherein the donor-derived T cells are derived from 1 donor, 2 donors, 3 donors, 4 donors, 5 donors, 6 donors, 7 donors, 8 donors, 9 donors, or 10 donors.

80. The method of any one of embodiments 1-77, wherein the engineered hypoimmunogenic T cells comprise stem cell-derived T cells.

81. The method of any one of embodiments 1-80, wherein the CAR comprises an anti-CD19 antigen binding domain, a CD8α hinge domain, a CD8α transmembrane domain, a 4-1BB costimulatory domain, and a CD3ζ intracellular signaling domain.

82. The method of embodiment 81, wherein the antigen binding domain comprises:
(i) a light chain amino acid sequence comprising a light chain CDR1, a light chain CDR2, and a light chain CDR3,
wherein the light chain CDR1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and
(ii) a heavy chain amino acid sequence comprising a heavy chain CDR1, a heavy chain CDR2, and a heavy chain CDR3 amino acid,
wherein the heavy chain CDR 1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28.

83. The method of embodiment 82, wherein the light chain amino acid sequence comprises a light chain variable region and the heavy chain amino acid sequence comprises a heavy chain variable region,
wherein the light chain variable region comprises an amino acid sequence at least 90% identical to SEQ ID NO: 20, and
wherein the heavy chain variable region comprises an amino acid sequence comprises an amino acid sequence at least 90% identical to SEQ ID NO: 25.

84. The method of any one of embodiments 81-83, wherein the CD8α hinge domain comprises an amino acid sequence at least 90% identical to SEQ ID NO: 9, the CD8α transmembrane domain comprises an amino acid sequence at least 90% identical to SEQ ID NO: 14, the 4-1BB costimulatory domain comprises an amino acid sequence at least 90% identical to SEQ ID NO: 16, and the CD3 intracellular signaling domain comprises an amino acid sequence at least 90% identical to SEQ ID NO: 18.

85. The method of any one of embodiments 81-84, wherein the antigen binding domain comprises a linker comprising the amino acid sequence at least 90% identical to SEQ ID NO: 24.

86. The method of any one of embodiments 81-85, wherein the antigen binding domain comprises a linker comprising the amino acid sequence of SEQ ID NO: 24.

87. The method of any one of embodiments 76-86 wherein the antigen binding domain comprises an amino acid sequence at least 90% identical to SEQ ID NO: 19.

88. The method of any one of embodiments 76-87, wherein the antigen binding domain comprises the amino acid sequence of SEQ ID NO: 19.

89. The method of any one of embodiments 1-88, wherein the engineered hypoimmunogenic T cells are B2Mindel/indel, CIITAindel/indel, and/or TRACindel/indel cells.

90. The method of embodiment 89, wherein the population of engineered hypoimmunogenic T cells is a heterogeneous population comprising B2Mindel/indel cells, CIITAindel/indel cells, TRACindel/indel cells, B2Mindel/indel/CIITAindel/indel cells, B2Mindel/indel/TRACindel/indel cells, CIITAindel/indel/TRACindel/indel cells, and/or B2Mindel/indel/CIITAindel/indel/TRACindel/indel cells.

91. The method of any one of embodiments 1-90, wherein the composition is administered intravenously.

92. The method of any one of embodiments 1-90, wherein the composition is administered to the nervous system of the subject.

93. The method of embodiment 92, wherein the nervous system is the central nervous system (CNS).

94. The method of embodiment 92, wherein the nervous system is the peripheral nervous system (PNS).

95. The method of any one of embodiments 1-94, wherein the composition is administered intrathecally.

96. The method of any one of embodiments 1-91, wherein the B cell malignancy is a lymphoma or a leukemia.

97. The method of any one of embodiments 1-96, wherein the B cell malignancy is selected from the group consisting of: Non-Hodgkin's Lymphoma (NHL), Chronic Lymphocytic Leukemia (CLL), large B cell lymphoma (LBCL), transplant ineligible large B cell lymphoma (LBCL), diffuse LBCL (DLBCL), high-grade B cell lymphoma (HGBCL), primary mediastinal B cell lymphoma (PMBCL), mantle cell lymphoma (MCL), follicular lymphoma (FL), marginal zone lymphoma (MZL), or small lymphocytic lymphoma (SLL).

98. The method of any one of embodiments 1-97, wherein the subject is diagnosed to have NHL or CLL.

99. The method of embodiment 98, wherein the subject is diagnosed to have NHL based on the World Health Organization 2016 criteria.

100. The method of embodiment 99, wherein the subject is confirmed to have NHL based on histological analysis of a biopsy sample collected from the subject.

101. The method of embodiment 99 or embodiment 100, wherein the subject has:
a single lesion measuring greater than 7.5 cm in diameter; or
a sum of the product of lymph node diameter is greater than 5000 mm³.

102. The method of embodiment 98, wherein the subject is diagnosed to have CLL based on the International Workshop on CLL (iwCLL) criteria.

103. The method of embodiment 102, wherein the subject has:
a lymph node greater than or equal to 10 cm in diameter; or
a lymph node greater than or equal to 5 cm in diameter and an absolute lymphocyte count greater than or equal to 25×109 lymphocytes/L.

104. The method of embodiment 98, wherein the subject is diagnosed as being a high risk CCL patient.

105. The method of embodiment 98 or 102, wherein method further comprises:
detecting one or more biomarkers, and
identifying the subject as being a high risk CCL patient based on the one or more biomarkers.

106. The method of embodiment 105, wherein the one or more biomarkers are selected from the group consisting of: del(17p), mutated TP53, unmutated immunoglobulin heavy-chain variable (IGHV) gene, and complex karyotype.

107. The method of any one of embodiments 1-106, wherein the subject previously received an alkylating agent, a purine nucleoside, chemoimmunotherapy, a Bruton's tyrosine kinase (BTK) inhibitor, a PI3K inhibitor, a B cell lymphoma 2 (BCL2) inhibitor, a CD20 monoclonal antibody, a bispecific monoclonal antibody, or a combination thereof.

108. The method of embodiment 107, wherein the alkylating agent comprises chlorambucil or cyclophosphamide.

109. The method of embodiment 107, wherein the purine nucleoside is selected from the group consisting of: fludarabine, pentostatin, and cladribine.

110. The method of embodiment 107, wherein the chemoimmunotherapy is selected from the group consisting of: FCR (fludarabine, cyclophosphamide, and rituximab), BR (bendamustine and rituximab), alemtuzumab, and lenalidomide.

111. The method of embodiment 107, wherein the BTK inhibitor is selected from the group consisting of: ibrutinib, acalabrutinib, zanubrutinib, and pirtobrutinib.

112. The method of embodiment 107, wherein the PI3K inhibitor comprises idelalisib or duvelisi.

113. The method of embodiment 107, wherein the BCL2 inhibitor comprises venetoclax.

114. The method of embodiment 107, wherein the CD20 monoclonal antibody comprises obinutuzumab.

115. The method of embodiment 107, wherein the bispecific monoclonal antibody is selected from the group consisting of Mosunetuzumab (CD20×CD3 mAb), Glofitamab (CD20⁽²⁾×CD3), Odronextamab (CD20×CD3 mAb), and Epcoritamab (CD20×CD3 mAb).

116. The method of embodiment 98, wherein the subject is diagnosed to have CLL and is further administered a BTK inhibitor.

117. The method of embodiment 116, wherein the BTK inhibitor is selected from the group consisting of: ibrutinib, acalabrutinib, zanubrutinib, and pirtobrutinib.

118. The method of embodiment 98, wherein the subject is diagnosed to have CLL and is administered the composition comprising the population of engineered hypoimmunogenic T cells as a first-line therapy.

119. The method of any one of embodiments 1-117, wherein the B cell malignancy or the B cell mediated disorder is relapsed or refractory following at least one therapeutic standard of care regimen.

120. The method of any one of embodiments 1-117, wherein the B cell malignancy or the B cell mediated disorder is relapsed or refractory following at least two therapeutic standard of care regimens.

121. The method of any one of embodiments 1-117, wherein the B cell malignancy or the B cell mediated disorder is relapsed or refractory following at least three therapeutic standard of care regimens.

122. The method of any one of embodiments 1-107, wherein the B cell malignancy or the B cell mediated disorder is relapsed or refractory following autologous stem cell transplant (ASCT).

123. The method of any one of embodiments 119-122, wherein the subject:
did not respond to a first line therapy;
had progressive B cell malignancy or B cell mediated disorder as a best response to a last administered therapeutic regimen; or
had stable disease as a best response to the last administered therapeutic regimen.

124. The method of embodiment 122, wherein the subject:
had disease progression or relapse within twelve months of ASCT; or
the subject had no response to or relapsed following administration of a last line of therapy.

125. The method of any one of embodiments 119-124, wherein the B cell mediated disorder is relapsed or refractory following a first line therapy comprising mycophenolate mofetil (MMF), cyclophosphamide, or a combination thereof.

126. The method of any one of embodiments 119-125, wherein the B cell mediated disorder is relapsed or refractory following a second line therapy comprising rituximab, belimumab, voclosporin, obinutuzumab, or a combination thereof.

127. The method of any one of embodiments 119-124, wherein the B cell mediated disorder is relapsed or refractory following at least two disease-modifying therapies selected from the group consisting of: MMF, azathioprine, cyclophosphamide, methotrexate, rituximab, belimumab, intravenous immunoglobulin (IVIg), and abatacept.

128. The method of any one of embodiments 119-126, wherein the subject does not achieve a complete renal response to the second line therapy within 6 months of initiation of the second line therapy.

129. The method of any one of embodiments 119-128, wherein the subject has disease activity despite maintenance on maximally tolerated doses of drugs that block the renin-angiotensin system (RAS).

130. The method of any one of embodiments 1-124, wherein the subject has adequate hepatic function, renal function, bone marrow function, cardiac function, and/or pulmonary function.

131. The method of any one of embodiments 1-97, wherein the B cell malignancy is relapsed and/or refractory.

132. The method of embodiment 131, wherein the B cell malignancy is relapsed and/or refractory NHL.

133. The method of embodiment 131, wherein the B cell malignancy is relapsed and/or refractory CLL.

134. The method of any one of embodiments 131-133, wherein the B cell malignancy is relapsed and/or refractory after at least 2 regimens of therapy.

135. The method of any one of embodiments 131-134, wherein the B cell malignancy is relapsed and/or refractory after at least 3 regimens of therapy.

136. The method of any one of embodiments 131-135, wherein the B cell malignancy is relapsed and/or refractory after autologous stem cell transplant (ASCT).

137. The method of any one of embodiments 131-136, wherein the B cell malignancy is relapsed and/or refractory, wherein relapsed and/or refractory comprises one or more of: no response to first line therapy, progressive disease as best response to most recent therapy regimen, stable disease as best response to most recent therapy with duration no longer than 6 months from last dose of therapy, disease progression or relapse within 12 months of ASCT, no response to or relapse after last line of therapy post-ASCT.

138. The method of any one of embodiments 1-91, wherein the B cell mediated disorder is an autoimmune condition.

139. The method of embodiment 138, wherein the autoimmune condition is selected from the group consisting of: systemic lupus erythematosus (SLE), extrarenal systemic lupus erythematosus (ERL), lupus nephritis (LN), CNS lupus, vasculitis, granulomatosis vasculitis, polyangiitis & microscopic polyangiitis vasculitis, anti-neutrophilic cytoplasmic autoantibody (ANCA) associated vasculitis (AAV), granulomatous polyangiitis, microscopic polyangiitis, multiple sclerosis (relapsing and/or progressive), pemphigus vulgaris, autoimmune blistering skin diseases, membranous nephropathy (MN), anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, autoimmune hemolytic anemia, amyloidosis, scleroderma, idiopathic inflammatory myositis, and immune-mediated necrotizing myopathy.

140. The method of embodiment 138 or embodiment 139, wherein the subject has:
a moderate to severe autoimmune condition;
a relapsed or refractory autoimmune condition; and/or
the presence of autoantibodies.

141. The method of any one of embodiments 1-140, wherein the subject is diagnosed to have systemic lupus erythematosus (SLE), extrarenal systemic lupus erythematosus (ERL), lupus nephritis (LN), ANCA-associated vasculitis (AAV), or membranous nephropathy (MN).

142. The method of any one of embodiments 1-141, wherein the subject has adequate organ function.

143. The method of any one of embodiments 1-142, wherein the subject has adequate hepatic function.

144. The method of embodiment 143, wherein adequate hepatic function comprises:
a total bilirubin level <1.5× upper limit of normal (ULN); and/or
alanine aminotransferase (ALT) or aspartate aminotransferase (AST)<3×ULN.

145. The method of embodiment 144, wherein the subject has disease-related muscle involvement causing elevated ALT or AST.

146. The method of embodiment 143, wherein adequate hepatic function comprises:
a total bilirubin level <3× upper limit of normal (ULN) and/or
alanine aminotransferase (ALT) or aspartate aminotransferase (AST)<5×ULN;
wherein the subject has liver involvement; and
wherein the total bilirubin level <3×ULN is not attributed to Gilbert's syndrome.

147. The method of any one of embodiments 1-144, wherein the subject has adequate bone marrow function.

148. The method of embodiment 147, wherein adequate bone marrow function comprises a hemoglobin level ≥8 g/da neutrophil count ≥1000/mm³ and/or a platelet count ≥50,000/mm³.

149. The method of any one of embodiments 1-148, wherein the subject has adequate pulmonary function.

150. The method of embodiment 149, wherein adequate pulmonary function comprises:
a baseline oxygen saturation >92% on room air;
no clinically significant pleural effusion; and/or
a forced expiratory volume in the first second/Forced Vital Capacity (FEV1/FVC)>70% as measured by spirometry.

151. The method of any one of embodiments 1-150, wherein the subject is eligible for lymphodepleting chemotherapy.

152. The method of embodiment 141, wherein the subject is diagnosed to have SLE based on 2019 EULAR/ACR Classification Criteria.

153. The method of embodiment 141, wherein the subject is confirmed to have Class III (A or A/C) or IV (A or A/C) LN based on histological analysis using ISN/RPS 2018 criteria with active or active/chronic lupus nephritis performed within 6 months of screening.

154. The method of embodiment 141, wherein the subject is confirmed to have Class V LN.

155. The method of embodiment 153, wherein the subject is further confirmed to have Class III or Class IV LN.

156. The method of any one of embodiments 1-155, wherein the subject has at least one elevated autoantibody.

157. The method of embodiment 156, wherein the at least one elevated autoantibody comprises anti-dsDNA, anti-nuclear autoantibody (ANA) or anti-Sm (Smith) autoantibody.

158. The method of embodiment 157, wherein the ANA comprises a titer of at least 1:80.

159. The method of embodiment 156, wherein the at least one elevated autoantibody comprises anit-PLA2R or anti-THSD7A.

160. The method of any one of embodiments 1-159, wherein the subject has a prothrombin time-international normalized ratio ≤2.5 with no disease-related coagulopathy.

161. The method of any one of embodiments 1-160, wherein the subject has an activated partial thromboplastin time ≤2.5×ULN.

162. The method of any one of embodiments 1-161, wherein the subject has an estimated GFR of ≥45 mL/min/1.73m2 as determined by a CKD-EPI equation.

163. The method of any one of embodiments 1-162, wherein the subject has proteinuria with a UPCR determined by cither spot urine collection or 24-hour urine collection of >1 g/dL and ≤7 g/dL.

164. The method of any one of embodiments 1-163, wherein the subject has a SLEDAI-2K score ≥8.

165. The method of embodiment 141, wherein the subject is diagnosed to have granulomatous with polyangiitis (GPA) or microscopic polyangiitis (MPA) based on the Chapel Hill Consensus Conference criteria.

166. The method of embodiment 141 or 165, wherein the subject is diagnosed with a disease flare with a BVAS >2 following the achievement of remission induced by a regimen consisting of anti-CD20 B cell depletion (e.g., rituximab or obinutuzumab) and glucocorticoids; cyclophosphamide and glucocorticoids; or anti-CD20 B cell depletion, cyclophosphamide and glucocorticoids.

167. The method of embodiment 166, wherein the disease flare is organ or life threatening.

168. The method of embodiment 166 or 167, wherein the subject has had at least one GPA or MPA flare, at least one prior induction regimen with rituximab, obinutuzumab or cyclophosphamide and a baseline BVAS of ≥1.

169. The method of any one of embodiments 165-168, wherein the subject has severe disease or a history of severe disease as defined by one or more major BVAS items.

170. The method of any one of embodiments 165-169, wherein the subject is positive for either PR3-ANCA or MPO-ANCA.

171. The method of any one of embodiments 1-170, wherein the subject is refractory to rituximab or has relapsed after prior rituximab therapy with evidence of reduced eGFR.

172. The method of any one of embodiments 1-171, wherein the subject has not received a prior CD19-directed therapy.

173. The method of any one of embodiments 1-171, wherein the subject has received a prior CD19-directed therapy.

174. The method of embodiment 172 or embodiment 173, wherein the prior CD19-directed therapy is or comprises a CD19-directed CAR T cell treatment.

175. The method of embodiment 172 or embodiment 173, wherein the prior CD19-directed therapy is or comprises a CD19-directed antibody treatment.

176. The method of embodiment 172 or embodiment 173, wherein the prior CD19-directed therapy is or comprises a CD19-directed CAR natural killer (NK) cell treatment.

177. The method of any one of embodiments 1-176, wherein the subject has received a prior bispecific antibody therapy.

178. The method of embodiment 177, wherein the bispecific antibody therapy is selected from the group consisting of: Mosunetuzumab (CD20×CD3 mAb), Glofitamab (CD20⁽²⁾×CD3), Odronextamab (CD20×CD3 mAb), and Epcoritamab (CD20×CD3 mAb).

179. The method of any one of embodiments 1-178, wherein the subject has not received a CD47-directed treatment.

180. The method of any one of embodiments 1-178, wherein the subject has received a CD47-directed treatment but has reduced levels of anti-CD47 antibodies.

181. The method of any one of embodiments 1-178, wherein the subject has received a CD47-directed treatment but has no detectable levels of anti-CD47 antibodies.

182. The method of any one of embodiments 1-178, wherein the subject received a CD47-directed treatment a period of time before administration of the composition, that period of time being a duration associated with reduced anti-CD47 antibody levels.

183. The method of any one of embodiments 1-178, wherein the method further comprises screening the subject for anti-CD47 antibody levels.

184. The method of any one of embodiments 1-178, wherein the subject does not have a history of primary central nervous system (CNS) lymphoma or CNS metastases.

185. The method of embodiment 184, wherein the absence of primary CNS lymphoma or CNS metastases is based on a brain magnetic resonance imaging (MRI) performed within 3 months prior to administering the composition to the subject.

186. The method of embodiment 184, wherein the absence of CNS lymphoma or CNS metastases is based on a lumbar puncture performed within 1 month prior to administering the composition to the subject.

187. The method of any one of embodiments 1-186, wherein the subject does not have a history of Richter's transformation of chronic leukemic lymphoma, small lymphocytic lymphoma, or lymphoplasmacytic lymphoma.

188. The method of any one of embodiments 1-187, wherein the subject docs not have a fungal, bacterial, viral, or other infection that is uncontrolled or requiring intravenous (IV) antimicrobials for management.

189. The method of any one of embodiments 1-187, wherein the subject has not been hospitalized for treatment of an infection within 28 days of baseline of >2 times within 1 year of administering the composition to the subject.

190. The method of any one of embodiments 1-188, wherein the subject does not have acute symptoms of COVID-19 infection.

191. The method of any one of embodiments 1-190, wherein the subject docs not have a known history of infection with HIV, hepatitis B virus, and/or hepatitis C virus.

192. The method of any one of embodiments 1-191, wherein the subject does not have an active autoimmune disease and/or any other disease requiring immunosuppressive therapy or corticosteroid therapy.

193. The method of embodiment 192, wherein the subject has not received greater than 10 mg/day of prednisone or equivalent dose of another corticosteroid.

194. The method of any one of embodiments 1-193, wherein the subject does not have a primary immunodeficiency or a history of a concomitant genetic syndrome associated with bone marrow failure.

195. The method of embodiment 194, wherein the primary immunodeficiency or the concomitant genetic syndrome associated with bone marrow failure comprises Fanconi anemia, Kostmann syndrome, or Shwachman-Diamond syndrome.

196. The method of any one of embodiments 1-195, wherein the subject has not been treated with a systemic cancer therapy within 14 days or five half-lives of administration of the composition.

197. The method of any one of embodiments 1-196, wherein the subject has not been treated with a systemic cancer therapy comprising a biologic therapy within 28 days of administration of the composition.

198. The method of any one of embodiments 1-197, wherein the subject has not been treated with any investigational product within 28 days of administration of the composition.

199. The method of any one of embodiments 1-198, wherein the subject has not been treated with radiotherapy within 14 days of administration of the composition.

200. The method of any one of embodiments 1-199, wherein the subject has not been treated with major surgery within 28 days of administration of the composition.

201. The method of any one of embodiments 1-200, wherein the subject has not been treated with an autologous hematopoietic stem cell transplant (HSCT) within six weeks of administration of the composition.

202. The method of any one of embodiments 1-201, wherein the subject has not been treated with an allogeneic HSCT at any point prior to administration of the composition.

203. The method of any one of embodiments 1-202, wherein the subject has not had a cardiac condition within one year of administration of the composition.

204. The method of embodiment 203, wherein the cardiac condition comprises myocardial infarction, cardiac angioplasty or stenting, cardiac arrhythmia requiring medication, unstable angina, New York Heart Association Class II or greater congestive heart failure, cardiac atrial or ventricular lymphoma involvement, or a clinically significant cardiac disease.

205. The method of any one of embodiments 1-202, wherein the subject has not had an ejection fraction ≤50%, arrythmias and any structural heart disease (including clinically significant pericardial effusion), as assessed by echocardiogram or multiple gated acquisition scan performed within 1 month of administration of the composition.

206. The method of any one of embodiments 1-204, wherein the subject does not have a history of thromboembolic disease.

207. The method of embodiment 206, wherein the subject does not have a history of non-line associated, clinically significant deep-vein thrombosis, proximal deep vein thrombosis, or pulmonary embolism requiring therapeutic anticoagulation within 6 months of administration of the composition.

208. The method of any one of embodiments 1-207, wherein the subject does not have a history or presence of a CNS disorder within 12 months of administration of the composition.

209. The method of embodiment 208, wherein the CNS disorder comprises a seizure disorder, a cerebrovascular ischemia/hemorrhage, a dementia, a cerebellar disease, or any autoimmune disease with CNS involvement.

210. The method of any one of embodiments 1-209, wherein the subject does not have clinically significant ascites.

211. The method of embodiment 210, wherein clinically significant ascites comprises physically positive ascites or ascites requiring puncture intervention or medication intervention for control.

212. The method of any one of embodiments 1-211, wherein the subject does not have a history of organ transplantation and/or is not awaiting organ transplantation.

213. The method of any one of embodiments 1-212, wherein the subject does not have an indwelling line or drain.

214. The method of embodiment 213, wherein the indwelling line or drain comprises a percutaneous nephrostomy tube, an indwelling Foley catheter, a biliary drain, a G/J-tube, a pleural catheter, a peritoneal catheter, or a pericardial catheter.

215. The method of any one of embodiments 1-214, wherein the subject has not received a live vaccine within 6 weeks of administration of the composition.

216. The method of any one of embodiments 1-215, wherein the subject does not have an unresolved toxicity greater than Grade 1 from a previous anticancer therapy.

217. The method of any one of embodiments 1-216, wherein the subject does not have a history of additional malignancy other than nonmelanoma skin cancer or carcinoma in situ.

218. The method of any one of embodiments 1-217, wherein the subject does not have a history of additional malignancy, or has been disease-free from the additional malignancy for at least two years prior to administration of the composition.

219. The method of any one of embodiments 1-218, wherein the subject does not have a severe immediate hypersensitivity to any of the components of the composition.

220. The method of any one of embodiments 1-219, wherein the subject does not have an admission or evidence of illicit drug use, drug abuse, or alcohol abuse.

221. The method of any one of embodiments 1-220, wherein the subject does not exhibit clinical evidence of significant unstable or uncontrolled acute or chronic disease not due to the B cell mediated disorder.

222. The method of embodiment 221, wherein the significant unstable or uncontrolled acute or chronic disease comprises cardiovascular, pulmonary, hematologic, gastrointestinal, hepatic, renal, neurological, malignancy, or infectious disease.

223. The method of any one of embodiments 1-222, wherein the subject does not have a history of an anaphylactic reaction to parenteral administration of contrast agents, human or murine proteins, or monoclonal antibodies.

224. The method of any one of embodiments 1-223, wherein the subject does not have a history of cancer, or the subject has been disease-free from a basal carcinoma of the skin, cervix, bladder, prostate or breast for at least five years prior to administration of the composition.

225. The method of embodiment 224, wherein the cancer comprises a hematologic malignancy, a solid tumor, or a carcinoma in situ.

226. The method of any one of embodiments 1-225, wherein the subject is administered physiologic replacement glucocorticoids, topical glucocorticoids, or inhaled glucocorticoids.

227. The method of any one of embodiments 1-226, wherein the subject is not administered glucocorticoids above physiologic replacement.

228. The method of embodiment 226 or embodiment 227, wherein physiologic replacement glucocorticoids comprises 5 mg prednisone daily or equivalent.

229. The method of any one of embodiments 1-225, wherein the subject has not received treatment with Leflunomide within 28 prior to administration of the composition.

230. The method of any one of embodiments 1-229, wherein the subject does not have an immunoglobulin A (IgA) deficiency (IgA level <10 mg/dL).

231. The method of any one of embodiments 1-230, wherein the subject does not have a grade≥3 laboratory abnormality, except those associated with the B cell mediated disorder.

232. The method of any one of embodiments 1-231, wherein the subject does not have a B cell mediated disorder related flare that needs immediate treatment.

233. The method of any one of embodiments 1-232, wherein the subject does not have a CNS lupus manifestation, a history of a CNS disorder, or a presence of a CNS disorder.

234. The method of embodiment 233, wherein the CNS disorder is or comprises primary seizure disorder, cerebrovascular ischemia/hemorrhage, dementia, or cerebellar disease.

235. The method of any one of embodiments 1-234, wherein the subject has not been diagnosed with anti-phospholipid antibody syndrome that is actively treated.

236. The method of any one of embodiments 1-235, wherein the subject has not been diagnosed with EGPA as defined by the Chapel Hill Consensus Conference criteria.

237. The method of any one of embodiments 1-236, wherein the subject does not have an alveolar hemorrhage within 4 weeks prior to administration of the composition.

238. The method of any one of embodiments 1-237, wherein the subject has not been diagnosed with a cerebrovascular accident caused by vasculitis or pachymeningitis.

239. The method of any one of embodiments 1-238, wherein the subject is 18-85 years of age.

240. The method of any one of embodiments 1-239, wherein the subject is 18-80 years of age.

241. The method of any one of embodiments 1-240, wherein the subject is 18-75 years of age.

242. The method of any one of embodiments 1-220, wherein the method further comprises:
administering to the subject a lymphodepleting chemotherapy regimen,
wherein the step of administering the lymphodepleting chemotherapy regimen occurs prior to the step of administering the composition.

243. The method of embodiment 242, wherein the composition is administered 2-7 days after completion of the lymphodepleting chemotherapy regimen.

244. The method of embodiment 242 or embodiment 243, wherein the composition is administered 3 days after completion of the lymphodepleting chemotherapy regimen.

245. The method of any one of embodiments 242-244, wherein the lymphodepleting chemotherapy is or comprises cyclophosphamide and fludarabine.

246. The method of embodiment 245, wherein the cyclophosphamide is administered at a dose of about 100 mg/m².

247. The method of embodiment 245, wherein the cyclophosphamide is administered at a dose of about 200 mg/m2.

248. The method of embodiment 245, wherein the cyclophosphamide is administered at a dose of about 250 mg/m2.

249. The method of embodiment 245, wherein the cyclophosphamide is administered at a dose of about 300 mg/m2.

250. The method of embodiment 245, wherein the cyclophosphamide is administered at a dose of about 400 mg/m2.

251. The method of embodiment 245, wherein the cyclophosphamide is administered at a dose of about 500 mg/m2.

252. The method of embodiment 245, wherein the cyclophosphamide is administered at a dose of about 1000 mg/m2.

253. The method of embodiment 245 or embodiment 251, wherein the cyclophosphamide is administered over approximately 60 minutes.

254. The method of any one of embodiments 245-253, wherein the cyclophosphamide is administered daily for 3 days.

255. The method of embodiment 254, wherein the cyclophosphamide is administered to the subject 5 days, 4 days, and 3 days prior to the administration of the composition.

256. The method of any one of embodiments 245-253, wherein the cyclophosphamide is administered to the subject once, 3 days prior to the administration of the composition.

257. The method of any one of embodiments 245-255, wherein one liter of 0.9% sodium chloride is administered within 24 hours prior to administration of cyclophosphamide.

258. The method of any one of embodiments 245-257, wherein the fludarabine is administered at a dose of about 10 mg/m².

259. The method of any one of embodiments 245-257, wherein the fludarabine is administered at a dose of about 15 mg/m².

260. The method of any one of embodiments 245-257, wherein the fludarabine is administered at a dose of about 20 mg/m².

261. The method of any one of embodiments 245-257, wherein the fludarabine is administered at a dose of about 25 mg/m².

262. The method of any one of embodiments 245-257, wherein the fludarabine is administered at a dose of about 30 mg/m².

263. The method of any one of embodiments 245-257, wherein the subject has moderate impairment of renal function, and wherein the fludarabine is administered at a dose of about 24 mg/m2 with monitoring.

264. The method of any one of embodiments 245-257, wherein the subject has a moderate impairment of renal function, and wherein the fludarabine is administered at a dose of about 20 mg/m² with monitoring.

265. The method of embodiment 262, wherein the subject has a creatinine clearance of about 50-70 mL/min/1.73 m².

266. The method of any one of embodiments 245-265, wherein the fludarabine is administered over approximately 30 minutes.

267. The method of any one of embodiments 245-266, wherein the fludarabine is administered daily for 3 days.

268. The method of embodiment 267, wherein the fludarabine is administered to the subject 5 days, 4 days, and 3 days prior to the administration of the composition.

269. The method of any one of embodiments 245-268, wherein one liter of 0.9% sodium chloride is administered at the completion of administration of fludarabine.

270. The method of any one of embodiments 245-269, wherein the cyclophosphamide and fludarabine are administered concurrently.

271. The method of any one of embodiments 242-270, wherein the lymphodepleting chemotherapy regimen is administered intravenously.

272. The method of any one of embodiments 242-271, wherein the lymphodepleting chemotherapy regimen further comprises administration of sodium 2-mercaptoethanesulfonate.

273. The method of any one of embodiments 1-272, wherein the subject has not received a live vaccine within 28 days of administration of the lymphodepleting chemotherapy.

274. The method of any one of embodiments 1-273, wherein the subject has not received an immunosuppressant therapy within a time period before the start of lymphodepleting chemotherapy,
wherein the time period comprises the duration of 5 half-lives of the immunosuppressant therapy, and
wherein the immunosuppressant therapy does not comprise corticosteroids used in screening.

275. The method of embodiment 274, wherein the immunosuppressant therapy comprises MMF, cyclophosphamide, voclosporin, azathioprine, rituximab, belimumab, obinutuzumab, or a combination thereof.

276. The method of embodiment 274 or 275, wherein
(i) the immunosuppressant therapy comprises MMF and the duration of 5 half-lives comprises 5 days,
(ii) the immunosuppressant therapy comprises cyclophosphamide and the duration of 5 half-lives comprises 3 days,
(iii) the immunosuppressant therapy comprises voclosporin and the duration of 5 half-lives comprises 7 days,
(iv) the immunosuppressant therapy comprises azathioprine and the duration of 5 half-lives comprises 1 day,
(v) the immunosuppressant therapy comprises rituximab and the duration of 5 half-lives comprises 95 days,
(vi) the immunosuppressant therapy comprises belimumab and the duration of 5 half-lives comprises 97 days, or
(vii) immunosuppressant therapy comprises obinutuzumab and the duration of 5 half-lives comprises 142 days.

277. The method of any one of embodiments 1-272, wherein the method does not comprise administering to the subject one or more corticosteroids for at least 5 days prior to administration of the composition.

278. The method of embodiment 277, wherein the one or more corticosteroids comprise greater than or equal to 5 mg/day of prednisone or an equivalent dose of another corticosteroid.

279. The method of any one of embodiments 1-277, wherein the subject is weaned to discontinuation of a severe autoimmune therapy over the course of no more than 21 days prior to administering the composition to the subject.

280. The method of embodiment 279, wherein the subject is weaned to 0 mg glucocorticoids.

281. The method of embodiment 279 or embodiment 280, wherein the subject is weaned to a physiologic equivalent range of ≤5 mg/day of prednisone.

282. The method of embodiment 277 or embodiment 278, wherein the subject has computed tomography (CT) scans with contrast that are contraindicating for administration of systemic corticosteroids.

283. The method of embodiment 282, wherein the subject undergoes MRI with contrast and noncontrast CT scans during the five days prior to administration of the composition.

284. The method of any one of embodiments 1-283, wherein the method does not comprise administering to the subject one or more corticosteroids or other immunosuppressive drugs for at least three months after administration of the composition.

285. The method of any one of embodiments 1-283, wherein the method comprises administering to the subject one or more corticosteroids or other immunosuppressive drugs after administration of the composition for management of composition-related toxicities.

286. The method of any one of embodiments 1-283, wherein the method does not comprise administering to the subject one or more corticosteroids, other immunosuppressive drugs, or other immunomodulatory medication at least one month prior to administration of the composition and at least two years after administration of the composition unless medically necessary.

287. The method of embodiments 286, wherein the immunomodulatory medication comprises non-steroidal anti-inflammatory agents.

288. The method of any one of embodiments 1-220 or 277-287, wherein the method does not comprise administering to the subject a lymphodepleting chemotherapy regimen prior to administration of the composition.

289. The method of any one of embodiments 1-288, wherein the method further comprises:
administering to the subject a premedication therapy regimen,
wherein the step of administering the premedication therapy regimen occurs prior to the step of administering the composition.

290. The method of embodiment 289, wherein the premedication therapy is or comprises acetaminophen (paracetamol) and/or antihistamine.

291. The method of embodiment 290, wherein the acetaminophen (paracetamol) is administered to the subject at a dosage of about 500 mg to about 1000 mg.

292. The method of embodiment 290, wherein the antihistamine comprises diphenhydramine and/or famotidine.

293. The method of any one of embodiments 289-292, wherein the premedication therapy regimen occurs about 1 hour prior to the step of administering the composition.

294. The method of embodiment 289 or embodiment 290, wherein the premedication therapy does not comprise corticosteroids, anakinra, or tocilizumab.

295. The method of any one of embodiments 1-294, wherein the method further comprises:
administering to the subject a post-treatment therapy regimen,
wherein the step of administering the post-treatment therapy regimen occurs after the step of administering the composition.

296. The method of embodiment 295, wherein the post-treatment therapy is or comprises tocilizumab.

297. The method of any one of embodiments 1-296, wherein the method further comprises:
administering to the subject: methotrexate, 6-MP, azathioprine sulphasalazine, mesalazine, olsalazine chloroquinine/hydroxychloroquine, pencillamine, aurothiomalate, azathioprine, colchicine, a corticosteroid (e.g., prednisolone), a beta-2 adrenoreceptor agonist (e.g., salbutamol, terbutaline, salmeteral), a xanthine (e.g., theophylline, aminophylline), cromoglycate, nedocromil, ketotifen, ipratropium and oxitropium, cyclosporin, FK506, rapamycin, mycophenolate mofetil, leflunomide, an NSAID (e.g., ibuprofen), a phosphodiesterase inhibitor, an adenosine agonist, an antithrombotic agent, a complement inhibitor, an adrenergic agent, an agent that interferes proinflammatory cytokine (e.g., TNF-α, IL-1) signaling (e.g., IRAK, NIK, IKK, p38 or MAP kinase inhibitors), an IL-1 β converting enzyme inhibitor, a TNF-α converting enzyme (TACE) inhibitor, a T-cell signaling inhibitor (e.g., a kinase inhibitor, a metalloproteinase inhibitor, sulfasalazine, azathioprine, 6-mercaptopurine, an angiotensin converting enzyme inhibitor), a soluble cytokine receptor and derivatives thereof (e.g., soluble p55 or p75 TNF receptors and the derivatives P75TNFRIgG (Enbrel™ and p55TNFRIgG (Lenercept)), sIL-IRI, SIL-IRII, sIL-6R), an anti-inflammatory cytokine (e.g., IL-4, IL-10, IL-11, IL-13, TGFP), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, CDC-801, Mesopram, or combinations thereof.

298. The method of any one of embodiments 1-296, further comprising:
collecting a sample from the subject after administration of the composition and determining:
   CAR T enumeration,
   CAR T cellular kinetics,
   CD47 expression,
   presence of replication competent lentivirus,
   T cell clonality,
   immunophenotype, and/or
   a cytokine profile.

299. The method of embodiment 298, wherein the method further comprises modulating a subsequent administration of the composition, a post-treatment therapy, and/or one or more additional anti-cancer therapies based on the CAR T enumeration, CAR T cellular kinetics, CD47 expression, presence of replication competent lentivirus, T cell clonality, immunophenotype, and/or cytokine profile.

300. The method of any one of embodiments 1-299, further comprising:
collecting a sample from the subject after administration of the composition and determining the presence of a:
   hypoimmunogenic T cell-mediated NK response,
   hypoimmunogenic T cell-mediated T response,
   hypoimmunogenic T cell-mediated humoral response, and/or
   humoral immunogenic response.

301. The method of embodiment 300, wherein the method further comprises modulating a subsequent administration of the composition, a post-treatment therapy, and/or one or more additional anti-cancer therapies based on the presence of the hypoimmunogenic T cell-mediated NK response, hypoimmunogenic T cell-mediated T response, hypoimmunogenic T cell-mediated humoral response, and/or humoral immunogenic response.

302. The method of any one of embodiments 1-301, further comprising:
collecting a sample from the subject after administration of the composition and performing:
single cell RNA sequencing,
a minimal residual disease (MRD) assay
a circulating tumor DNA (ctDNA) assay, and/or
bulk RNA sequencing.

303. The method of embodiment 302, wherein the method further comprises modulating a subsequent administration of the composition, a post-treatment therapy, and/or one or more additional anti-cancer therapies based on the single cell RNA sequencing, the MRD assay, the ctDNA assay, or the bulk RNA sequencing.

304. The method of any one of embodiments 1-303, further comprising:
collecting a sample from the subject after administration of the composition and determining the presence of cellular and/or humoral immune response directed towards one or more CD19-specific CAR peptides.

305. The method of embodiment 304, wherein the cellular and/or humoral immune response is determined using ELISPOT or immunohistochemistry analysis.

306. The method of embodiment 304 or embodiment 305, wherein the method further comprises modulating a subsequent administration of the composition, a post-treatment therapy, and/or one or more additional anti-cancer therapies based on the presence of cellular and/or humoral immune response.

307. The method of any one of embodiments 1-306, further comprising:
(i) obtaining first sequencing data for a first plurality of DNA molecules,
(ii) determining, based on the first sequencing data, a first cancer profile,
(iii) obtaining second sequencing data for a second plurality of DNA molecules,
(iv) determining, based on the second sequencing data, a second cancer profile, and
(v) detecting, based on a comparison of the first cancer profile and the second cancer profile a cancer status of the subject.

308. The method of embodiment 307, wherein the cancer status is a minimal residual disease (MRD) status.

309. The method of embodiment 307 or embodiment 308, wherein the MRD status is based on identification of cancer specific markers in the first cancer profile and identification of the same cancer specific markers in the second cancer profile.

310. The method of any of embodiments 307-309, wherein the identification of cancer specific markers in the first cancer profile, the second cancer profile, or both, comprises comparing the markers in the first cancer profile, the second cancer profile, or both to a reference cancer profile.

311. The method of embodiment 307, wherein the cancer status is a circulating tumor DNA (ctDNA) status.

312. The method of embodiment 307 or embodiment 311, wherein the ctDNA status is based on identification of cancer specific markers in the first cancer profile and identification of the same cancer specific markers in the second cancer profile.

313. The method of any one of embodiments 307, 311 and 312, wherein the identification of cancer specific markers in the first cancer profile, the second cancer profile, or both, comprises comparing the markers in the first cancer profile, the second cancer profile, or both to a reference cancer profile.

314. The method of any one of embodiments 307-313, wherein the method further comprises modulating a subsequent administration of the composition, a post-treatment therapy, and/or one or more additional anti-cancer therapies based in part on a change in the cancer status of the subject.

315. The method of any one of embodiments 1-306, further comprising:
(i) obtaining first sequencing data for a first plurality of DNA molecules,
(ii) determining, based on the first sequencing data, a first autoimmune disorder profile,
(iii) obtaining second sequencing data for a second plurality of DNA molecules,
(iv) determining, based on the second sequencing data, a second autoimmune disorder profile, and
(v) detecting, based on a comparison of the first autoimmune disorder profile and the second autoimmune disorder profile an autoimmune disorder status of the subject.

316. The method of embodiment 315, wherein the autoimmune disorder status is based on identification of autoimmune disorder specific markers in the first autoimmune disorder profile and identification of the same autoimmune disorder specific markers in the second autoimmune disorder profile.

317. The method of embodiment 315 or embodiment 316, wherein the identification of autoimmune disorder specific markers in the first autoimmune disorder profile, the second autoimmune disorder profile, or both, comprises comparing the markers in the first autoimmune disorder profile, the second autoimmune disorder profile, or both to a reference autoimmune disorder profile.

318. The method of embodiment 315, wherein the identification of autoimmune disorder specific markers in the first autoimmune disorder profile, the second autoimmune disorder profile, or both, comprises comparing the markers in the first autoimmune disorder profile, the second autoimmune disorder profile, or both to a reference autoimmune disorder profile.

319. The method of any one of embodiments 315-318, wherein the method further comprises modulating a subsequent administration of the composition, a post-treatment therapy, and/or one or more additional anti-autoimmune disorder therapies based in part on a change in the autoimmune disorder status of the subject.

320. The method of any one of embodiments 1-314, further comprising:
(i) obtaining first sequencing data for a first plurality of DNA molecules,
(ii) determining, based on the first sequencing data, a first B-cell/T-cell profile for the patient,
(iii) obtaining second sequencing data for a second plurality of DNA molecules,
(iv) determining, based on the second sequencing data, a second B-cell/T-cell profile for the patient,
(v) detecting, based on a comparison of the first B-cell/T-cell profile and the second B-cell/T-cell profile a change in the B-cell/T-cell status for the patient.

321. The method of embodiment 320, wherein the B-cell/T-cell status is the identity of B-cells and T-cells in the subject.

322. The method of embodiment 320 or embodiment 321, wherein the detection of the identity of the B-cells and T-cells in the subject is based on detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile and detection of the same rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the second B-cell/T-cell profile.

323. The method of any of embodiment 322, wherein the detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both, comprises comparing the detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both to a reference.

324. The method of any of embodiments 320-323, wherein the B-cell/T-cell status is the quantity of B-cells and T-cells in the subject.

325. The method of any of embodiments 320-324, wherein the detection of the quantity of the B-cells and T-cells in the subject is based on detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile and detection of the same rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the second B-cell/T-cell profile.

326. The method of embodiment 325, wherein the detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both, comprises comparing the detection of rearranged IgH (VDJ) receptor gene sequences, rearranged IgH (DJ) receptor gene sequences, rearranged IgK receptor gene sequences, rearranged IgL receptor gene sequences, translocated BCL1/IgH (J) receptor gene sequences, and/or translocated BCL2/IgH (J) receptor gene sequences in the first B-cell/T-cell profile, the second B-cell/T-cell profile, or both to a reference.

327. The method of any of embodiments 320-326, wherein the method further comprises modulating a subsequent administration of the composition, a post-treatment therapy, and/or one or more additional anti-cancer therapies based in part on a change in the subject's status, the identity of B-cells and T-cells in the subject, the quantity of B-cells and T-cells in the subject, or a combination thereof.

328. The method of any one of embodiments 1-327, further comprising:
(i) obtaining a first sample from the subject before administration of the composition;
(ii) obtaining a first set of sequencing data for a first plurality of DNA molecules obtained from the first sample;
(iii) determining a first cancer profile based on the first set of sequencing data;
(iv) obtaining a second sample from the subject after administration of the composition;
(v) obtaining a second set of sequencing data for a second plurality of DNA molecules obtained from the second sample;
(vi) determining a second cancer profile based on the second set of sequencing data; and
(vii) comparing the first cancer profile and the second cancer profile to determine a positive minimal residual disease (MRD) status or a negative MRD status in the subject.

329. The method of any one of embodiments 1-328, further comprising:
(i) obtaining a first sample from the subject before administration of the composition;
(ii) obtaining a first set of sequencing data for a first plurality of DNA molecules obtained from the first sample;
(iii) determining a first B cell receptor (BCR)/TCR profile based on the first set of sequencing data;
(iv) obtaining a second sample from the subject after administration of the composition;
(v) obtaining a second set of sequencing data for a second plurality of DNA molecules obtained from the second sample;
(vi) determining a second BCR/TCR profile based on the second set of sequencing data; and
(vii) comparing the first BCR/TCR profile and the second BCR/TCR profile to determine a positive MRD status or a negative MRD status in the subject.

330. The method of any one of embodiments 1-329, further comprising:
(i) obtaining a first sample from the subject before administration of the composition;
(ii) obtaining a first set of sequencing data for a first plurality of DNA molecules obtained from the first sample;
(iii) determining a first B cell/T cell profile based on the first set of sequencing data;
(iv) obtaining a second sample from the subject after administration of the composition;
(v) obtaining a second set of sequencing data for a second plurality of DNA molecules obtained from the second sample;
(vi) determining a second B cell/T cell profile based on the second set of sequencing data; and
(vii) comparing the first B cell/T cell profile and the second B Cell/T cell profile to determine a positive MRD status or negative MRD status in the subject.

331. The method of any one of embodiments 1-330, further comprising:
(i) obtaining a first sample from the subject before administration of the composition;
(ii) obtaining a first set of sequencing data for a first plurality of DNA molecules obtained from the first sample;
(iii) determining a first B cell/T cell quantity based on the first set of sequencing data;
(iv) obtaining a second sample from the subject after administration of the composition;
(v) obtaining a second set of sequencing data for a second plurality of DNA molecules obtained from the second sample;
(vi) determining a second B cell/T cell quantity based on the second set of sequencing data; and
(vii) comparing the first B cell/T cell quantity and the second B Cell/T cell quantity to determine a positive MRD status or negative MRD status in the subject.

332. The method of any one of embodiments 328-331, wherein if the subject is determined to have a positive MRD status, the subject is subsequently administered one or more additional anti-cancer therapies.

333. The method of embodiment 332, wherein the one or more additional anti-cancer therapies comprise a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

334. The method of any one of embodiments 1-333, further comprising:
collecting a sample from the subject after administration of the composition and determining disease progression.

335. The method of embodiment 334, wherein disease progression is determined by an increased diameter measurement of one or more malignant lesions as visualized via radiography.

336. The method of embodiment 334 or embodiment 335, wherein the subject is administered one or more additional anti-cancer therapies in the presence of disease progression.

337. The method of embodiment 336, wherein the one or more additional anti-cancer therapies comprise a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

338. The method of any one of embodiments 1-327, further comprising:
collecting a sample from the subject after administration of the composition and performing:
   single cell RNA sequencing,
   an autoantibody assay,
   a complement assay,
   a B cell activation assay, and/or
   a urine biomarker assay.

339. The method of embodiment 338, wherein the autoantibody assay comprises determining levels of: ANA (Hep2), anti-dsDNA, anti-Sm, anti-RNP, anti-Ro52, anti-Ro60, anti-La, anti-histone, anti-cardiolipin IgG, IgM, IgA, anti-B2GPI IgG, IgM, IgA, ANCA, anti-PR3, anti-MPO, or combinations thereof.

340. The method of embodiment 338, wherein the complement assay comprises determining levels of C3, C4, CH50, or combinations thereof.

341. The method of any one of embodiments 1-337, wherein the engineered hypoimmunogenic T cells elicit reduced immune recognition when administered to the subject relative to the control or wild-type cells.

342. The method of any one of embodiments 1-341, wherein the engineered hypoimmunogenic T cells elicit a reduced adaptive immune response when administered to the subject relative to the control or wild-type cells.

343. The method of any one of embodiments 1-342, wherein the engineered hypoimmunogenic T cells elicit a reduced T cell response when administered to the subject relative to the control or wild-type cells.

344. The method of any one of embodiments 1-343, wherein the engineered hypoimmunogenic T cells elicit a reduced innate immune response when administered to the subject relative to the control or wild-type cells.

345. The method of any one of embodiments 1-344, wherein the engineered hypoimmunogenic T cells elicit a reduced natural killer (NK) cell response when administered to the subject relative to the control or wild-type cells.

346. The method of any one of embodiments 1-345, wherein the engineered hypoimmunogenic T cells elicit a reduced macrophage response when administered to the subject relative to the control or wild-type cells.

347. The method of any one of embodiments 1-346, wherein the engineered hypoimmunogenic T cells elicit a reduced graft-versus-host reaction (GvHR) when administered to the subject relative to the control or wild-type cells.

348. The method of any one of embodiments 1-347, wherein the engineered hypoimmunogenic T cells persist in the subject for a longer duration relative to the control or wild-type cells.

349. The method of any one of embodiments 1-348, wherein the engineered hypoimmunogenic T cells have greater expansion in the subject relative to the control or wild-type cells.

350. The method of any one of embodiments 1-348, wherein the engineered hypoimmunogenic T cells elicit a depletion of B cells and/or plasmablasts.

351. The method of any one of embodiments 1-348, wherein the engineered hypoimmunogenic T cells increase depletion of B cells and/or plasmablasts, wherein the depletion is increased relative to a CD19-specific antibody therapy.

352. The method of any one of embodiments 1-349, wherein the subject has an improved:
(i) objective response;
(ii) duration of response;
(iii) time to next treatment;
(iv) progression-free survival;
(v) event-free survival;
(vi) overall survival;
(vii) MRD negativity; or
combination thereof,
relative to an untreated subject and/or a subject treated with a standard of care therapy.

353. The method of embodiment 352, wherein the event-free survival duration is at least about 6, 7, 8, 9, 10, 11, 12, 13, 14, 15, 16, 17, 18, 19, 20, 21, 22, 23, or 24 months.

354. The method of embodiment 353, wherein the event-free survival duration is about 10 months.

355. The method of embodiment 352 wherein the progression-free survival duration is at least about 6, 7, 8, 9, 10, 11, 12, 13, 14, 15, 16, 17, 18, 19, 20, 21, 22, 23, or 24 months.

356. The method of embodiment 355, wherein the progression-free survival duration is about 15 months.

357. The method of embodiment 352, wherein the CRR is about 30%, 35%, 40%, 45%, 50%, 55%, 60%, 65%, 70%, 75%, 80%, 85%, 90%, 95%, 97%, 98%, 99%, or 100%.

358. The method of embodiment 357, wherein the CRR is about 65%.

359. The method of embodiment 357, wherein the CRR is about 50%.

360. The method of embodiment 357, wherein the CRR is about 30%.

361. The method of embodiment 352, wherein the durable CRR is at least about 20%, 25%, 30%, 35%, 40%, 45%, 50%, 55%, 60%, 65%, 70%, 75%, 80%, 85%, 90%, 95%, 97%, 98%, 99%, or 100%.

362. The method of embodiment 361, wherein the durable CRR is about 30%.

363. The method of embodiment 361, wherein the durable CRR is about 40%.

364. The method of embodiment 361, wherein the durable CRR is about 50%.

365. The method of embodiment 361, wherein the durable CRR is about 60%.

366. The method of embodiment 352, wherein the ORR is about 30%, 35%, 40%, 45%, 50%, 55%, 60%, 65%, 70%, 75%, 80%, 85%, 90%, 95%, 97%, 98%, 99%, or 100%.

367. The method of embodiment 366, wherein the ORR is about 70%.

368. The method of embodiment 366, wherein the ORR is about 80%.

369. The method of embodiment 352, wherein the mDOR is at least about 6, 7, 8, 9, 10, 11, 12, 13, 14, 15, 16, 17, 18, 19, 20, 21, 22, 23, or 24 months.

370. The method of embodiment 369, wherein the mDOR is at least about 12 months.

371. The method of embodiment 352, wherein the MRD negativity is about 30%, 35%, 40%, 45%, 50%, 55%, 60%, 65%, 70%, 75%, 80%, 85%, 90%, 95%, 97%, 98%, 99%, or 100%.

372. The method of embodiment 371, wherein the MRD negativity is about 70%.

373. The method of any one of embodiments 352-372, wherein the B cell malignancy is LBCL and the composition comprising a population of engineered hypoimmunogenic T cells is a second-line therapy.

374. The method of any one of embodiments 352-372, wherein the B cell malignancy is transplant-ineligible LBCL and the composition comprising a population of engineered hypoimmunogenic T cells is a second-line therapy.

375. The method of any one of embodiments 352-372, wherein the B cell malignancy is LBCL and the composition comprising a population of engineered hypoimmunogenic T cells is a third-line therapy.

376. The method of any one of embodiments 352-372, wherein the B cell malignancy is CLL and the composition comprising a population of engineered hypoimmunogenic T cells is a third-line therapy.

377. The method of any one of embodiments 1-349, wherein the subject has an improved:
(i) disease activity status;
(ii) renal function status;
(iii) proteinuria status;
(iv) physician global assessment status;
(v) autoantibody profile;
(vi) serum complement status;
(vii) duration of drug free remission;
(viii) time to relapse;
(ix) Systemic Lupus Erythematosus Disease Activity Index (SLEDAI) 2000 (2K) response score;
(x) Birmingham Vasculitis Activity Score version 3 [BVAS v3] status;
or a combination thereof; and/or
(xi) complete renal response;
(xii) partial renal response;
(xiii) remission;
(xiv) modified DORIS remission;
or a combination thereof,
relative to an untreated subject and/or a subject treated with a standard of care therapy.

378. The method of embodiment 377, wherein the complete renal response comprises:
(i) a urine protein creatinine ratio UPCR of ≤0.5, and
(ii) an eGFR of ≥60 mL/min/1.73 m², lack of confirmed decrease from baseline of eGFR of >20%, or no treatment- or disease-related eGFR-associated event.

379. The method of embodiment 377, wherein the partial renal response comprises a ≥50% reduction in UPCR from baseline to a value <1, no confirmed decrease from baseline of eGFR of >20%, or no treatment- or disease-related eGFR-associated event.

380. The method of embodiment 377, wherein the partial renal response comprises a ≥50% reduction in UPCR from baseline to a value <3 if baseline UPCR was ≥3, no confirmed decrease from baseline of eGFR of >20%, or no treatment- or disease-related eGFR-associated event.

381. The method of any one of embodiments 1-352, further comprising:
(i) obtaining a biological sample from the subject; and
(ii) performing an analysis of the biological sample to determine,
   a CD19 expression status,
   a CD20 expression status,
   a CD22 expression status, or
   a combination thereof.

382. The method of any one of embodiments 1-352, further comprising:
(i) obtaining a biological sample from the subject; and
(ii) performing an analysis of the biological sample to determine:
   a donor-specific antibody evaluation status,
   a soluble marker of inflammation status,
   a soluble marker of B cell activation status,
   a serum immunoglobulin status,
   a proteinuria level status,
   a serum creatinine status,
   an estimated glomerular filtration status,
   an autoantibody status, or
   a combination thereof.

383. The method of embodiment 381, wherein the biological sample was collected prior to the step of administering the composition.

384. The method of embodiment 381, wherein the biological sample was collected after the step of administering the composition.

385. The method of any one of embodiments 381-384, wherein a positive
CD19 expression status,
CD20 expression status,
CD22 expression status, or
combination thereof,
   is prognostic of an improved,
(i) objective response;
(ii) duration of response;
(iii) time to next treatment;
(iv) progression-free survival;
(v) event-free survival;
(vi) overall survival; or
combination thereof,
relative to the subject prior to administration of the composition.

386. The method of any one of embodiments 381-384, wherein a positive
donor-specific antibody evaluation status,
soluble marker of inflammation status,
soluble marker of B cell activation status,
serum immunoglobulin status,
proteinuria level status,
serum creatinine status,
estimated glomerular filtration status,
autoantibody status, or
combination thereof,
   is prognostic of an improved,
(i) disease activity status;
(ii) renal function status;
(iii) proteinuria status;
(iv) physician global assessment status;
(v) autoantibody profile;
(vi) serum complement status;
(vii) duration of drug free remission;
(viii) time to relapse;
(ix) complete renal response;
(x) partial renal response;
(xi) Systemic Lupus Erythematosus Disease Activity Index (SLEDAI) 2000 (2K) response score;
(xii) remission;
(xiii) modified DORIS remission;
(xiv) Birmingham Vasculitis Activity Score version 3 [BVAS v3] status; or
combination thereof,
relative to the subject prior to administration of the composition.

387. The method of any one of embodiments 1-385, wherein the subject is a human.

388. The method of any one of embodiments 298-387, wherein the sample is or comprises a blood sample, a bone marrow biopsy sample, a liver biopsy sample, a tissue sample, primary cells, cell supernatants, cell lysates, platelets, serum, plasma, vitreous fluid, lymph fluid, synovial fluid, follicular fluid, seminal fluid, whole blood, plasma, serum, blood-derived cells, urine, cerebro-spinal fluid, saliva, sputum, tears, perspiration, mucus, tumor lysates, tissue extracts (e.g., homogenized tissue), tumor tissue, cellular extracts, or combinations thereof.

389. An article of manufacture comprising a composition of a cell therapy, or one of a plurality of compositions of a cell therapy, comprising T cells expressing an anti-CD 19 chimeric antigen receptor (CAR), and instructions for administering the cell therapy, wherein the instructions specify administering the T cell composition according to the methods of any of embodiments 1-387.

390. A pharmaceutical composition comprising a population of engineered hypoimmunogenic T cells comprising:
(a) reduced expression of beta-2-microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide relative to the control or wild-type T cells, and
(c) expression of a CD19-specific chimeric antigen receptor (CAR) encoded by a second exogenous polynucleotide.

391. The pharmaceutical composition of embodiment 390, further comprising one or more excipients comprising:
a cryopreservation medium;
an isotonic electrolyte solution; or
a combination thereof.

392. The pharmaceutical composition of embodiment 391, further comprising dimethylsulfoxide (DMSO).

393. The pharmaceutical composition of embodiment 391 or embodiment 392, further comprising human serum albumin (HSA).

394. The pharmaceutical composition of any one of embodiments 391-392, wherein the isotonic electrolyte solution comprises:
sodium chloride;
sodium gluconate;
sodium acetate trihydrate;
potassium chloride;
magnesium chloride; or
a combination thereof.

395. The pharmaceutical composition of embodiment 393 or embodiment 394, wherein the pharmaceutical composition comprises a DMSO concentration (v/v) of at least about 1%, at least about 2%, at least about 3%, at least about 4%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%.

396. The pharmaceutical composition of embodiment 393 or embodiment 394, wherein the pharmaceutical composition comprises a DMSO concentration (v/v) of about 7.5%.

397. The pharmaceutical composition of embodiment 393 or embodiment 394, wherein the pharmaceutical composition comprises a HSA concentration (w/v) of at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, or at least about 25%.

398. The pharmaceutical composition of embodiment 393 or embodiment 394, wherein the pharmaceutical composition comprises an HSA concentration (w/v) of about 0.3%.

399. The pharmaceutical composition of embodiment 393 or embodiment 394, wherein the pharmaceutical composition comprises a concentration of 7.5% DMSO (v/v) and 1.2% HSA (w/v).

400. The pharmaceutical composition of embodiment 393 or embodiment 394, wherein the pharmaceutical composition comprises a concentration of 7.5% DMSO (v/v) and 0.3% HSA (w/v).

401. The pharmaceutical composition of any one of embodiments 390-400, wherein the population of engineered hypoimmunogenic T cells makes up at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of the total cells in the pharmaceutical composition.

402. The pharmaceutical composition of any one of embodiments 390-400, wherein the population of hypoimmunogenic T cells make up about 20% to about 40%, about 30% to about 50%, about 40% to about 60%, about 50% to about 70%, about 60% to about 80%, about 70% to about 90%, or about 80% to about 99% of the total cells in the pharmaceutical composition.

403. The pharmaceutical composition of any one of embodiments 390-400, wherein the pharmaceutical composition comprises partially edited CAR+ cells, wherein the partially edited CAR+ cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules, or
(b) increase expression of CD47 encoded by a first exogenous polynucleotide, wherein the increased expression of (b) is relative to a control or wild-type T cell that does not comprise the modification, and express a CD19-specific chimeric antigen receptor (CAR) encoded by a second exogenous polynucleotide.

404. The method of any one of embodiments 1-388, wherein the B cell malignancy is positive for a protein involved in immune checkpoint signaling.

405. The method of embodiment 404, wherein the protein involved in immune checkpoint signaling is or comprises 4-1BB (CD137), 4-1BBL (CD137L), CTLA-4, PD-L1, PD-1, PD-L2, LAG-3, TIM-3, TIGIT, VISTA, B7-H3, B7-H4, BTLA, HVEM, GAL9, KIR, 2B4, CD160 (BY55), or CGEN-15049.

406. The method of embodiment 404 or 405, wherein the composition is administered to the subject at a higher dosage relative to the dosage given to a subject having or suspected of having a B cell malignancy negative for the protein involved in immune checkpoint signaling.

407. The method of any one of embodiments 404-406, wherein the composition is administered to the subject in combination with a checkpoint inhibitor.

408. The method of embodiment 407, wherein the checkpoint inhibitor is or comprises a small molecule, a biologic, a nucleic acid, a lipid, a carbohydrate, a metal, or combinations thereof.

409. The method of embodiment 407, wherein the checkpoint inhibitor targets 4-1BB (CD137), 4-1BBL (CD137L), CTLA-4, PD-L1, PD-1, PD-L2, LAG-3, TIM-3, TIGIT, VISTA, B7-H3, B7-H4, BTLA, HVEM, GAL9, KIR, 2B4, CD160 (BY55), or CGEN-15049.

410. The method of embodiment 407, wherein the checkpoint inhibitor is or comprises ipilimumab, tremelimumab, nivolumab, pembrolizumab, atezolizumab, avelumab, durvalumab, cemiplimab, AMP224, pidilizumab, BMS-936559, lirilumab, BY55, anti-OX40. anti-TIM3, or anti-LAG3.

### Certain Embodiments II

400. A method of treating a subject having or suspected of having chronic lymphocytic leukemia (CLL), the method comprising:
administering to the subject a composition comprising a population of engineered hypoimmunogenic T cells, wherein the hypoimmunogenic T cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules,
(b) increase expression of CD47 encoded by a first exogenous polynucleotide, wherein the increased expression of (b) is relative to a control or wild-type T cell that does not comprise the modification, and
(c) express a CD19-specific chimeric antigen receptor (CAR) encoded by a second exogenous polynucleotide.

401. The method of embodiment 400, wherein the composition is administered to the subject at a dosage of about 60×10⁶.

402. The method of embodiment 400, wherein the composition is administered to the subject at a dosage of about 120×10⁶.

403. The method of embodiment 400, wherein the composition is administered to the subject at a dosage of about 200×10⁶.

404. The method of embodiment 400, wherein the composition is administered to the subject at a dosage of about 400×10⁶.

405. The method of any one of embodiments 400-404, wherein the one or more modifications reduce expression of the one or more MHC class I molecules, the one or more MHC class II molecules, and/or the one or more TCR molecules in the hypoimmunogenic T cells, relative to control or wild-type T cells.

406. The method of any one of embodiments 400-405, wherein expression of the one or more MHC class I molecules, the one or more MHC class II molecules, and the one or more TCR molecules is reduced in the hypoimmunogenic T cells relative to the control or wild-type T cell.

407. The method of any one of embodiments 400-406, wherein the control or wild-type T cells do not comprise the modifications.

408. The method of any one of embodiments 400-407, wherein the one or more modifications reduce cell surface expression of the one or more MHC class I molecules relative to the control or wild-type T cell.

409. The method of any one of embodiments 400-408, wherein the one or more modifications reduce cell surface trafficking of the one or more MHC class I molecules relative to the control or wild-type T cell.

410. The method of any one of embodiments 400-409, wherein the one or more modifications reduce a function of the one or more MHC class I molecules.

411. The method of any one of embodiments 400-410, wherein the one or more MHC class I molecules is one or more human leukocyte antigen (HLA) class I molecules.

412. The method of embodiment 411, wherein the one or more HLA class I molecules is HLA-A, HLA-B, HLA-C, or a combination thereof.

413. The method of any one of embodiments 400-412, wherein the one or more molecules that regulate cell surface expression of the one or more MHC class I molecules, regulate cell surface trafficking of the one or more MHC class I molecules, and/or reduce a function of the one or more MHC class I molecules are beta-2 microglobulin (B2M).

414. The method of any one of embodiments 400-413, wherein the one or more modifications comprise a modification that inactivates or disrupts one or more alleles of B2M.

415. The method of embodiment 414, wherein the modification that inactivates or disrupts one or more alleles of B2M reduces mRNA expression of the B2M gene.

416. The method of embodiment 414 or embodiment 415, wherein the modification that inactivates or disrupts one or more alleles of B2M reduces protein expression of B2M.

417. The method of any one of embodiments 414-416, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the hypoimmunogenic T cell.

418. The method of any one of embodiments 414-417, wherein the modification that inactivates or disrupts one or more alleles of B2M comprises an indel in the B2M gene.

419. The method of any one of embodiments 414-418, wherein the modification that inactivates or disrupts one or more alleles of B2M comprises a frameshift mutation or a deletion of a contiguous stretch of genomic DNA of the B2M gene.

420. The method of any one of embodiments 400-419, wherein the one or more modifications reduce cell surface expression of the one or more MHC class II molecules relative to the control or wild-type cell.

421. The method of any one of embodiments 400-420, wherein the one or more modifications reduce cell surface trafficking of the one or more MHC class II molecules relative to the control or wild-type cell.

422. The method of any one of embodiments 400-421, wherein the one or more modifications reduce a function of the one or more MHC class II molecules.

423. The method of embodiment 422, wherein the function of the one more MHC class II molecules is antigen presentation.

424. The method of any one of embodiments 400-423, wherein the one or more MHC class II molecules is one or more human leukocyte antigen (HLA) class II molecules.

425. The method of embodiment 424, wherein the one or more HLA class II molecules is HLA-DP, HLA-DQ, HLA-DR, or a combination thereof.

426. The method of any one of embodiments 400-425, wherein the one or more molecules that regulate expression of the one or more MHC class II molecules are Class II Major Histocompatibility Complex Transactivator (CIITA).

427. The method of any one of embodiments 400-426, wherein the one or more modifications comprise a modification that inactivates or disrupts one or more alleles of CIITA.

428. The method of embodiment 427, wherein the modification that inactivates or disrupts one or more alleles of CIITA reduces mRNA expression of the CIITA gene.

429. The method of embodiment 427 or embodiment 428, wherein the modification that inactivates or disrupts one or more alleles of CIITA reduces protein expression of CIITA.

430. The method of any one of embodiments 427-429, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the hypoimmunogenic T cell.

431. The method of any one of embodiments 427-430, wherein the modification that inactivates or disrupts one or more alleles of CIITA comprises an indel in the CIITA gene.

432. The method of any one of embodiments 427-431, wherein the modification that inactivates or disrupts one or more alleles of CIITA comprises a frameshift mutation or a deletion of a contiguous stretch of genomic DNA of the CIITA gene.

433. The method of any one of embodiments 400-432, wherein expression of HLA-A, HLA-B, HLA-C, HLA-DP, HLA-DQ, and HLA-DR are reduced in the hypoimmunogenic T cell.

434. The method of any of embodiments 400-433, wherein the one or more modifications reduce cell surface protein expression of the one or more TCR molecules relative to the control or wild-type cell.

435. The method of any of embodiments 400-434, wherein the one or more modifications reduce cell surface trafficking of the one or more TCR molecules relative to the control or wild-type cell.

436. The method of any of embodiments 400-435, wherein the one or more modifications reduce a function of the one or more TCR molecules.

437. The method of embodiment 436, wherein the function of the one or more TCR molecules is antigen recognition.

438. The method of any of embodiments 400-437, wherein the one or more TCR molecules is T cell receptor alpha constant (TRAC), T cell receptor beta constant (TRBC), or a combination thereof.

439. The method of any of embodiments 400-438, wherein the one or more TCR molecules comprise TRAC.

440. The method of any of embodiments 400-439, wherein the one or more modifications comprise a modification that inactivates or disrupts one or more alleles of TRAC.

441. The method of embodiment 440, wherein the modification that inactivates or disrupts one or more alleles of TRAC reduces mRNA expression of the TRAC gene.

442. The method of embodiment 440 or 441, wherein the modification that inactivates or disrupts one or more alleles of TRAC reduces protein expression of TRAC.

443. The method of any of embodiments 440-442, wherein the modification that inactivates or disrupts one or more alleles of TRAC comprises:
inactivation or disruption of one allele of the TRAC gene;
inactivation or disruption of both alleles of the TRAC gene; or
inactivation or disruption of all TRAC coding alleles in the hypoimmunogenic T cell.

444. The method of any of embodiments 440-443, wherein the modification inactivation or disruption that inactivates or disrupts one or more alleles of TRAC comprises an indel in the TRAC gene.

445. The method of any of embodiments 440-444, wherein the inactivation or disruption comprises a frameshift mutation or a deletion of a contiguous stretch of genomic DNA of the TRAC gene.

446. The method of any one of embodiments 400-445 wherein the one or more modifications increase expression of one or more tolerogenic factors encoded by a third exogenous polynucleotide, wherein the increased expression of the one or more tolerogenic factors is relative to a control or wild-type T cell, and wherein the one or more tolerogenic factors comprise CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or a combination thereof.

447. A method of treating a subject having or suspected of having chronic lymphocytic leukemia (CLL), the method comprising:
administering to the subject a composition comprising a population of engineered hypoimmunogenic T cells comprising,
(a) reduced expression of beta-2 microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide, wherein the increased expression of (b) is relative to the control or wild-type T cells, and
(c) expression of a CD19-specific chimeric antigen receptor (CAR) encoded by a second exogenous polynucleotide.

448. The method of embodiment 447, wherein the composition is administered to the subject at a dosage of about 60×10⁶.

449. The method of embodiment 447, wherein the composition is administered to the subject at a dosage of about 120×10⁶.

450. The method of embodiment 447, wherein the composition is administered to the subject at a dosage of about 200×10⁶.

451. The method of embodiment 447, wherein the composition is administered to the subject at a dosage of about 400×10⁶.

452. The method of any one of embodiments 400-451, wherein the engineered hypoimmunogenic T cells comprise CD8+ and/or CD4+ T cells.

453. The method of any one of embodiments 400-452, wherein the engineered hypoimmunogenic T cells comprise allogeneic T cells.

454. The method of any one of embodiments 400-453, wherein the engineered hypoimmunogenic T cells comprise primary T cells.

455. The method of any one of embodiments 400-454, wherein the engineered hypoimmunogenic T cells comprise donor-derived T cells.

456. The method of embodiment 455, wherein the donor-derived T cells are derived from 1 donor, 2 donors, 3 donors, 4 donors, 5 donors, 6 donors, 7 donors, 8 donors, 9 donors, or 10 donors.

457. The method of any one of embodiments 400-456, wherein the engineered hypoimmunogenic T cells comprise stem cell-derived T cells.

458. The method of any one of embodiments 400-457, wherein the CAR comprises an anti-CD19 antigen binding domain, a CD8× hinge domain, a CD8α transmembrane domain, a 4-1BB costimulatory domain, and a CD3 intracellular signaling domain.

459. The method of embodiment 458, wherein the antigen binding domain comprises:
(i) a light chain amino acid sequence comprising a light chain CDR1, a light chain CDR2, and a light chain CDR3,
wherein the light chain CDR 1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and
(ii) a heavy chain amino acid sequence comprising a heavy chain CDR1, a heavy chain CDR2, and a heavy chain CDR3 amino acid,
wherein the heavy chain CDR1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28.

460. The method of embodiment 459, wherein the light chain amino acid sequence comprises a light chain variable region and the heavy chain amino acid sequence comprises a heavy chain variable region,
wherein the light chain variable region comprises an amino acid sequence at least 90% identical to SEQ ID NO: 20, and
wherein the heavy chain variable region comprises an amino acid sequence comprises an amino acid sequence at least 90% identical to SEQ ID NO: 25.

461. The method of any one of embodiments 458-460, wherein the CD8α hinge domain comprises an amino acid sequence at least 90% identical to SEQ ID NO: 9, the CD8α transmembrane domain comprises an amino acid sequence at least 90% identical to SEQ ID NO: 14, the 4-1BB costimulatory domain comprises an amino acid sequence at least 90% identical to SEQ ID NO: 16, and the CD3ζ intracellular signaling domain comprises an amino acid sequence at least 90% identical to SEQ ID NO: 18.

462. The method of any one of embodiments 458-461, wherein the antigen binding domain comprises a linker comprising the amino acid sequence at least 90% identical to SEQ ID NO: 24.

463. The method of any one of embodiments 458-462, wherein the antigen binding domain comprises a linker comprising the amino acid sequence of SEQ ID NO: 24.

464. The method of any one of embodiments 458-463, wherein the antigen binding domain comprises an amino acid sequence at least 90% identical to SEQ ID NO: 19.

465. The method of any one of embodiments 458-464, wherein the antigen binding domain comprises the amino acid sequence of SEQ ID NO: 19.

466. The method of any one of embodiments 400-465, wherein the engineered hypoimmunogenic T cells are B2M^{indel/indel}, CIITA^{indel/indel}, and/or TRAC^{indel/indel} cells.

467. The method of embodiment 466, wherein the population of engineered hypoimmunogenic T cells is a heterogeneous population comprising B2M^{indel/indel} cells, CIITA^{indel/indel} cells, TRAC^{indel/indel} cells, B2M^{indel/indel}/CIITA^{indel/indel} cells, B2M^{indel/indel}/TRAC^{indel/indel} cells, CIITA^{indel/indel}/TRAC^{indel/indel} cells, and/or B2M^{indel/indel}/CIITA^{indel/indel}/TRAC^{indel/indel} cells.

468. A method of treating a subject having or suspected of having non-hodgkin lymphoma (NHL), the method comprising:
administering to the subject a composition comprising a population of engineered hypoimmunogenic T cells, wherein the hypoimmunogenic T cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules,
(b) increase expression of CD47 encoded by a first exogenous polynucleotide, wherein the increased expression of (b) is relative to a control or wild-type T cell that does not comprise the modification, and
(c) express a CD19-specific chimeric antigen receptor (CAR) encoded by a second exogenous polynucleotide.

469. The method of embodiment 468, wherein the composition is administered to the subject at a dosage of about 60×10⁶.

470. The method of embodiment 468, wherein the composition is administered to the subject at a dosage of about 120×10⁶.

471. The method of embodiment 468, wherein the composition is administered to the subject at a dosage of about 200×10⁶.

472. The method of embodiment 468, wherein the composition is administered to the subject at a dosage of about 400×10⁶.

473. The method of any one of embodiments 468-472, wherein the one or more modifications reduce expression of the one or more MHC class I molecules, the one or more MHC class II molecules, and/or the one or more TCR molecules in the hypoimmunogenic T cells, relative to control or wild-type T cells.

474. The method of any one of embodiments 468-473, wherein expression of the one or more MHC class I molecules, the one or more MHC class II molecules, and the one or more TCR molecules is reduced in the hypoimmunogenic T cells relative to the control or wild-type T cell.

475. The method of any one of embodiments 468-474, wherein the control or wild-type T cells do not comprise the modifications.

476. The method of any one of embodiments 468-475, wherein the one or more modifications reduce cell surface expression of the one or more MHC class I molecules relative to the control or wild-type T cell.

477. The method of any one of embodiments 468-476, wherein the one or more modifications reduce cell surface trafficking of the one or more MHC class I molecules relative to the control or wild-type T cell.

478. The method of any one of embodiments 468-477, wherein the one or more modifications reduce a function of the one or more MHC class I molecules.

479. The method of any one of embodiments 468-478, wherein the one or more MHC class I molecules is one or more human leukocyte antigen (HLA) class I molecules.

480. The method of embodiment 479, wherein the one or more HLA class I molecules is HLA-A, HLA-B, HLA-C, or a combination thereof.

481. The method of any one of embodiments 468-480, wherein the one or more molecules that regulate cell surface expression of the one or more MHC class I molecules, regulate cell surface trafficking of the one or more MHC class I molecules, and/or reduce a function of the one or more MHC class I molecules are beta-2 microglobulin (B2M).

482. The method of any one of embodiments 468-481, wherein the one or more modifications comprise a modification that inactivates or disrupts one or more alleles of B2M.

483. The method of embodiment 482, wherein the modification that inactivates or disrupts one or more alleles of B2M reduces mRNA expression of the B2M gene.

484. The method of embodiment 482 or embodiment 483, wherein the modification that inactivates or disrupts one or more alleles of B2M reduces protein expression of B2M.

485. The method of any one of embodiments 482-484, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the hypoimmunogenic T cell.

486. The method of any one of embodiments 482-485, wherein the modification that inactivates or disrupts one or more alleles of B2M comprises an indel in the B2M gene.

487. The method of any one of embodiments 482-486, wherein the modification that inactivates or disrupts one or more alleles of B2M comprises a frameshift mutation or a deletion of a contiguous stretch of genomic DNA of the B2M gene.

488. The method of any one of embodiments 468-487, wherein the one or more modifications reduce cell surface expression of the one or more MHC class II molecules relative to the control or wild-type cell.

489. The method of any one of embodiments 468-488, wherein the one or more modifications reduce cell surface trafficking of the one or more MHC class II molecules relative to the control or wild-type cell.

490. The method of any one of embodiments 468-489, wherein the one or more modifications reduce a function of the one or more MHC class II molecules.

491. The method of embodiment 490, wherein the function of the one more MHC class II molecules is antigen presentation.

492. The method of any one of embodiments 468-491, wherein the one or more MHC class II molecules is one or more human leukocyte antigen (HLA) class II molecules.

493. The method of embodiment 492, wherein the one or more HLA class II molecules is HLA-DP, HLA-DQ, HLA-DR, or a combination thereof.

494. The method of any one of embodiments 468-493, wherein the one or more molecules that regulate expression of the one or more MHC class II molecules are Class II Major Histocompatibility Complex Transactivator (CIITA).

495. The method of any one of embodiments 468-494, wherein the one or more modifications comprise a modification that inactivates or disrupts one or more alleles of CIITA.

496. The method of embodiment 495, wherein the modification that inactivates or disrupts one or more alleles of CIITA reduces mRNA expression of the CIITA gene.

497. The method of embodiment 495 or embodiment 496, wherein the modification that inactivates or disrupts one or more alleles of CIITA reduces protein expression of CIITA.

498. The method of any one of embodiments 495-497, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the hypoimmunogenic T cell.

499. The method of any one of embodiments 495-498, wherein the modification that inactivates or disrupts one or more alleles of CIITA comprises an indel in the CIITA gene.

500. The method of any one of embodiments 495-499, wherein the modification that inactivates or disrupts one or more alleles of CIITA comprises a frameshift mutation or a deletion of a contiguous stretch of genomic DNA of the CIITA gene.

501. The method of any one of embodiments 468-500, wherein expression of HLA-A, HLA-B, HLA-C, HLA-DP, HLA-DQ, and HLA-DR are reduced in the hypoimmunogenic T cell.

502. The method of any of embodiments 468-501, wherein the one or more modifications reduce cell surface protein expression of the one or more TCR molecules relative to the control or wild-type cell.

503. The method of any of embodiments 468-502, wherein the one or more modifications reduce cell surface trafficking of the one or more TCR molecules relative to the control or wild-type cell.

504. The method of any of embodiments 468-503, wherein the one or more modifications reduce a function of the one or more TCR molecules.

505. The method of embodiment 504, wherein the function of the one or more TCR molecules is antigen recognition.

506. The method of any of embodiments 468-505, wherein the one or more TCR molecules is T cell receptor alpha constant (TRAC), T cell receptor beta constant (TRBC), or a combination thereof.

507. The method of any of embodiments 468-506, wherein the one or more TCR molecules comprise TRAC.

508. The method of any of embodiments 468-507, wherein the one or more modifications comprise a modification that inactivates or disrupts one or more alleles of TRAC.

509. The method of embodiment 508, wherein the modification that inactivates or disrupts one or more alleles of TRAC reduces mRNA expression of the TRAC gene.

510. The method of embodiment 508 or 509, wherein the modification that inactivates or disrupts one or more alleles of TRAC reduces protein expression of TRAC.

511. The method of any of embodiments 508-510, wherein the modification that inactivates or disrupts one or more alleles of TRAC comprises:
inactivation or disruption of one allele of the TRAC gene;
inactivation or disruption of both alleles of the TRAC gene; or
inactivation or disruption of all TRAC coding alleles in the hypoimmunogenic T cell.

512. The method of any of embodiments 508-511, wherein the modification inactivation or disruption that inactivates or disrupts one or more alleles of TRAC comprises an indel in the TRAC gene.

513. The method of any of embodiments 508-512, wherein the inactivation or disruption comprises a frameshift mutation or a deletion of a contiguous stretch of genomic DNA of the TRAC gene.

514. The method of any one of embodiments 468-513 wherein the one or more modifications increase expression of one or more tolerogenic factors encoded by a third exogenous polynucleotide, wherein the increased expression of the one or more tolerogenic factors is relative to a control or wild-type T cell, and wherein the one or more tolerogenic factors comprise CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or a combination thereof.

515. A method of treating a subject having or suspected of having non-hodgkin lymphoma (NHL), the method comprising:
administering to the subject a composition comprising a population of engineered hypoimmunogenic T cells comprising,
(a) reduced expression of beta-2 microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide, wherein the increased expression of (b) is relative to the control or wild-type T cells, and
(c) expression of a CD19-specific chimeric antigen receptor (CAR) encoded by a second exogenous polynucleotide.

516. The method of embodiment 515, wherein the composition is administered to the subject at a dosage of about 60×10⁶.

517. The method of embodiment 515, wherein the composition is administered to the subject at a dosage of about 120×10⁶.

518. The method of embodiment 515, wherein the composition is administered to the subject at a dosage of about 200×10⁶.

519. The method of embodiment 515, wherein the composition is administered to the subject at a dosage of about 400×10⁶.

520. The method of any one of embodiments 468-519, wherein the engineered hypoimmunogenic T cells comprise CD8+ and/or CD4+ T cells.

521. The method of any one of embodiments 468-520, wherein the engineered hypoimmunogenic T cells comprise allogeneic T cells.

522. The method of any one of embodiments 468-521, wherein the engineered hypoimmunogenic T cells comprise primary T cells.

523. The method of any one of embodiments 468-522, wherein the engineered hypoimmunogenic T cells comprise donor-derived T cells.

524. The method of embodiment 523, wherein the donor-derived T cells are derived from 1 donor, 2 donors, 3 donors, 4 donors, 5 donors, 6 donors, 7 donors, 8 donors, 9 donors, or 10 donors.

525. The method of any one of embodiments 468-524, wherein the engineered hypoimmunogenic T cells comprise stem cell-derived T cells.

526. The method of any one of embodiments 468-525, wherein the CAR comprises an anti-CD19 antigen binding domain, a CD8α hinge domain, a CD8α transmembrane domain, a 4-1BB costimulatory domain, and a CD3ζ intracellular signaling domain.

527. The method of embodiment 526, wherein the antigen binding domain comprises:
(i) a light chain amino acid sequence comprising a light chain CDR1, a light chain CDR2, and a light chain CDR3,
wherein the light chain CDR1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and
(ii) a heavy chain amino acid sequence comprising a heavy chain CDR1, a heavy chain CDR2, and a heavy chain CDR3 amino acid,
wherein the heavy chain CDR1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28.

528. The method of embodiment 527, wherein the light chain amino acid sequence comprises a light chain variable region and the heavy chain amino acid sequence comprises a heavy chain variable region,
wherein the light chain variable region comprises an amino acid sequence at least 90% identical to SEQ ID NO: 20, and
wherein the heavy chain variable region comprises an amino acid sequence comprises an amino acid sequence at least 90% identical to SEQ ID NO: 25.

529. The method of any one of embodiments 526-528, wherein the CD8× hinge domain comprises an amino acid sequence at least 90% identical to SEQ ID NO: 9, the CD8α transmembrane domain comprises an amino acid sequence at least 90% identical to SEQ ID NO: 14, the 4-1BB costimulatory domain comprises an amino acid sequence at least 90% identical to SEQ ID NO: 16, and the CD35 intracellular signaling domain comprises an amino acid sequence at least 90% identical to SEQ ID NO: 18.

530. The method of any one of embodiments 526-529, wherein the antigen binding domain comprises a linker comprising the amino acid sequence at least 90% identical to SEQ ID NO: 24.

531. The method of any one of embodiments 526-530, wherein the antigen binding domain comprises a linker comprising the amino acid sequence of SEQ ID NO: 24.

532. The method of any one of embodiments 526-531, wherein the antigen binding domain comprises an amino acid sequence at least 90% identical to SEQ ID NO: 19.

533. The method of any one of embodiments 526-532, wherein the antigen binding domain comprises the amino acid sequence of SEQ ID NO: 19.

534. The method of any one of embodiments 468-533, wherein the engineered hypoimmunogenic T cells are B2M^{indel/indel}, CIITA^{indel/indel}, and/or TRAC^{indel/indel} cells.

535. The method of embodiment 534, wherein the population of engineered hypoimmunogenic T cells is a heterogeneous population comprising B2M^{indel/indel} cells, CIITA^{indel/indel} cells, TRAC^{indel/indel} cells, B2M^{indel/indel}/CIITA^{indel/indel} cells, B2M^{indel/indel}/TRAC^{indel/indel} cells, CIITA^{indel/indel}/TRAC^{indel/indel} cells, and/or B2M^{indel/indel}/CIITA^{indel/indel}/TRAC^{indel/indel} cells.

536. A method of treating a subject having or suspected of having systemic lupus crythematosus (SLE), the method comprising:
administering to the subject a composition comprising a population of engineered hypoimmunogenic T cells, wherein the hypoimmunogenic T cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules,
(b) increase expression of CD47 encoded by a first exogenous polynucleotide, wherein the increased expression of (b) is relative to a control or wild-type T cell that does not comprise the modification, and
(c) express a CD19-specific chimeric antigen receptor (CAR) encoded by a second exogenous polynucleotide.

537. The method of embodiment 536, wherein the composition is administered to the subject at a dosage of about 60×10⁶.

538. The method of embodiment 536, wherein the composition is administered to the subject at a dosage of about 90×10⁶.

539. The method of embodiment 536, wherein the composition is administered to the subject at a dosage of about 120×10⁶.

540. The method of any one of embodiments 536-539, wherein the one or more modifications reduce expression of the one or more MHC class I molecules, the one or more MHC class II molecules, and/or the one or more TCR molecules in the hypoimmunogenic T cells, relative to control or wild-type T cells.

541. The method of any one of embodiments 536-540, wherein expression of the one or more MHC class I molecules, the one or more MHC class II molecules, and the one or more TCR molecules is reduced in the hypoimmunogenic T cells relative to the control or wild-type T cell.

542. The method of any one of embodiments 536-541, wherein the control or wild-type T cells do not comprise the modifications.

543. The method of any one of embodiments 536-542, wherein the one or more modifications reduce cell surface expression of the one or more MHC class I molecules relative to the control or wild-type T cell.

544. The method of any one of embodiments 536-543, wherein the one or more modifications reduce cell surface trafficking of the one or more MHC class I molecules relative to the control or wild-type T cell.

545. The method of any one of embodiments 536-544, wherein the one or more modifications reduce a function of the one or more MHC class I molecules.

546. The method of any one of embodiments 536-545, wherein the one or more MHC class I molecules is one or more human leukocyte antigen (HLA) class I molecules.

547. The method of embodiment 546, wherein the one or more HLA class I molecules is HLA-A, HLA-B, HLA-C, or a combination thereof.

548. The method of any one of embodiments 536-547, wherein the one or more molecules that regulate cell surface expression of the one or more MHC class I molecules, regulate cell surface trafficking of the one or more MHC class I molecules, and/or reduce a function of the one or more MHC class I molecules are beta-2 microglobulin (B2M).

549. The method of any one of embodiments 536-548, wherein the one or more modifications comprise a modification that inactivates or disrupts one or more alleles of B2M.

550. The method of embodiment 549, wherein the modification that inactivates or disrupts one or more alleles of B2M reduces mRNA expression of the B2M gene.

551. The method of embodiment 549 or embodiment 550, wherein the modification that inactivates or disrupts one or more alleles of B2M reduces protein expression of B2M.

552. The method of any one of embodiments 549-551, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the hypoimmunogenic T cell.

553. The method of any one of embodiments 549-552, wherein the modification that inactivates or disrupts one or more alleles of B2M comprises an indel in the B2M gene.

554. The method of any one of embodiments 549-553, wherein the modification that inactivates or disrupts one or more alleles of B2M comprises a frameshift mutation or a deletion of a contiguous stretch of genomic DNA of the B2M gene.

555. The method of any one of embodiments 536-554, wherein the one or more modifications reduce cell surface expression of the one or more MHC class II molecules relative to the control or wild-type cell.

556. The method of any one of embodiments 536-555, wherein the one or more modifications reduce cell surface trafficking of the one or more MHC class II molecules relative to the control or wild-type cell.

557. The method of any one of embodiments 536-556, wherein the one or more modifications reduce a function of the one or more MHC class II molecules.

558. The method of embodiment 557, wherein the function of the one more MHC class II molecules is antigen presentation.

559. The method of any one of embodiments 536-558, wherein the one or more MHC class II molecules is one or more human leukocyte antigen (HLA) class II molecules.

560. The method of embodiment 559, wherein the one or more HLA class II molecules is HLA-DP, HLA-DQ, HLA-DR, or a combination thereof.

561. The method of any one of embodiments 536-560, wherein the one or more molecules that regulate expression of the one or more MHC class II molecules are Class II Major Histocompatibility Complex Transactivator (CIITA).

562. The method of any one of embodiments 536-561, wherein the one or more modifications comprise a modification that inactivates or disrupts one or more alleles of CIITA.

563. The method of embodiment 562, wherein the modification that inactivates or disrupts one or more alleles of CIITA reduces mRNA expression of the CIITA gene.

564. The method of embodiment 562 or embodiment 563, wherein the modification that inactivates or disrupts one or more alleles of CIITA reduces protein expression of CIITA.

565. The method of any one of embodiments 562-564, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the hypoimmunogenic T cell.

566. The method of any one of embodiments 562-565, wherein the modification that inactivates or disrupts one or more alleles of CIITA comprises an indel in the CIITA gene.

567. The method of any one of embodiments 562-566, wherein the modification that inactivates or disrupts one or more alleles of CIITA comprises a frameshift mutation or a deletion of a contiguous stretch of genomic DNA of the CIITA gene.

568. The method of any one of embodiments 536-567, wherein expression of HLA-A, HLA-B, HLA-C, HLA-DP, HLA-DQ, and HLA-DR are reduced in the hypoimmunogenic T cell.

569. The method of any of embodiments 536-568, wherein the one or more modifications reduce cell surface protein expression of the one or more TCR molecules relative to the control or wild-type cell.

570. The method of any of embodiments 536-569, wherein the one or more modifications reduce cell surface trafficking of the one or more TCR molecules relative to the control or wild-type cell.

571. The method of any of embodiments 536-570, wherein the one or more modifications reduce a function of the one or more TCR molecules.

572. The method of embodiment 571, wherein the function of the one or more TCR molecules is antigen recognition.

573. The method of any of embodiments 536-572, wherein the one or more TCR molecules is T cell receptor alpha constant (TRAC), T cell receptor beta constant (TRBC), or a combination thereof.

574. The method of any of embodiments 536-573, wherein the one or more TCR molecules comprise TRAC.

575. The method of any of embodiments 536-574, wherein the one or more modifications comprise a modification that inactivates or disrupts one or more alleles of TRAC.

576. The method of embodiment 575, wherein the modification that inactivates or disrupts one or more alleles of TRAC reduces mRNA expression of the TRAC gene.

577. The method of embodiment 575 or 576, wherein the modification that inactivates or disrupts one or more alleles of TRAC reduces protein expression of TRAC.

578. The method of any of embodiments 575-577, wherein the modification that inactivates or disrupts one or more alleles of TRAC comprises:
inactivation or disruption of one allele of the TRAC gene;
inactivation or disruption of both alleles of the TRAC gene; or
inactivation or disruption of all TRAC coding alleles in the hypoimmunogenic T cell.

579. The method of any of embodiments 575-578, wherein the modification inactivation or disruption that inactivates or disrupts one or more alleles of TRAC comprises an indel in the TRAC gene.

580. The method of any of embodiments 575-579, wherein the inactivation or disruption comprises a frameshift mutation or a deletion of a contiguous stretch of genomic DNA of the TRAC gene.

581. The method of any one of embodiments 536-580, wherein the one or more modifications increase expression of one or more tolerogenic factors encoded by a third exogenous polynucleotide, wherein the increased expression of the one or more tolerogenic factors is relative to a control or wild-type T cell, and wherein the one or more tolerogenic factors comprise CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or a combination thereof.

582. A method of treating a subject having or suspected of having systemic lupus erythematosus (SLE), the method comprising:
administering to the subject a composition comprising a population of engineered hypoimmunogenic T cells comprising,
(a) reduced expression of beta-2 microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide, wherein the increased expression of (b) is relative to the control or wild-type T cells, and
(c) expression of a CD19-specific chimeric antigen receptor (CAR) encoded by a second exogenous polynucleotide.

583. The method of embodiment 582, wherein the composition is administered to the subject at a dosage of about 60×10⁶.

584. The method of embodiment 582, wherein the composition is administered to the subject at a dosage of about 90×10⁶.

585. The method of embodiment 582, wherein the composition is administered to the subject at a dosage of about 120×10⁶.

586. The method of any one of embodiments 536-585, wherein the engineered hypoimmunogenic T cells comprise CD8+ and/or CD4+ T cells.

587. The method of any one of embodiments 536-586, wherein the engineered hypoimmunogenic T cells comprise allogeneic T cells.

588. The method of any one of embodiments 536-587, wherein the engineered hypoimmunogenic T cells comprise primary T cells.

589. The method of any one of embodiments 536-588, wherein the engineered hypoimmunogenic T cells comprise donor-derived T cells.

590. The method of embodiment 589, wherein the donor-derived T cells are derived from 1 donor, 2 donors, 3 donors, 4 donors, 5 donors, 6 donors, 7 donors, 8 donors, 9 donors, or 10 donors.

591. The method of any one of embodiments 536-590, wherein the engineered hypoimmunogenic T cells comprise stem cell-derived T cells.

592. The method of any one of embodiments 536-591, wherein the CAR comprises an anti-CD19 antigen binding domain, a CD8α hinge domain, a CD8× transmembrane domain, a 4-1BB costimulatory domain, and a CD3ζ intracellular signaling domain.

593. The method of embodiment 592, wherein the antigen binding domain comprises:
(i) a light chain amino acid sequence comprising a light chain CDR1, a light chain CDR2, and a light chain CDR3,
wherein the light chain CDR1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and
(ii) a heavy chain amino acid sequence comprising a heavy chain CDR1, a heavy chain CDR2, and a heavy chain CDR3 amino acid,
wherein the heavy chain CDR1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28.

594. The method of embodiment 593, wherein the light chain amino acid sequence comprises a light chain variable region and the heavy chain amino acid sequence comprises a heavy chain variable region,
wherein the light chain variable region comprises an amino acid sequence at least 90% identical to SEQ ID NO: 20, and
wherein the heavy chain variable region comprises an amino acid sequence comprises an amino acid sequence at least 90% identical to SEQ ID NO: 25.

595. The method of any one of embodiments 592-594, wherein the CD8α hinge domain comprises an amino acid sequence at least 90% identical to SEQ ID NO: 9, the CD8α transmembrane domain comprises an amino acid sequence at least 90% identical to SEQ ID NO: 14, the 4-1BB costimulatory domain comprises an amino acid sequence at least 90% identical to SEQ ID NO: 16, and the CD3ζ intracellular signaling domain comprises an amino acid sequence at least 90% identical to SEQ ID NO: 18.

596. The method of any one of embodiments 592-595, wherein the antigen binding domain comprises a linker comprising the amino acid sequence at least 90% identical to SEQ ID NO: 24.

597. The method of any one of embodiments 592-596, wherein the antigen binding domain comprises a linker comprising the amino acid sequence of SEQ ID NO: 24.

598. The method of any one of embodiments 592-597, wherein the antigen binding domain comprises an amino acid sequence at least 90% identical to SEQ ID NO: 19.

599. The method of any one of embodiments 592-598, wherein the antigen binding domain comprises the amino acid sequence of SEQ ID NO: 19.

600. The method of any one of embodiments 536-599, wherein the engineered hypoimmunogenic T cells are B2M^{indel/indel}, CIITA^{indel/indel}, and/or TRAC^{indel/indel} cells.

601. The method of embodiment 600, wherein the population of engineered hypoimmunogenic T cells is a heterogeneous population comprising B2M^{indel/indel} cells, CIITA^{indel/indel} cells, TRAC^{indel/indel} cells, B2M^{indel/indel}/CIITA^{indel/indel} cells, B2M^{indel/indel}/TRAC^{indel/indel} cells, CIITA^{indel/indel}/TRAC^{indel/indel} cells, and/or B2M^{indel/indel}/CIITA^{indel/indel}/TRAC^{indel/indel} cells.

602. A method of treating a subject having or suspected of having lupus nephritis, the method comprising:
administering to the subject a composition comprising a population of engineered hypoimmunogenic T cells, wherein the hypoimmunogenic T cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules,
(b) increase expression of CD47 encoded by a first exogenous polynucleotide, wherein the increased expression of (b) is relative to a control or wild-type T cell that does not comprise the modification, and
(c) express a CD19-specific chimeric antigen receptor (CAR) encoded by a second exogenous polynucleotide.

603. The method of embodiment 602, wherein the composition is administered to the subject at a dosage of about 60×10⁶.

604. The method of embodiment 602, wherein the composition is administered to the subject at a dosage of about 90×10⁶.

605. The method of embodiment 602, wherein the composition is administered to the subject at a dosage of about 120×10⁶.

606. The method of any one of embodiments 602-605, wherein the one or more modifications reduce expression of the one or more MHC class I molecules, the one or more MHC class II molecules, and/or the one or more TCR molecules in the hypoimmunogenic T cells, relative to control or wild-type T cells.

607. The method of any one of embodiments 602-606, wherein expression of the one or more MHC class I molecules, the one or more MHC class II molecules, and the one or more TCR molecules is reduced in the hypoimmunogenic T cells relative to the control or wild-type T cell.

608. The method of any one of embodiments 602-607, wherein the control or wild-type T cells do not comprise the modifications.

609. The method of any one of embodiments 602-608, wherein the one or more modifications reduce cell surface expression of the one or more MHC class I molecules relative to the control or wild-type T cell.

610. The method of any one of embodiments 602-609, wherein the one or more modifications reduce cell surface trafficking of the one or more MHC class I molecules relative to the control or wild-type T cell.

611. The method of any one of embodiments 602-610, wherein the one or more modifications reduce a function of the one or more MHC class I molecules.

612. The method of any one of embodiments 602-611, wherein the one or more MHC class I molecules is one or more human leukocyte antigen (HLA) class I molecules.

613. The method of embodiment 612, wherein the one or more HLA class I molecules is HLA-A, HLA-B, HLA-C, or a combination thereof.

614. The method of any one of embodiments 602-613, wherein the one or more molecules that regulate cell surface expression of the one or more MHC class I molecules, regulate cell surface trafficking of the one or more MHC class I molecules, and/or reduce a function of the one or more MHC class I molecules are beta-2 microglobulin (B2M).

615. The method of any one of embodiments 602-614, wherein the one or more modifications comprise a modification that inactivates or disrupts one or more alleles of B2M.

616. The method of embodiment 615, wherein the modification that inactivates or disrupts one or more alleles of B2M reduces mRNA expression of the B2M gene.

617. The method of embodiment 615 or embodiment 616, wherein the modification that inactivates or disrupts one or more alleles of B2M reduces protein expression of B2M.

618. The method of any one of embodiments 615-617, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the hypoimmunogenic T cell.

619. The method of any one of embodiments 615-618, wherein the modification that inactivates or disrupts one or more alleles of B2M comprises an indel in the B2M gene.

620. The method of any one of embodiments 615-619, wherein the modification that inactivates or disrupts one or more alleles of B2M comprises a frameshift mutation or a deletion of a contiguous stretch of genomic DNA of the B2M gene.

621. The method of any one of embodiments 602-620, wherein the one or more modifications reduce cell surface expression of the one or more MHC class II molecules relative to the control or wild-type cell.

622. The method of any one of embodiments 602-621, wherein the one or more modifications reduce cell surface trafficking of the one or more MHC class II molecules relative to the control or wild-type cell.

623. The method of any one of embodiments 602-622, wherein the one or more modifications reduce a function of the one or more MHC class II molecules.

624. The method of embodiment 623, wherein the function of the one more MHC class II molecules is antigen presentation.

625. The method of any one of embodiments 602-624, wherein the one or more MHC class II molecules is one or more human leukocyte antigen (HLA) class II molecules.

626. The method of embodiment 625, wherein the one or more HLA class II molecules is HLA-DP, HLA-DQ, HLA-DR, or a combination thereof.

627. The method of any one of embodiments 602-626, wherein the one or more molecules that regulate expression of the one or more MHC class II molecules are Class II Major Histocompatibility Complex Transactivator (CIITA).

628. The method of any one of embodiments 602-627, wherein the one or more modifications comprise a modification that inactivates or disrupts one or more alleles of CIITA.

629. The method of embodiment 628, wherein the modification that inactivates or disrupts one or more alleles of CIITA reduces mRNA expression of the CIITA gene.

630. The method of embodiment 628 or embodiment 629, wherein the modification that inactivates or disrupts one or more alleles of CIITA reduces protein expression of CIITA.

631. The method of any one of embodiments 628-630, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the hypoimmunogenic T cell.

632. The method of any one of embodiments 628-631, wherein the modification that inactivates or disrupts one or more alleles of CIITA comprises an indel in the CIITA gene.

633. The method of any one of embodiments 628-632, wherein the modification that inactivates or disrupts one or more alleles of CIITA comprises a frameshift mutation or a deletion of a contiguous stretch of genomic DNA of the CIITA gene.

634. The method of any one of embodiments 602-633, wherein expression of HLA-A, HLA-B, HLA-C, HLA-DP, HLA-DQ, and HLA-DR are reduced in the hypoimmunogenic T cell.

635. The method of any of embodiments 602-634, wherein the one or more modifications reduce cell surface protein expression of the one or more TCR molecules relative to the control or wild-type cell.

636. The method of any of embodiments 602-635, wherein the one or more modifications reduce cell surface trafficking of the one or more TCR molecules relative to the control or wild-type cell.

637. The method of any of embodiments 602-636, wherein the one or more modifications reduce a function of the one or more TCR molecules.

638. The method of embodiment 637, wherein the function of the one or more TCR molecules is antigen recognition.

639. The method of any of embodiments 602-638, wherein the one or more TCR molecules is T cell receptor alpha constant (TRAC), T cell receptor beta constant (TRBC), or a combination thereof.

640. The method of any of embodiments 602-639, wherein the one or more TCR molecules comprise TRAC.

641. The method of any of embodiments 602-640, wherein the one or more modifications comprise a modification that inactivates or disrupts one or more alleles of TRAC.

642. The method of embodiment 641, wherein the modification that inactivates or disrupts one or more alleles of TRAC reduces mRNA expression of the TRAC gene.

643. The method of embodiment 641 or 642, wherein the modification that inactivates or disrupts one or more alleles of TRAC reduces protein expression of TRAC.

644. The method of any of embodiments 641-643, wherein the modification that inactivates or disrupts one or more alleles of TRAC comprises:
inactivation or disruption of one allele of the TRAC gene;
inactivation or disruption of both alleles of the TRAC gene; or
inactivation or disruption of all TRAC coding alleles in the hypoimmunogenic T cell.

645. The method of any of embodiments 641-644, wherein the modification inactivation or disruption that inactivates or disrupts one or more alleles of TRAC comprises an indel in the TRAC gene.

646. The method of any of embodiments 641-645, wherein the inactivation or disruption comprises a frameshift mutation or a deletion of a contiguous stretch of genomic DNA of the TRAC gene.

647. The method of any one of embodiments 602-646, wherein the one or more modifications increase expression of one or more tolerogenic factors encoded by a third exogenous polynucleotide, wherein the increased expression of the one or more tolerogenic factors is relative to a control or wild-type T cell, and wherein the one or more tolerogenic factors comprise CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or a combination thereof.

648. A method of treating a subject having or suspected of having lupus nephritis, the method comprising:
administering to the subject a composition comprising a population of engineered hypoimmunogenic T cells comprising,
(a) reduced expression of beta-2 microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide, wherein the increased expression of (b) is relative to the control or wild-type T cells, and
(c) expression of a CD19-specific chimeric antigen receptor (CAR) encoded by a second exogenous polynucleotide.

649. The method of embodiment 648, wherein the composition is administered to the subject at a dosage of about 60×10⁶.

650. The method of embodiment 648, wherein the composition is administered to the subject at a dosage of about 90×10⁶.

651. The method of embodiment 648, wherein the composition is administered to the subject at a dosage of about 120×10⁶.

652. The method of any one of embodiments 602-651, wherein the engineered hypoimmunogenic T cells comprise CD8+ and/or CD4+ T cells.

653. The method of any one of embodiments 602-652, wherein the engineered hypoimmunogenic T cells comprise allogeneic T cells.

654. The method of any one of embodiments 602-653, wherein the engineered hypoimmunogenic T cells comprise primary T cells.

655. The method of any one of embodiments 602-654, wherein the engineered hypoimmunogenic T cells comprise donor-derived T cells.

656. The method of embodiment 655, wherein the donor-derived T cells are derived from 1 donor, 2 donors, 3 donors, 4 donors, 5 donors, 6 donors, 7 donors, 8 donors, 9 donors, or 10 donors.

657. The method of any one of embodiments 602-656, wherein the engineered hypoimmunogenic T cells comprise stem cell-derived T cells.

658. The method of any one of embodiments 602-657, wherein the CAR comprises an anti-CD19 antigen binding domain, a CD8α hinge domain, a CD8α transmembrane domain, a 4-1BB costimulatory domain, and a CD3ζ intracellular signaling domain.

659. The method of embodiment 658, wherein the antigen binding domain comprises:
(i) a light chain amino acid sequence comprising a light chain CDR1, a light chain CDR2, and a light chain CDR3,
wherein the light chain CDR1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and
(ii) a heavy chain amino acid sequence comprising a heavy chain CDR1, a heavy chain CDR2, and a heavy chain CDR3 amino acid,
wherein the heavy chain CDR1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28.

660. The method of embodiment 659, wherein the light chain amino acid sequence comprises a light chain variable region and the heavy chain amino acid sequence comprises a heavy chain variable region,
wherein the light chain variable region comprises an amino acid sequence at least 90% identical to SEQ ID NO: 20, and
wherein the heavy chain variable region comprises an amino acid sequence comprises an amino acid sequence at least 90% identical to SEQ ID NO: 25.

661. The method of any one of embodiments 658-660, wherein the CD8α hinge domain comprises an amino acid sequence at least 90% identical to SEQ ID NO: 9, the CD8α transmembrane domain comprises an amino acid sequence at least 90% identical to SEQ ID NO: 14, the 4-1BB costimulatory domain comprises an amino acid sequence at least 90% identical to SEQ ID NO: 16, and the CD3ζ intracellular signaling domain comprises an amino acid sequence at least 90% identical to SEQ ID NO: 18.

662. The method of any one of embodiments 658-661, wherein the antigen binding domain comprises a linker comprising the amino acid sequence at least 90% identical to SEQ ID NO: 24.

663. The method of any one of embodiments 658-662, wherein the antigen binding domain comprises a linker comprising the amino acid sequence of SEQ ID NO: 24.

664. The method of any one of embodiments 658-663, wherein the antigen binding domain comprises an amino acid sequence at least 90% identical to SEQ ID NO: 19.

665. The method of any one of embodiments 658-664, wherein the antigen binding domain comprises the amino acid sequence of SEQ ID NO: 19.

666. The method of any one of embodiments 602-665, wherein the engineered hypoimmunogenic T cells are B2M^{indel/indel}, CIITA^{indel/indel}, and/or TRAC^{indel/indel} cells.

667. The method of embodiment 666, wherein the population of engineered hypoimmunogenic T cells is a heterogeneous population comprising B2M^{indel/indel} cells, CIITA^{indel/indel} cells, TRAC^{indel/indel} cells, B2M^{indel/indel}/CIITA^{indel/indel} cells, B2M^{indel/indel}/TRAC^{indel/indel} cells, CIITA^{indel/indel}/TRAC^{indel/indel} cells, and/or B2M^{indel/indel}/CIITA^{indel/indel}/TRAC^{indel/indel} cells.

668. A method of treating a subject having or suspected of having ANCA-associated vasculitis (AAV), the method comprising:
administering to the subject a composition comprising a population of engineered hypoimmunogenic T cells, wherein the hypoimmunogenic T cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules,
(b) increase expression of CD47 encoded by a first exogenous polynucleotide, wherein the increased expression of (b) is relative to a control or wild-type T cell that does not comprise the modification, and
(c) express a CD19-specific chimeric antigen receptor (CAR) encoded by a second exogenous polynucleotide.

669. The method of embodiment 668, wherein the composition is administered to the subject at a dosage of about 60×10⁶.

670. The method of embodiment 668, wherein the composition is administered to the subject at a dosage of about 90×10⁶.

671. The method of embodiment 668, wherein the composition is administered to the subject at a dosage of about 120×10⁶.

672. The method of any one of embodiments 668-671, wherein the one or more modifications reduce expression of the one or more MHC class I molecules, the one or more MHC class II molecules, and/or the one or more TCR molecules in the hypoimmunogenic T cells, relative to control or wild-type T cells.

673. The method of any one of embodiments 668-672, wherein expression of the one or more MHC class I molecules, the one or more MHC class II molecules, and the one or more TCR molecules is reduced in the hypoimmunogenic T cells relative to the control or wild-type T cell.

674. The method of any one of embodiments 668-673, wherein the control or wild-type T cells do not comprise the modifications.

675. The method of any one of embodiments 668-674, wherein the one or more modifications reduce cell surface expression of the one or more MHC class I molecules relative to the control or wild-type T cell.

676. The method of any one of embodiments 668-675, wherein the one or more modifications reduce cell surface trafficking of the one or more MHC class I molecules relative to the control or wild-type T cell.

677. The method of any one of embodiments 668-676, wherein the one or more modifications reduce a function of the one or more MHC class I molecules.

678. The method of any one of embodiments 668-677, wherein the one or more MHC class I molecules is one or more human leukocyte antigen (HLA) class I molecules.

679. The method of embodiment 678, wherein the one or more HLA class I molecules is HLA-A, HLA-B, HLA-C, or a combination thereof.

680. The method of any one of embodiments 668-679, wherein the one or more molecules that regulate cell surface expression of the one or more MHC class I molecules, regulate cell surface trafficking of the one or more MHC class I molecules, and/or reduce a function of the one or more MHC class I molecules are beta-2 microglobulin (B2M).

681. The method of any one of embodiments 668-680, wherein the one or more modifications comprise a modification that inactivates or disrupts one or more alleles of B2M.

682. The method of embodiment 681, wherein the modification that inactivates or disrupts one or more alleles of B2M reduces mRNA expression of the B2M gene.

683. The method of embodiment 681 or embodiment 682, wherein the modification that inactivates or disrupts one or more alleles of B2M reduces protein expression of B2M.

684. The method of any one of embodiments 681-683, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the hypoimmunogenic T cell.

685. The method of any one of embodiments 681-684, wherein the modification that inactivates or disrupts one or more alleles of B2M comprises an indel in the B2M gene.

686. The method of any one of embodiments 681-685, wherein the modification that inactivates or disrupts one or more alleles of B2M comprises a frameshift mutation or a deletion of a contiguous stretch of genomic DNA of the B2M gene.

687. The method of any one of embodiments 668-686, wherein the one or more modifications reduce cell surface expression of the one or more MHC class II molecules relative to the control or wild-type cell.

688. The method of any one of embodiments 668-687, wherein the one or more modifications reduce cell surface trafficking of the one or more MHC class II molecules relative to the control or wild-type cell.

689. The method of any one of embodiments 668-688, wherein the one or more modifications reduce a function of the one or more MHC class II molecules.

690. The method of embodiment 689, wherein the function of the one more MHC class II molecules is antigen presentation.

691. The method of any one of embodiments 668-690, wherein the one or more MHC class II molecules is one or more human leukocyte antigen (HLA) class II molecules.

692. The method of embodiment 691, wherein the one or more HLA class II molecules is HLA-DP, HLA-DQ, HLA-DR, or a combination thereof.

693. The method of any one of embodiments 668-692, wherein the one or more molecules that regulate expression of the one or more MHC class II molecules are Class II Major Histocompatibility Complex Transactivator (CIITA).

694. The method of any one of embodiments 668-693, wherein the one or more modifications comprise a modification that inactivates or disrupts one or more alleles of CIITA.

695. The method of embodiment 694, wherein the modification that inactivates or disrupts one or more alleles of CIITA reduces mRNA expression of the CIITA gene.

696. The method of embodiment 694 or embodiment 695, wherein the modification that inactivates or disrupts one or more alleles of CIITA reduces protein expression of CIITA.

697. The method of any one of embodiments 694-696, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the hypoimmunogenic T cell.

698. The method of any one of embodiments 694-697, wherein the modification that inactivates or disrupts one or more alleles of CIITA comprises an indel in the CIITA gene.

699. The method of any one of embodiments 694-698, wherein the modification that inactivates or disrupts one or more alleles of CIITA comprises a frameshift mutation or a deletion of a contiguous stretch of genomic DNA of the CIITA gene.

700. The method of any one of embodiments 668-699, wherein expression of HLA-A, HLA-B, HLA-C, HLA-DP, HLA-DQ, and HLA-DR are reduced in the hypoimmunogenic T cell.

701. The method of any of embodiments 668-700, wherein the one or more modifications reduce cell surface protein expression of the one or more TCR molecules relative to the control or wild-type cell.

702. The method of any of embodiments 668-701, wherein the one or more modifications reduce cell surface trafficking of the one or more TCR molecules relative to the control or wild-type cell.

703. The method of any of embodiments 668-702, wherein the one or more modifications reduce a function of the one or more TCR molecules.

704. The method of embodiment 703, wherein the function of the one or more TCR molecules is antigen recognition.

705. The method of any of embodiments 668-704, wherein the one or more TCR molecules is T cell receptor alpha constant (TRAC), T cell receptor beta constant (TRBC), or a combination thereof.

706. The method of any of embodiments 668-705, wherein the one or more TCR molecules comprise TRAC.

707. The method of any of embodiments 668-706, wherein the one or more modifications comprise a modification that inactivates or disrupts one or more alleles of TRAC.

708. The method of embodiment 707, wherein the modification that inactivates or disrupts one or more alleles of TRAC reduces mRNA expression of the TRAC gene.

709. The method of embodiment 707 or 708, wherein the modification that inactivates or disrupts one or more alleles of TRAC reduces protein expression of TRAC.

710. The method of any of embodiments 707-709, wherein the modification that inactivates or disrupts one or more alleles of TRAC comprises:
inactivation or disruption of one allele of the TRAC gene;
inactivation or disruption of both alleles of the TRAC gene; or
inactivation or disruption of all TRAC coding alleles in the hypoimmunogenic T cell.

711. The method of any of embodiments 707-710, wherein the modification inactivation or disruption that inactivates or disrupts one or more alleles of TRAC comprises an indel in the TRAC gene.

712. The method of any of embodiments 707-711, wherein the inactivation or disruption comprises a frameshift mutation or a deletion of a contiguous stretch of genomic DNA of the TRAC gene.

713. The method of any one of embodiments 668-712, wherein the one or more modifications increase expression of one or more tolerogenic factors encoded by a third exogenous polynucleotide, wherein the increased expression of the one or more tolerogenic factors is relative to a control or wild-type T cell, and wherein the one or more tolerogenic factors comprise CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, or a combination thereof.

714. A method of treating a subject having or suspected of having ANCA-associated vasculitis (AAV), the method comprising:
administering to the subject a composition comprising a population of engineered hypoimmunogenic T cells comprising,
(a) reduced expression of beta-2 microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide, wherein the increased expression of (b) is relative to the control or wild-type T cells, and
(c) expression of a CD19-specific chimeric antigen receptor (CAR) encoded by a second exogenous polynucleotide.

715. The method of embodiment 714, wherein the composition is administered to the subject at a dosage of about 60×10⁶.

716. The method of embodiment 714, wherein the composition is administered to the subject at a dosage of about 90×10⁶.

717. The method of embodiment 714, wherein the composition is administered to the subject at a dosage of about 120×10⁶.

718. The method of any one of embodiments 668-717, wherein the engineered hypoimmunogenic T cells comprise CD8+ and/or CD4+ T cells.

719. The method of any one of embodiments 668-718, wherein the engineered hypoimmunogenic T cells comprise allogeneic T cells.

720. The method of any one of embodiments 668-719, wherein the engineered hypoimmunogenic T cells comprise primary T cells.

721. The method of any one of embodiments 668-720, wherein the engineered hypoimmunogenic T cells comprise donor-derived T cells.

722. The method of embodiment 721, wherein the donor-derived T cells are derived from 1 donor, 2 donors, 3 donors, 4 donors, 5 donors, 6 donors, 7 donors, 8 donors, 9 donors, or 10 donors.

723. The method of any one of embodiments 668-722, wherein the engineered hypoimmunogenic T cells comprise stem cell-derived T cells.

724. The method of any one of embodiments 668-723, wherein the CAR comprises an anti-CD19 antigen binding domain, a CD8α hinge domain, a CD8α transmembrane domain, a 4-1BB costimulatory domain, and a CD33ζ intracellular signaling domain.

725. The method of embodiment 724, wherein the antigen binding domain comprises:
(i) a light chain amino acid sequence comprising a light chain CDR1, a light chain CDR2, and a light chain CDR3,
wherein the light chain CDR1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and
(ii) a heavy chain amino acid sequence comprising a heavy chain CDR1, a heavy chain CDR2, and a heavy chain CDR3 amino acid,
wherein the heavy chain CDR1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28.

726. The method of embodiment 725, wherein the light chain amino acid sequence comprises a light chain variable region and the heavy chain amino acid sequence comprises a heavy chain variable region,
wherein the light chain variable region comprises an amino acid sequence at least 90% identical to SEQ ID NO: 20, and
wherein the heavy chain variable region comprises an amino acid sequence comprises an amino acid sequence at least 90% identical to SEQ ID NO: 25.

727. The method of any one of embodiments 724-726, wherein the CD8α hinge domain comprises an amino acid sequence at least 90% identical to SEQ ID NO: 9, the CD8α transmembrane domain comprises an amino acid sequence at least 90% identical to SEQ ID NO: 14, the 4-1BB costimulatory domain comprises an amino acid sequence at least 90% identical to SEQ ID NO: 16, and the CD3ζ intracellular signaling domain comprises an amino acid sequence at least 90% identical to SEQ ID NO: 18.

728. The method of any one of embodiments 724-727, wherein the antigen binding domain comprises a linker comprising the amino acid sequence at least 90% identical to SEQ ID NO: 24.

729. The method of any one of embodiments 724-728, wherein the antigen binding domain comprises a linker comprising the amino acid sequence of SEQ ID NO: 24.

730. The method of any one of embodiments 724-729, wherein the antigen binding domain comprises an amino acid sequence at least 90% identical to SEQ ID NO: 19.

731. The method of any one of embodiments 724-730, wherein the antigen binding domain comprises the amino acid sequence of SEQ ID NO: 19.

732. The method of any one of embodiments 668-731, wherein the engineered hypoimmunogenic T cells are B2M^{indel/indel}, CIITA^{indel/indel}, and/or TRAC^{indel/indel} cells.

733. The method of embodiment 732, wherein the population of engineered hypoimmunogenic T cells is a heterogeneous population comprising B2M^{indel/indel} cells, CIITA^{indel/indel} cells, TRAC^{indel/indel} cells, B2M^{indel/indel}/CIITA^{indel/indel} cells, B2M^{indel/indel}/TRAC^{indel/indel} cells, CIITA^{indel/indel}/TRAC^{indel/indel} cells, and/or B2M^{indel/indel}/CIITA^{indel/indel}/TRAC^{indel/indel} cells.

### EQUIVALENTS

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a subject having or suspected of having a B cell malignancy or a B cell mediated disorder, the method comprising:
administering to the subject a composition comprising a population of engineered hypoimmunogenic T cells, wherein the hypoimmunogenic T cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules,
(b) increase expression of CD47 encoded by a first exogenous polynucleotide,
wherein the increased expression of (b) is relative to a control or wild-type T cell that does not comprise the modification, and
(c) express a CD19-specific chimeric antigen receptor (CAR) encoded by a second exogenous polynucleotide.

2. A method of treating a subject having or suspected of having a B cell malignancy or a B cell mediated disorder, the method comprising:
administering to the subject a composition comprising a population of engineered hypoimmunogenic T cells comprising:
(a) reduced expression of beta-2 microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide,
wherein the increased expression of (b) is relative to the control or wild-type T cells, and
(c) expression of a CD19-specific chimeric antigen receptor (CAR) encoded by a second exogenous polynucleotide.

3. The method of claim 1 or claim 2, wherein the composition is administered to the subject at a dosage of about 5×10⁶ cells to about 400×10⁶ cells.

4. The method of any one of the claims 1-3, wherein the composition is administered to the subject at a dosage of at least about 30×10⁶ cells, at least about 60×10⁶ cells, at least about 90×10⁶ cells, at least about 120×10⁶ cells, or at least about 200×10⁶ cells.

5. The method of any one of claims 1-4, wherein the composition is administered to the subject at a dosage of about 5×10⁶ CAR+ cells to about 400×10⁶ CAR+ cells.

6. The method of any one of claims 1-5, wherein the composition is administered to the subject at a dosage of at least about 30×10⁶ CAR+ cells, at least about 60×10⁶ CAR+ cells, at least about 90×10⁶ CAR+ cells, at least about 120×10⁶ CAR+ cells, or at least about 200×10⁶ CAR+ cells.

7. The method of any one of claims 1-6, wherein a total volume of about 1.25 mL to about 100 mL of the composition is administered.

8. The method of any one of claims 1-7, wherein the composition comprises a concentration of engineered hypoimmunogenic T cells of at least 1.25×10⁶ cells/mL.

9. The method of any one of claims 1-8, wherein the CAR comprises an anti-CD19 antigen binding domain, a CD8α hinge domain, a CD8α transmembrane domain, a 4-1BB costimulatory domain, and a CD3ζ intracellular signaling domain.

10. The method of claim 9, wherein the antigen binding domain comprises:
(i) a light chain amino acid sequence comprising a light chain CDR1, a light chain CDR2, and a light chain CDR3,
wherein the light chain CDR 1 comprises the amino acid sequence of SEQ ID NO: 21, the light chain CDR2 comprises the amino acid sequence of SEQ ID NO: 22, and the light chain CDR3 comprises the amino acid sequence of SEQ ID NO: 23, and
(ii) a heavy chain amino acid sequence comprising a heavy chain CDR1, a heavy chain CDR2, and a heavy chain CDR3 amino acid,
wherein the heavy chain CDR1 comprises the amino acid sequence of SEQ ID NO: 26, the heavy chain CDR2 comprises the amino acid sequence of SEQ ID NO: 27, and the heavy chain CDR3 comprises the amino acid sequence of SEQ ID NO: 28.

11. The method of claim 10, wherein the light chain amino acid sequence comprises a light chain variable region and the heavy chain amino acid sequence comprises a heavy chain variable region,
wherein the light chain variable region comprises an amino acid sequence at least 90% identical to SEQ ID NO: 20, and
wherein the heavy chain variable region comprises an amino acid sequence comprises an amino acid sequence at least 90% identical to SEQ ID NO: 25.

12. The method of any one of claims 1-11, wherein the engineered hypoimmunogenic T cells are B2M^{indel/indel}, CIITA^{indel/indel}, and/or TRAC^{indel/indel} cells.

13. The method of any one of claims 1-12, wherein the B cell malignancy is a lymphoma or a leukemia.

14. The method of any one of claims 1-13, wherein the B cell malignancy is selected from the group consisting of: Non-Hodgkin's Lymphoma (NHL), Chronic Lymphocytic Leukemia (CLL), large B cell lymphoma (LBCL), transplant ineligible large B cell lymphoma (LBCL), diffuse LBCL (DLBCL), high-grade B cell lymphoma (HGBCL), primary mediastinal B cell lymphoma (PMBCL), mantle cell lymphoma (MCL), follicular lymphoma (FL), marginal zone lymphoma (MZL), or small lymphocytic lymphoma (SLL).

15. The method of any one of claims 1-14, wherein the B cell malignancy or the B cell mediated disorder is relapsed or refractory following at least one therapeutic standard of care regimen.

16. The method of any one of claims 1-15, wherein the B cell malignancy or the B cell mediated disorder is relapsed or refractory following autologous stem cell transplant (ASCT).

17. The method of any one of claims 13-16, wherein the B cell malignancy is relapsed and/or refractory NHL.

18. The method of any one of claims 13-16, wherein the B cell malignancy is relapsed and/or refractory CLL.

19. The method of any one of claims 1-12, wherein the B cell mediated disorder is an autoimmune condition.

20. The method of claim 19, wherein the autoimmune condition is selected from the group consisting of: systemic lupus erythematosus (SLE), extrarenal systemic lupus erythematosus (ERL), lupus nephritis (LN), CNS lupus, vasculitis, granulomatosis vasculitis, polyangiitis & microscopic polyangiitis vasculitis, anti-neutrophilic cytoplasmic autoantibody (ANCA) associated vasculitis (AAV), granulomatous polyangiitis, microscopic polyangiitis, multiple sclerosis (relapsing and/or progressive), pemphigus vulgaris, autoimmune blistering skin diseases, membranous nephropathy (MN), anti-NMDA receptor neuropathy, neuromyelitis optica, idiopathic thrombocytopenia purpura, autoimmune hepatitis, type 1 diabetes mellitus, rheumatoid arthritis, juvenile rheumatoid arthritis, chronic inflammatory demyelinating polyneuropathy, polymyositis/dermatomyositis, stiff persons disease, anti-NMDA receptor encephalitis, anti-synthetase autoimmune syndromes, anti-phospholipid antibody syndrome, Sjogren's syndrome, cryoglobulinemia, focal segmental glomerulosclerosis, rapidly progressive glomerulopathy, autoimmune hemolytic anemia, amyloidosis, scleroderma, idiopathic inflammatory myositis, and immune-mediated necrotizing myopathy.

21. The method of claim 19 or claim 20, wherein the subject has:
a moderate to severe autoimmune condition;
a relapsed or refractory autoimmune condition; and/or
the presence of autoantibodies.

22. The method of any one of claims 1-21, wherein the subject is diagnosed to have systemic lupus erythematosus (SLE), extrarenal systemic lupus erythematosus (ERL), lupus nephritis (LN), ANCA-associated vasculitis (AAV), or membranous nephropathy (MN).

23. The method of any one of claims 1-22, wherein the subject is 18-85 years of age.

24. The method of any one of claims 1-23, wherein the method further comprises:
administering to the subject a lymphodepleting chemotherapy regimen,
wherein the step of administering the lymphodepleting chemotherapy regimen occurs prior to the step of administering the composition.

25. The method of claim 24, wherein the composition is administered 2-7 days after completion of the lymphodepleting chemotherapy regimen.

26. The method of claim 24 or claim 25, wherein the composition is administered 3 days after completion of the lymphodepleting chemotherapy regimen.

27. The method of any one of claims 24-26, wherein the lymphodepleting chemotherapy is or comprises cyclophosphamide and fludarabine.

28. The method of any one of claims 1-27, further comprising:
collecting a sample from the subject after administration of the composition and determining the presence of cellular and/or humoral immune response directed towards one or more CD19-specific CAR peptides.

29. The method of claim 28, wherein the method further comprises modulating a subsequent administration of the composition, a post-treatment therapy, and/or one or more additional anti-cancer therapies based on the presence of cellular and/or humoral immune response.

30. The method of any one of claims 1-29, further comprising:
(i) obtaining a biological sample from the subject; and
(ii) performing an analysis of the biological sample to determine,
a CD19 expression status,
a CD20 expression status,
a CD22 expression status, or
a combination thereof.

31. The method of any one of claims 1-30, further comprising:
(i) obtaining a biological sample from the subject; and
(ii) performing an analysis of the biological sample to determine:
a donor-specific antibody evaluation status,
a soluble marker of inflammation status,
a soluble marker of B cell activation status,
a serum immunoglobulin status,
a proteinuria level status,
a serum creatinine status,
an estimated glomerular filtration status,
an autoantibody status, or
a combination thereof.

32. An article of manufacture comprising a composition of a cell therapy, or one of a plurality of compositions of a cell therapy, comprising T cells expressing an anti-CD 19 chimeric antigen receptor (CAR), and instructions for administering the cell therapy, wherein the instructions specify administering the T cell composition according to the methods of any of claims 1-387.

33. A pharmaceutical composition comprising a population of engineered hypoimmunogenic T cells comprising:
(a) reduced expression of beta-2-microglobulin (B2M), Class II Major Histocompatibility Complex Transactivator (CIITA), and T cell receptor alpha constant (TRAC) relative to a control or wild-type T cells,
(b) increased expression of CD47 encoded by a first exogenous polynucleotide relative to the control or wild-type T cells, and
(c) expression of a CD19-specific chimeric antigen receptor (CAR) encoded by a second exogenous polynucleotide.

34. The pharmaceutical composition of claim 33, wherein the pharmaceutical composition comprises partially edited CAR+ cells, wherein the partially edited CAR+ cells comprise one or more modifications that:
(a) inactivate or disrupt one or more alleles of: (i) one or more major histocompatibility complex (MHC) class I molecules and/or one or more molecules that regulate expression of the one or more MHC class I molecules, (ii) one or more MHC class II molecules and/or one or more molecules that regulate expression of the one or more MHC class II molecules, and/or (iii) one or more T-cell receptor (TCR) molecules and/or one or more molecules that regulate expression of the one or more TCR molecules, or
(b) increase expression of CD47 encoded by a first exogenous polynucleotide, wherein the increased expression of (b) is relative to a control or wild-type T cell that does not comprise the modification, and express a CD19-specific chimeric antigen receptor (CAR) encoded by a second exogenous polynucleotide.
